(19) United States
(12) Patent Application Publication
(10) Pub. No.: US 2011/0046127 A1
(43) Pub. Date: Feb. 24, 2011

(54) Imidazopyridazines for Use as Protein Kinase Inhibitors

(76) Inventors: Paolo Pevarello, Bresso(MI) (IT); Ana Maria Garcia Collazo, Madrid (ES); Antonio Rodriguez Hergueta, Madrid (ES); Carl-Gustaf Pierre Saluste, Madrid (ES); Francisco Javier Ramos Lima, Madrid (ES); Esther Gonzalez Cantalapiedra, Madrid (ES); Julen Oyarzabal Santamarina, Madrid (ES)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/741,765

(22) PCT Filed: Nov. 10, 2008
(86) PCT No.: PCT/GB2008/003744

§ 371 Date: Jul. 21, 2010

(30) Foreign Application Priority Data
Nov. 8, 2007 (EP) 07380308.2
Jun. 12, 2008 (GB) 0810792.2

(51) Int. Cl. A61K 31/5377; C07D 487/04; A61K 31/5025; C07D 413/14; A61P 35/00

(52) U.S. Cl. 514/230.8; 544/236; 514/248; 544/117

(57) ABSTRACT

There is provided compounds of formula (I): wherein Z, M, R¹, X, R³, R⁴ and R⁵ have meanings given in the description, an pharmaceutically-acceptable esters, amides, solvates or salts thereof, which compounds are useful in the treatment of diseases in which inhibition of a protein kinase (e.g. a PIM family kinase or PI3-K) is desired and/or required, an particularly in the treatment of cancer.

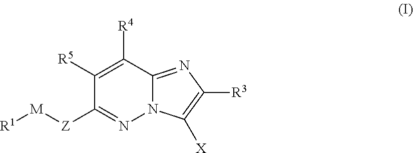

### FIELD OF THE INVENTION

This invention relates to novel pharmaceutically-useful compounds, which compounds are useful as inhibitors of protein kinases (such as the PIM family kinases). The compounds are of potential utility in the treatment of diseases such as cancer. The invention also relates to the use of such compounds as medicaments, to pharmaceutical compositions containing them, and to synthetic routes for their production.

### BACKGROUND OF THE INVENTION

The malfunctioning of protein kinases (PKs) is the hallmark of numerous diseases. A large share of the oncogenes and proto-oncogenes involved in human cancers code for PKs. The enhanced activities of PKs are also implicated in many non-malignant diseases, such as benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis. PKs are also implicated in inflammatory conditions and in the multiplication of viruses and parasites. PKs may also play a major role in the pathogenesis and development of neurodegenerative disorders.

For a general reference to PKs malfunctioning or disregulation see, for instance, Current Opinion in Chemical Biology 1999, 3, 459-465.

PIM-1 is the protooncogene activated by murine leucemia virus (Provirus Integration site for Moloney murine leucemia virus—MoMuLV) that induces T-cell lymphoma [Cuypers, H. T., et. al. Cell, 1984, 37, 141-150].

The expression of the protooncogene produces a non-transmembrane serine/threonine kinase of 313 residues, including a kinase domain consisting of 253 amino acid residues. Two isoforms are known through alternative initiation (p44 and p33) [Saris, C. J. M. et al. EMBO J. 1991, 10, 655-664].

PIM-1, PIM-2 and PIM-3 phosphorylate protein substrates that are important in cancer neogenesis and progression. For example, PIM-1 phosphorylates inter alia p21, Bad, c-myb, Cdc 25A and elF4B (see e.g. Quian, K. C. et al, J. Biol. Chem. 2005, 280(7), 6130-6137, and references cited therein).

Two PIM-1 homologs have been described [Baytel, D. Biochem. Biophys. Acta 1998, 1442, 274-285; Feldman, J. et al. J. Biol. Chem. 1998, 273, 16535.16543]. PIM-2 and PIM-3 are respectively 58% and 69% identical to PIM-1 at the amino acid level. PIM-1 is mainly expressed in thymus, testis, and cells of the hematopoietic system [Mikkers, H.; Nawijn, M.; Allen, J.; Brouwers, C.; Verhoeven, E.; Jonkers, J.; Bems, Mol. Cell. Biol. 2004, 24, 6104; Bachmann, M.; Moroy, T. Int. J. Biochem. Cell Biol. 2005, 37, 726-730. 6115]. PIM-1 expression is directly induced by STAT (Signal Transducers and Activators of Transcription) transcription factors, and PIM-1 expression is induced by many cytokine signalling pathways such as interleukins (IL), granulocyte-macrophage colony stimulating factor (GM-CSF), α- and γ-interferon, erythropoietin, and prolactin [Wang, Z et al. J. Vet. Sci. 2001, 2, 167-179].

PIM-1 has been implicated in lymphoma development. Induced expression of PIM-1 and the protooncogene c-myc synergise to increase the incidence of lymphomagenesis [Breuer, M. et al. Nature 1989, 340, 61-63; van Lohuizen M. et al. Cell, 1991, 65, 737-752]. PIM-1 functions in cytokine signalling pathways and has been shown to play a role in T cell development [Schmidt, T. et al. EMBO J. 1998, 17, 5349-5359; Jacobs, H. et al. JEM 1999, 190, 1059-1068]. Signalling through gp130, a subunit common to receptors of the IL-6 cytokine family, activates the transcription factor STAT3 and can lead to the proliferation of hematopioetic cells [Hirano, T. et al. Oncogene 2000, 19, 2548-2556]. A kinase-active PIM-1 appears to be essential for the gp130-mediated STAT3 proliferation signal. In cooperation with the c-myc PIM-1 can promote STAT3-mediated cell cycle progression and antiapoptosis [Shirogane, T. et sl., immunity, 1999, 11, 709-719]. PIM-1 also appears to be necessary for IL-3-stimulated growth in bone marrow-derived mast cells [Domen, J. et al., Blood, 1993, 82, 1445-1452] and survival of FDCP1 cells after IL-3 withdrawal [Lilly, M. et al., Oncogene, 1999, 18, 4022-4031].

Additionally, control of cell proliferation and survival by PIM-1 may be effected by means of its phosphorylation of the well-established cell cycle regulators cdc25 [Mochizuki, T. et al., J. Biol. Chem. 1999, 274, 18659-18666] and/or p21(Cip1/WAF1) [Wang Z. et al. Biochim. Biophys. Acta 2002, 1593, 45-55] or phosphorylation of heterochromatin protein 1, a molecule involved in chromatin structure and transcriptional regulation [Koike, N. et al, FEBS Lett. 2000, 467, 17-21].

Mice deficient for all three PIM genes showed an impaired response to hematopoietic growth factors and demonstrated that PIM proteins are required for efficient proliferation of peripheral T lymphocyes. In particular, it was shown that PIM function is required for efficient cell cycle induction of T cells in response to synergistic T-cell receptor and IL-2 signalling. A large number of interaction partners and substrates of PIM-1 have been identified, suggesting a pivotal role for PIM-1 in cell cycle control, proliferation, as well as in cell survival.

The oncogenic potential of this kinase has been first demonstrated in E μ PIM-1 transgenic mice in which PIM-1 over-expression is targeted to the B-cell lineage which leads to formation of B-cell tumors [van Lohuizen, M. et al.; Cell 1989, 56, 673-682. Subsequently PIM-1 has been reported to be over-expressed in a number of prostate cancers, erythroleukemias, and several other types of human leukemias [Roh, M. et al.; Cancer Res. 2003, 63, 8079-8084; Valdman, A. et al; Prostate 2004, 60, 367-371;

For example, chromosomal translocation of PIM-1 leads to overexpression of PIM-1 in diffuse large cell lymphoma. [Akasaka, H. et al.; Cancer Res. 2000, 60, 2335-2341]. Furthermore, a number of missense mutations in PIM-1 have been reported in lymphomas of the nervous system and AIDS-induced non-Hodgkins' lymphomas that probably affect PIM-1 kinase activity or stability [Pasqualucci, L. et al, Nature 2001, 412, 341-346; Montesinos-Rongen, M. et al., Blood 2004, 103, 1869-1875; Gaidano, G. et al., Blood 2003, 102, 1833-184]. Thus, the strong linkage between reported overexpression data and the occurrence of PIM-1 mutations in cancer suggests a dominant role of PIM-1 in tumorigenesis.

Several other protein kinases have been described in the literature, in which the activity and/or elevated activity of such protein kinases have been implicated in diseases such as cancer, in a similar manner to PIM-1, PIM-2 and PIM-3. Such protein kinases include PI3-K, CDK-2, SRC and GSK-3.

There is a constant need to provide alternative and/or more efficacious inhibitors of protein kinases, and particularly inhibitors of CDK-2, SRC, GSK-3, PI3-K, PIM-1, PIM-2 and/or PIM-3. Such modulators are expected to offer alternative and/or improved approaches for the management of medical conditions associated with activity and/or elevated activity of CDK-2, SRC, GSK-3, PI3-K, PIM-1, PIM-2 and/or PIM-3 protein kinases.

The listing or discussion of a prior-published document in this specification should not necessarily be taken as an acknowledgement that the document is part of the state of the art or is common general knowledge.

International patent applications WO 2007/064797 and WO 2004/058769 disclose various compounds that may be useful in the treatment of cancer. However, there is no mention in these documents of imidazopyridazines.

US patent application US 2007/0093490 and US 2007/0049591 both disclose inter alia imidazo[1,2-b]pyridazines that may be useful as kinase inhibitors. However, such imidazo[1,2-b]pyridazines are necessarily directly substituted in the 3-position with an aromatic group.

International patent application WO 2005/066177 discloses various imidazopyridazines that may be useful for controlling parasites. However, there is no mention in this document that the compounds disclosed therein may be useful as protein kinase inhibitors and further this document only discloses imidazo[1,2-b]pyridazines that are substituted in the 2-position with an aromatic group.

International patent application WO 2007/0136736 discloses various compounds that may be useful as Lck inhibitors, and therefore useful in the treatment of diseases such as rejection reaction in organ transplantation, autoimmune diseases, asthma and atopic dermatitis. However, there is no mention in this document that the compounds disclosed therein may be useful as inhibitors of cancer-related protein kinases.

U.S. Pat. No. 1,135,893 discloses various imidazopyridazines that may be useful anti-inflammatory compounds. However, this document does not disclose compounds that are substituted on the pyridazine ring of the bicyclic ring system with an aromatic group (attached via a linker or otherwise). International patent application WO 2007/038314 discloses various compounds that may be useful in the treatment of inter alia inflammatory or immune diseases. However, there is no specific disclosure in this document of imidazo[1,2-b]pyridazines that are substituted in the 6-position with an aromatic group and/or substituted in the 2-position.

International patent application WO 89/01333 discloses various imidazopyridazines for use in the treatment of inter alfa anxiety syndrome. However, this document only discloses compounds that are substituted in the 2-position with an aromatic, or other cyclic, group (attached via a linker or otherwise). International patent application WO 2007/110437 discloses imidazopyridazines that may also be useful in the treatment of inter alia anxiety. However, this document primarily relates to imidazopyridazines substituted in the 2-position with an aromatic group.

US patent application US 2001/0007867 discloses compounds that may be useful as antagonists of corticotropin releasing factor. However, there is no disclosure in this document of imidazopyridazines that are substituted at the 6-position with an aromatic group (attached via a linker or otherwise). Further still, such imidazopyridazines may only be substituted at the 3-position with an aromatic group.

German patent application DE 19912636 discloses various polyaza-bicyclic heterocyclic compounds that may be useful as inhibitors of adenosine monophosphate deaminase or adenosine deaminase. However, there is no disclosure in that document of imidazopyridazines.

European patent application EP 0 490 587 discloses inter alfa imidazopyridazines, which may be useful as angiotensin II antagonists, and therefore of potential use in the treatment of e.g. hypertension. However, this document only relates to 6,5-bicyclic compounds that are substituted on the 5-membered ring with a biphenyl moiety. Further, there is no mention that the compounds disclosed therein may be useful as protein kinase inhibitors.

International patent applications WO 2008/079880, WO 2008/058126, WO 2008/052734, WO 052733, WO 2008/008539 and WO 2007/095588 and European patent applications EP 0 562 440, EP 1 466 527 and EP 1 123 936 all disclose various 6,5-bicycles. However, there is no specific disclosure in any of these applications of imidazopyridazines substituted at the 3-position and 6-position with certain linker groups.

Various imidazopyridazines have been disclosed for use as medicaments in inter alia Australian Journal of Chemistry (1994), 47(11), 1989-99; Journal of Heterocyclic Chemistry (1998), 35(5), 1205-1217; Australian Journal of Chemistry (1992), 45(8), 1281-300; and international patent applications WO 2006/128692 and WO 2006/128693. However, none of these documents mention that the compounds disclosed therein may be useful as inhibitors of protein kinases, and therefore of use in the treatment of diseases such as cancer. Various other imidazopyridazines have also been disclosed in the CAS registry database, but to which compounds no use has apparently been ascribed.

### DISCLOSURE OF THE INVENTION

According to the invention, there is provided a compound of formula I,

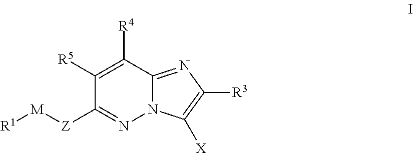

wherein:
Z represents a direct bond, —(CH₂)ₙ—O—, —(CH₂)ₙ—S—, —(CH₂)ₙ—N(R^{a})—, —(CH₂)ₙ—C(O)—, —(CH₂)ₙ—C(O)O—, —(CH₂)ₙ—S(O)—, —(CH₂)ₙ—SO₂—, —(CH₂)ₙ—N(R^{a})—SO₂—, —(CH₂)ₙ—SO₂—N(R^{a})—, —(CH₂)ₙ—N(R^{a})—CO—, —(CH₂)ₙ—NH—CO—NH— or —(CH₂)ₙ—CO—N(R^{a})—;
n represents, on each occasion when mentioned above, 0, 1 or 2;
M represents a direct bond or C₁₋₆ alkylene optionally substituted by one or more substituents selected from halo, —OR^{b}, —SR^{b} and —N(R^{b})₂;
R¹ represents aryl or heteroaryl, both of which are optionally substituted by one or more substituents selected from B¹ (e.g. R¹ represents aryl, monocyclic heteroaryl or bicyclic heteroaryl, all of which are optionally substituted by one or more substituents selected from B¹, B² and B³, respectively);
X represents C₃₋₆ cycloalkyl, heterocycloalkyl (which latter two groups are optionally substituted by one or more substituents selected from B⁴ and B⁵, respectively) or -G-R²;
G represents —(CH₂)ₘ—O—, —(CH₂)ₘ—S—, —(CH₂)ₘ—N(R^{d})—, —(CH₂)ₘ—C(O)—, —(CH₂)ₘ—C(O)O—, —(CH₂)ₘ—S(O)—, —(CH₂)ₘ—SO₂—, —(CH₂)ₘ—N(R^{d})—SO₂—, —(CH₂)ₘ—SO₂—N(R^{d})—, —(CH₂)ₘ—N(R^{d})—CO—, —(CH₂)ₘ—CO—N(R^{d})—, —(CH₂)ₘ—NH—CO—NH— or C₁₋₈ alkylene optionally substituted by one or more substituents selected from A¹;
m represents, on each occasion when used herein, 0, 1 or 2;
R² represents hydrogen, C₁₋₈ alkyl (optionally substituted by one or more substituents selected from A²) or -T-Q;
T represents a direct bond or a C₁₋₃ alkylene linker group optionally substituted by one or more substituents selected from A³;
Q represents C₃₋₆ cycloalkyl, heterocycloalkyl (which latter two groups are optionally substituted by one or more substituents selected from B⁶ and B⁷, respectively), aryl or heteroaryl (which latter two groups are optionally substituted by one or more substituents selected from B⁸ and B⁹, respectively);
A¹, A² and A³ independently represent halo, —OR^{e}, —S—C₁₋₄ alkyl, —N(R^{e})₂, —C(O)₂R^{e}, —C(O)N(R^{e})₂, —N(R^{e})—C(O)—R^{e}, —C(O)R^{e}, —CN, —SO₂N(R^{e})₂, phenyl (optionally substituted by one or more halo or —OR^{e} substituents) and/or C₁₋₄ alkyl (optionally substituted by one or more substituents selected from halo);

B¹, B², B³, B⁴, B⁵, B⁶, B⁷, B⁸ and B⁹ independently represent, on each occasion when used herein, halo, —OR^{e}, —C(O)₂R^{e}, —C(O)R^{e}, —C(O)N(R^{e})₂, —N(R^{e})—C(O)—R^{e}, —CN, —S(O)₂R^{e}, —S(O)₂N(R^{e})₂, —N(R^{e})₂ and/or C₁₋₄ alkyl (optionally substituted by one or more substituents selected from halo, —OR^{e} and —C(O)₂R^{e}); or, B⁴, B⁵, B⁶ and B⁷ may alternatively and independently represent ═O;

R³, R⁴ and R⁵ independently represent hydrogen, halo, —R^{j}, —OR^{f}, —SR^{f}, cyano or —N(R^{f})₂;
R^{a}, R^{b}, R^{d}, R^{e} and R^{f} independently represent, on each occasion when used herein, hydrogen and/or C₁₋₄ alkyl optionally substituted with one or more substituents selected from halo and —OR^{h}; or
any two R^{e} groups, when attached to the same nitrogen atom may be linked together to form (together with the requisite nitrogen atom to which those R^{e} groups are necessarily attached) a 3- to 8-membered (e.g. a 5- or 6-membered) ring optionally containing a further one or two heteroatoms, which ring optionally contains one to three unsaturations (e.g. triple or, preferably, double bonds) and is optionally substituted by one or more substituents selected from ═O and C₁₋₃ alkyl (optionally substituted by one or more fluoro atoms);
R^{j} represents, on each occasion when used herein, hydrogen, aryl, heteroaryl, C₃₋₆ cycloalkyl, heterocycloalkyl and/or C₁₋₄ alkyl, which latter five groups are optionally substituted with one or more substituents selected from halo, C₁₋₄ alkyl and —OR^{h};
R^{h} represents, on each occasion when used herein, hydrogen or C₁₋₄ alkyl optionally substituted by one or more halo atoms;
or a pharmaceutically acceptable ester, amide, solvate or salt thereof,
provided that when:
(I) R⁴ and R⁵ represent hydrogen, Z represents —S—, R¹ represents unsubstituted phenyl, X represents -G-R²:
(i) M represents a direct bond, then when:
   (A) R³ represents hydrogen and G represents —C(O)—;
   (B) R³ represents —CH₃ and G represents —(CH₂)—NH—C(O)—, then R² does not represent unsubstituted phenyl;
   (ii) M represents —CH₂—, R³ represents tert-butyl and G represents —O—, then R² does not represent —CH₃;
      (II) R⁴ and R⁵ represent hydrogen, Z and M represent direct bonds, R¹ represents (3,5-dimethyl)pyrazol-1-yl, R³ represents —OCH₃, X represents -G-R² and G represents —SO₂, then R² does not represent unsubstituted 1,3,4-triazol-2-yl or 1,2,4-triazol-3-yl substituted at the 1-position with B⁹, in which B⁹ represents —C(O)N(R^{e})₂ and each R^{e} represents ethyl;
      (III) R⁴ and R⁵ represent hydrogen, Z represents —O—, R³ represents tert-butyl, X represents -G-R², G represents —O— and R² represents —CH₃, then:
(i) when M represents a direct bond, then R¹ does not represent 2-methoxyphenyl;
   (ii) when M represents —CH₂—, then R¹ does not represent unsubstituted phenyl;
      (IV) R⁴ and R⁵ represent hydrogen, R³ represents —CF₃, X represents -G-R², G represents —CH₂—:
(a) R² represents (4-n-propyl)pyrrolidin-2-one and M represents —CH₂—, then:
(i) when Z represents —N(H)—, R¹ does not represent (2,4-dimethoxy)phenyl;
(ii) when Z represents —O—, R¹ does not represent unsubstituted phenyl;
(b) R² represents (4-CH═CF₂)pyrrolidin-2-one (i.e. 4-(2,2-difluoroethenyl)pyrrolidin-2-one), and M and Z both represent direct bonds, then R¹ does not represent unsubstituted 3-pyridyl, 3-thienyl or phenyl;
   (V) Z and M represent direct bonds, R³ and R⁵ represent hydrogen, X represents -G-R², G represents —CH₂— and R² represents 4-morpholinyl, then:
(i) when R⁴ represents methyl, then R¹ does not represent 3-methoxyphenyl or unsubstituted phenyl; or
(ii) when R⁴ represents hydrogen, then R¹ does not represent 4-chlorophenyl or unsubstituted phenyl;
   (VI) Z and M represent direct bonds, R³ and R⁵ represent hydrogen, X represents -G-R², G represents —CH₂— substituted by A¹ in which A¹ represents —N(CH₃)₂, and R² represents hydrogen (so forming a —CH₂—N(CH₃)₂ group), then R¹ does not represent unsubstituted phenyl when R⁴ represents hydrogen or methyl;
   (VII) Z and M represent direct bonds, R³ and R⁵ represent hydrogen, R⁴ represents —CF₃, R¹ represents 4-trifluoromethylphenyl, X represents -G-R², G represents —C≡C— (i.e. ethynylene), then R² does not represent 2-(NH₂)-pyrimidin-5-yl, 5-(S(O)₂NH₂)-thien-2-yl or 6-(NH₂)-pyrid-3-yl;
   (VIII) Z and M represent direct bonds, R³, R⁴ and R⁵ represent hydrogen, X represents -G-R², G represents —CH₂— substituted by A¹ in which A¹ represents —C(O)₂R^{e}, R² represents unsubstituted phenyl, then:
(i) when R^{e} represents ethyl, then R¹ does not represent 4-chlorophenyl, 4-methoxyphenyl or unsubstituted phenyl;
(ii) when R^{e} represents hydrogen, then R¹ does not represent 4-chlorophenyl or 4-methoxyphenyl;
   (IX) Z and M represent direct bonds, R³, R⁴ and R⁵ represent hydrogen, X represents -G-R², G represents —CH₂—, then when:
(i) R¹ represents 3-trifluoromethylphenyl, then R² does not represent H, or ethyl;
(ii) R¹ represents unsubstituted 3-pyridyl, then R² does not represent H or methyl;
   (X) Z and M represent direct bonds, R³, R⁴ and R⁵ represent hydrogen, X represents -G-R², G represents —CH₂— and R² represents H, then R¹ does not represent unsubstituted phenyl,
   which compounds, esters, amides, solvates and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

By “pharmaceutically acceptable ester, amide, solvate or salt thereof”, we include salts of pharmaceutically acceptable esters or amides, and solvates of pharmaceutically acceptable esters, amides or salts. For instance, pharmaceutically acceptable esters and amides such as those defined herein may be mentioned, as well as pharmaceutically acceptable solvates or salts.

Pharmaceutically acceptable esters and amides of the compounds of the invention are also included within the scope of the invention. Pharmaceutically acceptable esters and amides of compounds of the invention may be formed from corresponding compounds that have an appropriate group, for example an acid group (e.g. when X represents -G-R², G represents —C(O)O— and R² represents H), converted to the appropriate ester (e.g. a corresponding compound in which R² is not hydrogen) or amide (e.g. a corresponding compound in which G represents —C(O)N(R^{d})—). For example, pharmaceutically acceptable esters (of carboxylic acids of compounds of the invention) that may be mentioned include optionally substituted C₁₋₆ alkyl, C₅₋₁₀ aryl and/or C₅₋₁₀ aryl-C₁₋₆ alkyl-esters. Pharmaceutically acceptable amides (of carboxylic acids of compounds of the invention) that may be mentioned include those of the formula —C(O)N(R^{z1})R^{z2}, in which R^{z1} and R^{z2} independently represent optionally substituted C₁₋₆ alkyl, C₅₋₁₀ aryl, or C₅₋₁₀ aryl-C₁₋₆ alkylene-. Preferably, C₁₋₆ alkyl groups that may be mentioned in the context of such pharmaceutically acceptable esters and amides are not cyclic, e.g. linear and/or branched.

Preferably, specific esters and amides of compounds of the invention that may be mentioned include esters and amides of compounds of the invention in which, when X represents -G-R², G represents —C(O)O— and R² represents H. Hence, such groups may represent —CO₂R^{x} (wherein R^{x} represents C₁₋₄ alkyl optionally substituted by one or more halo atoms or —OR^{h}) or —C(O)N(R^{h})₂, wherein, in each case, R^{h} is as hereinbefore defined.

Further compounds of the invention that may be mentioned include carbamate, carboxamido or ureido derivatives, e.g. such derivatives of existing amino functional groups.

For the purposes of this invention, therefore, prodrugs of compounds of the invention are also included within the scope of the invention.

The term “prodrug” of a relevant compound of the invention includes any compound that, following oral or parenteral administration, is metabolised in vivo to form that compound in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration.

Prodrugs of compounds of the invention may be prepared by modifying functional groups present on the compound in such a way that the modifications are cleaved, in vivo when such prodrug is administered to a mammalian subject. The modifications typically are achieved by synthesising the parent compound with a prodrug substituent. Prodrugs include compounds of the invention wherein a hydroxyl, amino, sulfhydryl, carboxy or carbonyl group in a compound of the invention is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elesevier, New York-Oxford (1985).

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise stated, the term C_{1-q} alkyl (where q is the upper limit of the range) defined herein may be straight-chain or, when there is a sufficient number of carbon atoms, be branched-chain, saturated or unsaturated (so forming, for example, an alkenyl or alkynyl group).

Unless otherwise stated, the term C_{1-q} alkylene (where q is the upper limit of the range) defined herein may be straight-chain or, when there is a sufficient number of carbon atoms, be saturated or unsaturated (so forming, for example, an alkenylene or alkynylene linker group). However, such C_{1-q} alkylene groups may not be branched.

C_{3-q} cycloalkyl groups (where q is the upper limit of the range) that may be mentioned may be monocyclic or bicyclic alkyl groups, which cycloalkyl groups may further be bridged (so forming, for example, fused ring systems such as three fused cycloalkyl groups). Such cycloalkyl groups may be saturated or unsaturated containing one or more double or triple bonds (forming for example a cycloalkenyl or cycloalkynyl group). Substituents may be attached at any point on the cycloalkyl group. Further, where there is a sufficient number (i.e. a minimum of four) such cycloalkyl groups may also be part cyclic.

The term “halo”, when used herein, includes fluoro, chloro, bromo and iodo.

Heterocycloalkyl groups that may be mentioned include non-aromatic monocyclic and bicyclic heterocycloalkyl groups in which at least one (e.g. one to four) of the atoms in the ring system is other than carbon (i.e. a heteroatom), and in which the total number of atoms in the ring system is between five and ten. Such heterocycloalkyl groups may also be bridged. Further, such heterocycloalkyl groups may be saturated or unsaturated containing one or more double and/or triple bonds, forming for example a C_{2-q} heterocycloalkenyl (where q is the upper limit of the range) or a C_{7-q} heterocycloalkynyl group. C_{2-q} heterocycloalkyl groups that may be mentioned include 7-azabicyclo[2.2.1]heptanyl, 6-azabicyclo[3.1.1]heptanyl, 6-azabicyclo[3.2.1]-octanyl, 8-azabicyclo-[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo-[3.2.1]octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form.

For the avoidance of doubt, the term “bicyclic” (e.g. when employed in the context of heterocycloalkyl groups) refers to groups in which the second ring of a two-ring system is formed between two adjacent atoms of the first ring. The term “bridged” (e.g. when employed in the context of cycloalkyl or heterocycloalkyl groups) refers to monocyclic or bicyclic groups in which two non-adjacent atoms are linked by either an alkylene or heteroalkylene chain (as appropriate).

Aryl groups that may be mentioned include C₆₋₁₂ (e.g. C₆₋₁₀) aryl groups. Such groups may be monocyclic, bicyclic or tricyclic and have between 6 and 12 (e.g. 6 and 10) ring carbon atoms, in which at least one ring is aromatic. C₆₋₁₀ aryl groups include phenyl, naphthyl and the like, such as 1,2,3,4-tetrahydronaphthyl. The point of attachment of aryl groups may be via any atom of the ring system. For example, when the aryl group is polycyclic the point of attachment may be via atom including an atom of a non-aromatic ring. However, when aryl groups are polycyclic (e.g. bicyclic or tricyclic), they are preferably linked to the rest of the molecule via an aromatic ring.

Unless otherwise specified, the term “heteroaryl” when used herein refers to an aromatic group containing one or more heteroatom(s) (e.g. one to four heteroatoms) preferably selected from N, O and S. Heteroaryl groups include those which have between 5 and 10 members and may be monocyclic, bicyclic or tricyclic, provided that at least one of the rings is aromatic (so forming, for example, a mono-, bi-, or tricyclic heteroaromatic group). When the heteroaryl group is polycyclic the point of attachment may be via atom including an atom of a non-aromatic ring. However, when heteroaryl groups are polycyclic (e.g. bicyclic or tricyclic), they are preferably linked to the rest of the molecule via an aromatic ring. Heteroaryl groups that may be mentioned include 3,4-dihydro-1H-isoquinolinyl, 1,3-dihydroisoindolyl, 1,3-dihydroisoindolyl (e.g. 3,4-dihydro-1H-isoquinolin-2-yl, 1,3-dihydroisoindol-2-yl, 1,3-dihydroisoindol-2-yl; i.e. heteroaryl groups that are linked via a non-aromatic ring), or, preferably, acridinyl, benzimidazolyl, benzodioxanyl, benzodioxepinyl, benzodioxolyl (including 1,3-benzodioxolyl), benzofuranyl, benzofurazanyl, benzothiadiazolyl (including 2,1,3-benzothiadiazolyl), benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form.

It may be specifically stated that the heteroaryl group is monocyclic or bicyclic. In the case where it is specified that the heteroaryl is bicyclic, then it may be consist of a five-, six- or seven-membered monocyclic ring (e.g. a monocyclic heteroaryl ring) fused with another a five-, six- or seven-membered ring (e.g. a monocyclic aryl or heteroaryl ring).

Heteroatoms that may be mentioned include phosphorus, silicon, boron and, preferably, oxygen, nitrogen and sulfur.

For the avoidance of doubt, in cases in which the identity of two or more substituents in a compound of the invention may be the same, the actual identities of the respective substituents are not in any way interdependent. For example, in the situation in which there is more than one A¹ substituent present, then those A¹ substituents may be the same or different. Further, in the case where there are two A¹ substituents present, in which one represents —OR^{e} and the other represents —C(O)₂R^{e}, then those R^{e} groups are not to be regarded as being interdependent. Similarly, in specific case when R³ represents —N(R^{f})₂, then those two R^{f} groups may be the same or different.

Linker groups, for example as defined by G (when X represents -G-R²) and Z are specified with hyphens (“-”s) at the respective ends, depicting the points of attachment with the rest of the compound of formula I. For the avoidance of doubt, in relation to the linker groups defined by G and Z, the first hyphen of the linking moiety is the point at which that moiety links to the requisite 5,5-bicycle of formula I (and the last hyphen depicts the linking point to —R², in the case of the G linker group, or -M-R¹, in the case of the Z linker group). For example, when Z represents —(CH₂)ₙ—N(R^{e})—, it is the —(CH₂)ₙ— portion that is attached to the 5,5-bicycle of formula I.

For the avoidance of doubt, when a term such as “R^{a} to R^{f}” is employed herein, this will be understood by the skilled person to mean R^{a}, R^{b}, R^{d}, R^{e} and R^{f}, inclusively. Likewise, a term such as “B¹ to B⁹” when employed herein, will be understood by the skilled person to mean B¹, B², B³, B⁴, B⁵, B⁶, B⁷, B⁸ and B⁹, inclusively.

The skilled person will appreciate that in certain preferred embodiments of the compounds of the invention, some or all of the provisos (I) to (X) above will become redundant.

Compounds of the invention that may be mentioned include those in which:

R⁴ represents hydrogen, halo, —R^{f}, —OR^{f}, —SR^{f} or cyano;
R^{j} represents R^{f} as defined herein;
R³, R⁴ and R⁵ independently represent hydrogen, halo, —R^{f}, —OR^{f}, —SR^{f}, cyano or —N(R^{f})₂;
when R^{j} represents aryl, heteroaryl, C₃₋₆ cycloalkyl or heterocycloalkyl, then such groups are optionally substituted by one or more substituents selected from —OR^{h} preferably, halo (e.g. fluoro) and C₁₋₄ alkyl (e.g. C₁₋₂ alkyl, such as methyl);
when R^{j} represents C₁₋₄ alkyl, then it is optionally substituted with one or more substituents selected from halo and —OR^{h} (preferably halo, e.g. fluoro);
B¹, B², B³, B⁴, B⁵, B⁶, B⁷, B⁸ and B⁹ independently represent, on each occasion when used herein, halo, —OR^{e}, —C(O)₂R^{e}, —C(O)R^{e}, —C(O)N(R^{e})₂, —CN, —S(O)₂R^{e}, —S(O)₂N(R^{e})₂, —N(R^{e})₂ and/or C₁₋₄ alkyl (optionally substituted by one or more substituents selected from halo, —OR^{e} and —C(O)₂R^{e}); or,
B⁴, B⁵, B⁶ and B⁷ may alternatively and independently represent ═O.

Further preferred compounds of the invention that may be mentioned include those in which:

any two R^{e} groups are linked together, they a 5- or 6-membered ring optionally containing a further two or, preferably, one heteroatom (selected from oxygen and, preferably, nitrogen), which ring optionally contains a double bond, and is optionally substituted by one or more substituents selected from ═O and C₁₋₃ alkyl (e.g. methyl), so forming for example a morpholinyl, piperidinyl or, preferably, a piperazinyl (e.g. 4-methyl-piperazin-1-yl) or pyrazolyl (e.g. a 3-methyl-5-oxo-4,5-dihydropyrazol-1-yl);
more preferably, any two R^{e} groups are not linked together.

Particularly preferred compounds of the invention include those in which:

X represents -G-R²;
G represents —(CH₂)ₘ—O—, —(CH₂)ₘ—S—, —(CH₂)ₘ—N(R^{d})—, —(CH₂)ₘ—C(O)—, —(CH₂)ₘ—C(O)O—, —(CH₂)ₘ—S(O)—, —(CH₂)ₘ—SO₂—, —(CH₂)ₘ—N(R^{d})—SO₂—, —(CH₂)ₘ—SO₂—N(R^{d})—, —(CH₂)ₘ—N(R^{d})—CO—, —(CH₂)ₘ—CO—N(R^{d})— or —(CH₂)ₘ—NH—CO—NH—;
R² represents hydrogen, C₁₋₈ alkyl (optionally substituted by one or more substituents selected from A²) or, most preferably, -T-Q;
where it is stated herein that C_{1-q} alkyl groups (where q is the upper limit) are optionally substituted by one or more halo atoms, then those halo atoms are preferably fluoro.

Further compounds of the invention that may be mentioned include those in which:

X represents -G-R²; or
X represents cycloalkyl (e.g. C₃₋₆ cycloalkyl) or heterocycloalkyl (which latter two groups are optionally substituted by one or more substituents selected from B⁴ and B⁵, respectively);
both of Z and M do not (and preferably Z does not) represent a direct bond (i.e. at least one of Z and M (e.g. Z) represent a linker group other than a direct bond);
Z represents a direct bond, —(CH₂)ₙ—O—, —(CH₂)ₙ—S—, —(CH₂)ₙ—N(R^{a})—, —(CH₂)ₙ—S(O)—, —(CH₂)ₙ—SO₂—, —(CH₂)ₙ—N(R^{a})—SO₂—, —(CH₂)ₙ—SO₂—N(R^{a})—, —(CH₂)ₙ—N(R^{a})—CO—, —(CH₂)ₙ—NH—CO—NH— or —(CH₂)ₙ—CO—N(R^{a})—;
Z represents —(CH₂)ₙ—C(O)—, —(CH₂)ₙ—C(O)O— or, preferably, —(CH₂)ₙ—O—, —(CH₂)ₙ—S—, —(CH₂)ₙ—N(R^{a})—, —(CH₂)ₙ—S(O)—, —(CH₂)ₙ—SO₂—, —(CH₂)ₙ—N(R^{a})—SO₂—, —(CH₂)ₙ—SO₂—N(R^{a})—, —(CH₂)ₙ—N(R^{a})—CO—, —(CH₂)ₙ—NH—CO—NH— or —(CH₂)ₙ—CO—N(R^{a})—;
when Z represents a direct bond, then M preferably represents C₁₋₈ alkylene optionally substituted by one or more substituents selected from halo, —OR^{b}, —SR^{b} and —N(R^{b})₂;
when G represents —CH₂—, R² represents -T-Q, T represents a direct bond, then Q preferably represents C₃₋₆ cycloalkyl, aryl or heteroaryl, all of which are optionally substituted as hereinbefore defined;
when G represents optionally substituted C₁₋₈ alkylene, then R² preferably represents -T-Q;
for instance, when G represents C₁₋₈ alkylene (e.g. —CH₂—), then when such a group is substituted by A¹, then A¹ preferably represents —N(R^{e})₂ or, more preferably, halo, —OR^{e}, —S—C₁₋₄ alkyl, —C(O)N(R^{e})₂, —N(R^{e})—C(O)—R^{e}, —C(O)R^{e}, —CN, —SO₂N(R^{e})₂, phenyl (optionally substituted by one or more halo or —OR′ substituents) and/or C₁₋₄ alkyl (optionally substituted by one or more halo substituents);
when G represents unsaturated C₁₋₈ alkylene (e.g. —C≡C—), R² represents -T-Q, T represents a direct bond, then Q preferably represents C₃₋₆ cycloalkyl, heterocycloalkyl or aryl, all of which are optionally substituted as hereinbefore defined.

Further compounds of the invention that may be mentioned include those in which:

when X represents -G-R², then G represents —(CH₂)ₘ—O—, —(CH₂)ₘ—S—, —(CH₂)ₘ—N(R^{d})—, —(CH₂)ₘ—C(O)—, —(CH₂)ₘ—C(O)O—, —(CH₂)ₘ—S(O)—, —(CH₂)ₘ—SO₂—, —(CH₂)ₘ—SO₂—N(R^{d})—, —(CH₂)ₘ—N(R^{d})—CO—, —(CH₂)ₘ—CO—N(R^{d})— or C₁₋₈ alkylene optionally substituted by one or more substituents selected from A¹;
Z represents a direct bond, —(CH₂)ₙ—O—, —(CH₂)ₙ—S—, —(CH₂)ₙ—S(O)—, —(CH₂)ₙ—SO₂—, —(CH₂)ₙ—N(R^{a})—SO₂—, —(CH₂)ₙ—SO₂—N(R^{a})—, —(CH₂)ₙ—N(R^{a})—CO—, —(CH₂)ₙ—NH—CO—NH— or —(CH₂)ₙ—CO—N(R^{a})—;
when Z represents —(CH₂)ₙ—N(R^{a}), then n represents 1 or 2;
R¹ represents optionally substituted aryl or, preferably, optionally substituted heteroaryl (especially optionally substituted bicyclic heteroaryl), in which the optional substituents are as defined herein.

Further compounds of the invention that may be mentioned include those in which:

Z represents a direct bond, —(CH₂)ₙ—O—, —(CH₂)ₙ—S—, —(CH₂)ₙ—N(R^{a})—, or, more preferably, —(CH₂)ₙ—S(O)—, —(CH₂)ₙ—SO₂—, —(CH₂)ₙ—N(R^{a})—SO₂—, —(CH₂)ₙ—SO₂—N(R^{a})—, —(CH₂)ₙ—N(R^{a})—CO—, —(CH₂)ₙ—NH—CO—NH— or —(CH₂)ₙ—CO—N(R^{a})—;
when Z represents —(CH₂)ₙ—O— or —(CH₂)ₙ—S—, then n preferably represents 1 or 2, M preferably represents C₂₋₈ alkylene optionally substituted as defined herein and/or when R² represents optionally substituted C₁₋₈ alkyl, then it preferably represents C₂₋₈ (e.g. C₂₋₄) alkyl optionally substituted as defined herein;
when Z represents —(CH₂), —N(R^{a})—, then n preferably represents 1 or 2 and/or M preferably represents a direct bond or C₂₋₈ alkylene optionally substituted as defined herein;

G represents —(CH₂)ₘ—N(R^{d})—, —(CH₂)ₘ—C(O)O—, —(CH₂)ₘ—S(O)—, —(CH₂)ₘ—N(R^{d})—SO₂—, —(CH₂)ₘ—SO₂—N(R^{d})—, —(CH₂)ₘ—CO—N(R^{d})— or —(CH₂)ₘ—NH—CO—NH—;

when G represents —(CH₂)ₘ—N(R^{d})—CO—, then m represents 2 or, preferably, 0;
when G represents optionally substituted C₁₋₈ alkylene, R² represents -T-Q and T represents a direct bond, then Q represents C₃₋₆ cycloalkyl or, more preferably, aryl or heteroaryl, all of which groups are optionally substituted as defined herein;
when G represents —(CH₂)ₘ—O—, —(CH₂)ₘ—S(O)₂— or —(CH₂)ₘ—C(O)—, then m preferably represents 1 or 2;
when G represents —S(O)₂—, R² represents -T-Q and T represents a direct bond, then Q represents C₃₋₆ cycloalkyl, heterocycloalkyl or, more preferably, aryl, all of which groups are optionally substituted as defined herein;
when G represents —C(O)— or —CH₂—N(R^{d})—CO—, R² represents -T-Q and T represents a direct bond, then Q preferably represents C₃₋₆ cycloalkyl, heterocycloalkyl or heteroaryl, all of which are optionally substituted as defined herein;
when G represents —O—, then R² preferably represents hydrogen or -T-Q.

Preferred aryl and heteroaryl groups that R¹ and Q may independently represent include optionally substituted 1,3-dihydroisoindolyl, 3,4-dihydro-1H-isoquinolinyl, 1,3-dihydroisoindolyl or, preferably, optionally substituted phenyl, naphthyl, pyrrolyl, furanyl, thienyl, imidazolyl, oxazolyl, isoxazolyl, thiazolyl, pyrazolyl, pyridyl, indazolyl, indolyl, indolinyl, isoindolinyl, quinolinyl, isoquinolinyl, quinolizinyl, benzoxazolyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl. Particularly preferred groups include optionally substituted 1,3-dihydroisoindolyl (e.g. 1,3-dihydroisoindol-2-yl), 3,4-dihydro-1H-isoquinolin-2-yl, 1,3-dihydroisoindol-2-yl, thiazolyl (e.g. 2-thiazolyl) or, more preferably, optionally substituted phenyl, pyridyl (e.g. 2-pyridyl, 3-pyridyl or 4-pyridyl), furanyl (e.g. 3-furanyl or, preferably, 2-furanyl), thienyl (e.g. 2-thienyl), imidazolyl (e.g. 1-imidazolyl), pyrazinyl (e.g. 2-pyrazinyl), pyrazolyl (e.g. 3- or, preferably, 4-pyrazolyl), pyrrolyl (e.g. 1-pyrrolyl or, preferably, 2-pyrrolyl) and indolyl (e.g. 5-indolyl or, preferably, 6-indolyl).

Preferred monocyclic heteroaryl groups that R¹ may represent include 5- or 6-membered rings, containing one to three (e.g. one or two) heteroatoms selected from sulfur, oxygen and nitrogen. Preferred bicyclic heteroaryl groups that R¹ may represent include 8- to 12-(e.g. 9- or 10-) membered rings containing one to four (e.g. one to three, or, preferably, one to two) heteroatoms selected from sulfur, oxygen and nitrogen. Further, bicyclic rings may consist of benzene rings fused with a monocyclic heteroaryl group (as hereinbefore defined), e.g. a 6- or, preferably 5-membered monocyclic heteroaryl group optionally containing two, or, preferably, one heteroatom selected from sulfur, oxygen and nitrogen.

Preferred heterocycloalkyl groups that Q may independently represent 4- to 8-membered (e.g. 5- or 6-membered) heterocycloalkyl groups, which groups preferably contain one or two heteroatoms (e.g. sulfur or, preferably, nitrogen and/or oxygen heteroatoms), so forming for example, an optionally substituted pyrrolidinyl, piperidinyl, morpholinyl or tetrahydropyranyl group (most preferably, Q, in this instance, represents morpholinyl, such as 4-morpholinyl).

Preferred C₃₋₆ cycloalkyl groups that Q may independently represent include optionally substituted cyclohexyl, cyclopentyl, cyclobutyl or cyclopropyl.

Preferred substituents on aryl, heteroaryl, C₁₋₈ alkyl, C₃₋₆ cycloalkyl or heterocycloalkyl groups that R¹, R² or Q (as appropriate) may represent include:

—C(O)—N(R^{z11})₂; or, preferably,
═O (e.g. in the case of cycloalkyl or heterocycloalkyl groups);

### —CN;

halo (e.g. fluoro, chloro or bromo);
C₁₋₄ alkyl, which alkyl group may be cyclic, part-cyclic, unsaturated or, preferably, linear or branched (e.g. C₁₋₄ alkyl (such as ethyl, n-propyl, isopropyl, t-butyl or, preferably, n-butyl or methyl), all of which are optionally substituted with one or more halo (e.g. fluoro) groups (so forming, for example, fluoromethyl, difluoromethyl or, preferably, trifluoromethyl);
aryl (e.g. phenyl), if appropriate (e.g. when the substitutent is on an alkyl group, thereby forming e.g. a benzyl group);

### —OR^{z1};

### —C(O)R^{z2};

### —C(O)OR^{z3};

### —N(R^{z4})R^{z5};

### —S(O)₂R^{z6};

### —S(O)₂N(R^{z7})R^{z8};

### —N(R^{z9})R^{z10};

### N(R^{z11})—C(O)—R^{z12}

wherein each R^{z1} to R^{z12} independently represent, on each occasion when used herein, H or C₁₋₄ alkyl (e.g. ethyl, n-propyl, t-butyl or, preferably, n-butyl, methyl or isopropyl) optionally substituted by one or more halo (e.g. fluoro) groups (so forming e.g. a trifluoromethyl group). Further, any two R^{z} groups (e.g. R^{z4} and R^{z5}), when attached to the same nitrogen heteroatom may also be linked together to form a ring such as one hereinbefore defined in respect of corresponding linkage of two R^{e} groups.

More preferred compounds of the invention include those in which:

Z represents a direct bond, —(CH₂)ₙ—O—, —(CH₂)ₙ—S—, —(CH₂)ₙ—N(R^{a})—, —(CH₂)ₙ—N(R^{a})—CO— or —(CH₂)ₙ—CO—N(R^{a})—;
n represents 0;
M represents a direct bond or C₁₋₃ (e.g. C₁₋₂) alkylene (e.g. —CH₂—CH₂— or —CH₂—), which alkylene group may be saturated (so forming, for example, an ethynylene linker group);
when R¹ represents aryl, then it preferably represents optionally substituted phenyl;
when R¹ represents monocyclic heteroaryl, then it preferably represents optionally substituted imidazolyl (e.g. 1-imidazolyl) or, R¹ more preferably, represents optionally substituted pyridyl (e.g. 2-pyridyl, 3-pyridyl or 4-pyridyl), furanyl (e.g. 3- or, preferably, 2-furanyl), thienyl (e.g. 2-thienyl), imidazolyl (e.g. 1-imidazolyl), pyrazinyl (e.g. 2-pyrazinyl), pyrazolyl (e.g. 4-pyrazolyl) or pyrrolyl (e.g. 1- or, preferably, 2-pyrrolyl);
when R¹ represents bicyclic heteroaryl, then it may represent 3,4-dihydro-1H-isoquinolinyl, 1,3-dihydroisoindolyl (e.g. 4-dihydro-1H-isoquinolin-2-yl, 1,3-dihydroisoindol-2-yl), but preferably represents indolyl (e.g. 5- or, preferably, 6-indolyl);
X represents optionally substituted (i.e. by B⁴) C₃₋₆ cycloalkyl (such as cyclohexyl, cyclopentyl, cyclobutyl or cyclopropyl), optionally substituted (i.e. by B⁵) heterocycloalkyl (such as piperidinyl, e.g. 1-piperidinyl, or morpholinyl, e.g. 4-morpholinyl) or -G-R²;
G represents —(CH₂)ₘ—O—, —(CH₂)ₘ—SO₂N(R^{d})—, —(CH₂)ₘ—N(R^{d})—SO₂—, —(CH₂)ₘ—SO₂— or, preferably, —(CH₂)ₘ—N(R^{d})—, —(CH₂)ₘ—C(O)—, —(CH₂)ₘ—C(O)O—, —(CH₂)ₘ—C(O)—N(R^{d})—, —(CH₂)ₘ—N(R^{d})—SO₂—, —(CH₂)ₘ—N(R^{d})—C(O)—, —(CH₂)ₘ—NH—C(O)—NH— or C₁₋₆ (e.g. C₁₋₄) alkylene (e.g. —C≡C—CH₂—CH₂— (i.e. but-1-ynylene), —C≡C—CH₂— (i.e. prop-1-ynylene), —C≡C— (i.e. ethynylene) or —CH₂—);
m represents 0 or 1;
when G represents —(CH₂)ₘ—N(R^{d})—, then m may represent 0 or 1;
when G represents —(CH₂)ₘ—C(O)—, —(CH₂)ₘ—C(O)O—, —(CH₂)ₘ—C(O)—N(R^{d})—, —(CH₂)ₘ—N(R^{d})—SO₂—, —(CH₂)ₘ—N(R^{d})—C(O)— or —(CH₂)ₘ—NH—C(O)—NH—, then m preferably represents 0;
R² represents hydrogen, optionally substituted (i.e. by A²) C₁₋₅ alkyl (e.g. pentyl, propyl (such as n-propyl or isopropyl), ethyl or methyl) or -T-Q;
T represents a direct bond or C₁₋₂ alkylene (e.g. —CH₂—);
Q represents optionally substituted (i.e. by B⁶) C₃₋₆ cycloalkyl (e.g. cyclohexyl, cyclopentyl, cyclobutyl or cyclopropyl), optionally substituted (i.e. by B⁷) heterocycloalkyl (such as morpholinyl, e.g. 4-morpholinyl, tetrahydropyranyl, e.g. tetrahydropyran-4-yl), optionally substituted (i.e. by B⁸) aryl (such as phenyl or naphthyl, e.g. 2-naphthyl), optionally substituted (i.e. by B⁹) heteroaryl (such as 1,3-dihydroisoindolyl, pyrazolyl, e.g. 3-pyrazolyl, thiazolyl, e.g. 2-thiazolyl, preferably, indolyl, e.g. 5-indolyl, furanyl, e.g. 2-furanyl, benzofuranyl, e.g. 2-benzofuranyl or pyridyl, e.g. 3-, 5- or, preferably, 2-pyridyl);
A¹ to A³ independently represent halo or, preferably, —OR^{e}, —N(R^{e})—C(O)—R^{e} and/or —N(R^{e})₂;
B¹ to B⁹ independently represent —N(R^{e})₂, —N(R^{e})C(O)R^{e}, preferably, —C(O)N(R^{e})₂, —S(O)₂N(R^{e})₂, more preferably, halo (e.g. fluoro or chloro), —OR^{e}, —C(O)₂R^{e}, —C(O)R^{e}, —CN, —S(O)₂R^{e} and/or C₁₋₃ (e.g. C₁₋₂) alkyl (e.g. —CH₃) optionally substituted by one or more substituents selected from —C(O)₂R^{e} (so forming, for example, a carboxymethyl group) and, preferably, halo (e.g. fluoro; so forming for example a trifluoromethyl group) and —OR^{e} (so forming, for example, a hydroxymethyl group);
R³, R⁴ and R⁵ independently represent hydrogen, halo (e.g. fluoro or chloro), R^{j} or —OR^{f};
R^{a}, R^{b}, R^{d}, R^{e} and R^{f} independently represent hydrogen or C₁₋₃ (e.g. C₁₋₂) alkyl (e.g. methyl or ethyl) optionally substituted by one or more halo (e.g. fluoro) atoms (so forming, for example, a trifluoromethyl group);
or any two R^{e} groups are linked together as defined herein;
R^{j} represents C₃₋₆ (e.g. C₄₋₅) cycloalkyl (e.g. cyclopentyl) or, preferably, hydrogen or C₁₋₃ (e.g. C₁₋₂) alkyl (e.g. methyl or ethyl) optionally substituted by one or more halo (e.g. fluoro) atoms (so forming, for example, a trifluoromethyl group);
R^{h} represents hydrogen or C₁₋₂ alkyl optionally substituted by one or more fluoro atoms.

Further preferred compounds of the invention include those in which when G represents:

—(CH₂)ₘC(O)—, then R² preferably represents -T-Q, in which T is a direct bond, and Q preferably represents heteroaryl or, preferably, heterocycloalkyl, optionally substituted as defined herein;
—(CH₂)ₘC(O)O—, then R² preferably represents hydrogen or optionally substituted C₁₋₄ alkyl;
—(CH₂)ₘ—CO—N(R^{d})—, then R² preferably represents optionally substituted C₁₋₄ alkyl or -T-Q;
—(CH₂)ₘ—CO—N(R^{d})— and R² represents -T-Q, in which T is a direct bond, then Q preferably represents optionally substituted C₃₋₆ cycloalkyl, optionally substituted heterocycloalkyl, optionally substituted aryl or optionally substituted heteroaryl;
—(CH₂)ₘ—CO—N(R^{d})— and R² represents -T-Q, in which T represents —CH₂, then Q preferably represents optionally substituted aryl;
—(CH₂)ₘ—N(R^{d})—, then R² preferably represents hydrogen, optionally substituted C₁₋₆ (e.g. C₁₋₄) alkyl or -T-Q;
—(CH₂)ₘ—N(R^{d})— and R² represents -T-Q, in which T is a direct bond, then Q preferably represents optionally substituted heterocycloalkyl, optionally substituted C₃₋₆ cycloalkyl, optionally substituted aryl or optionally substituted heteroaryl;
—(CH₂)ₘ—N(R^{d})— and R² represents -T-Q, in which T represents —CH₂—, then Q preferably represents optionally substituted C₃₋₆ cycloalkyl, optionally substituted aryl or optionally substituted heteroaryl;
—(CH₂)ₘ—N(R^{d})—CO—, then R² preferably represents optionally substituted C₁₋₄ alkyl or -T-Q, in which T is preferably a direct bond, and Q preferably represents optionally substituted aryl or optionally substituted heteroaryl;
—(CH₂)ₘ—NH—CO—NH, then R² preferably represents optionally substituted C₁₋₄ alkyl or -T-Q, in which T is preferably a direct bond, and Q preferably represents optionally substituted aryl;
—(CH₂)ₘ—N(R^{d})—SO₂—, then R² preferably represents optionally substituted C₁₋₄ alkyl or -T-Q, in which T is preferably a direct bond, and Q preferably represents optionally substituted aryl;
C₁₋₈ alkylene, then R² preferably represents -T-Q;
—CH₂—, then R² preferably represents -T-Q, in which T is a direct bond, and Q preferably represents optionally substituted aryl, or, more preferably, optionally substituted heterocycloalkyl (such as morpholinyl, e.g. 4-morpholinyl), optionally substituted C₃₋₆ cycloalkyl or optionally substituted heteroaryl;
—C≡C—, —C≡C—CH₂— or —C≡C—CH₂—CH₂—, then R² preferably represents -T-Q, in which T is a direct bond, and Q preferably represents optionally substituted aryl, wherein, in each case above, the optional substituents, as well as the definitions of R², Q, etc, are as defined herein (for example substituents A¹ to A³ or B¹ to B⁹ are as defined herein, as well as the definition of e.g. heterocycloalkyl, heteroaryl or aryl, when Q represents such a group).

Preferred compounds of the invention include those in which:

B¹, B² and B³ independently represent —S(O)₂R^{e}, —N(R^{e})₂ preferably, —S(O)₂N(R^{e})₂ or, more preferably, halo (e.g. chloro or fluoro), —OR^{e} and/or C₁₋₃ (e.g. C₁₋₂) alkyl (e.g. methyl) optionally substituted by one or more halo (e.g. fluoro) substituents (so forming, for example, a trifluoromethyl group);
B⁴ to B⁹ independently represent —N(R^{e})₂, —N(R^{e})C(O)R^{e}, preferably, —C(O)N(R^{e})₂ or, more preferably, halo (e.g. fluoro or chloro), —C(O)₂R^{e}, —C(O)R^{e}, —CN, —S(O)₂R^{e} and/or C₁₋₃ (e.g. C₁₋₂) alkyl (e.g. —CH₃) optionally substituted by one or more substituents selected from fluoro, —C(O)₂R^{e} and, preferably, —OR^{e} (so forming, for example, a carboxymethyl, a trifluoromethyl or, preferably, a hydroxymethyl group);
R^{a}, R^{b} and R^{d} independently represent hydrogen or C₁₋₃ (e.g. C₁₋₂) alkyl (e.g. methyl);
R^{e} and R^{f} independently represent hydrogen or C₁₋₂ alkyl (e.g. —CH₃ or —CH₂CH₃) optionally substituted by one or more fluoro atoms (so forming, for example, a trifluoromethyl group); or
any two R^{e} groups (e.g. when part of a —N(R^{e})₂ moiety may be linked together to form an optionally substituted 5- or 6-membered ring as defined herein;
R^{j} represents C₃₋₆ (e.g. C₄₋₅) cycloalkyl (e.g. cyclopentyl) or, preferably, hydrogen or C₁₋₂ alkyl (e.g. —CH₂CH₃ or, preferably, CH₃) optionally substituted by one or more fluoro atoms (so forming, for example, a trifluoromethyl group);
R³, R⁴ and R⁵ independently represent cyclopentyl or, preferably, hydrogen, fluoro, chloro, —CH₃ or —OCH₃.

More preferred compounds of the invention include those in which:

B¹, B² and B³ independently represent piperazin-1-yl (e.g. 4-methylpiperazin-1-yl), —S(O)₂CH₃, preferably, —S(O)₂NH₂ or, more preferably, chloro, fluoro, —OCH₃, —OH, —CF₃ and/or —CH₃;
G represents —CH₂—C(O)O—, —CH₂—C(O)N(H)—, —C(O)N(CH₃)— preferably, —CH₂NH—, —CH₂N(H)—C(O)—, —CH₂—O—, —S(O)₂—, —S(O)₂N(H)— or, more preferably, —C(O)—, —C(O)O—, —CO—NH—, —CH₂—NH—, —NH—, —N(CH₃)—, —NHSO₂—, —NH—CO—, —NH—CO—NH—, —CH₂—, —C≡C—, —C≡C—CH₂— or —C≡C—CH₂—CH₂—;
A¹ to A³ (e.g. A²) independently represent —OCH₃, —N(H)—C(O)CH₃ or —N(H)CH₃;
B⁵ represents —CH₃;
B⁶ represents —OH;
B⁸ represents piperazin-1-yl (e.g. 4-methylpiperazin-1-yl), pyrazol-1-yl (e.g. 4,5-dihydropyrazol-1-yl or, preferably, 3-methyl-5-oxo-4,5-dihydropyrazol-1-yl), —N(CH₃)₂, —N(H)CH₃, —N(H)C(O)CH₃, —CH₂COOH, —CF₃, preferably, —C(O)N(H)CH₃, —C(O)N(CH₃)₂ or, more preferably, —OCF₃, —OCH₃, —C(O)₂H, halo (e.g. fluoro or chloro), —SO₂CH₃, —CH₂OH, —CN, —C(O)CH₃, —C(O)OCH₂CH₃ or —OH;
B⁹ represents —CH₃.

Preferred compounds of the invention include those in which:

Z represents a direct bond, —(CH₂)ₙ—O— or, preferably, —(CH₂)ₙ—N(R^{a})—;
n represents 0;
R^{a} represents methyl or, preferably, hydrogen;
M represents a direct bond or, preferably, C₁₋₂ alkylene (e.g. —CH₂CH₂— or, preferably, —CH₂—);
—Z-M together represent a direct bond, C₁₋₃ alkylene (e.g. —CH₂CH₂—), preferably, —O—CH₂—, —O—, —N(H)—, —N(CH₃)—CH₂—, or, more preferably, —N(H)—CH₂—;
Z and M do not both represent a direct bond;
R¹ represents: a nine- or ten-membered bicyclic heteroaryl group (e.g. 1,3-dihydroisoindolyl, 3,4-dihydro-1H-isoquinolinyl, 1,3-dihydroisoindolyl and indolyl) optionally substituted by one or more B¹ substituents, but which groups are preferably unsubstituted; a five- or six-membered monocyclic heteroaryl group (in which there are/is two or one heteroatom(s) preferably selected from nitrogen and oxygen; so forming for example a furanyl, pyrrolyl, imidazolyl and pyridyl group, e.g. 2-furanyl, 3-furanyl, 1-pyrrolyl, 1-imidazolyl, 3-pyridyl and 4-pyridyl) optionally substituted by one or more (e.g. one or two) B¹ substitutents (but preferably unsubstituted); or, R¹ preferably represents phenyl optionally substituted by one or more (e.g. one or two) substitutents (preferably substituted in the para- and/or meta-position) selected from B¹;
B¹ represents —S(O)₂R^{e}, —N(R^{e})₂ preferably, C₁₋₄ (e.g. C₁₋₂) alkyl (e.g. methyl), —OR^{e}, —S(O)₂N(R^{e})₂ or, more preferably, halo (e.g. fluoro or, preferably, chloro);
X represents -G-R²;
G represents —(CH₂)ₘ—O—, —(CH₂)ₘ—SO₂N(R^{d})—, —(CH₂)ₘ—N(R^{d})—SO₂—, —(CH₂)ₘ—SO₂— or, preferably, —(CH₂)ₘ—C(O)—, —(CH₂)ₘ—C(O)O—, —(CH₂)ₘ—N(R^{d})—, —(CH₂)ₘ—N(R^{d})—CO—, —(CH₂)ₘ—CO—N(R^{d})—, —(CH₂)ₘ—NH—CO—NH— or C₁₋₃ alkylene (e.g. —CH₂— or, preferably, —C≡C— or —C≡C—CH₂—);
m represents 1 or, preferably, 0;
R^{d} represents C₁₋₂ alkyl. (e.g. methyl) or, preferably, hydrogen;
R² represents hydrogen, optionally substituted (i.e. by A²) C₁₋₄ (e.g. C₁₋₂) alkyl (e.g. ethyl) or -T-Q;
T represents a direct bond or —CH₂—;
Q represents C₃₋₆ (e.g. C₃₋₄) cycloalkyl (optionally substituted by one or more B⁶ substituents) or, Q more preferably represents heterocycloalkyl (e.g. morpholinyl, e.g. 4-morpholinyl), aryl (e.g. phenyl) or heteroaryl (e.g. 1,3-dihydroisoindolyl, such as 1,3-dihydroisoindol-2-yl, thiazolyl, such as 2-thiazolyl, pyrazolyl, such as 3-pyrazolyl, pyridyl, such as 3- or 5-pyridyl, or, preferably, indolyl, such as 6- or, preferably, 5-indolyl), all of which are optionally substituted as defined herein (i.e. by B⁷, B⁸ and B⁹, respectively);
when G represents —(CH₂)ₘ—O—, then R² preferably represents hydrogen (and m preferably represents 1);
when G represents —(CH₂)ₘ—SO₂N(R^{d})—, then R² preferably represents C₁₋₄ (e.g. C₁₋₂ alkyl, such as ethyl) or -T-Q (in which T is preferably a direct bond and Q is aryl, such as phenyl, optionally substituted as defined herein);
when G represents —(CH₂)ₘ—N(R^{d})—SO₂—, then R² may represent optionally substituted C₁₋₄ (e.g. C₁₋₂) alkyl (e.g. ethyl), or, R² in this instance preferably represents -T-Q (in which T is preferably a direct bond and Q is aryl, such as phenyl, optionally substituted as defined herein);
when G represents —(CH₂)ₘ—SO₂—, then R² preferably represents -T-Q (in which T is preferably a direct bond and Q is heteroaryl or, preferably, heterocycloalkyl, in which the point of attachment is via a heteroatom, such as nitrogen, e.g. T is preferably a 4-morpholinyl group);
when G represents —(CH₂)ₘ—C(O)—, and R² represents -T-Q (e.g. in which T represents a direct bond), then Q may represent optionally substituted aryl (e.g. phenyl) or, Q more preferably represents optionally substituted heteroaryl or, particularly optionally substituted heterocycloalkyl as defined herein (and the point of attachment of the heteroaryl or heterocycloalkyl group is via a heteroatom);
when G represents —(CH₂)ₘ—C(O)O—, then R² preferably represents hydrogen or optionally substituted C₁₋₄ (e.g. C₁₋₂) alkyl (e.g. methyl or, preferably, ethyl);
when G represents —(CH₂)ₘ—N(R^{d})—, then R² may represent -T-Q (in which T is C₁₋₂ alkylene, such as —CH₂— and Q is preferably aryl, such as phenyl) but however, R² in this instance preferably represents hydrogen;
when G represents —(CH₂)ₘ—N(R^{d})—CO—, then R² may represent -T-Q (in which T is a direct bond) and Q represents optionally substituted aryl (e.g. phenyl), but R² preferably represents optionally substituted C₁₋₄ (e.g. C₁₋₂) alkyl (e.g. ethyl);
when G represents —(CH₂)ₘ—CO—N(R^{d})—, then R² preferably represents optionally substituted C₁₋₄ (e.g. C₁₋₂) alkyl (e.g. ethyl optionally substituted by A¹) or -T-Q, in which Q preferably represents optionally substituted aryl or heteroaryl as defined herein;
when G represents —(CH₂)ₘ—NH—CO—NH—, then R² may represent C₃₋₆ (e.g. C₄₋₅) cycloalkyl (e.g. cyclopentyl) or, particularly, optionally substituted C₁₋₄ (e.g. C₁₋₂) alkyl (e.g. ethyl), or, R² in this instance more preferably represents -T-Q (in which T is preferably a direct bond), in which Q represents optionally substituted aryl as defined herein;
when G represents C₁₋₃ alkylene (e.g. —CH₂— or, preferably, —C≡C— or —C≡C—CH₂), then R² preferably represents -T-Q, in which T preferably represents a single bond and Q represents optionally substituted heterocycloalkyl (e.g. morpholinyl, such as 4-morpholinyl; in which the point of attachment is preferably via a heteroatom) or, Q preferably represents optionally substituted aryl as defined herein (e.g. unsubstituted phenyl);

A¹, A² and A³ independently represent —OR^{e} (e.g. —OCH₃) or —N(R^{e})—C(O)R^{e} (e.g. —N(H)—C(O)CH₃);

B¹ to B⁹ (e.g. B⁹) independently represent —N(R^{e})₂, —S(O)₂R^{e}, —N(R^{e})C(O)R^{e}, preferably, halo (e.g. chloro or fluoro), —CN, C₁₋₄ (e.g. C₁₋₂) alkyl (e.g. methyl optionally substituted by one or more fluoro, —OH and/or —COOH substituents, so forming for example a —CH₂OH, —CH₂—COOH or —CF₃ group), —OR^{e}, —S(O)₂N(R^{e})₂, —C(O)N(R^{e})₂ or, more preferably, —C(O)₂R^{e} (e.g. —C(O)₂CH₂CH₃ or —C(O)₂CH₃) or —C(O)R^{e} (e.g. —C(O)CH₃);
R^{e} represents hydrogen or C₁₋₂ alkyl (e.g. ethyl or methyl); or
any two R^{e} groups (e.g. those of the —N(R^{e})₂ moiety) may be linked together as hereinbefore defined (e.g. to form a 5- or 6-membered ring, such as a piperazinyl or 4,5-dihydropyrazolyl group);
R³ represents C₁₋₂ alkyl (e.g. methyl) or, preferably, hydrogen;
R⁴ represents hydrogen or C₁₋₂ alkyl (e.g. methyl);
R⁵ independently represents C₃₋₆ (e.g. C₄₋₅) cycloalkyl (e.g. cyclopentyl) or, preferably, hydrogen or C₁₋₂ alkyl (e.g. methyl);
at least two of R³, R⁴ and R⁵ represent hydrogen and the other represents C₃₋₆ (e.g. C₄₋₅) cycloalkyl (e.g. cyclopentyl) or, preferably C₁₋₂ alkyl (e.g. methyl) or hydrogen.

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I which process comprises:

(i) for compounds of formula I in which X represents C₃₋₆ cycloalkyl or heterocycloalkyl (both of which are optionally substituted as defined herein) or -G-R², reaction of a corresponding compound of formula II,

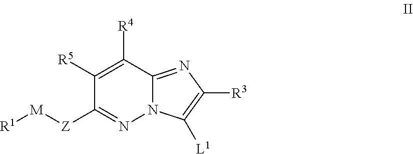

wherein L¹ represents a suitable leaving group, such as iodo, bromo, chloro or a sulfonate group (e.g. —OS(O)₂CF₃, —OS(O)₂CH₃ or —OS(O)₂PhMe), and Z, M, R¹, R³, R⁴ and R⁵ are as hereinbefore defined, with a compound of formula III,

L²-X^{a} III

wherein L² represents a suitable group such as —B(OH)₂, —B(OR^{wx})₂ or —Sn(R^{wx})₃, in which each R^{wx} independently represents a C₁₋₆ alkyl group, or, in the case of —B(OR^{wx})₂, the respective R^{wx} groups may be linked together to form a 4- to 6-membered cyclic group (such as a 4,4,5,5-tetramethyl-1,3,2-dioxaborolan-2-yl group), and X^{a} represents C₃₋₆ cycloalkyl, heterocycloalkyl (which latter two groups are optionally substituted by one or more substituents selected from B⁴ and B⁵) or -G-R². Alternatively, for compounds of formula I in which X represents -G-R², and G represents optionally substituted C₂₋₈ alkynylene (in which the point of attachment of a triple bond is a to the requisite 6,5-bicycle) compounds of formula III in which L² represents hydrogen and X^{a} represents -G-R² in which G represents C₂₋₈ alkynylene (in which the point of attachment of a triple bond is α to L²) optionally substituted by one or more substituents selected from A¹, may be employed. This reaction may be performed, for example in the presence of a suitable catalyst system, e.g. a metal (or a salt or complex thereof) such as Pd, CuI, Pd/C, PdCl₂, Pd(OAc)₂, Pd(Ph₃P)₂Cl₂, Pd(Ph₃P)₄ (i.e. palladium tetrakistriphenylphosphine), Pd₂(dba)₃ or NiCl₂ and a ligand such as t-Bu₃P, (C₆H₁₁)₃P, Ph₃P, AsPh₃, P(o-Tol)₃, 1,2-bis(diphenylphosphino)ethane, 2,2′-bis(di-tert-butylphosphino)-1,1′-biphenyl, 2,2′-bis(diphenylphosphino)-1,1′-bi-naphthyl, 1,1′-bis(diphenyl-phosphino-ferrocene), 1,3-bis(diphenylphosphino)propane, xantphos, or a mixture thereof, together with a suitable base such as, Na₂CO₃, K₃PO₄, Cs₂CO₃, NaOH, KOH, K₂CO₃, CsF, Et₃N, (i-Pr)₂NEt, t-BuONa or t-BuOK (or mixtures thereof) in a suitable solvent such as dioxane, toluene, ethanol, dimethylformamide, ethylene glycol dimethyl ether, water, dimethylsulfoxide, acetonitrile, dimethylacetamide, N-methylpyrrolidinone, tetrahydrofuran or mixtures thereof. The reaction may also be carried out for example at room temperature or above (e.g. at a high temperature such as the reflux temperature of the solvent system). Alternative reaction conditions include microwave irradiation conditions, for example at elevated temperature of above 150° C. (and which reaction may be performed in the presence of a suitable solvent, such as dimethylsulfoxide). Alternative L² groups that may be mentioned include alkali metal groups (e.g. lithium) and halo groups, which may be converted to a magnesium halide (i.e. a Grignard reagent), in which the magnesium may undergo a ‘trans-metallation’ reaction, thereby being exchanged with, for example, zinc. The skilled person will appreciate that various compounds of formula I in which the groups as defined by —Z-M-R¹ represent similar moieties may also be prepared in a similar manner;
(ii) for compounds of formula I in which X represents -G-R², G represents —(CH₂)ₘ—N(R^{d})— or —(CH₂)ₘ—O— and R² represents optionally substituted C₁₋₈ alkyl or -T-Q, reaction of a corresponding compound of formula I in which R² represents H, with a compound of formula IV,

R^{2x}-L¹ IV

wherein R^{2x} represents C₁₋₈ alkyl (optionally substituted by one or more substituents selected from A²) or -T-Q, and L¹, T and Q are as hereinbefore defined, and for example at around room temperature or above in the presence of a suitable base (e.g. pyridine, triethylamine, dimethylaminopyridine, diisopropylamine, sodium hydroxide, or mixtures thereof), an appropriate solvent (e.g. pyridine, dichloromethane, chloroform, tetrahydrofuran, dimethylformamide, triethylamine, dimethylsulfoxide, water or mixtures thereof) and, in the case of biphasic reaction conditions, optionally in the presence of a phase transfer catalyst. The skilled person will appreciate that the —(CH₂)ₘ—N(R^{d})— group, e.g. when R^{d} represents hydrogen, may need to be protected (and subsequently deprotected) in order to effect this transformation. The skilled person will also appreciate that alternative reaction conditions may be employed, for example when reaction with a compound of formula IV in which R^{2x} represents -T-Q and T represents a single bond occurs, reaction conditions such as those described in respect of process step (i) above may be employed. Further, the skilled person will also appreciate which values of R^{2x} in compounds of formula IV (for obtaining compounds of formula I) would be suitable in such a process step. Further, the skilled person will appreciate that compounds of formula I in which Z represents —(CH₂)ₙ—N(R^{a})— or —(CH₂)ₙ—O— may be prepared in a similar manner;
(iii) for compounds of formula I in which Z represents —(CH₂)ₙ—O—, —(CH₂)ₙ—S— or —(CH₂)ₙ—N(R^{a})— in which n represents 0, or, for compounds of formula I in which Z and M represent direct bonds and R¹ represents optionally substituted heteroaryl or heterocycloalkyl in which the point of attachment to the requisite 6,5-bicycle of formula I is via a heteroatom (such as a nitrogen heteroatom), reaction of a compound of formula V,

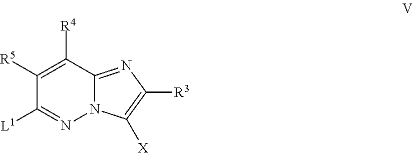

wherein L¹, X, R³, R⁴ and R⁵ are as hereinbefore defined, with (for the preparation of compounds of formula I in which Z represents —(CH₂)ₙ—O—, —(CH₂)ₙ—S— or —(CH₂)ₙ—N(R^{a})— in which n represents 0) a compound of formula VI,

H—Z^{a}-M-R¹ VI

wherein Z^{a} represents —O—, —S— or —N(R^{a})—, and R^{a}, R¹ and M are as hereinbefore defined, or with (for the preparation of compounds of formula I in which Z and M represent direct bonds and R¹ represents optionally substituted heteroaryl or heterocycloalkyl), a compound of formula VIA,

R^{1a}—H VIA

wherein R^{1a} represents a heteroaryl or heterocycloalkyl group, both of which are optionally substituted by one or more substituents selected from B¹, and in which the hydrogen atom depicted in the compound of formula VIA is attached to the heteroatom of the heteroaryl or heterocycloalkyl moiety, which heteroatom is to be attached to the requisite bicycle of the compound of formula I, which reactions may be performed under standard conditions, for example, such as those hereinbefore described in respect of process step (i) above, or, optionally in the presence of an appropriate metal catalyst (or a salt or complex thereof) such as Cu, Cu(OAc)₂, CuI (or CuI/diamine complex), copper tris(triphenyl-phosphine)bromide, Pd(OAc)₂, tris(dibenzylideneacetone)dipalladium(0) (Pd₂(dba)₃) or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N-dimethylethylenediamine, Na₂CO₃, K₂CO₃, K₃PO₄, Cs₂CO₃, t-BuONa or t-BuOK (or a mixture thereof, optionally in the presence of 4 Å molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof). Preferably, the reaction is carried out in the presence of NaH and dioxane (in the absence of a metal catalyst), for example at elevated temperature such as at reflux. This reaction may be carried out under microwave irradiation reaction conditions, for example a described in process step (i) above. Alternatively, the reaction may be performed as described herein, under such microwave irradiation reaction conditions, but in the absence of other reagents such as catalyst, base and even solvent (i.e. the reaction mixture may contain only compound of formula V and compound of formula VI or VIA). Furthermore, the skilled person will appreciate that a similar reaction may be performed in the instance where X in the compound of formula I represents a heteroaryl or heterocycloalkyl moiety (i.e. by reaction of a compound of formula II with an appropriate heteroaryl or heterocycloalkyl group). Further, the skilled person will appreciate that various compounds of formula I in which the groups as defined by X represent similar moieties may also be prepared in a similar manner;
(iv) compounds of formula I in which X represents -G-R², in which G represents —(CH₂)ₘ—N(R^{d})—SO₂—, —(CH₂)ₘ—N(R^{d})—CO— or —(CH₂)ₘ—NH—C(O)—NH— may be prepared by reaction of a corresponding compound of formula I in which G represents —(CH₂)ₘ—N(R^{d})—, R² represents hydrogen and R^{d} is as hereinbefore defined (or, in the case of the formation of the urea compound, represents hydrogen), with either a compound of formula VII,

L¹-Q¹-R² VII

wherein L¹ is as hereinbefore defined and preferably represents chloro, Q¹ represents —S(O)₂—, —C(O)— or —C(O)NH— (alternatively, in the case where Q¹ represents —C(O)—, L¹ may represent —O—C(O)—R², so forming an appropriate carboxylic acid anhydride), and R² is as hereinbefore defined; or, for the preparation of compounds of formula I in which X represents -G-R², and G represents —(CH₂)ₘ—NH—C(O)—NH—, with a compound of formula VIII,

O═C═N—R² VIII

wherein R² is as hereinbefore defined, under standard reaction conditions (for both reactions), for example such as those hereinbefore described in respect of process step (ii) above. The skilled person will appreciate that similar groups defined by —Z-M-R¹ in the compound of formula I may also be prepared in a similar manner;
(v) compounds of formula I in which X represents -G-R², G represents —NH— and R² represents optionally substituted C₁₋₈ alkyl, may be prepared by the reductive amination of a corresponding compound of formula I in which G represents —NH— and R² represents hydrogen, with a compound of formula IX,

R^{2b}—CHO IX

wherein R^{2b} represents C₁₋₇ alkyl optionally substituted by one or more substituents selected from A², and A² is as hereinbefore defined, under standard reaction conditions, for example in the presence of sodium cyanoborohydride or sodium triacetoxyborohydride, optionally in the presence of a suitable solvent such as an alcohol (e.g. ethanol or methanol);
(vi) compounds of formula I in which X represents -G-R² and G represents —CH₂—NH— may be prepared by a reductive amination of a compound of formula X,

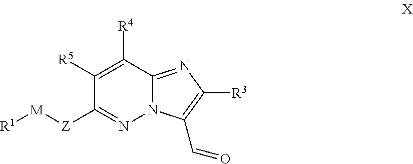

wherein Z, M, R¹, R³, R⁴ and R⁵ are as hereinbefore defined, with a compound of formula XI,

R²—NH₂ XI

wherein R² is as hereinbefore defined, for example under conditions such as those described hereinbefore in respect of process step (v) above;
(vii) compounds of formula I in which X represents -G-R², G represents —CH₂—O— and R² represents hydrogen may be prepared by reduction of a corresponding compound of formula X as hereinbefore defined, in the presence of a suitable reducing agent, for example, a borohydride such as NaBH₄, LiBH₄ or LiAlH₄, in the presence of a suitable solvent, e.g. an alcohol (e.g. methanol or ethanol);
(viii) compounds of formula I in which X represents -G-R², and G represents —(CH₂)ₘ—C(O)N(R^{d})— may be prepared by reaction of a corresponding compound of formula I but in which G represents —(CH₂)ₘ—C(O)O— (and R² represents optionally substituted C₁₋₈ alkyl or, preferably, hydrogen) with a compound of formula XII,

H(R^{d})N—R² XII

wherein R^{d} and R² are as hereinbefore defined, under standard amide coupling reaction conditions, for example in the presence of a suitable coupling reagent (e.g. 1,1′-carbonyldiimidazole, N,N′-dicyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluoro-phosphate, 2-(1H-benzotriazol-1-yl)-1,1,3,3-tetramethyluronium hexa-fluorophosphate (i.e. O-(1H-benzotriazol-1-yl)-N,N,N′,N′-tetramethyluronium hexafluorophosphate), benzotriazol-1-yloxytris-pyrrolidinophosphonium hexa-fluorophosphate, bromo-tris-pyrrolidinophosphonium hexafluorophosphate, 2-(1H-benzotriazol-1-yl)-1,1,3,3-tetramethyluronium tetra-fluorocarbonate, 1-cyclohexylcarbodiimide-3-propyloxymethyl polystyrene, O-(7-azabenzotriazol-1-yl)-N,N,N′,N′-tetramethyluronium hexafluorophosphate, O-benzotriazol-1-yl-N,N,N′,N′-tetramethyluronium tetrafluoroborate), optionally in the presence of a suitable base (e.g. sodium hydride, sodium bicarbonate, potassium carbonate, pyridine, triethylamine, dimethylaminopyridine, diisopropylamine, sodium hydroxide, potassium tert-butoxide and/or lithium diisopropylamide (or variants thereof) and an appropriate solvent (e.g. tetrahydrofuran, pyridine, toluene, dichloromethane, chloroform, acetonitrile, dimethylformamide, trifluoromethylbenzene, dioxane or triethylamine). Such reactions may be performed in the presence of a further additive such as 1-hydroxybenzotriazole hydrate. Alternatively, the carboxylic acid group may be converted under standard conditions to the corresponding acyl chloride (e.g. in the presence of SOCl₂ or oxalyl chloride), which acyl chloride is then reacted with a compound of formula XII, for example under similar conditions to those mentioned above. Alternatively still, when a carboxylic acid ester group is converted to a carboxylic acid amide, the reaction may be performed in the presence of a suitable reagent such as trimethylaluminium (and the relevant compound of formula XII);
(ix) for compounds of formula I in which there is a —CH₂— group present, reduction of a corresponding compound of formula I in which there is a —CH(OH)— group present, for example, in the presence of a suitable silicon based reducing agent such as (CH₃)₂SiCl₂ and optionally in the presence of an additive such as NaI;
(x) for compounds of formula I in which X represents -G-R², G represents methylene substituted by —OH, and R² represents optionally substituted C₁₋₈ alkyl or -T-Q, reaction of a compound of formula X as defined above with a compound of formula XIII,

R^{2y}-M¹ XIII

wherein M¹ represents an appropriate alkali metal group (e.g. sodium, potassium or, especially, lithium), a —Mg-halide or a zinc-based group (e.g. a zinc halide group) and R^{2y} represents C₁₋₈ alkyl (optionally substituted by one or more A² substituents) or -T-Q, and A², T and Q are as hereinbefore defined, under appropriate reaction conditions, for example under an inert atmosphere, in the presence of a suitable anhydrous solvent (e.g. an anhydrous polar aprotic solvent such as tetrahydrofuran, diethyl ether and the like);
(xi) compounds of formula I in which there is a —NH₂ group present (e.g. when X represents -G-R², and -G-R² represents —NH₂) may be prepared by the reduction of a corresponding compound of formula I in which there is a —NO₂ group present, under standard reaction conditions known to those skilled in the art, for example in the presence of a suitable reducing agent, for example reduction by catalytic hydrogenation (e.g. in the presence of a palladium catalyst in a source of hydrogen) or employing an appropriate reducing agent (such as trialkylsilane, e.g. triethylsilane or tin(II) chloride dihydrate). The skilled person will appreciate that where the reduction is performed in the presence of a —C(O)— group (e.g. when T represents —C(O)—), a chemoselective reducing agent may need to be employed;
(xii) preferably for compounds of formula I in which X represents -G-R² and G represents —C(O)O—, intramolecular cyclisation reaction of a compound of formula XIV,

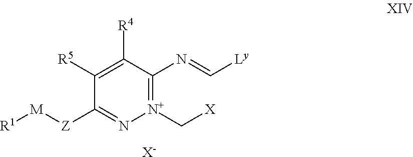

or a free base, or derivative thereof, wherein X⁻ represents an acid counterion (such as a halide counterion, e.g. Br⁻), L^{y} represents an appropriate leaving group such as —N(R^{s1})₂ (in which each R^{s1} independently represents C₁₋₆ alkyl, so forming, for example, a —N(CH₃)₂ group), and Z, M, R¹, R⁴, R⁵ and X are as hereinbefore defined (and X preferably represents -G-R² in which G represents —C(O)O—), under standard reaction conditions known to those skilled in the art, for example in the presence of a suitable solvent (e.g. acetonitrile), optionally in the presence of a base (such as an amine base, such as diisopropylethylamine) and at around room temperature;
(xiii) for compounds of formula I in which there is a carboxylic acid group present (e.g. in which X represents —COOH), hydrolysis of a corresponding compound of formula I in which there is a corresponding ester group present (e.g. in which X represents —COOC₁₋₈ alkyl, such as —COO-ethyl), under standard conditions;
(xiv) for compounds of formula I in which there is a hydroxy group present on an aromatic ring (e.g. if there is a B⁸ or B⁹ substituent present), reaction of a corresponding compound of formula I in which there is a methoxy group present on such an aromatic ring, under standard methyl ether cleavage reaction conditions, for example in the presence of BBr₃ or the like;
(xv) for compounds of formula I in which there is a —CH₂—NH₂ group present (e.g. when X represents —CH₂—NH₂), reduction of a compound of formula I in which there is a corresponding cyano (i.e —C≡N) group, under standard conditions, for example under catalytic hydrogenation conditions, such in the presence of a hydrogen source and a precious metal catalyst (e.g. Raney Nickel) and optionally in the presence of a suitable solvent (e.g. an alcoholic solvent such as ethanol);
(xvi) for compounds of formula I in which X represents -G-R², G represents —(CH₂)ₘ—N(R^{d})—C(O)—, and R² is other than hydrogen, reaction of a compound of formula I in which X represents —(CH₂)ₘ—N(R^{d})H, with a compound of formula XIVA,

R^{2a}—C(O)OH XIVA

wherein R^{2a} represents R², provided that it does not represent hydrogen, under standard amide coupling reaction conditions, for example such as those hereinbefore described in respect of process step (viii) above;
(xvii) for compounds of formula I in which there is a —CH₂OH group present (e.g. for compounds in which X represents —CH₂OH), reduction of a compound of formula I in which there is a corresponding —C(O)OR² group present (in which R² is preferably optionally substituted C₁₋₈ alkyl, such as ethyl), under standard conditions, for example in the presence of LiAlH₄ or another suitable reducing agent (such as LiBH₄ or borane);
(xviii) for compounds of formula I in which there is a —CH₂— moiety attached to a heteroaryl or heterocycloalkyl moiety via a heteroatom, such as a nitrogen heteroatom (e.g. when X represents —CH₂-het^{a}, in which het^{a} represents a heteroaryl or heterocycloalkyl group linked via a heteroatom, for example X may represent —CH₂-[4-morpholinyl]), reaction of a compound of formula I in which there is a corresponding —CH₂—OH moiety present (e.g. a compound of formula I in which X represents —CH₂—OH) with a compound of formula VIA as hereinbefore defined, under standard reaction conditions, for example such as those which first involve the conversion of the —OH moiety to a suitable leaving group (e.g. by first performing a reaction in the presence of N-bromosuccinimide, or the like, and triphenylphosphine, or the like, in the presence of a suitable solvent such as DMF, after which the compound of formula VIA may be added to the reaction mixture);
(xix) for compounds of formula I in which X represents —C(O)OR², reaction of a compound of formula XIX as defined hereinafter, with a compound of formula XIVB,

R³—C(═O)—C(L¹)(H)—C(O)OR² XIVB

wherein L¹, R² and R³ are as defined herein, under standard reaction conditions such as those described herein;
(xx) reaction of a corresponding compound of formula XVII as defined hereinafter, but in which L^{1a} represents —Z-M-R¹, with a compound of formula XVIII as hereinafter defined (and in particular those in which X represents —C(O)-T-Q), under similar reaction conditions to those described herein, but which favour the formation of the compound of formula I (rather than an intermediate, such as a compound of formula XIV as defined above);
(xxi) for compounds of formula I in which X represents -G-R², and G represents —S(O)₂N(R^{d})—, reaction of a compound of formula XIVC,

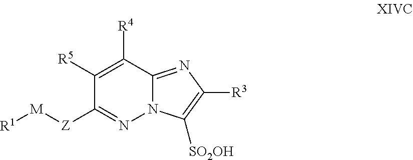

wherein R¹, R³, R⁴, R⁵, Z and M are as hereinbefore defined, with a compound of formula XII as hereinbefore defined.

Compounds of formula II in which L¹ represents halo may be prepared by reaction of a corresponding compound of formula XV,

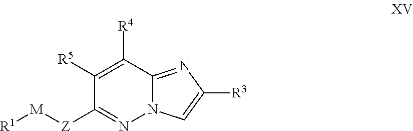

wherein Z, M, R¹, R³, R⁴ and R⁵ are as hereinbefore defined, under standard conditions known to those skilled in the art, for example by reaction in the presence of a source of halide ions, for instance an electrophile that provides a source of iodide ions includes iodine, diiodoethane, diiodotetrachloroethane or, preferably, N-iodosuccinimide, a source of bromide ions includes N-bromosuccinimide and bromine, and a source of chloride ions includes N-chlorosuccinimide, chlorine and iodine monochloride, for instance in the presence of a suitable solvent, such as an alcohol (e.g. methanol) optionally in the presence of a suitable base, such as a weak inorganic base, e.g. sodium bicarbonate.

Other compounds of formula II may also be prepared under standard conditions, for instance such as those described herein. For example, for synthesis of compounds of formula II in which L¹ represents a sulfonate group, reaction of a compound corresponding to a compound of formula II but in which L¹ represents —OH with an appropriate sulfonyl halide, under standard reaction conditions, such as in the presence of a base (e.g. as hereinbefore described in respect of preparation of compounds of formula I (process step (ii)).

Compounds of formula V may be prepared by reaction of a compound of formula XVA,

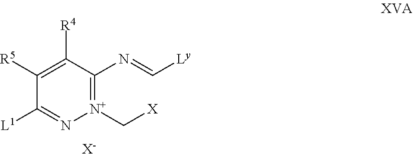

wherein L¹, R⁴, R⁵, L^{y}, X and X⁻ are as hereinbefore defined, under reaction conditions such as those hereinbefore described in respect of preparation of compounds of formula I (process step (xii) above).

Compounds of formula X may be prepared by reaction with a corresponding compound of formula XV as hereinbefore defined, with dimethylformamide, under standard conditions, and optionally in the presence of oxalyl chloride, phosgene or the like, in optionally in the presence of a further solvent other than DMF (e.g. dichloromethane).

Compounds of formula XIII may be prepared by reaction of a corresponding compound of formula XVI,

R^{2y}L^{x} XVI

wherein L^{x} represents halo, and R^{2y} is as hereinbefore defined, by, in the case of the formation of a compound of formula XIII in which:
(i) M¹ represents a —Mg-halide, employing magnesium or a suitable reagent such as a mixture of C₁₋₆ alkyl-Mg-halide and ZnCl₂ or LiCl, under standard Grignard conditions known to those skilled in the art (e.g. optionally in the presence of a catalyst (e.g. FeCl₃));
(ii) M¹ represents lithium, forming the corresponding lithiated compound under halogen-lithium exchange reaction conditions known to those skilled in the art (e.g. employing n-BuLi or t-BuLi in the presence of an anhydrous suitable solvent (e.g. a polar aprotic solvent such as THF)).

The skilled person will also appreciate that the magnesium of the Grignard reagent or the lithium of the lithiated species may be exchanged to a different metal (i.e. a transmetallation reaction may be performed), for example to zinc (e.g. using ZnCl₂), so forming for example, the corresponding compound of formula XIII in which M¹ represent a zinc-based group;

Compounds of formula XIV or XVA may be prepared by reaction of a compound of formula XVII,

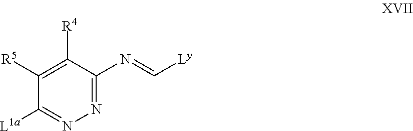

wherein L^{1a} represents —Z-M-R¹ (for the preparation of compounds of formula XIV) or L¹ (for the preparation of compounds of formula XVA), and Z, M, R¹, R⁴, R⁵ and L^{y} are as hereinbefore defined, with a compound of formula XVIII,

L¹-CH₂—X XVIII

wherein L¹ is as hereinbefore defined (and preferably represents bromo) and X is as hereinbefore defined, under standard conditions, for example, in the presence of a suitable solvent, such as acetonitrile, and preferably at elevated temperature, for example at reflux. The skilled person will appreciate that similar compounds of formulae XIV or XVA but in which X represents a different group may be prepared by this method. For example a compound corresponding to a compound of formula XIV or XVA but in which X represents —C≡N may be prepared by reaction of a compound of formula XVII with a compound of formula L¹-CH₂—CN (in which L¹ in this instance is preferably bromo).

Compounds of formula XIVC may be prepared by reaction of a corresponding compound of formula XV as hereinbefore defined, with a reagent for the introduction of the sulfonic acid group, such as oleum.

Compounds of formula XV in which R³ represents R^{j} as hereinbefore defined (e.g. hydrogen or C₁₋₄ alkyl optionally substituted by one or more substituents selected from halo and —OR^{h}), reaction of a compound of formula XIX,

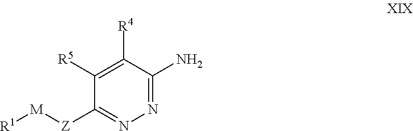

wherein Z, M, R¹, R⁴ and R⁵ are as hereinbefore defined, with a compound of formula XX,

Cl—CH₂—C(O)—R^{3a} XX

wherein R^{3a} represents R^{j} as hereinbefore defined (e.g. hydrogen or C₁₋₄ alkyl optionally substituted by one or more substituents selected from halo and —OR^{h} (and R^{h} is as hereinbefore defined)), under standard conditions known to those skilled in the art. For example, the compound of formula XX may already be present in water, and hence, the reaction may be performed in the presence of water as a solvent, optionally in the presence of a further solvent, such as an alcohol (e.g. n-butanol), for example at room temperature or, preferably, elevated temperature such as at reflux (other reaction conditions include those described in Stanovnik et al, 1967, 23, 2739-2746);

Compounds of formula XVII may be prepared by reaction of a compound of formula XXA,

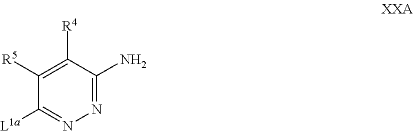

wherein L^{1a}, R⁴ and R⁵ are as hereinbefore defined, with a compound of formula XXB,

L^{y}-C(═O)H XXB

or a derivative thereof, wherein L^{y} is as hereinbefore defined (and preferably represents —N(CH₃)₂, thereby forming N,N-dimethylformamide or a derivative thereof, such as N,N-dimethylformamide diethyl acetal), under standard condensation reaction conditions such as reaction at elevated temperature (e.g. reflux) under an inert atmosphere.

Compounds of formulae XIX and XXA may be prepared by reaction of a compound of formula XXI,

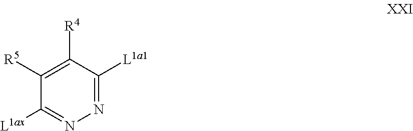

wherein L^{1a1} represents a suitable leaving group, such as one hereinbefore defined in respect of L¹ (e.g. chloro), L^{1ax} represents —Z-M-R¹ (for the preparation of compounds of formula XIX) or L^{1a} (for the preparation of compounds of formula XXA) and L^{1a}, Z, M, R¹, R⁴ and R⁵ are as hereinbefore defined, with ammonia, or a suitable derivative thereof (e.g. ammonium hydroxide/hydroxylamine), under standard reaction conditions (aromatic nucleophilic reaction conditions), for example such as those hereinbefore described in respect of preparation of compounds of formula I (process step (iii) above), e.g. under microwave irradiation reaction conditions.

Compounds of formulae III, IV, V, VI, VIA, VII, VIII, IX, XI, XII, XIVA, XIVB, XVI, XVII, XVIII, XX, XXB and XXI (as well as some compounds of e.g. formulae II, X, XIVC, XVA, XXA and XIX) are either commercially available, are known in the literature, or may be obtained either by analogy with the processes described herein, or by conventional synthetic procedures, in accordance with standard techniques, from available starting materials using appropriate reagents and reaction conditions. Further, the skilled person will appreciate that where reactions to introduce the “—Z-M-R¹” moiety of compounds of formula I is described, similar reactions may be performed to introduce the “—X” moiety (e.g. when X represents “-G-R²”) in compounds of formula I and vice versa. Further, processes to prepare compounds of formula I may be described in the literature, for example in:
Werber, G. et al.; J. Heterocycl. Chem.; EN; 14; 1977; 823-827;
Andanappa K. Gadad et al. Bioorg. Med. Chem. 2004, 12, 5651-5659;
Paul Heinz et al. Monatshefte für Chemie, 1977, 108, 665-680;
M. A. El-Sherbeny et al. Boll. Chim. Farm. 1997, 136, 253-256;
Nicolaou, K. C.; Bulger, P. G.; Sarlah, D. Angew. Chem. Int. Ed. 2005, 44, 2-49;
Bretonnet et al. J. Med. Chem. 2007, 50, 1872;
Asunción Marín et al. Farmaco 1992, 47 (1), 63-75;
Severinsen, R. et al. Tetrahedron 2005, 61, 5565-5575;
Nicolaou, K. C.; Bulger, P. G.; Sarlah, D. Angew. Chem. Int. Ed. 2005, 44, 2-49;
M. Kuwahara et al., Chem. Pharm Bull., 1996, 44, 122;
Wipf, P.; Jung, J.-K. J. Org. Chem. 2000, 65(20), 6319-6337;
Shintani, R.; Okamoto, K. Org. Lett. 2005, 7 (21), 4757-4759;
Nicolaou, K. C.; Bulger, P. G.; Sarlah, D. Angew. Chem. Int. Ed. 2005, 44, 2-49;
J. Kobe et al., Tetrahedron, 1968, 24, 239;
P. F. Fabio, A. F. Lanzilotti and S. A. Lang, Journal of Labelled Compounds and Pharmaceuticals, 1978, 15, 407;
F. D. Bellamy and K. Ou, Tetrahedron Lett., 1985, 25, 839;
M. Kuwahara et al., Chem. Pharm Bull., 1996, 44, 122;
A. F. Abdel-Magid and C. A Maryanoff. Synthesis, 1990, 537;
M. Schlósser et al. Organometallics in Synthesis. A Manual, (M. Schlosser, Ed.), Wiley &Sons Ltd: Chichester, UK, 2002, and references cited therein;
L. Wengwei et al., Tetrahedron Lett., 2006, 47, 1941;
M. Plotkin et al. Tetrahedron Lett., 2000, 41, 2269;
Seyden-Penne, J. Reductions by the Alumino and Borohydrides, VCH, NY, 1991;
O. C. Dermer, Chem. Rev., 1934, 14, 385;
N. Defacqz, et al., Tetrahedron Lett., 2003, 44, 9111;
S. J. Gregson et al., J. Med. Chem., 2004, 47, 1161;
A. M. Abdel Magib, et al., J. Org. Chem., 1996, 61, 3849;
A. F. Abdel-Magid and C. A Maryanoff. Synthesis, 1990, 537;
T. Ikemoto and M. Wakimasu, Heterocycles, 2001, 55, 99;
E. Abignente et al., II Farmaco, 1990, 45, 1075;
T. Ikemoto et al., Tetrahedron, 2000, 56, 7915;
T. W. Greene and P. G. M. Wuts, Protective Groups in Organic Synthesis, Wiley, NY, 1999;
S. Y. Han and Y.-A. Kim. Tetrahedron, 2004, 60, 2447;
J. A. H. Lainton et al., J. Comb. Chem., 2003, 5, 400; or
Wiggins, J. M. Synth. Commun., 1988, 18, 741.

The substituents Z, M, R¹, X, R³, R⁴ and R⁵ in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, etherifications, halogenations or nitrations. Such reactions may result in the formation of a symmetric or asymmetric final compound of the invention or intermediate. The precursor groups can be changed to a different such group, or to the groups defined in formula I, at any time during the reaction sequence. For example, in cases in which X represents -G-R², in which G represents —C(O)O— and R² is a substituent other than hydrogen, so forming an ester group, the skilled person will appreciate that at any stage during the synthesis (e.g. the final step), the relevant ester group may be hydrolysed to form a carboxylic acid functional group (i.e. a compound in which the relevant R² group represents hydrogen). Similarly one halo group in a compound of formula I, or intermediate thereto, may be exchanged for another halo group, for instance a chloro substituent may be replaced with an iodo substituent by reaction in the presence of a suitable reagent such as potassium iodide under reaction conditions known to those skilled in the art. Specific nitration reactions that may be mentioned include nitration directly onto the aromatic 6,5-bicycle of formula I (e.g. to prepare compounds corresponding to compounds of formulae I or V, but in which X represents —NO₂, the nitration of a corresponding compound of formula I but in which X represents hydrogen may be performed), e.g. by reaction of the aromatic bicycle with a mixture of sulfuric and nitric acid at low temperatures (below 5° C., e.g. at about 0° C.).

Compounds of the invention bearing a carboxyester functional group may be converted into a variety of derivatives according to methods well known in the art to convert carboxyester groups into carboxamides, N-substituted carboxamides, N,N-disubstituted carboxamides, carboxylic acids, and the like. The operative conditions are those widely known in the art and may comprise, for instance in the conversion of a carboxyester group into a carboxamide group, the reaction with ammonia or ammonium hydroxide in the presence of a suitable solvent such as a lower alcohol, dimethylformamide or a mixture thereof; preferably the reaction is carried out with ammonium hydroxide in a methanol/dimethylformamide mixture, at a temperature ranging from about 50° C. to about 100° C. Analogous operative conditions apply in the preparation of N-substituted or N,N-disubstituted carboxamides wherein a suitable primary or secondary amine is used in place of ammonia or ammonium hydroxide. Likewise, carboxyester groups may be converted into carboxylic acid derivatives through basic or acidic hydrolysis conditions, widely known in the art. Further, amino derivatives of compounds of the invention may easily be converted into the corresponding carbamate, carboxamido or ureido derivatives.

Compounds of the invention may be isolated from their reaction mixtures using conventional techniques (e.g. recrystallisations).

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Synthesis”, 3^{rd} edition, T. W. Greene & P. G. M. Wutz, Wiley-Interscience (1999).

### Medical and Pharmaceutical Uses

Compounds of the invention are indicated as pharmaceuticals. According to a further aspect of the invention there is provided a compound of the invention, as hereinbefore defined but without provisos (II), (VIII) and (X), for use as a pharmaceutical.

Compounds of the invention may inhibit protein kinases, such as CDK-2, SRC, GSK-3, and in particular may inhibit PI3-K or a PIM family kinase such as PIM-1, PIM-2 and/or PIM-3, for example as may be shown in the tests described below and/or in tests known to the skilled person. Thus, the compounds of the invention may be useful in the treatment of those disorders in an individual in which the inhibition of such protein kinases (e.g. a PIM family kinase such as PIM-1 and/or PIM-2) is desired and/or required.

The term “inhibit” may refer to any measurable reduction and/or prevention of catalytic protein kinase (e.g. CDK-2, SRC, GSK-3 or, preferably, PI3-K or a PIM family kinase such as PIM-1, PIM-2 and/or PIM-3) activity. The reduction and/or prevention of protein kinase activity may be measured by comparing the protein kinase activity in a sample containing a compound of the invention and an equivalent sample of protein kinase (e.g. CDK-2, SRC, GSK-3 or, preferably, PI3-K or a PIM family kinase such as PIM-1, PIM-2 and/or PIM-3) in the absence of a compound of the invention, as would be apparent to those skilled in the art. The measurable change may be objective (e.g. measurable by some test or marker, for example in an in vitro or in vivo assay or test, such as one described hereinafter, or otherwise another suitable assay or test known to those skilled in the art) or subjective (e.g. the subject gives an indication of or feels an effect).

Compounds of the invention may be found to exhibit 50% inhibition of a protein kinase (e.g. CDK-2, SRC, GSK-3 or, preferably, PI3-K or a PIM family kinase such as PIM-1, PIM-2 and/or PIM-3) at a concentration of 100 μM or below (for example at a concentration of below 50 μM, or even below 10 μM), when tested in an assay (or other test), for example as described hereinafter, or otherwise another suitable assay or test known to the skilled person.

Compounds of the invention are thus expected to be useful in the treatment of a disorder in which a protein kinase (and particularly CDK-2, SRC, GSK-3 or, preferably, PI3-K or a PIM family kinase such as PIM-1, PIM-2 and/or PIM-3) is known to play a role and which are characterised by or associated with an overall elevated activity of that protein kinase (due to, for example, increased amount of the kinase or increased catalytic activity of the kinase). Such disorders include cancer (particularly lymphomas or a cancer as described hereinafter), inflammatory diseases (such as asthma, allergy and Chrohn's disease), immunosuppression (such as transplantation rejection and autoimmune diseases), and other associated diseases. Other associated diseases that may be mentioned (particularly due to the key role of kinases in the regulation of cellular proliferation) include other cell proliferative disorders and/or non-malignant diseases, such as benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis.

As stated above, the compounds of the invention may be useful in the treatment of cancer. More, specifically, the compounds of the invention may therefore be useful in the treatment of a variety of cancer including, but not limited to: carcinoma such as cancer of the bladder, breast, colon, kidney, liver, lung (including small cell lung cancer), esophagus, gall-bladder, ovary, pancreas, stomach, cervix, thyroid, prostate and skin, as well as squamous cell carcinoma; hematopoietic tumors of lymphoid lineage, including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including melanoma, seminoma, teratocarcinoma, osteosarcoma, xeroderma pigmentosum, keratoxanthoma, thyroid follicular cancer and Kaposi's sarcoma.

Further, the protein kinases (e.g. CDK-2, SRC, GSK-3 or, more particularly, PI3-K or a PIM family kinase such as PIM-1, PIM-2 and/or PIM-3) may also be implicated in the multiplication of viruses and parasites. They may also play a major role in the pathogenesis and development of neurodegenerative disorders. Hence, compounds of the invention may also be useful in the treatment of viral conditions, parasitic conditions, as well as neurodegenerative disorders.

Compounds of the invention are indicated both in the therapeutic and/or prophylactic treatment of the above-mentioned conditions.

According to a further aspect of the present invention, there is provided a method of treatment of a disease which is associated with the inhibition of protein kinase (e.g. CDK-2, SRC, GSK-3 or, preferably, PI3-K or a PIM family kinase such as PIM-1, PIM-2 and/or PIM-3) is desired and/or required (e.g. cancer or another disease as mentioned herein), which method comprises administration of a therapeutically effective amount of a compound of the invention, as hereinbefore defined but without the provisos, to a patient suffering from, or susceptible to, such a condition.

“Patients” include mammalian (including human) patients.

The term “effective amount” refers to an amount of a compound, which confers a therapeutic effect on the treated patient. The effect may be objective (e.g. measurable by some test or marker) or subjective (e.g. the subject gives an indication of or feels an effect).

Compounds of the invention may be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like. The type of pharmaceutical formulation may be selected with due regard to the intended route of administration and standard pharmaceutical practice. Such pharmaceutically acceptable carriers may be chemically inert to the active compounds and may have no detrimental side effects or toxicity under the conditions of use.

Such formulations may be prepared in accordance with standard and/or accepted pharmaceutical practice. Otherwise, the preparation of suitable formulations may be achieved non-inventively by the skilled person using routine techniques and/or in accordance with standard and/or accepted pharmaceutical practice.

According to a further aspect of the invention there is thus provided a pharmaceutical formulation including a compound of the invention, as hereinbefore defined but without provisos (II), (VIII) and (X), in admixture with a pharmaceutically acceptable adjuvant, diluent or carrier.

Depending on e.g. potency and physical characteristics of the compound of the invention (i.e. active ingredient), pharmaceutical formulations that may be mentioned include those in which the active ingredient is present in at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The amount of compound of the invention in the formulation will depend on the severity of the condition, and on the patient, to be treated, as well as the compound(s) which is/are employed, but may be determined non-inventively by the skilled person.

The invention further provides a process for the preparation of a pharmaceutical formulation, as hereinbefore defined, which process comprises bringing into association a compound of the invention, as hereinbefore defined but without provisos (II), (VIII) and (X), or a pharmaceutically acceptable ester, amide, solvate or salt thereof with a pharmaceutically-acceptable adjuvant, diluent or carrier.

Compounds of the invention may also be combined with other therapeutic agents that are inhibitors of protein kinases (e.g. CDK-2, SRC, GSK-3 or, preferably, PI3-K or a PIM family kinase such as PIM-1, PIM-2 and/or PIM-3) and/or useful in the treatment of a cancer and/or a proliferative disease. Compounds of the invention may also be combined with other therapies.

According to a further aspect of the invention, there is provided a combination product comprising:
(A) a compound of the invention, as hereinbefore defined but without the provisos (for example without provisos (II), (VIII) and (X)); and
(B) another therapeutic agent that is useful in the treatment of cancer and/or a proliferative disease,
   wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined but without the provisos (for example, without provisos (II), (VIII) and (X)), another therapeutic agent that is useful in the treatment of cancer and/or a proliferative disease, and a pharmaceutically-acceptable adjuvant, diluent or carrier; and
(2) a kit of parts comprising components:
(a) a pharmaceutical formulation including a compound of the invention, as hereinbefore defined but without the provisos (for example, without provisos (II), (VIII) and (X)), in admixture with a pharmaceutically-acceptable adjuvant, diluent or carrier; and
(b) a pharmaceutical formulation including another therapeutic agent that is useful in the treatment of cancer and/or a proliferative disease in admixture with a pharmaceutically-acceptable adjuvant, diluent or carrier,
   which components (a) and (b) are each provided in a form that is suitable for administration in conjunction with the other.

The invention further provides a process for the preparation of a combination product as hereinbefore defined but without the provisos (for example, without provisos (II), (VIII) and (X)), which process comprises bringing into association a compound of the invention, as hereinbefore defined but without the provisos, or a pharmaceutically acceptable ester, amide, solvate or salt thereof with the other therapeutic agent that is useful in the treatment of cancer and/or a proliferative disease, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Depending on the disorder, and the patient, to be treated, as well as the route of administration, compounds of the invention may be administered at varying therapeutically effective doses to a patient in need thereof. However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a compound of the invention.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

Compounds of the invention may have the advantage that they are effective inhibitors of protein kinases (such as CDK-2, SRC, GSK-3 or, preferably, PI3-K or a PIM family kinase such as PIM-1, PIM-2 and/or PIM-3).

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

### EXAMPLES/BIOLOGICAL TESTS

### PIM-1 Biochemical Assay

The biochemical assay to measure PIM-1 activity relies on the ADP Hunter assay kit (DiscoveRx Corp., Cat. # 90-0077), that determines the amount of ADP as direct product of the kinase enzyme activity.

The enzyme has been expressed and purified in-house as a recombinant human protein with a C-terminal histidine tag. The protein is active and stable.

Assay conditions were as indicated by the kit manufacturers with the following adaptations for the kinase activity step:
Kinase assay buffer and assay volume stay as recommended (15 mM HEPES, pH 7.4, 20 mM NaCl, 1 mM EGTA, 0.02% Tween 20, 10 mM MgCl₂ and 0.1 mg/ml bovine γ-globulins/75 μl assay volume)
Incubation time and temperature: 60 min at 30° C.
PIM-1 concentration: 50 pg/μl
ATP concentration: 100 μM
PIM-1 substrate peptide: PIMtide (ARKRRRHPSGPPTA)
Peptide concentration: 60 μM
Positive control for kinase activity inhibition: 1-10 μM Staurosporine
DMSO concentration have to stay below 2% during the kinase reaction

Assays were performed in either 96 or 384-well plates. The final outcome of the coupled reactions provided by the kit is the release of the fluorescent product Resorufin and has been measured with a multilabel HTS counter VICTOR V (PerkinElmer) using an excitation filter at 544 nm and an emission filter at 580 nm.

### PIM-2 Biochemical Assay

The biochemical assay to measure PIM-2 activity relies on the ADP Hunter assay kit (DiscoveRx Corp., Cat. # 90-0077), that determines the amount of ADP as direct product of the kinase enzyme activity.

The enzyme has been expressed and purified in-house as a recombinant human protein with a N-terminal histidine tag. The protein is active and stable.

Assay conditions were as indicated by the kit manufacturers with the following adaptations for the kinase activity step:
Kinase assay buffer and assay volume stay as recommended (15 mM HEPES, pH 7.4, 20 mM NaCl, 1 mM EGTA, 0.02% Tween 20, 10 mM MgCl₂ and 0.1 mg/ml bovine γ-globulins/20 μl assay volume)
Incubation time and temperature: 30 min at 30° C.
PIM-2 concentration: 350 pg/μl
ATP concentration: 100 μM
PIM-1 substrate peptide: PIMtide (ARKRRRHPSGPPTA)
Peptide concentration: 100 μM
Positive control for kinase activity inhibition: 1-10 μM Staurosporine
DMSO concentration have to stay below 2% during the kinase reaction

Assays were performed in either 96 or 384-well plates. The final outcome of the coupled reactions provided by the kit is the release of the fluorescent product Resorufin and has been measured with a multilabel HTS counter VICTOR V (PerkinElmer) using an excitation filter at 544 nm and an emission filter at 580 nm.

The compound names given herein were generated with MDL ISIS/DRAW 2.5 SP 2, Autonom 2000.

The invention is illustrated by way of the following examples.

### General Experimental Conditions

Compounds were analyzed on HPLC-MS (Agilent 1100 Series) with ESI+ (API 2000) and equipped with different brands of C18 columns. Analysis of final compounds was performed using RP-C18 Gemini column, (150×4.6 mm, 5 um), eluting with 5%-100% of B in 15 min, flow rate=1 mL/min (B═CH₃CN+0.1% formic acid; A=H₂O+0.1% formic acid).

MW calculated is an isotopic average and the “found mass” refers to the most abundant isotope detected in the LC-MS.

NMR was recorded in a Bruker Avance II 300 spectrometer.

### Intermediate 1

### 3-Chloro-6-(dimethylamino-methyleneamino)-1-ethoxycarbonylmethyl-pyridazin-1-ium bromide

N,N-Dimethyl-N′-(6-chloro-pyridazin-3-yl)-formamidine [Zupan M. et al. J. Org. Chem., 37, 2960, 1972] (7.09 g, 42.1 mmol) was dissolved in acetonitrile (100 mL) and ethylbromoacetate (3.1 mL, 126.2 mmol) was added, and the reaction was stirred overnight at reflux temperature. The solvent was partially removed up to 1/3 and diethyl ether was added. The resulting solid was filtered off, washed with diethyl ether and dried to give 11.4 g of 3-chloro-6-(dimethylamino-methyleneamino)-1-ethoxycarbonylmethylpyridazin-1-ium bromide (77% yield).

¹H NMR (300 MHz, CDCl₃): δ 10.40 (1H, s), 9.78 (1H, d, J=9.7 Hz), 7.85 (1H, d, J=9.7 Hz), 5.22 (2H, s), 4.36 (2H, q, J=7.1 Hz), 3.74 (3H, s), 3.30 (3H, s), 1.40 (3H, t, J=7.1 Hz).

LCMS: 271 ([M]−80), (MW: 351.63).

### Intermediate 2

### 6-Chloro-imidazo[1,2-b]pyridazine-3-carboxylic acid ethyl ester

3-Chloro-6-(dimethylamino-methyleneamino)-1-ethoxycarbonylmethyl-pyridazin-1-ium bromide (11.40 g, 32.40 mmol) was dissolved in acetonitrile (200 mL) and diisopropylethylamine (15.22 mL, 64.81 mmol) was added. The reaction mixture was stirred for 4 hours at room temperature. The solvent was removed in vacuo and the residue was triturated from water to give 5.77 g of 6-chloro-imidazo[1,2-b]pyridazine-3-carboxylic acid ethyl ester as a pale brown solid (77% yield).

¹H NMR (300 MHz, CDCl₃): δ 8.36 (1H, s), 8.01 (1H, d, J=9.4 Hz), 7.27 (1H, d, J=9.4 Hz), 4.46 (2H, q, J=7.1 Hz), 1.37 (3H, t, J=7.1 Hz).

LCMS: 226 [M+1]. (MW: 225.64)

### General Procedure A for the Preparation of Examples 1-3

A mixture of 6-chloro-imidazo[1,2-b]pyridazine-3-carboxylic acid ethyl ester (1 eq) and the appropriate amine (e.g. 4-fluorobenzylamine) (2.2 eq) in 1,4-dioxane (about 1.5 mL/mmol) was heated at 160° C. for several hours (from 9 to 14 hours depending upon the corresponding amine) under microwave irradiation. On cooling, the solvent was removed in vacuo, saturated aqueous solution of sodium hydrogen carbonate (about 20 mL) was added and the mixture was extracted with ethyl acetate (4×). The combined organic fractions were dried (sodium sulphate), the solvent removed in vacuo and the residue was purified by column chromatography on flash silica gel to give the desired product (e.g. 6-(4-fluorobenzylamino)-imidazo[1,2-b]pyridazine-3-carboxylic acid ethyl ester).

### Example 1

### 6-(4-Fluoro-benzylamino)-imidazo[1,2-b]pyridazine-3-carboxylic acid ethyl ester

The title compound was obtained in 58% yield after purification by column chromatography on flash silica gel (ethyl acetate).

¹H NMR (300 MHz, CDCl₃): δ 8.11 (1H, s), 7.69 (1H, d, J=9.6 Hz), 7.42 (2H, dd, J=8.4, 5.5 Hz), 7.02 (2H, t J=8.7 Hz), 6.56 (1H, d, J=9.6 Hz), 4.76 (1H, t, J=4.8 Hz), 4.58 (2H, d, J=5.5 Hz), 4.40 (2H, q, J=7.1 Hz), 1.39 (3H, t, J=7.1 Hz).

LCMS: 315 [M+1], (MW: 314.32).

### Example 2

### 6-(4-Methoxy-benzylamino)-imidazo[1,2-b]pyridazine-3-carboxylic acid ethyl ester

The title compound was obtained in 62% yield after purification by flash chromatography on silica gel (ethyl acetate).

¹H NMR (300 MHz, CDCl₃): δ 8.13 (1H, s), 7.69 (1H, d, J=9.7 Hz), 7.37 (2H, d, J=8.6), 6.89 (2H, t, J=8.6 Hz), 6.59 (1H, d, J=9.7 Hz), 4.81 (1H, t, J=4.8 Hz), 4.56 (2H, d, J=5.4 Hz), 4.43 (2H, q, J=7.1 Hz), 3.81 (3H, s), 1.42 (3H, t, J=7.1 Hz).

LCMS: 327 [M+1], (MW: 326.36).

### Example 3

### 6-[(Furan-2-ylmethyl)-amino]-imidazo[1,2-b]pyridazine-3-carboxylic acid ethyl ester

The title compound was obtained in 49% yield after purification by flash column chromatography on silica gel (ethyl acetate).

¹H NMR (300 MHz, CDCl₃): δ 8.11 (1H, s), 7.69 (1H, d, J=9.6 Hz), 7.35 (1H, s), 6.60 (1H, d, J=9.7 Hz), 6.41 (1H, d, J=2.9 Hz), 6.36 (1H, dd, J=2.9, 1.5 Hz), 4.86 (1H, t, J=4.1 Hz), 4.62 (2H, d, J=5.5 Hz), 4.40 (2H, q, J=7.1 Hz), 1.40 (3H, t, J=7.1 Hz).

LCMS: 287 [M+1], (MW: 286.29).

### Example 4

### 6-(4-Sulfamoyl-benzylamino)-imidazo[1,2-b]pyridazine-3-carboxylic acid ethyl ester

A mixture of 6-chloro-imidazo[1,2-b]pyridazine-3-carboxylic acid ethyl ester (0.45 g, 1.99 mmol), 4-homosulfanilamide hydrochloride (0.975 g, 4.38 mmol) and diisopropylethylamine (0.76 mL, 4.38 mmol) in 1,4-dioxane (5 mL) was heated at 160° C. for 11 hours under microwave irradiation. The solvent was removed in vacuo, saturated aqueous solution of sodium hydrogen carbonate (15 mL) was added and the mixture was extracted with ethyl acetate (4×50 mL). The combined organic fractions were dried (sodium sulphate), the solvent removed in vacuo and the residue was purified by flash column chromatography on silica gel (ethyl acetate/methanol 10:0 to 9.5:0.5) to give 0.35 g (47% yield) of 6-(4-sulfamoyl-benzylamino)-imidazo[1,2-b]pyridazine-3-carboxylic acid ethyl ester as a brown solid.

LCMS: 376 [M+1], (MW: 375.41).

### Example 5

### 6-(3,4-Dichloro-benzylamino)-imidazo[1,2-b]pyridazine-3-carboxylic acid ethyl ester

A mixture of 6-chloro-imidazo[1,2-b]pyridazine-3-carboxylic acid ethyl ester (1.08 g, 4.8 mmol) and 3,4-dichlorobenzylamine (1.38 mL, 10.2 mmol) in dry dimethylsulphoxide (8 mL) was heated at 160° C. for 2 hours and at 180° C. for 0.5 hours under microwave irradiation. The solvent was removed in vacuo and water was added (10 mL). Then, 28% aqueous ammonium hydroxide was added up to pH 11 and the mixture was extracted with ethyl acetate (4×200 mL). The combined organic fractions were dried (sodium sulphate), the solvent removed in vacuo and the residue was triturated from diethylether to give 1.28 g of 6-(3,4-dichloro-benzylamino)-imidazo[1,2-b]pyridazine-3-carboxylic acid ethyl ester as a yellow solid (71% yield).

¹H NMR (300 MHz, CDCl₃): δ 8.12 (1H, s), 7.69 (1H, d, J=9.6 Hz), 7.59 (1H, d, J=1.8 Hz), 7.38 (1H, d, J=8.2 Hz), 7.31 (1H, dd, J=8.2, 1.8 Hz), 6.60 (1H, d, J=9.6 Hz), 5.11 (1H, t, J=5.4 Hz), 5.57 (2H, d, J=5.8 Hz), 4.41 (2H, q, J=7.1 Hz), 1.40 (3H, t, J=7.1 Hz).

LCMS: 365 [M+1], (MW: 365.22).

### General Procedure B for the Preparation of Examples 6-9

A mixture of the appropriate 6-aminosubstituted-imidazo[1,2-b]pyridazine-3-carboxylic acid ethyl ester derivative (e.g. 6-(4-fluorobenzylamino)-imidazo[1,2-b]pyridazine-3-carboxylic acid ethyl ester) (1 eq) in ethanol (about 5 mL/mmol) and aqueous 4N potassium hydroxide (about 5 mL/mmol) was stirred at room temperature for several hours (from 2 to 4 hours depending upon the corresponding ester derivative). The ethanol was removed in vacuo, the resulting mixture was cooled at 0° C. and acetic acid was added dropwise up to pH 5. The resulting solid was filtered off, washed with water and dried to afford the desired acid (e.g. 6-(4-fluorobenzylamino)-imidazo[1,2-b]pyridazine-3-carboxylic acid).

### Example 6

### 6-(4-Fluoro-benzylamino)-imidazo[1,2-b]pyridazine-3-carboxylic acid

The title compound was obtained as a brown solid in 68% yield.

¹H NMR (300 MHz, DMSO-d₆): δ 12.55 (1H, bs), 7.96 (1H, s), 7.83 (1H, d, J=9.7 Hz), 7.73 (1H, t, J=5.6 Hz), 7.54 (2H, dd, J=8.1, 5.8 Hz), 7.13 (2H, t J=8.8 Hz), 6.86 (1H, d, J=9.7 Hz), 4.58 (2H, d, J=5.6 Hz).

LCMS: 287 [M+1], (MW: 286.27).

### Example 7

### 6-(4-Methoxy-benzylamino)-imidazo[1,2-b]pyridazine-3-carboxylic acid

The title compound was obtained as a brown solid in 69% yield.

¹H NMR (300 MHz, CDCl₃): [symbol] 11.75 (1H, bs), 8.16 (1H, s), 7.74 (1H, d, J=9.7 Hz), 7.24 (2H, d, J=8.5 Hz), 6.85 (2H, d, J=8.5 Hz), 6.86 (1H, d, J=9.7 Hz), 5.18 (1H, s) 4.42 (2H, d, J=5.2 Hz), 3.74 (3H, s).

LCMS: 299 [M+1], (MW: 298.30).

### Example 8

### 6-[(Furan-2-ylmethyl)-amino]-imidazo[1,2-b]pyridazine-3-carboxylic acid

The title compound was obtained as a brown solid in 76% yield.

¹H NMR (300 MHz, DMSO-d₆): δ 7.95 (1H, s), 7.83 (1H, d, J=9.7 Hz), 7.72 (1H, t, J=5.4 Hz), 7.59 (1H, d, J=0.6 Hz), 6.88 (1H, d, J=9.7 Hz), 6.54 (1H, d, J=3.1 Hz), 6.39 (1H, dd, J=3.0, 1.7 Hz), 4.46 (2H, d, J=5.4 Hz).

### Example 9

### 6-(4-Sulfamoyl-benzylamino)-imidazo[1,2-b]pyridazine-3-carboxylic acid

The title compound was obtained as a brown solid in 30% yield.

¹H NMR (300 MHz, DMSO-d₆): δ 12.54 (1H, bs), 7.96 (1H, s), 7.84 (1H, d, J=9.6 Hz), 7.83 (1H, t, J=4.7 Hz), 7.76 (2H, d, J=7.7 Hz), 7.68 (2H, d, J=7.7 Hz), 7.28 (2H, s), 6.87 (1H, d, J=9.6 Hz), 4.52 (2H, d, J=4.7 Hz).

LCMS: 348 [M+1], (MW: 347.35).

### Example 10

### 6-(3,4-Dichloro-benzylamino)-imidazo[1,2-b]pyridazine-3-carboxylic acid hydro-chloride salt

A mixture of 6-(3,4-dichloro-benzylamino)-imidazo[1,2-b]pyridazine-3-carboxylic acid ethyl ester (1.20 g, 5.45 mmol) and 12N hydrochloric acid (30 mL) in acetic acid (15 mL) was refluxed for 4 hours under nitrogen. Then, more hydrochloric acid (12N, 8 mL) was added and the mixture was refluxed 2 hours more. The solvent was removed in vacuo to afford a vitreous solid that was triturated from water to afford 847 mg of 6-(3,4-dichloro-benzylamino)-imidazo[1,2-b]pyridazine-3-carboxylic acid hydrochloride salt (70% yield) as a white solid.

¹H NMR (300 MHz, DMSO-d₆): δ 12.60 (1H, bs), 7.97 (1H, s), 7.85 (1H, d, J=9.7 Hz), 7.84 (1H, d, J=1.9 Hz), 7.82 (1H, t, J=5.9 Hz), 7.57 (1H, d, J=8.2 Hz), 7.49 (1H, dd, J=8.2, 1.9 Hz), 6.87 (1H, d, J=9.7 Hz), 4.44 (2H, d, J=5.9 Hz).

LCMS: 337 ([M+1]-35), (MW: 373.63).

### General Procedure C for the Preparation of Examples 11-16

A mixture of the appropriate acid (e.g. 6-(4-fluorobenzylamino)-imidazo[1,2-b]pyridazine-3-carboxylic acid) (1 eq), 1-hydroxybenzotriazole hydrate (2.6 eq), O-(1H-benzotriazol-1-yl)-N,N,N′,N′-tetramethyluronium hexafluorophosphate (2.2 eq) and triethylamine (3 eq) in dry N,N-dimethylformamide (8 mL/mmol) was stirred for 4 hours at 60° C. The appropriate amine (e.g. 4-methoxyaniline) (3 eq) was added and the mixture was stirred at 60° C. for 18 hours (or otherwise stated, depending upon the corresponding amine). The solvent was removed in vacuo and the residue was purified (using different purification methods) to give the desired product (e.g. 6-(4-fluorobenzylamino)-imidazo[1,2-b]pyridazine-3-carboxylic acid (4-methoxyphenyl)-amide).

### Example 11

### 6-(4-Fluoro-benzylamino)-imidazo[1,2-b]pyridazine-3-carboxylic acid (4-methoxy-phenyl)-amide

The title compound was obtained as a grey solid after the crude reaction mixture was treated with saturated aqueous solution of sodium hydrogen carbonate of (42% yield).

¹H NMR (300 MHz, DMSO-d₆): [symbol] 10.50 (1H, bs), 8.11 (1H, t, J=5.3 Hz), 8.03 (1H, s), 7.97 (1H, d, J=9.8 Hz), 7.46 (2H, dd, J=8.3, 5.6 Hz), 7.36 (2H, d, J=8.9 Hz), 7.18 (2H, t, J=8.8 Hz), 7.00 (1H, d, J=9.8 Hz), 6.90 (2H, d, J=8.9 Hz), 4.63 (2H, d, J=5.5 Hz), 3.75 (3H, s).

LCMS: 392 [M+1], tR=10.81 min, (MW: 391.41).

### Example 12

### 6-(4-Fluoro-benzylamino)-imidazo[1,2-b]pyridazine-3-carboxylic acid (3-methoxy-phenyl)-amide

The title compound was obtained as a brown solid after treatment with saturated aqueous solution of sodium hydrogen carbonate of the crude reaction mixture (27% yield).

¹H NMR (300 MHz, DMSO-d₆): [symbol] 10.70 (1H, s), 8.09 (1H, t, J=5.4 Hz), 8.05 (1H, s), 7.97 (1H, d, J=9.7 Hz), 7.48 (2H, dd, J=8.1, 5.7 Hz), 7.32 (1H, s), 7.22-7.14 (3H, m), 6.99 (1H, d, J=9.7 Hz), 6.89 (1H, d, J=8.1 Hz), 6.69 (1H, dd, J=8.1, 2.3 Hz), 4.63 (2H, d, J=5.3 Hz), 3.69 (3H, s).

LCMS: 392 [M+1], tR=11.34 min, (MW: 391.41).

### Example 13

### 4-{[6-(4-Methoxy-benzylamino)-imidazo[1,2-b]pyridazine-3-carbonyl]-amino}-benzoic acid ethyl ester

The title compound was obtained as a white solid after purification by column chromatography on flash silica gel (ethyl acetate and methanol) (27% yield).

¹H NMR (300 MHz, DMSO-d₆): [symbol] 10.93 (1H, s), 8.08 (2H, s), 7.96 (1H, d, J=9.8 Hz), 7.89 (2H, d, J=8.7 Hz), 7.56 (2H, d, J=8.7 Hz), 7.36 (2H, d, J=8.6 Hz), 7.01 (1H, d, J=9.8 Hz), 6.93 (2H, d, J=8.6 Hz), 4.59 (2H, bs), 4.29 (2H, d, J=7.1 Hz), 3.72 (3H, s), 1.29 (3H, d, J=7.1 Hz).

LCMS: 446 [M+1], tR=12.31 min, (MW: 445.48).

### Example 14

### 3-{[6-(4-Methoxy-benzylamino)-imidazo[1,2-b]pyridazine-3-carbonyl]-amino}-benzoic acid ethyl ester

The title compound was obtained as a white solid after purification by flash column chromatography on silica gel (ethyl acetate and ethyl acetate/methanol 10:0 to 9.5:0.5) (48% yield).

¹H NMR (300 MHz, DMSO-d₆): δ 10.90 (1H, s), 8.35 (1H, s), 8.06 (1H, s), 8.03 (1H, d, J=6.0 Hz), 7.96 (1H, d, J=9.7 Hz), 7.69 (1H, dd, J=6.6, 1.2 Hz), 7.64 (1H, d, J=8.9 Hz), 7.47 (1H, t, J=7.9 Hz), 7.37 (2H, d, J=8.5 Hz), 7.00 (1H, d, J=9.7 Hz), 6.90 (2H, d, J=8.5 Hz), 4.59 (2H, bs), 4.27 (2H, d, J=7.1 Hz), 3.71 (3H, s), 1.24 (3H, d, J=7.1 Hz).

LCMS: 446 [M+1], tR=12.08 min, (MW: 445.48).

### Example 15

### 6-[(Furan-2-ylmethyl)-amino]-imidazo[1,2-b]pyridazine-3-carboxylic acid (3,4-dimethoxy-phenyl)-amide

The title compound was obtained as a white solid after purification by reverse phase column chromatography (mixtures of water/acetonitrile) followed by trituration from water (14% yield).

¹H NMR (300 MHz, DMSO-d₆): [symbol] 10.72 (1H, s), 8.10 (1H, t, J=5.4 Hz), 8.04 (1H, s), 7.96 (1H, d, J=9.8 Hz), 7.59 (1H, d, J=0.7 Hz), 7.33 (1H, d, J=2.1 Hz), 7.17 (1H, dd, J=8.6, 2.2 Hz), 6.96 (1H, d, J=2.1 Hz), 6.94 (1H, d, J=9.8 Hz), 6.41-6.37 (2H, m), 4.66 (2H, d, J=5.4 Hz), 3.74 (3H, s), 3.68 (3H, s).

LCMS: 394 [M+1], tR=9.28 min, (MW: 393.41).

### Example 16

### 6-[(Furan-2-ylmethyl)-amino]-imidazo[1,2-b]pyridazine-3-carboxylic acid phenyl-amide

The title compound was obtained as a white solid after purification by flash column chromatography on silica gel (ethyl acetate/methanol 10:0 to 9.5:0.5) (7% yield).

¹H NMR (300 MHz, acetone-d₆): [symbol] 10.74 (1H, s), 8.11 (1H, s), 7.87 (1H, d, J=9.8 Hz), 7.70 (2H, d, J=8.5 Hz), 7.49 (1H, s), 7.33 (1H, d, J=2.1 Hz), 7.34 (2H, t, J=7.9 Hz), 7.11 (1H, d, J=7.4 Hz), 7.05 (1H, d, J=9.8 Hz), 6.39 (2H, dd, J=2.5, 12.2 Hz), 4.79 (2H, d, J=5.5 Hz).

LCMS: 334 [M+1], (MW: 333.35).

### General Procedure D for the Preparation of Examples 17-25

A mixture of 6-(3,4-dichloro-benzylamino)-imidazo[1,2-b]pyridazine-3-carboxylic acid hydrochloride salt (1 eq), 1-hydroxybenzotriazole hydrate (about 2.6 eq), O-(1H-benzotriazol-1-yl)-N,N,N′,N′-tetramethyluronium hexaflorophos-phate (about 2.2 eq) and triethylamine (about 4 eq) in dry N,N-dimethylformamide (10 mL/mmol) was stirred for several hours (from 1 to 4 hours) at 60° C. The appropriate amine (e.g. 5-aminoindole) (about 3 eq) was added and the mixture was stirred at 60° C. for 18 hours (or otherwise stated, depending upon the corresponding amine). The solvent was removed in vacuo and the residue was purified (using different purification methods) to give the desired product (e.g. 6-(3,4-dichloro-benzylamino)-imidazo[1,2-b]pyridazine-3-carboxylic acid (1H-indol-5-yl)-amide).

### Example 17

### 6-(3,4-Dichloro-benzylamino)-imidazo[1,2-b]pyridazine-3-carboxylic acid (1H-indol-5-yl)-amide

The title compound was obtained as a brown solid after purification by recrystallization from ethanol/water (82% yield).

¹H NMR (300 MHz, DMSO-d₆): δ 11.07 (1H, s), 10.39 (1H, s), 8.18 (1H, t, J=5.6 Hz), 8.03 (1H, s), 7.98 (1H, d, J=9.7 Hz), 7.70 (1H, d, J=1.7 Hz), 7.67, (1H, s), 7.58 (1H, d, J=8.3 Hz), 7.40 (1H, dd, J=8.3, 1.7 Hz), 7.34-7.33 (1H, m), 7.29 (1H, d, J=8.6 Hz), 7.00 (1H, d, J=9.7 Hz), 6.91 (1H, dd, J=8.6, 1.8 Hz), 6.37 (1H, s), 4.68 (2H, d, J=5.6 Hz).

LCMS: 451 [M+1], tR=10.92 min, (MW: 451.32).

### Example 18

### 6-(3,4-Dichloro-benzylamino)-imidazo[1,2-b]pyridazine-3-carboxylic acid phenyl-amide

The title compound was obtained as a white solid after purification by flash column chromatography on silica gel (dichloromethane/methanol 10:0 to 9.5:0.5) (46% yield).

¹H NMR (300 MHz, DMSO-d₆): δ 10.47 (1H, s), 8.17 (1H, t, J=5.6 Hz), 8.05 (1H, d, J=0.6 Hz), 7.99 (1H, d, J=10.05 Hz), 7.70 (1H, s), 7.58 (1H, d, J=8.2 Hz), 7.40-7.36 (3H, m), 7.30 (2H, t, J=7.7 Hz), 7.10 (1H, t, J=7.5 Hz), 7.01 (1H, d, J=10.0 Hz), 4.67 (2H, d, J=5.6 Hz).

LCMS: 412 [M+1], tR=12.59 min, (MW: 412.28).

### Example 19

### 6-(3,4-Dichloro-benzylamino)-imidazo[1,2-b]pyridazine-3-carboxylic acid (2-methoxy-ethyl)-amide

The title compound was obtained as a white solid (38% yield) after purification by reverse phase column chromatography (mixtures of acetonitrile/water) followed by trituration from acetonitrile.

¹H NMR (300 MHz, DMSO-d₆): δ 8.78 (1H, t, J=5.35 Hz), 8.05 (1H, t, J=5.6 Hz), 7.90 (1H, d, J=9.5 Hz), 7.89 (1H, s), 7.70 (1H, d, J=1.7 Hz), 7.63 (1H, d, J=8.3 Hz), 7.41 (1H, dd, J=8.3, 1.70 Hz), 6.90 (1H, d, J=9.5 Hz), 4.53 (2H, d, J=5.6 Hz), 3.49 (2H, dt, J=5.3, 5.1 Hz), 3.49 (2H, t, J=5.1 Hz), 3.22 (3H, s).

LCMS: 394 [M+1], tR=10.08 min, (MW 394.26).

### Example 20

### 6-(3,4-Dichloro-benzylamino)-imidazo[1,2-b]pyridazine-3-carboxylic acid (2-acetylamino-ethyl)-amide

The title compound was obtained as a white solid after purification by trituration from acetonitrile (28% yield).

¹H NMR (300 MHz, DMSO-d₆): δ 8.52 (1H, t, J=5.7 Hz), 8.08 (1H, t, J=5.7 Hz), 8.02 (1H, t, J=5.6 Hz), 7.90 (1H, d, J=8.1 Hz), 7.89 (1H, s), 7.65 (1H, d, J=8.1 Hz), 7.64 (1H, d, J=1.7 Hz), 7.41 (1H, dd, J=8.3, 1.7 Hz), 6.92 (1H, d, J=9.7 Hz), 4.54 (2H, d, J=5.7 Hz), 3.35 (2H, q, J=7.1 Hz), 3.16 (2H, q, J=6.1 Hz), 1.78 (3H, s)

LCMS: 421 [M+1], tR=8.13 min, (MW: 421.29).

### Example 21

### 4-{[6-(3,4-Dichloro-benzylamino)-imidazo[1,2-b]pyridazine-3-carbonyl]-amino}-benzoic acid ethyl ester

The title compound was obtained as a brown solid after purification by trituration from ethanol (60% yield).

¹H NMR (300 MHz, DMSO-d₆): δ 10.71 (1H, s), 8.18 (1H, t, J=5.5 Hz), 8.09 (1H, s), 7.99 (1H, d, J=9.7 Hz), 7.91 (2H, d, J=8.6 Hz), 7.72 (1H, d, J=1.3 Hz), 7.59 (1H, d, J=8.2 Hz), 7.54 (2H, d, J=8.6 Hz), 7.40 (1H, dd, J=8.2, 1.3 Hz), 7.02 (1H, d, J=9.7 Hz), 4.68 (2H, d, J=5.5 Hz), 4.30 (2H, q, J=7.1 Hz), 1.32 (3H, t, 7.15 Hz).

LCMS: 484 [M+1], tR=13.46 min, (MW: 484.35).

### Example 22

### 6-(3,4-Dichloro-benzylamino)-imidazo[1,2-b]pyridazine-3-carboxylic acid (3-acetylphenyl)amide

The title compound was obtained as a brown solid after purification by trituration from acetonitrile/water (72% yield).

¹H NMR (300 MHz, DMSO-d₆): δ 10.52 (1H, s), 8.09 (1H, s), 8.01 (1H, t, J=5.4 Hz), 7.92 (1H, s), 7.84 (1H, d, J=9.80 Hz), 7.82 (1H, d, J=8.4 Hz), 7.58-7.53 (2H, m), 7.45-7.23 (3H, m), 6.85 (1H, d, J=9.8 Hz), 4.55 (2H, d, J=5.4 Hz), 2.40 (3H, s).

LCMS: 454 [M+1], tR=11.96 min, (MW: 454.32).

### Example 23

### 6-(3,4-Dichloro-benzylamino)-imidazo[1,2-b]pyridazine-3-carboxylic acid pyridin-3-yl-amide

The title compound was obtained as a white solid (10% yield) after purification by flash column chromatography on silica gel (ethyl acetate/methanol 10:0 to 9.5:0.5) followed by semi-preparative high pressure liquid chromatography (RP-C18 Gemini; 150×10 mm, 5 um; 30-70% B in 10 min, flow rate 6 mL/min; B: acetonitrile+0.1% formic acid; A: water+0.1% formic acid).

¹H NMR (300 MHz, acetone-d₆): δ 10.59 (1H, s), 8.71 (1H, d, J=2.5 Hz), 8.32 (1H, dd, J=4.7, 1.3 Hz), 8.14 (1H, s), 8.11 (1H, s), 8.00 (1H, dd, J=5.3, 2.9 Hz), 7.91 (1H, d, J=9.7 Hz), 7.73 (1H, s), 7.56-7.49 (1H, m), 7.43 (1H, bs), 7.33 (1H, dd, J=8.2, 4.8 Hz), 7.12 (1H, d, J=9.8 Hz), 4.89 (2H, d, J=5.2 Hz).

LCMS: 413 [M+1], tR=8.90 min, (MW: 413.37).

### Example 24

### 6-(3,4-Dichloro-benzylamino)-imidazo[1,2-b]pyridazine-3-carboxylic acid (1-methyl-1H-pyrazol-3-yl)-amide

The title compound was obtained as a white solid (4% yield) after purification by flash column chromatography on silica gel (ethyl acetate/methanol 10:0 to 9.5:0.5) followed by semi-preparative high pressure liquid chromatography (RP-C18 Gemini; 150×10 mm, 5 um; 30-70% B in 10 min, flow rate 6 mL/min; B: acetonitrile+0.1% formic acid; A: water+0.1% formic acid).

¹H NMR (300 MHz, Acetone-d₆): δ 7.98 (1H, s), 7.70 (1H, d, J=9.8 Hz), 7.67 (1H, d, J=1.5 Hz), 7.41 (1H, d, J=2.3 Hz), 7.31-7.27 (2H, m), 6.87 (1H, d, J=9.8 Hz), 6.51 (1H, d, J=2.3 Hz), 4.55 (2H, s), 3.77 (3H, s).

LCMS: 416 [M+1], tR=10.67 min, (MW: 416.27).

### Example 25

### 6-(3,4-Dichloro-benzylamino)-imidazo[1,2-b]pyridazine-3-carboxylic acid ethylamide

The title compound was prepared from 6-(3,4-dichloro-benzylamino)-imidazo[1,2-b]pyridazine-3-carboxylic acid hydrochloride salt and ethylamine (2M solution in tetrahydrofuran) (6 eq) as a brown solid after stirring the reaction mixture for 18 hours at room temperature followed by trituration from acetonitrile/water (78% yield).

¹H NMR (300 MHz, DMSO-d₆): δ 8.34 (1H, t, J=4.7 Hz), 8.20 (1H, t, J=5.7 Hz), 7.98 (1H, s), 7.94 (1H, d, J=8.9 Hz), 7.67 (1H, d, J=0.9 Hz), 7.63 (1H, d, J=8.9 Hz), 7.37 (1H, dd, J=8.3, 0.9 Hz), 7.01 (1H, d, J=9.8 Hz), 4.56 (2H, d, J=5.5 Hz), 3.25 (2H, p, J=7.1 Hz), 0.98 (3H, t, J=7.1 Hz).

LCMS: 364 [M+1], tR=9.94 min, (MW: 364.24).

### Example 26

### [6-(3,4-Dichloro-benzylamino)-imidazo[1,2-b]pyridazin-3-yl]-morpholin-4-yl-methanone

A mixture of 6-(3,4-dichloro-benzylamino)-imidazo[1,2-b]pyridazine-3-carboxylic acid hydrochloride salt (50 mg, 13 mmol), 1-hydroxybenzotriazole hydrate (43 mg, 0.29 mmol), N-(3-Dimethylaminopropyl)-N′-ethylcarbodiimide hydrochloride (57 mg, 0.29 mmol) and triethylamine (0.055 mL, 0.39 mmol) in dry N,N-dimethylformamide (1 mL) was stirred for 1 hour at room temperature. Morpholine (0.034 mL, 0.39 mmol) was added and the mixture was stirred at room temperature for 18 hours. The solvent was removed in vacuo and the crude product was purified by flash column chromatography on silicagel (dichloromethane/methanol 9.9:0.1 to 9.5:0.5) to afford a white solid that was suspended in a saturated aqueous solution of potassium carbonate (2 mL). The mixture was stirred for 3 hours at room temperature and the resulting white solid was filtered off, washed with water and dried to give 33 mg of [6-(3,4-dichloro-benzylamino)-imidazo[1,2-b]pyridazin-3-yl]-morpholin-4-yl-methanone (61% yield).

¹H NMR (300 MHz, DMSO-d₆): δ 7.80 (1H, d, J=9.7 Hz), 7.72 (1H, t, J=5.7 Hz), 7.64 (1H, d, J=1.9 Hz), 7.58 (1H, s), 7.58 (1H, d, J=8.2 Hz), 7.39 (1H, dd, J=8.2, 1.9 Hz), 6.80 (1H, d, J=9.7 Hz), 4.43 (2H, d, J=5.7 Hz), 3.49 (4H, bs).

LCMS: 406 [M+1], tR=8.13 min, (MW: 406.27).

### Example 27

### 6-(4-Sulfamoyl-benzylamino-imidazo[1,2-b]pyridazine-3-carboxylic acid (6-methoxy-pyridin-3-yl)-amide

A mixture of 6-(4-sulfamoyl-benzylamino)-imidazo[1,2-b]pyridazine-3-carboxylic acid (50 mg, 0.14 mmol), 1-hydroxybenzotriazole hydrate (79 mg, 0.43 mmol), O-(1H-benzotriazol-1-yl)-N,N,N′,N′-tetramethyluronium hexafluorophosphate (16 mg, 0.43 mmol) and triethylamine (0.10 mL, 0.72 mmol) in dry N,N-dimethylformamide (2 mL) was stirred for 4 hours at 60° C. 5-Amino-2-methoxypyridine (54 mg, 0.43 mmol) was added and the mixture was stirred for 18 hours at 60° C. The solvent was removed in vacuo, saturated aqueous solution of sodium hydrogen carbonate (2 mL) was added, the mixture was stirred for 1 hour at 0° C. and the resulting red solid was filtered off, washed with water and dried to give 59 mg of 6-(4-sulfamoyl-benzylamino-imidazo[1,2-b]pyridazine-3-carboxylic acid (6-methoxy-pyridin-3-yl)-amide (90% yield).

¹H NMR (300 MHz, DMSO-d₆): [symbol] 10.41 (1H, s), 8.35 (1H, d, J=2.5 Hz), 8.18 (1H, t, J=5.4 Hz), 8.04 (1H, s), 7.98 (1H, d, J=9.8 Hz), 7.75 (2H, d, J=7.7 Hz), 7.61 (1H, dd, J=8.8, 2.5 Hz), 7.56 (2H, d, J=8.1 Hz), 7.31 (2H, s), 7.00 (1H, d, J=9.8 Hz), 6.82 (1H, d, J=8.8 Hzr), 4.70 (2H, d, J=5.4 Hz), 3.84 (3H, s).

LCMS: 454 [M+1], tR=7.84 min, (MW: 453.48).

### Example 28

### 6-(4-Sulfamoyl-benzylamino-imidazo[1,2-b]pyridazine-3-carboxylic acid phenyl-amide

A mixture of 6-(4-sulfamoyl-benzylamino)-imidazo[1,2-b]pyridazine-3-carboxylic acid (50 mg, 0.14 mmol), 1-hydroxybenzotriazole hydrate (79 mg, 0.43 mmol), O-(1H-benzotriazol-1-yl)-N,N,N′,N′-tetramethyluronium hexafluorophosphate (16 mg, 0.43 mmol) and triethylamine (0.10 mL, 0.72 mmol) in dry N,N-dimethylformamide (2 mL) was stirred for 4 hours at 60° C. Aniline (0.040 mL, 0.43 mmol) was added and the mixture was stirred for 18 hours at 60° C. The solvent was removed in vacuo and the residue was purified by column chromatography on silica gel (ethyl acetate/methanol 10:0 to 9.5:0.5) to give 11 mg of 6-(4-sulfamoyl-benzylamino-imidazo[1,2-b]pyridazine-3-carboxylic acid phenyl-amide as a white solid (19% yield).

¹H NMR (300 MHz, DMSO-d₆): [symbol] 10.47 (1H, s), 8.14 (1H, t, J=5.1 Hz), 7.99 (1H, s), 7.93 (1H, d, J=10.0 Hz), 7.73 (2H, d, J=8.1 Hz), 7.53 (2H, d, J=8.1 Hz), 7.29-7.22 (6H, m), 7.03 (1H, d, J=7.0 Hz), 6.96 (1H, d, J=10.0 Hz), 4.66 (2H, s).

LCMS: 423 [M+1], tR=8.48 min, (MW: 422.47).

### General Procedure E for the Preparation of Examples 29-31

A mixture of the appropriate carboxylic acid ethyl ester derivative (e.g. 4-{[6-(3,4-dichloro-benzylamino)-imidazo[1,2-b]pyridazine-3-carbonyl]-amino}-benzoic acid ethyl ester) (1 eq) in ethanol (about 20 mL/mmol) and aqueous 4N potassium hydroxide (about 20 mL/mmol) was stirred at room temperature for 24 hours. The ethanol was removed in vacuo, the resulting mixture was cooled at 0° C. and acetic acid was added dropwise up to pH 5. The resulting solid was filtered off, washed with water and dried to afford the desired acid (e.g. 4-{[6-(3,4-dichloro-benzylamino)-imidazo[1,2-b]pyridazine-3-carbonyl]amino}-benzoic acid).

### Example 29

### 4-{[6-(3,4-Dichloro-benzylamino)-imidazo[1,2-b]pyridazine-3-carbonyl]-amino}-benzoic acid

The title compound was obtained as a brown solid (93% yield).

¹H NMR (300 MHz, DMSO-d₆): δ ppm: 12.79 (1H, bs), 10.66 (1H, s), 8.19 (1H, t, J=5.3 Hz), 8.08 (1H, s), 7.99 (1H, d, J=9.7 Hz), 7.88 (2H, d, J=8.5 Hz), 7.71 (1H, s), 7.60 (1H, d, J=8.2 Hz), 7.49 (2H, d, J=8.5 Hz), 7.40 (1H, d, J=8.25 Hz), 7.02 (1H, d, J=9.7 Hz), 4.68 (2H, d, J=5.3 Hz).

LCMS: 456 [M+1], tR=10.31 min, (MW: 456.29).

### Example 30

### 4-({[6-(4-Methoxy-benzylamino)-imidazo[1,2-b]pyridazine-3-carbonyl]-amino}-benzoic acid

The title compound was obtained as a white solid (93% yield).

¹H NMR (300 MHz, DMSO-d₆): δ ppm: 12.78 (1H, bs), 10.89 (1H, s), 8.08 (2H, s), 7.97 (1H, d, J=9.8 Hz), 7.87 (2H, d, J=8.5 Hz), 7.53 (2H, d, J=8.5 Hz), 7.36 (2H, d, J=8.5 Hz), 7.01 (1H, d, J=9.8 Hz), 6.93 (2H, d, J=8.5 Hz), 4.59 (2H, d, J=5.2 Hz), 3.71 (3H, s).

LCMS: 418 [M+1], tR=9.30 min, (MW: 417.43).

### Example 31

### 3-{[6-(4-Methoxy-benzylamino)-imidazo[1,2-b]pyridazine-3-carbonyl]-amino}-benzoic acid

The title compound was obtained as a white solid (93% yield).

¹H NMR (300 MHz, DMSO-d₆): δ ppm: 13.04 (1H, bs), 10.84 (1H, s), 8.35 (1H, s), 8.06 (1H, s), 8.04 (1H, t, J=5.3 Hz), 7.96 (1H, d, J=9.8 Hz), 7.68 (1H, d, J=7.6 Hz), 7.56 (1H, d, J=7.9 Hz), 7.43 (1H, t, J=7.9 Hz), 7.36 (2H, d, J=8.5 Hz), 7.01 (1H, d, J=9.8 Hz), 6.89 (2H, d, J=8.5 Hz), 4.59 (2H, d, J=4.8 Hz), 3.70 (3H, s).

LCMS: 418 [M+1], tR=9.27 min, (MW: 417.43).

### General Procedure F for the Preparation of Examples 32-33

A mixture of the appropriate methoxyaryl derivative (e.g. 6-(4-fluoro-benzylamino)-imidazo[1,2-b]pyridazine-3-carboxylic acid (4-methoxy-phenyl)-amide) (1 eq) in dry dichloromethane (about 10 mL/mmol) and boron tribromide (1M solution in dichloromethane) (10 eq) was stirred at room temperature for 18 hours. The resulting mixture was cooled at 0° C. and methanol (10 mL/mmol) was added, the mixture was stirred at room temperature for 1 hour and the solvent was removed in vacuo. The resulting residue was dissolved in methanol (50 mL/mmol) the mixture was stirred at room temperature for 1 hour and the solvent was removed in vacuo. The residue was suspended in water (10 mL/mmol) and 28% aqueous ammonium hydroxide was added up to pH 11 The resulting solid was filtered off, washed with water and dried to afford the desired phenol derivative (e.g. 6-(4-fluoro-benzylamino)-imidazo[1,2-b]pyridazine-3-carboxylic acid (4-hydroxy-phenyl)-amide).

### Example 32

### 6-(4-Fluoro-benzylamino)-imidazo[1,2-b]pyridazine-3-carboxylic acid (4-hydroxy-phenyl)-amide

The title compound was obtained as a white solid (85% yield).

¹H NMR (300 MHz, DMSO-d₆): 6 ppm: 10.39 (1H, s), 9.30 (1H, s), 8.09 (1H, t, J=5.6 Hz), 7.99 (1H, s), 7.95 (1H, d, J=9.8 Hz), 7.44 (2H, dd, J=8.4, 5.6 Hz), 7.19 (2H, d, J=8.6 Hz), 7.15 (2H, t, J=8.8 Hz), 6.98 (1H, d, J=9.8 Hz), 6.69 (2H, d, J=8.6 Hz), 4.60 (2H, d, J=4.5 Hz).

LCMS: 378 [M+1], tR=8.76 min, (MW: 377.38).

### Example 33

### 6-(4-Fluoro-benzylamino)-imidazo[1,2-b]pyridazine-3-carboxylic acid (3-hydroxy-phenyl)-amide

The title compound was obtained as a brown solid (30% yield).

¹H NMR (300 MHz, methanol-d₄): δ ppm: 8.09 (s, 1H), 7.82 (1H, d, J=9.8 Hz), 7.44 (2H, dd, J=8.5, 5.4 Hz), 7.26 (1H, t, J=2.2 Hz), 7.11-7.01 (3H, m), 6.99 (1H, d, J=9.8 Hz), 6.89 (1H, dd, J=7.7, 1.5 Hz), 6.58 (1H, dd, J=8.1, 2.2 Hz), 4.68 (2H, s).

LCMS: 378 [M+1], tR=9.54 min, (MW: 377.38).

### Intermediate 3

### 6-Chloro-imidazo[1,2-b]pyridazine-3-carboxylic acid phenylamide

A 2M solution of trimethylaluminum (2.31 mL, 4.63 mmol) in hexanes was added dropwise to a solution of aniline (0.42 mL, 4.63 mmol) in dry dichloromethane (40 mL) at room temperature. The mixture was stirred for 2 hours at room temperature and then 6-chloro-imidazo[1,2-b]pyridazine-3-carboxylic acid ethyl ester (0.475 g, 2.10 mmol) was added and the mixture was refluxed for 18 hours under nitrogen. On cooling, the reaction was quenched with 0.5N aqueous solution of hydrochloric acid (5 mL) and extracted with dichloromethane (4×250 mL). The combined organic fractions were dried (magnesium sulphate), the solvent removed in vacuo and the residue was recrystallized from ethyl acetate to give 0.506 g of 6-chloro-imidazo[1,2-b]pyridazine-3-carboxylic acid phenylamide as a yellow solid (88% yield).

¹H NMR (300 MHz, CDCl₃): δ 10.14 (1H, s), 8.63 (1H, s), 8.17 (1H, d, J=9.5 Hz), 7.77 (2H, d, J=7.8 Hz), 7.43 (2H, t, J=7.8 Hz), 7.33 (1H, d, J=9.5 Hz), 7.21 (1H, t, J=7.4 Hz).

LCMS: 273 [M+1], (MW: 272.70).

### General Procedure G for the Preparation of Examples 34-36

A mixture of the appropriate alcohol (e.g. benzyl alcohol) (2.2 eq) and sodium hydride (60% in mineral oil) (2.6 eq) in dry 1,4-dioxane (about 8 mL/mmol) was stirred for 30 minutes at room temperature. Then, 6-chloro-imidazo[1,2-b]pyridazine-3-carboxylic acid phenylamide (1 eq) was added portionwise and the mixture was refluxed (or otherwise stated, depending upon the corresponding alcohol) for several hours (from 6 to 18 hours, depending upon the corresponding alcohol). The solvent was removed in vacuo and the resulting solid was purified by recrystallization (hexane/ethyl acetate mixtures) to give the desired product (e.g. 6-benzyloxy-imidazo[1,2-b]pyridazine-3-carboxylic acid phenylamide).

### Example 34

### 6-Benzyloxy-imidazo[1,2-b]pyridazine-3-carboxylic acid phenylamide

The title compound was obtained as a yellow solid after stirring the reaction mixture for 18 hours at room temperature (71% yield).

¹H NMR (300 MHz, DMSO-d₆): δ 10.30 (1H, s), 8.28 (1H, d, J=9.7 Hz), 8.27 (1H, s,), 7.71 (2H, d, J=7.8 Hz), 7.56 (2H, d, J=6.3 Hz), 7.44-7.35 (5H, m), 7.22 (1H, d, J=9.7 Hz), 7.13 (1H, t, J=7.3 Hz), 5.61 (2H, s).

LCMS: 345 [M+1], tR=12.71 min, (MW: 344.37).

### Example 35

### 6-Phenoxy-imidazo[1,2-b]pyridazine-3-carboxylic acid phenylamide

The title compound was obtained as a yellow solid after refluxing the reaction mixture for 6 hours (66% yield).

¹H NMR (300 MHz, DMSO-d₆): δ 9.72 (1H, s), 8.44 (1H, d, J=9.7 Hz), 8.29 (1H, s), 7.61-7.59 (1H, m), 7.50-7.45 (5H, m), 7.26 (2H, t, J=7.8 Hz), 7.07 (1H, t, J=7.4 Hz), 6.94 (2H, d, J=7.7 Hz).

LCMS: 331 [M+1], tR=12.39 min, (MW: 330.35).

### Example 36

### 6-(4-Fluoro-phenoxy-imidazo[1,2-b]pyridazine-3-carboxylic acid phenyl-amide

The title compound was obtained as a yellow solid after refluxing the reaction mixture for 18 hours (79% yield).

¹H NMR (300 MHz, DMSO-d₆): δ 9.67 (1H, s), 8.30 (1H, d, J=9.7 Hz), 8.26 (1H, s), 7.58 (2H, dd, J=9.2, 4.5 Hz), 7.41 (1H, d, J=9.7 Hz), 7.41 (2H, t, J=8.7 Hz), 7.27 (2H, t, J=7.7 Hz), 7.10-7.06 (3H, m).

LCMS: 349 [M+1], tR=12.30 min, (MW: 348.34).

### General Procedure H for the Preparation of Examples 37-38

A mixture of the appropriate heterocycle (e.g. pyrrole) (2.2 eq) and sodium hydride (60% in mineral oil) (2.6 eq) in dry 1,4-dioxane (about 10 mL/mmol) was stirred for 30 minutes at room temperature. Then, 6-chloro-imidazo[1,2-b]pyridazine-3-carboxylic acid phenylamide (1 eq) was added portionwise and the mixture was refluxed for about 6 hours. On cooling at 0° C., water was added (30 mL/mmol), the mixture was stirred for 30 minutes at room temperature and the resulting solid was filtered off, washed with water and dried to afford the desired product (e.g. 6-pyrrol-1-yl-imidazo[1,2-b]pyridazine-3-carboxylic acid phenylamide) after using different purification methods.

### Example 37

### 6-Pyrrol-1-yl-imidazo[1,2-b]pyridazine-3-carboxylic acid phenylamide

The title compound was obtained as a yellow solid after purification by flash column chromatography on silica gel (dichloromethane/methanol 10:0 to 9.9:0.1) (61% yield).

¹H NMR (300 MHz, DMSO-d₆): δ 10.38 (1H, s), 8.50 (1H, d, J=9.8 Hz), 8.39 (1H, s), 8.00 (1H, d, J=9.8 Hz), 7.78-7.62 (5H, m), 7.42 (2H, t, J=7.8 Hz), 6.48 (2H, t, J=1.9 Hz).

LCMS: 304 [M+1], tR=11.83 min, (MW: 303.33).

### Example 38

### 6-Imidazol-1-yl-imidazo[1,2-b]pyridazine-3-carboxylic acid phenylamide

The title compound was obtained as a yellow solid after washing the resulting solid with hexanes (58% yield).

¹H NMR (300 MHz, DMSO-d₆): δ 10.31 (1H, s), 8.71 (1H, s), 8.63 (1H, d, J=9.7 Hz), 8.48 (1H, s), 8.09-8.05 (2H, m), 7.81 (2H, d, J=8.1 Hz), 7.45 (2H, t, J=6.9 Hz), 7.30 (1H, s), 7.19 (1H, t, J=6.9 Hz).

LCMS: 305 [M+1], tR=7.35 min, (MW: 304.31).

### General Procedure I for the Preparation of Examples 39-41

A mixture of the appropriate arylamine (e.g. aniline) (2.2 eq) and sodium hydride (60% in mineral oil) (2.6 eq) in dry 1,4-dioxane (about 10 mL/mmol) was stirred for 30 minutes at room temperature. Then, 6-chloro-imidazo[1,2-b]pyridazine-3-carboxylic acid phenylamide (1 eq) was added portionwise and the mixture was refluxed for 18 hours. On cooling, the solvent was removed in vacuo and the residue was purified by column chromatography on flash silica gel (ethyl acetate/ethanol 10:0.1 to 10:0.5) followed by recrystallization from ethyl acetate to give the desired product (e.g. 6-phenylamino-imidazo[1,2-b]pyridazine-3-carboxylic acid phenylamide).

### Example 39

### 6-Phenylamino-imidazo[1,2-b]pyridazine-3-carboxylic acid phenylamide

The title compound was obtained as a white solid (16% yield).

¹H NMR (300 MHz, DMSO-d₆): δ 10.39 (1H, s), 9.67 (1H, s), 8.12 (1H, s), 8.08 (1H, d, J=9.7 Hz), 7.63 (2H, d, J=7.7 Hz), 7.46 (2H, d, J=7.6 Hz), 7.37-7.30 (4H, m), 7.14 (1H, t, J=5.9 Hz), 7.10 (2H, d, J=9.7 Hz).

LCMS: 330 [M+1], tR=11.37 min, (MW: 329.36).

### Example 40

### 6-(4-Methoxy-phenylamino)-imidazo[1,2-b]pyridazine-3-carboxylic acid phenyl-amide

The title compound was obtained as a brown solid (30% yield).

¹H NMR (300 MHz, DMSO-d₆): δ 10.45 (1H, s), 9.44 (1H, s), 8.08 (1H, s), 8.04 (1H, d, J=9.7 Hz), 7.48 (2H, d, J=8.9 Hz), 7.30-7.25 (4H, m), 7.11 (1H, t, J=7.3 Hz), 7.04 (1H, d, J=9.7 Hz), 7.48 (2H, d, J=8.9 Hz), 3.76 (3H, s).

LCMS: 360 [M+1], tR=11.04 min, (MW: 359.39).

### Example 41

### 6-(Pyridin-3-ylamino)-imidazo[1,2-b]pyridazine-3-carboxylic acid phenyl-amide

The title compound was obtained as a brown solid (34% yield).

¹H NMR (300 MHz, DMSO-d₆): δ 10.05 (1H, s), 9.65 (1H, s), 8.67 (1H, d, J=2.5 Hz), 8.11 (1H, dd, J=4.6, 1.2 Hz), 7.99 (1H, dd, J=8.2, 2.5 Hz), 7.96 (1H, s), 7.92 (1H, d, J=9.8 Hz), 7.34 (2H, d, J=7.7 Hz), 7.15 (2H, t, J=7.7 Hz), 7.12 (1H, dd, J=8.2, 1.5 Hz), 6.92 (1H, d, J=9.8 Hz), 6.91 (1H, t, J=7.5 Hz).

LCMS: 331 [M+1], tR=7.04 min, (MW: 330.35).

### General Procedure J for the Preparation of Examples 42-44

A mixture of the appropriate amine (e.g. (3,4-dichlorobenzyl)methylamine) (2.5 eq) and 6-chloro-imidazo[1,2-b]pyridazine-3-carboxylic acid phenylamide (1 eq) in dry 1,4-dioxane (about 3 mL/mmol) was heated at 160° C. for about 16 hours under microwave irradiation. On cooling, the solvent was removed in vacuo and the residue was purified (using different purification methods) to give the desired product (e.g. 6-[(3,4-dichloro-benzyl)-methyl-amino]-imidazo[1,2-b]pyridazine-3-carboxylic acid phenylamide).

### Example 42

### 6-[(3,4-Dichloro-benzyl)-methyl-amino]-imidazo[1,2-b]pyridazine-3-carboxylic acid phenylamide

The title compound was obtained as a white solid after purification by flash column chromatography on silica gel (dichloromethane/methanol 10:0 to 9.9:0.1) followed by recrystallization of the resulting solid from diethyl ether/ethyl acetate (61% yield).

¹H NMR (300 MHz, DMSO-d₆): δ 10.58 (1H, s), 8.14 (1H, s), 8.08 (1H, d, J=10.0 Hz), 7.63 (1H, d, J=1.9 Hz), 7.60 (1H, d, J=8.3 Hz), 7.51 (1H, d, J=7.8 Hz), 7.35-7.28 (4H, m), 7.11 (1H, t, J=7.3 Hz), 7.01 (1H, d, J=10.0 Hz), 4.92 (2H, s), 3.31 (3H, s).

LCMS: 426 [M+1], tR=13.59 min, (MW: 426.31).

### Example 43

### 6-(3,4-Dihydro-1H-isoquinolin-2-yl)-imidazo[1,2-b]pyridazine-3-carboxylic acid phenylamide

The title compound was obtained as a white solid after purification by recrystallization from ethyl acetate (99% yield).

¹H NMR (300 MHz, DMSO-d₆): 6 ppm: 10.80 (1H, s), 8.14 (1H, s), 8.12 (1H, d, J=10.1 Hz), 7.80 (2H, d, J=7.8 Hz), 7.54 (1H, d, J=10.1 Hz), 7.45 (2H, t, J=7.8 Hz), 7.29-7.22 (4H, m), 7.11 (1H, t, J=7.4 Hz), 4.88 (2H, s), 3.92 (2H, t, J=5.8 Hz), 3.05 (2H, t, J=5.8 Hz).

LCMS: 370 [M+1], tR=13.03 min, (MW: 369.43).

### Example 44

### 6-(1,3-Dihydro-isoindol-2-yl)-imidazo[1,2-b]pyridazine-3-carboxylic acid phenylamide

The title compound was obtained as a white solid after purification by flash column chromatography on silica gel (ethyl acetate) (78% yield).

¹H NMR (300 MHz, DMSO-d₆): δ 10.92 (1H, s), 8.14 (1H, d, J=8.8 Hz), 8.13 (1H, s), 7.80 (2H, d, J=7.9 Hz), 7.48-7.43 (4H, m), 7.39-7.36 (2H, m), 7.23-7.14 (2H, m), 5.01 (4H, s).

LCMS: 356 [M+1], tR=12.81 min, (MW: 355.40).

### General Procedure K for the Preparation of Examples 45-48

A mixture of 6-chloro-imidazo[1,2-b]pyridazine-3-carboxylic acid phenylamide (1 eq), the appropriate boronic acid (e.g. phenylboronic acid) (2.5 eq), palladium (II) acetate (0.1 eq), 1,1′-bis(diphenylphosphino)ferrocene (0.2 eq), potassium carbonate (5 eq) and water (about 3 mL/mmol) in degassed N,N-dimethylformamide (about 30 mL/mmol) was heated at 100° C. for 6 hours. On cooling, the solvent was removed in vacuo and the residue was purified (using different purification methods) to give the desired product (e.g. 6-phenyl-imidazo[1,2-b]pyridazine-3-carboxylic acid phenylamide).

### Example 45

### 6-Phenyl-imidazo[1,2-b]pyridazine-3-carboxylic acid phenylamide

The title compound was obtained as a white solid after purification by flash column chromatography on silica gel (hexane/ethyl acetate 7:3) followed by recrystallization from ethyl acetate (22% yield).

¹H NMR (300 MHz, DMSO-d₆): δ 10.68 (1H, s), 8.48 (1H, d, J=9.6 Hz), 8.46 (1H, s), 8.19 (2H, dd, J=8.0 Hz, 1.50), 8.08 (1H, d, J=9.6 Hz), 7.77 (2H, d, J=8.6 Hz), 7.69-7.59 (3H, m), 7.43 (2H, t, J=7.9 Hz), 7.16 (1H, t, J=7.4 Hz).

LCMS: 315 [M+1], tR=12.72 min, (MW: 314.35).

### Example 46

### 6-(4-Methoxy-phenyl)-imidazo[1,2-b]pyridazine-3-carboxylic acid phenyl-amide

The title compound was obtained as a white solid after purification by flash column chromatography on silica gel (dichloromethane/methanol 10:0 to 9.5:0.5) (56% yield).

¹H NMR (300 MHz, DMSO-d₆): δ 10.69 (1H, s), 8.42 (1H, d, J=9.6 Hz), 8.42 (1H, s), 8.15 (2H, d, J=8.9 Hz), 8.04 (1H, d, J=9.6 Hz), 7.77 (2H, d, J=8.5 Hz), 7.44 (2H, t, J=7.9 Hz), 7.22 (2H, d, J=8.9 Hz), 7.17 (1H, t, J=7.3 Hz), 3.88 (3H, s).

LCMS: 345 [M+1], tR=12.74 min, (MW: 344.38).

### Example 47

### 6-Furan-3-yl-imidazo[1,2-b]pyridazine-3-carboxylic acid phenylamide

The title compound was obtained as a brown solid after purification by flash column chromatography on silica gel (ethyl acetate/ethanol 10:0 to 9:1) followed by recrystallization from ethyl acetate (39% yield).

¹H NMR (300 MHz, DMSO-d₆): δ 10.53 (1H, s), 8.73 (1H, s), 8.43 (1H, d, J=9.5 Hz), 8.40 (1H, s), 7.97 (1H, t, J=1.6 Hz), 7.92 (1H, d, J=9.5 Hz), 7.79 (2H, d, J=7.7 Hz), 7.44 (2H, t, J=7.7 Hz), 7.17 (1H, t, J=7.4 Hz), 7.16 (1H, d, J=1.8 Hz).

LCMS: 305 [M+1], tR=11.63 min, (MW: 304.31).

### Example 48

### 6-(1H-Indol-5-yl)-imidazo[1,2-b]pyridazine-3-carboxylic acid phenylamide

The title compound was obtained as a white solid after purification by flash column chromatography on silica gel (dichloromethane/methanol 10:0 to 9:1) (38% yield).

¹H NMR (300 MHz, DMSO-d₆): δ 11.45 (1H, s), 10.89 (1H, s), 8.42 (1H, d, J=9.6 Hz), 8.41 (2H, bs), 8.13 (1H, d, J=9.6 Hz), 7.94 (1H, d, J=8.5 Hz), 7.80 (2H, d, J=8.0 Hz), 7.66 (1H, d, J=8.5 Hz), 7.50 (1H, s), 7.45 (2H, t, J=7.8 Hz), 7.17 (1H, t, J=7.2 Hz), 6.63 (1H, s).

LCMS: 354 [M+1], tR=11.99 min, (MW: 353.39).

### Intermediate 4

### 6-chloro-imidazo[1,2-b]pyridazine-3-carbonitrile

N,N-Dimethyl-N′-(pyridazinyl-3)-formamidine (1.00 g, 5.41 mmol) was dissolved in acetonitrile (15 mL) and bromoacetonitrile (1.13 mL, 16.25 mmol) was added. The reaction was stirred overnight at reflux temperature. The solvent was removed in vacuo, the residue was dissolved in acetonitrile (15 mL) and diisopropylethylamine (6.0 mL, 35.60 mmol) was added. The mixture was stirred for 4 hours at room temperature and the solvent was removed in vacuo to give a residue that was purified by flash column chromatography on silica gel (ethyl acetate) to afford 0.75 g of 6-chloro-imidazo[1,2-b]pyridazine-3-carbonitrile as a yellow solid (71% yield).

¹H NMR (300 MHz, CDCl₃): δ 8.23 (1H, s), 8.02 (1H, d, J=9.5 Hz), 7.31 (1H, d, J=9.5 Hz).

LCMS: 179 [M+1], (MW: 178.58).

### Intermediate 5

### 6-(3,4-Dichloro-benzylamino)-imidazo[1,2-b]pyridazine-3-carbonitrile

A mixture of 6-chloro-imidazo[1,2-b]pyridazine-3-carbonitrile (0.35 g, 1.96 mmol) and 3,4-dichlorobenzylamine (0.57 mL, 0.759 mmol) in 1,4-dioxane (3 mL) was heated at 160° C. for 14 hours under microwave irradiation. On cooling, the solvent was removed in vacuo, aqueous saturated solution of sodium hydrogen carbonate (15 mL) was added and the mixture was extracted with ethyl acetate (4×50 mL). The combined organic fractions were dried (sodium sulphate), the solvent removed in vacuo and the residue was purified by flash column chromatography on silica gel (ethyl acetate) to give 0.352 g of 6-(3,4-dichloro-benzylamino)-imidazo[1,2-b]pyridazine-3-carbonitrile as a yellow solid (55% yield).

¹H NMR (300 MHz, CDCl₃): δ 7.94 (1H, s), 7.71 (1H, d, J=9.7 Hz), 7.50 (1H, d, J=1.8 Hz), 7.43 (1H, d, J=8.2 Hz), 7.29 (1H, dd, J=8.2, 1.8 Hz), 6.65 (1H, d, J=9.7 Hz), 4.96 (1H, t, J=4.5 Hz), 4.54 (2H, d, J=5.8 Hz).

LCMS: 318 [M+1], (MW: 318.17).

### Example 49

### (3-Aminomethyl-imidazo[1,2-b]pyridazin-6-yl)-(3,4-dichloro-benzyl)-amine

6-(3,4-Dichloro-benzylamino)-imidazo[1,2-b]pyridazine-3-carbonitrile (0.328 g, 1.03 mmol) was dissolved in ethanol (10 mL) and ethyl acetate (10 mL). The mixture was reacted on the H-cube™ hydrogenation apparatus (Raney/Nickel-cartridge, temperature=50° C., pressure=30 bar, flow rate 1 mL/min). The solvent was removed in vacuo to give (3-aminomethyl-imidazo[1,2-b]pyridazin-6-yl)-(3,4-dichloro-benzyl)-amine as a yellow solid, which was used without further purification (0.138 g, 43% yield).

LCMS: 322 [M+1], (MW: 322.20).

### Example 50

### N-[6-(3,4-Dichloro-benzylamino)-imidazo[1,2-b]pyridazin-3-ylmethyl]-benzamide

A mixture of (3-aminomethyl-imidazo[1,2-b]pyridazin-6-yl)-(3,4-dichloro-benzyl)-amine (95 mg, 0.295 mmol) and benzoyl chloride (0.044 mL, 0.383 mmol) in dry pyridine (1 mL) was stirred for 18 hours at room temperature. The solvent was removed in vacuo and the residue was purified by flash column chromatography on silica gel (hexane/ethyl acetate 7:3 to 1:1) to give N-[6-(3,4-dichloro-benzylamino)-imidazo[1,2-b]pyridazin-3-ylmethyl]-benzamide as a white solid (48 mg; 38%).

¹H NMR (300 MHz, DMSO-d₆): δ 8.76 (1H, t, J=5.2 Hz), 7.83 (2H, d, J=7.5 Hz), 7.70 (1H, d, J=9.7 Hz), 7.66 (1H, s), 7.54 (1H, t, J=5.8 Hz), 7.51 (1H, d, J=6.8 Hz), 7.45 (2H, dd, J=7.8, 3.4 Hz), 7.41-7.33 (2H, m), 7.31 (1H, s), 6.67 (1H, d, J=9.7 Hz), 4.68 (2H, d, J=5.2 Hz), 4.44 (2H, d, J=5.8 Hz).

LCMS: 426 [M+1], tR=7.88 min, (MW: 426.31).

### Example 51

### 6-(3,4-Dichloro-benzylamino)-imidazo[1,2-b]pyridazine-3-carboxylic acid 3,4-dichlorobenzylamide

A mixture of 6-chloro-imidazo[1,2-b]pyridazine-3-carboxylic acid ethyl ester (50 mg, 0.22 mmol) and 3,4-dichlorobenzylamine (0.064 mL, 0.48 mmol) in water (1 mL) was heated at 150° C. for 1 hour under microwave irradiation. On cooling, the resulting solid was filtered off, washed with water and dried to give 13 mg of 6-(3,4-dichloro-benzylamino)-imidazo[1,2-b]pyridazine-3-carboxylic acid 3,4-dichlorobenzylamide as a white solid (16% yield).

¹H NMR (300 MHz, DMSO-d₆): δ 8.86 (1H, t, J=5.4 Hz), 8.11 (1H, t, J=5.4 Hz), 7.95-7.91 (2H, m), 7.51-7.48 (2H, m), 7.40-7.38 (2H, m), 7.23 (1H, d, J=8.4 Hz), 7.12 (1H, d, J=9.4 Hz), 6.94 (1H, d, J=9.7 Hz), 4.98 (2H, d, J=5.6 Hz), 4.94 (2H, d, J=5.6 Hz).

LCMS: 494 [M+1], tR=13.06 min, (MW: 495.20).

### Example 52

### [6-(3,4-Dichloro-benzylamino)-imidazo[1,2-b]pyridazin-3-yl]-methanol

A solution of lithium aluminum hydride (84 mg, 2.19 mmol) in dry tetrahydrofuran (10 mL) was cooled at 0° C. Then, 6-(3,4-dichloro-benzylamino)-imidazo[1,2-b]pyridazine-3-carboxylic acid ethyl ester (400 mg, 1.095 mmol) was added portionwise and the mixture was stirred for 1 hour at room temperature. On cooling at 0° C., water (0.5 mL) was added and the mixture was filtered off. The solvent was removed in vacuo to give 283 mg of [6-(3,4-dichloro-benzylamino)-imidazo[1,2-b]pyridazin-3-yl]-methanol as a white solid (80% yield).

¹H NMR (300 MHz, DMSO-d₆): δ 7.69 (1H, d, J=1.2 Hz), 7.68 (1H, d, J=9.7 Hz), 7.58 (1H, d, J=8.2 Hz), 7.52 (1H, t, J=5.7 Hz), 7.42 (1H, dd, J=8.2, 1.2 Hz), 7.31 (1H, s), 6.66 (1H, d, J=9.7 Hz), 5.01 (1H, t, J=5.5 Hz), 4.64 (2H, d, J=5.5 Hz), 4.44 (2H, d, J=5.7 Hz).

LCMS: 323 [M+1], (MW: 323.18).

### Example 53

### (3,4-Dichloro-benzyl)-(3-morpholin-4-ylmethyl-imidazo[1,2-b]pyridazin-6-yl)-amine

A mixture of [6-(3,4-dichloro-benzylamino)-imidazo[1,2-b]pyridazin-3-yl]-methanol (50 mg, 0.23 mmol), triphenylphosphine (98 mg, 0.37 mmol) and N-bromosuccinimide (67 mg, 0.37 mmol) in 1,4-dioxane (2 mL) and N,N′-dimethylformamide (0.5 mL) was stirred for 4 hours at room temperature. Then, morpholine (0.080 mL, 0.92 mmol) was added and the mixture was stirred for 18 hours at room temperature. The solvent was removed in vacuo and 2N aqueous solution of sodium hydroxide (0.5 mL) was added and the mixture was extracted with ethyl acetate (4×50 mL). The combined organic fractions were dried (sodium sulphate), the solvent removed in vacuo and the residue was purified by flash column chromatography on silica gel (ethyl acetate/methanol 10:0 to 7:3) to give 11 mg of (3,4-dichloro-benzyl)-(3-morpholin-4-ylmethyl-imidazo[1,2-b]pyridazin-6-yl)-amine as a brown oil (18% yield).

¹H NMR (300 MHz, CDCl₃): δ 7.66 (1H, d, J=9.6 Hz), 7.51 (1H, d, J=1.8 Hz), 7.45 (1H, s), 7.42 (1H, d, J=8.2 Hz), 7.25 (1H, dd, J=8.3, 1.8 Hz), 6.48 (1H, d, J=9.6 Hz), 4.95 (1H, t, J=4.7 Hz), 4.54 (2H, d, J=5.7 Hz), 3.85 (2H, s), 3.60-3.65 (4H, m), 2.47-2.44 (4H, m).

LCMS: 392 [M+1], tR=5.83 min, (MW: 392.29).

### Intermediate 6

### N′-(6-Chloro-4-methyl-pyridazin-3-yl)-N,N-dimethyl-formamidine and N′-(6-chloro-5-methyl-pyridazin-3-yl)-N,N-dimethyl-formamidine

An unresolved mixture of 3-amino-6-chloro-5-methylpyridazine and 3-amino-6-chloro-4-methylpyridazine (4.0 g, 27.90 mmol) was dissolved in N,N′-dimethylformamide diethylacetal (14.32 mL, 83.60 mmol) and refluxed for 4 hours under nitrogen. The solvent was removed in vacuo to give 5.42 g of an unresolved mixture of N′-(6-chloro-4-methyl-pyridazin-3-yl)-N,N-dimethyl-formamidine and N′-(6-chloro-3-methyl-pyridazin-3-yl)-N,N-dimethyl-formamidine as a brown solid which was used without further purification (98% combined yield).

¹H NMR (300 MHz, CDCl₃): δ 8.47 (1H, s), 8.39 (1H, s), 7.07 (1H, s), 6.91 (1H, s), 3.06 (6H, s), 3.04 (6H, s), 2.25 (3H, s), 2.20 (3H, s).

LCMS: 199 [M+1], (MW: 198.65).

### Intermediate 7

### 6-Chloro-8-methyl-imidazo[1,2-b]pyridazine-3-carboxylic acid ethyl ester and 6-chloro-7-methyl-imidazo[1,2-b]pyridazine-3-carboxylic acid ethyl ester

An unresolved mixture of N′-(6-chloro-4-methyl-pyridazin-3-yl)-N,N-dimethyl-formamidine and N′-(6-chloro-3-methyl-pyridazin-3-yl)-N,N-dimethyl-formamidine (6.60 g, 33.20 mmol) and ethylbromoacetate (11.3 mL, 99.50 mmol) in acetonitrile (15.0 mL) was refluxed for 18 hours. The solvent was removed in vacuo and the residue was dissolved in acetonitrile (10 mL). N,N-diisopropylethylamine (17.9 mL, 102.80 mmol) was added at 0° C. The reaction mixture was stirred at room temperature for 3 hours. Then, the solvent was removed in vacuo. The crude mixture was filtered through a silica gel pad using dichloromethane and the solvent removed in vacuo. The obtained residue was purified by flash column chromatography on silica gel (hexanes/ethyl acetate 3:7) to give 0.957 g of 6-chloro-8-methyl-imidazo[1,2-b]pyridazine-3-carboxylic ethyl ester (12% yield) and 1.326 g of 6-chloro-7-methyl-imidazo[1,2-b]pyridazine-3-carboxylic ethyl ester (17% yield).

6-Chloro-8-methyl-imidazo[1,2-b]pyridazine-3-carboxylic ethyl ester: ¹H NMR (300 MHz, CDCl₃): δ 8.29 (1H, s), 7.09 (1H, s), 4.45 (2H, q, J=7.1 Hz), 2.70 (3H, s), 1.40 (3H, t, J=7.1 Hz).

LCMS: 240 [M+1], (MW: 239.66).

6-Chloro-7-methyl-imidazo[1,2-b]pyridazine-3-carboxylic ethyl ester: ¹H NMR (300 MHz, CDCl₃): δ 8.29 (1H, s), 7.85 (1H, s), 4.43 (2H, q, J=7.1 Hz), 2.50 (3H, s), 1.41 (3H, t, J=7.1 Hz).

LCMS: 240 [M+1], (MW: 239.66).

### Intermediate 8

### 6-Chloro-8-methyl-imidazo[1,2-b]pyridazine-3-carboxylic acid phenylamide

A solution of trimethylaluminium (2M in hexane) (0.114 mL, 0.23 mmol) was slowly added at room temperature to a solution of aniline (0.021 mL, 0.23 mmol) in dry dichloromethane (5 mL) under argon. The mixture was stirred for 30 minutes at room temperature. Then, 6-chloro-8-methyl-imidazo[1,2-b]pyridazine-3-carboxylic ethyl ester (0.050 g, 0.21 mmol) was added and the reaction mixture was refluxed for 18 hours. On cooling, the reaction was quenched with 0.2 M aqueous solution of hydrochloric acid (10 mL) and extracted with dichloromethane (2×20 mL). The combined organic layers were dried (magnesium sulphate) and concentrated in vacuo to afford a white solid. The crude was purified by flash column chromatography on silica gel (dichloromethane/methanol 9.95:0.05 to 9.8:0.2) to give 0.040 g of 6-chloro-8-methyl-imidazo[1,2-b]pyridazine-3-carboxylic acid phenylamide (67% yield).

¹H NMR (300 MHz, CDCl₃): δ 10.20 (1H, s), 8.53 (1H, s), 7.75 (1H, d, J=0.9 Hz), 7.72 (1H, s), 7.39 (2H, t, J=7.7 Hz), 7.15 (1H, t, J=7.4 Hz), 7.14-7.08 (1H, m), 2.74 (3H, s).

LCMS: 287 [M+1], (MW: 286.72).

### Example 54

### 6-(3,4-Dichloro-benzylamino)-8-methyl-imidazo[1,2-b]pyridazine-3-carboxylic acid phenylamide

Sodium tert-butoxide (0.027 g, 0.03 mmol), (R)-(+)-2,2′-bis(diphenylphosphino)-1,1′-binaphthyl (0.008 g, 0.01 mmol) and tris(dibenzylideneacetone)dipalladium(0) (0.0063 g, 0.01 mmol) were added to a solution of 6-chloro-8-methyl-imidazo[1,2-b]pyridazine-3-carboxylic acid phenylamide (0.040 g, 0.139 mmol) in dry 1,4-dioxane (5.0 mL) at room temperature. The reaction mixture was refluxed for 6 hours. The solvent was removed in vacuo and the residue was dissolved in ethyl acetate (10 mL). The organic phase was washed with water (4×10 mL), dried (magnesium sulphate) and the solvent removed in vacuo. The resulting yellow oil was purified by flash column chromatography on silica gel (dichloromethane/methanol 10:0.05 to 10:0.4) followed by semi-preparative HPLC (Gemini C18 (150×10 mm; 5 μm), Solvent A: water with 0.1% formic acid; Solvent B: acetonitrile with 0.1% formic acid. Gradient: 40% of A to 0% of A) to give 0.097 g of 6-(3,4-dichloro-benzylamino)-8-methyl-imidazo[1,2-b]pyridazine-3-carboxylic acid phenylamide as a white solid (16% yield).

¹H NMR (300 MHz, DMSO-d₆): δ 10.50 (1H, s), 8.06 (1H, t, J=5.5 Hz), 8.02 (1H, s), 7.69 (1H, d, J=1.4 Hz), 7.59 (1H, d, J=8.4 Hz), 7.38 (2H, d, J=8.4 Hz), 7.31 (2H, t, J=7.4 Hz), 7.11 (1H, t, J=7.4 Hz), 6.84 (1H, s), 4.67 (2H, d, J=5.5 Hz), 2.51 (3H, d, J=1.4 Hz)

LCMS: 426 [M+1], tR=13.07 min, (MW: 426.30).

### Intermediate 9

### 6-Chloro-7-methyl-imidazo[1,2-b]pyridazine-3-carboxylic acid phenylamide

A solution of trimethylaluminium (2M in hexane) (1.350 mL, 2.71 mmol) was slowly added at room temperature to a solution of aniline (0.245 mL, 2.71 mmol) in dry dichloromethane (40 mL) under argon and the mixture was stirred for 30 minutes at room temperature. Then, 6-chloro-7-methyl-imidazo[1,2-b]pyridazine-3-carboxylic ethyl ester (0.50 g, 2.08 mmol) was added and the reaction mixture was refluxed for 18 hours. Then, an additional amount of the solution of trimethylaluminium (2M in hexane) (1.350 mL, 2.71 mmol) and the dichloromethane (40 mL) solution of aniline (0.245 mL, 2.71 mmol) were added to the refluxing reaction mixture which was refluxed for 5 more hours. On cooling, the reaction was quenched with 0.2M aqueous solution of hydrochloric acid (40 mL) and extracted with dichloromethane (4×50 mL). The combined organic fractions were dried (magnesium sulphate) and the solvent removed in vacuo to afford 0.545 g of 6-chloro-7-methyl-imidazo[1,2-b]pyridazine-3-carboxylic acid phenylamide as a white solid (91% yield).

¹H NMR (300 MHz, CDCl₃): δ 10.08 (1H, s), 8.53 (1H, s), 8.03 (1H, s), 7.73 (2H, d, J=7.7 Hz), 7.39 (2H, t, J=7.7 Hz), 7.16 (1H, t, J=7.7 Hz), 2.55 (3H, s).

LCMS: 287 [M+1], (MW: 286.72).

### Example 55

### 6-(3,4-Dichloro-benzylamino)-7-methyl-imidazo[1,2-b]pyridazine-3-carboxylic acid phenylamide

Sodium tert-butoxide (0.101 g, 1.04 mmol), (R)-(+)-2,2′-bis(diphenylphosphino)-1,1′-binaphthyl (0.029 g, 0.05 mmol) and tris(dibenzylideneacetone)dipalladium(0) (0.024 g, 0.026 mmol) were added to a solution of 6-chloro-7-methyl-imidazo[1,2-b]pyridazine-3-carboxylic acid phenylamide (0.150 g, 0.52 mmol) in dry 1,4-dioxane (8.0 mL) at room temperature. The reaction mixture was heated at 100° C. for 1 hour under microwave irradiation. On cooling, the crude mixture was diluted with ethyl acetate (25 mL) and water (25 mL) and extracted with ethyl acetate (4×20 mL). The combined organic fractions were dried (magnesium sulphate) and the solvent removed in vacuo. The residue was purified by column chromatography on flash silica gel (dichloromethane/methanol 9.95:0.05 to 9.6:0.4) to afford 0.043 g of 6-(3,4-dichloro-benzylamino)-7-methyl-imidazo[1,2-b]pyridazine-3-carboxylic acid phenylamide as a white solid (19% yield).

¹H NMR (300 MHz, DMSO-d₆): δ 10.25 (1H, s), 8.00 (1H, s), 7.90 (1H, s), 7.66 (1H, d, J=1.4 Hz), 7.63 (1H, t, J=5.5 Hz), 7.52 (1H, d, J=8.3 Hz), 7.41-7.22 (5H, m), 7.16-7.04 (1H, m), 4.72 (2H, d, J=5.5 Hz), 2.35 (3H, s).

LCMS: 426 [M+1], tR=11.75 min, (MW: 426.30).

### General Procedure L for the Preparation of Examples 56-61

A mixture of 6-chloro-7-methyl-imidazo[1,2-b]pyridazine-3-carboxylic acid phenylamide (1 eq) and the appropriate amine (e.g. 4-fluorobenzylamine) (3.0 eq) in the appropriate solvent (N,N′-dimethylformamide or N,N′ dimethylacetamide) (about 9 mL/mmol) was heated at 100° C. for several hours (from 24 to 72 hours depending upon the corresponding amine). On cooling, the solvent was removed in vacuo, the residue dissolved in dichloromethane (about 10 mL) and washed with water (about 10 mL). The organic fraction was dried (magnesium sulphate) and the solvent removed in vacuo. The residue was purified by column chromatography on flash silica gel (dichloromethane/methanol mixtures) to give the desired product (e.g. 6-(4-fluoro-benzylamino)-7-methyl-imidazo[1,2-b]pyridazine-3-carboxylic acid phenylamide).

### Example 56

### 6-(4-Fluoro-benzylamino)-7-methyl-imidazo[1,2-b]pyridazine-3-carboxylic acid phenylamide

The title compound was obtained as a white solid after purification by column chromatography on flash silica gel (dichloromethane/methanol 9.95:0.05 to 9.6:0.4) (28% yield).

¹H NMR (300 MHz, DMSO-d₆): δ 10.38 (1H, s), 8.00 (1H, s), 7.89 (1H, d, J=0.9 Hz), 7.60 (1H, t, J=5.8 Hz), 7.41 (2H, dd, J=5.6 and 8.5 Hz), 7.28 (2H, s), 7.26 (2H, s), 7.1-7.04 (3H, m), 4.72 (2H, d, J=5.8 Hz), 2.35 (3H, s).

LCMS: 376 [M+1], tR=10.79 min, (MW: 375.4).

### Example 57

### 6-(4-Methoxy-benzylamino)-7-methyl-imidazo[1,2-b]pyridazine-3-carboxylic acid phenylamide

The title compound was obtained as a white solid after purification by flash column chromatography on silica gel (dichloromethane/methanol 9.95:0.05 to 9.6:0.4) (59% yield).

¹H NMR (300 MHz, DMSO-d₆): δ 10.46 (1H, s), 8.00 (1H, s), 7.88 (1H, s), 7.55 (1H, t, J=5.8 Hz), 7.34-7.25 (6H, m), 7.14-7.03 (1H, m), 6.85 (2H, d, J=8.6 Hz), 4.67 (2H, d, J=5.8 Hz), 3.68 (3H, s), 2.34 (3H, s).

LCMS: 388 [M+1], tR=10.48 min, (MW: 387.4).

### Example 58

### 6-[(Furan-2-ylmethyl)-amino]-7-methyl-imidazo[1,2-b]pyridazine-3-carboxylic acid phenylamide

The title compound was obtained as a brown solid after purification by flash column chromatography on silica gel (dichloromethane/methanol 9.95:0.05 to 9.6:0.4) followed by recrystallization from methanol (18% yield).

¹H NMR (300 MHz, DMSO-d₆): δ 10.60 (1H, s), 8.03 (1H, s), 7.87 (1H, d, J=0.9 Hz), 7.57 (1H, t, J=5.8 Hz), 7.56-7.46 (3H, m), 7.33 (2H, t, J=7.6 Hz), 7.10 (1H, t, J=7.6 Hz), 6.35 (1H, dd, J=1.9, 3.1 Hz), 6.30 (1H, d, J=3.1 Hz), 4.69 (2H, d, J=5.8 Hz), 2.28 (3H, s).

LCMS: 348 [M+1], tR=9.83 min, (MW: 347.3).

### Example 59

### 7-Methyl-6-[(pyridin-4-ylmethyl)-amino]imidazo[1,2-b]pyridazine-3-carboxylic acid phenylamide

The title compound was obtained as a white solid (4% yield) after purification by flash column chromatography on silica gel (dichloromethane/methanol 10:0.05 to 10:0.4) followed by semi-preparative HPLC (Gemini C18 (150×10 mm; 5 μm), Solvent A: water with 0.1% formic acid; Solvent B: acetonitrile with 0.1% formic acid. Gradient: 95% of A to 70% of A).

¹H NMR (300 MHz, DMSO-d₆): δ 10.20 (1H, s), 8.43 (2H, dd, J=1.4, 4.6 Hz), 8.00 (1H, s), 7.92 (1H, d, J=1.0 Hz), 7.66 (1H, t, J=5.8 Hz), 7.37 (2H, d, J=5.8 Hz), 7.31-7.21 (4H, m), 7.08 (1H, d, J=6.9 Hz), 4.76 (2H, d, J=5.8 Hz), 2.38 (3H, s).

LCMS: 359 [M+1], (MW: 358.4).

### Example 60

### 6-(3-Fluoro-4-methyl-benzylamino)-7-methyl-imidazo[1,2-b]pyridazine-3-carboxylic acid phenylamide

The title compound was obtained as a white solid after purification by flash column chromatography on silica gel (dichloromethane/methanol 10:0.05 to 10:0.5) (35% yield).

¹H NMR (300 MHz, DMSO-d₆): δ 10.34 (1H, s), 8.00 (1H, s), 7.89 (1H, s), 7.60 (1H, t, J=5.8 Hz), 7.30-7.22 (4H, m), 7.21-7.04 (4H, m), 4.70 (2H, d, J=5.8 Hz), 2.35 (3H, s), 2.15 (3H, s).

LCMS: 390 [M+1], tR=11.40 min, (MW: 389.4).

### Example 61

### 6-(3-Fluoro-benzylamino)-7-methyl-imidazo[1,2-b]pyridazine-3-carboxylic acid phenylamide

The title compound was obtained as a white solid after purification by column chromatography on flash silica gel (dichloromethane/methanol 9.95:0.05 to 9.6:0.4) (53% yield).

¹H NMR (300 MHz, DMSO-d₆): δ 10.33 (1H, s), 8.00 (1H, s), 7.90 (1H, s), 7.63 (1H, t, J=5.8 Hz), 7.40-7.16 (7H, m), 7.15-6.97 (2H, m), 4.75 (2H, d, J=5.8 Hz), 2.36 (3H, s).

LCMS: 376 [M+1], tR=10.70 min, (MW: 375.4).

### Example 62

### 6-(4-Methoxy-benzylamino)-8-methyl-imidazo[1,2-b]pyridazine-3-carboxylic acid ethyl ester

A mixture of 6-chloro-8-methyl-imidazo[1,2-b]pyridazine-3-carboxylic ethyl ester (0.400 g, 1.67 mmol) and 4-methoxybenzylamine (1.09 mL, 8.34 mmol) in N,N′-dimethylacetamide (4 mL) was stirred at 100° C. for 48 hours. The solvent was removed in vacuo and the residue was dissolved in dichloromethane (20 mL). The organic fraction was washed with water (2×20 ml), dried (magnesium sulphate) and the solvent removed in vacuo. The residue was purified by column chromatography on flash silica gel (dichloromethane/methanol 9.95:0.05 to 9.6:0.4) to give a yellow oil that was purified by reverse phase column chromatography (mixtures of acetonitrile/water) to give 0.284 mg of 6-(4-methoxy-benzylamino)-8-methyl-imidazo[1,2-b]pyridazine-3-carboxylic acid ethyl ester as a white solid (50% yield).

¹H NMR (300 MHz, CDCl₃): δ 8.11 (1H, s), 7.34 (2H, d, J=8.3 Hz), 6.86 (2H, d, J=8.3 Hz), 6.46 (1H, s), 4.79 (1H, s), 4.52 (2H, d, J=4.5 Hz), 4.41 (2H, q, J=7.1 Hz), 3.78 (3H, s), 2.58 (3H, s), 1.39 (3H, t, J=7.1 Hz).

LCMS: 341 [M+1], (MW: 340.3).

### Example 63

### 6-(4-Methoxy-benzylamino)-8-methyl-imidazo[1,2-b]pyridazine-3-carboxylic acid

6-(4-Methoxy-benzylamino)-8-methyl-imidazo[1,2-b]pyridazine-3-carboxylic acid ethyl ester (0.284 g, 0.83 mmol) was dissolved in ethanol (6 mL) and 4M aqueous solution of potassium hydroxide (4 mL) was added. The mixture was stirred at room temperature overnight. The ethanol was removed in vacuo and the aqueous solution was extracted with ethyl acetate (5 mL) and the aqueous layer cooled at 0° C. Then, acetic acid was added up to pH 5 and the resulting solid was filtered off, washed with water and dried to afford 0.170 g of 6-(4-methoxy-benzylamino)-8-methyl-imidazo[1,2-b]pyridazine-3-carboxylic acid as a white solid (65% yield).

¹H NMR (300 MHz, DMSO-d₆): δ 12.49 (1H, s), 7.94 (1H, s), 7.57 (1H, t, J=5.7 Hz), 7.39 (2H, t, J=5.7 Hz), 6.92-6.83 (2H, m), 6.69 (1H, d, J=1.0 Hz), 4.37 (2H, d, J=5.7 Hz), 3.72 (3H, s), 2.42 (3H, d, J=1.0 Hz).

LCMS: 313 [M+1], (MW: 312.3).

### Example 64

### 3-{[6-(4-Methoxy-benzylamino)-8-methyl-imidazo[1,2-b]pyridazine-3-carbonyl]-amino}-benzoic acid ethyl ester

N-(3-Dimethylaminopropyl)-N′-ethylcarbodiimide hydrochloride (0.246 g, 1.28 mmol) and 1-hydroxybenzotriazole (0.196 g, 1.28 mmol) were added to a solution of 6-(4-methoxy-benzylamino)-8-methyl-imidazo[1,2-b]pyridazine-3-carboxylic acid (0.200 g, 0.64 mmol) in N,N′-dimethylformamide (10.0 mL). The mixture was stirred for 4 hours at 60° C. Then, ethyl 3-aminobenzoate (0.191 mL, 1.28 mmol) was added and the mixture was stirred at 60° C. for 18 hours. The solvent was removed in vacuo and the residue was dissolved in dichloromethane (20 mL). The solution was washed with a saturated solution of sodium hydrogen carbonate (2×10 mL) and dried (magnesium sulphate) and the solvent was removed in vacuo. The residue was triturated from acetonitrile to give a white solid which was purified by reverse phase column chromatography (mixtures of acetonitrile/water) to afford 0.158 g of 3-{[6-(4-methoxy-benzylamino)-8-methyl-imidazo[1,2-b]pyridazine-3-carbonyl]-amino}-benzoic acid ethyl ester as a white solid (53% yield).

¹H NMR (300 MHz, CDCl₃): δ 10.93 (1H, s), 8.34 (1H, s), 8.04 (1H, s), 7.92 (1H, t, J=5.2 Hz), 7.69 (1H, d, J=7.8 Hz), 7.60 (1H, d, J=8.4 Hz), 7.45 (1H, t, J=7.8 Hz), 7.36 (2H, d, J=8.4 Hz), 6.89 (2H, d, J=8.4 Hz), 6.82 (1H, s), 4.57 (2H, d, J=5.2 Hz), 4.27 (2H, q, J=7.1 Hz), 3.70 (3H, s), 2.49 (3H, s), 1.25 (3H, t, J=7.1 Hz).

LCMS: 460 [M+1], tR=12.63 min, (MW: 459.5).

### Example 65

### 3-{[6-(4-Methoxy-benzylamino)-8-methyl-imidazo[1,2-b]pyridazine-3-carbonyl]-amino}-benzoic acid

3-{[6-(4-Methoxy-benzylamino)-8-methyl-imidazo[1,2-b]pyridazine-3-carbonyl]-amino}-benzoic acid ethyl ester (0.025 g, 0.05 mmol) was dissolved in ethanol (2 mL) and 4M aqueous solution of potassium hydroxide (1 mL) was added. The mixture was stirred at room temperature for 18 hours. The ethanol was removed in vacuo and the aqueous mixture was extracted with ethyl acetate (10 mL) and the aqueous fraction cooled at 0° C. Then, acetic acid was added up to pH 5 and the resulting solid was filtered off, washed with water and dried to give a white solid which was purified by reverse phase column chromatography (mixtures of acetonitrile/water) to afford 0.017 g of 3-{[6-(4-methoxy-benzylamino)-8-methyl-imidazo[1,2-b]pyridazine-3-carbonyl]-amino}-benzoic acid as a white solid (72% yield).

¹H NMR (300 MHz, methanol-d₄): δ 8.26 (1H, s), 8.08 (1H, s), 7.77 (1H, d, J=7.8 Hz), 7.58 (1H, d, J=8.4 Hz), 7.45-7.28 (3H, m), 6.84 (2H, d, J=8.4 Hz), 6.78 (1H, d, J=1.0 Hz), 4.62 (2H, s), 3.73 (3H, s), 2.53 (3H, d, J=1.0 Hz).

LCMS: 432 [M+1], tR=9.81 min, (MW: 431.4).

### Example 66

### 6-(4-Methoxy-benzylamino)-8-methyl-imidazo[1,2-b]pyridazine-3-carboxylic acid (3-methylcarbamoyl-phenyl)-amide

A solution of trimethylaluminium (2M in hexane) (0.065 mL, 0.13 mmol) was added at room temperature to a solution of methylamine (2M in tetrahydrofuran) (0.066 mL, 0.13 mmol) in dichloromethane (5 mL) under argon. The mixture was stirred at room temperature for 30 minutes and 3-{[6-(4-methoxy-benzylamino)-8-methyl-imidazo[1,2-b]pyridazine-3-carbonyl]amino}-benzoic acid ethyl ester (0.030 g, 0.065 mmol) was added. The mixture was refluxed for 18 hours. Then, an additional reagents mixture, prepared in a similar fashion as described previously, of trimethylaluminium (2M in hexane) (0.065 mL, 0.130 mmol) and methylamine (2M in tetrahydrofuran) (0.066 mL, 0.131 mmol) in dichloromethane (5 mL), was added to the refluxing reaction mixture. The mixture was refluxed for 24 hours and then, a third additional mixture, as described previously, of trimethylaluminium (2M in hexane) (0.065 mL, 0.130 mmol) and methylamine (2M in tetrahydrofuran) (0.066 mL, 0.131 mmol) in dichloromethane (5 mL) was added to the refluxing reaction mixture. The mixture was refluxed for 24 hours. On cooling, the reaction was quenched with 0.1M aqueous solution of hydrochloric acid (80 mL) and extracted with dichloromethane (4×). The combined organic fractions were dried (magnesium sulphate) and concentrated in vacuo to give a residue that was triturated from acetonitrile. The resulting solid was filtered off and washed with cold acetonitrile to afford 0.016 g of 6-(4-methoxy-benzylamino)-8-methyl-imidazo[1,2-b]pyridazine-3-carboxylic acid (3-methylcarbamoyl-phenyl)-amide as a white solid (55% yield).

¹H NMR (300 MHz, DMSO-d₆): δ 10.86 (1H, s), 8.41 (1H, d, J=4.5 Hz), 8.11 (1H, s), 8.03 (1H, s), 7.94 (1H, t, J=5.3 Hz), 7.53 (2H, d, J=7.6 Hz), 7.40 (1H, d, J=7.3 Hz), 7.34 (2H, d, J=8.8 Hz), 6.94-6.78 (3H, m), 4.58 (2H, d, J=5.3 Hz), 3.69 (3H, s), 2.77 (3H, d, J=4.5 Hz).

LCMS: 445 [M+1], tR=9.17 min, (MW: 444.4).

### Example 67

### 6-(4-Methoxy-benzylamino)-8-methyl-imidazo[1,2-b]pyridazine-3-carboxylic acid (3-dimethylcarbamoyl-phenyl)-amide

A solution of trimethylaluminium (2M in hexane) (0.130 mL, 0.26 mmol) was added at room temperature to a 2M solution in tetrahydrofuran of dimethylamine (0.130 mL, 0.26 mmol) in dry dichloromethane (5 mL) under argon. The mixture was stirred at room temperature for 30 minutes and 3-{[6-(4-methoxy-benzylamino)-8-methyl-imidazo[1,2-b]pyridazine-3-carbonyl]-amino}-benzoic acid ethyl ester (0.040 g, 0.087 mmol) was added. The mixture was refluxed for 18 hours. Then, an additional mixture, prepared in similar fashion as described previously, of trimethylaluminium (2M in hexane) (0.130 mL, 0.26 mmol) and dimethylamine (2M in tetrahydrofuran) (0.130 mL, 0.26 mmol), in dichloromethane (5 mL) was added to the refluxing reaction mixture. The reaction mixture was refluxed for 24 hours. On cooling, the reaction was quenched with 0.1M aqueous solution of hydrochloric acid (80 mL) and extracted with dichloromethane (4×). The combined organic fractions were dried (magnesium sulphate) and concentrated in vacuo to give a residue that was purified by flash column chromatography on silica gel (ethyl acetate/methanol 100:0 to 80:20) followed by semi-preparative HPLC (Gemini C18 (150×10 mm; 5 μm), Solvent A: water with 0.1% formic acid; Solvent B: acetonitrile with 0.1% formic acid. Gradient: 80% of A to 40% of A) to afford 0.005 g of 6-(4-methoxy-benzylamino)-8-methyl-imidazo[1,2-b]pyridazine-3-carboxylic acid (3-dimethylcarbamoyl-phenyl)-amide as a white solid (12% yield).

¹H NMR (600 MHz, methanol-d₄): δ 8.06 (1H, d, J=8.0 Hz), 7.54 (1H, s), 7.41 (1H, d, J=6.8 Hz), 7.38-7.28 (3H, m), 7.14 (1H, d, J=7.0 Hz), 6.88 (2H, d, J=7.7 Hz), 6.76 (1H, d, J=8.8 Hz), 4.59 (2H, d, J=8.8 Hz), 3.75 (3H, s), 3.09 (3H, s), 2.95 (3H, s), 2.52 (3H, s).

LCMS: 459 [M+1], tR=9.55 min, (MW: 458.5).

### Intermediate 10

### 6-Chloro-5-cyclopentyl-pyridazin-3-ylamine

3,6-dichloro-4-cyclopentyl-pyridazine (synthesized following U.S. Pat. No. 6,255,305 B1) (0.500 g, 2.30 mmol) in ethanol (1.0 mL) and 32% aqueous solution of ammonium hydroxide (2.0 mL) was heated at 155° C. for 1.5 hours under microwave irradiation. On cooling, the solvent was removed in vacuo and the residue was dissolved in dichloromethane (10 mL) and washed with water (10 mL). The organic fraction was dried (magnesium sulphate) and the solvent removed in vacuo to give a residue that was triturated from ethyl acetate. The resulting solid was filtered off, washed with diethyl ether and dried to afford 0.170 g of 6-chloro-5-cyclopentyl-pyridazin-3-ylamine as a white solid (38% yield).

¹H NMR (300 MHz, CDCl₃): δ 6.67 (1H, s), 4.95 (2H, bs), 3.21 (1H, p, J=8.2 Hz), 2.22-2.03 (2H, m), 1.93-1.63 (4H, m), 1.63-1.4 (2H, m).

LCMS: 198 [M+1], (MW: 197.6).

### Intermediate 11

### N′-(6-Chloro-5-cyclopentyl-pyridazin-3-yl)-N,N-dimethyl-formamidine

A mixture of 6-chloro-5-cyclopentyl-pyridazin-3-ylamine (0.300 g, 1.50 mmol) and N,N′-dimethylformamide diethylacetal (0.780 mL, 4.50 mmol) was refluxed for 4 hours under nitrogen. On cooling, the solvent was removed in vacuo to give 0.390 g of N′-(6-chloro-4-cyclopentyl-pyridazin-3-yl)-N,N-dimethyl-formamidine as a orange oil, which was used without further purification (99% yield).

¹H NMR (300 MHz, CDCl₃): δ 8.54 (1H, s), 7.03 (1H, s), 3.29-3.14 (1H, m), 3.11 (6H, bs), 2.17-1.99 (2H, m), 1.85-1.65 (4H, m), 1.62-1.48 (2H, m).

LCMS: 253 [M+1], (MW: 252.7)

### Intermediate 12

### 6-Chloro-7-cyclopentyl-imidazo[1,2-b]pyridazine-3-carboxylic acid ethyl ester

A mixture of N′-(6-chloro-4-cyclopentyl-pyridazin-3-yl)-N,N-dimethyl-formamidine (0.390 g, 1.54 mmol) and ethylbromoacetate (0.513 mL, 4.63 mmol) in acetonitrile (4.0 mL) was refluxed for 8 hours. The solvent was removed in vacuo and the residue was dissolved in acetonitrile (5 mL). N,N-diisopropylethylamine (0.591 mL, 3.39 mmol) was added at room temperature and the mixture was stirred at room temperature for 18 hours. The solvent was removed in vacuo and the residue was triturated from water. The resulting solid was filtered off, washed with water and dried to afford 0.251 g of 6-chloro-7-cyclopentyl-imidazo[1,2-b]pyridazine-3-carboxylic acid ethyl ester as a white solid (55% yield).

¹H NMR (300 MHz, CDCl₃): δ 8.24 (1H, s), 7.85 (1H, s), 4.38 (2H, q, J=7.1 Hz), 3.42-3.23 (1H, m), 2.24-2.05 (2H, m), 1.87-1.66 (4H, m), 1.62-1.49 (2H, m), 1.36 (3H, t, J=7.1 Hz).

LCMS: 294 [M+1], (MW: 293.7).

### Intermediate 13

### 6-Chloro-7-cyclopentyl-imidazo[1,2-b]pyridazine-3-carboxylic acid phenyl-amide

A solution of trimethylaluminium (2M in hexane) (1.276 mL, 2.55 mmol) was slowly added at room temperature to a solution of aniline (0.231 mL, 2.55 mmol) in dry dichloromethane (15 mL) under argon. The mixture was stirred at room temperature for 30 minutes and 6-chloro-7-cyclopentyl-imidazo[1,2-b]pyridazine-3-carboxylic acid ethyl ester (0.250 g, 0.85 mmol) was added. The reaction mixture was refluxed for 1.5 hours. The reaction was quenched with 0.1M aqueous solution of hydrochloric acid (40 mL) and extracted with dichloromethane (4×20 mL). The combined organic layers were dried (magnesium sulphate) and the solvent removed in vacuo to give a residue which was triturated from ethanol. The resulting solid was filtered off, washed with ethanol and dried to afford 0.205 g of 6-chloro-7-cyclopentyl-imidazo[1,2-b]pyridazine-3-carboxylic acid phenylamide as a white solid (70% yield).

¹H NMR (300 MHz, CDCl₃): δ 10.10 (1H, s), 8.51 (1H, d, J=1.1 Hz), 8.02 (1H, s), 7.72 (2H, d, J=8.5 Hz), 7.37 (2H, t, J=7.4 Hz), 7.14 (1H, J=7.4 Hz), 3.39 (1H, p, J=8.0 Hz), 2.27-2.17 (2H, m), 1.96-1.74 (4H, m), 1.72-1.58 (2H, m).

LCMS: 341 [M+1], (MW: 340.8).

### Example 68

### 7-Cyclopentyl-6-(4-fluoro-benzylamino)-imidazo[1,2-b]pyridazine-3-carboxylic acid phenylamide

A mixture of 6-chloro-7-cyclopentyl-imidazo[1,2-b]pyridazine-3-carboxylic acid phenylamide (0.060 g, 0.17 mmol) and 4-fluorobenzylamine (0.101 mL, 0.88 mmol) in N,N′-dimethylacetamide (4 mL) was stirred at 100° C. for 48 hours. On cooling, the solvent was removed in vacuo to give a brown oily residue which was dissolved in dichloromethane (20 mL). The organic fraction was washed with water (2×10 mL), dried (magnesium sulphate) and the solvent removed in vacuo to give a yellow residue which was triturated from acetonitrile. The resulting solid was filtered off, washed with cold acetonitrile and dried to afford 0.032 g of 7-cyclopentyl-6-(4-fluoro-benzylamino)-imidazo[1,2-b]pyridazine-3-carboxylic acid phenylamide as a white solid (42% yield).

¹H NMR (300 MHz, acetone-d₆): δ 10.41 (1H, s), 8.03 (1H, s), 7.72 (1H, d, J=0.8 Hz), 7.52 (2H, dd, J=5.6, 8.5 Hz), 7.46-7.37 (2H, m), 7.30-7.20 (2H, m), 7.15-7.01 (3H, m), 6.96 (1H, t, J=5.4 Hz), 4.90 (2H, d, J=5.4 Hz), 3.27 (1H, p, J=7.9 Hz), 2.33-2.16 (2H, m), 1.96-1.63 (6H, m).

LCMS: 430 [M+1], tR=13.11 min, (MW: 429.4).

### Example 69

### 7-Cyclopentyl-6-(4-methoxy-benzylamino)-imidazo[1,2-b]pyridazine-3-carboxylic acid phenylamide

A mixture of 6-chloro-7-cyclopentyl-imidazo[1,2-b]pyridazine-3-carboxylic acid phenylamide (0.060 g, 0.17 mmol) and 4-methoxybenzylamine (0.115 mL, 0.88 mmol) in N,N′-dimethylacetamide (4 mL) was stirred at 100° C. for 48 hours. On cooling, the solvent was removed in vacuo to give a brown oily residue which was dissolved in dichloromethane (20 mL). The organic fraction was washed with water (2×10 mL), dried (magnesium sulphate) and the solvent removed in vacuo to give a yellow residue which was triturated from acetonitrile. The resulting solid was filtered off, washed with cold acetonitrile and dried to afford 0.054 g of 7-cyclopentyl-6-(4-methoxy-benzylamino)-imidazo[1,2-b]pyridazine-3-carboxylic acid phenylamide (70% yield).

¹H NMR (300 MHz, CDCl₃): δ 10.55 (1H, s), 8.28 (1H, s), 7.75 (1H, s), 7.53 (2H, d, J=7.6 Hz), 7.41-7.24 (4H, m), 7.10 (1H, t, J=7.4 Hz), 6.95 (2H, d, J=8.6 Hz), 5.10 (1H, t, J=4.6 Hz), 4.68 (2H, d, J=4.6 Hz), 3.83 (3H, s), 2.93 (1H, p, J=7.5 Hz), 2.20-2.02 (2H, m), 1.97-1.59 (6H, m).

LCMS: 442 [M+1], tR=12.79 min, (MW: 441.5).

### Intermediate 14

### 6-chloro-7-methylimidazo[1,2-b]pyridazine and 6-chloro-8-methylimidazo-[1,2-b]pyridazine

To a water suspension of an unresolved mixture of 3-amino-6-chloro-5-methylpyridazine and 3-amino-6-chloro-4-methylpyridazine (2.0 g, 13.90 mmol) at 80° C., chloroacetaldehyde (50% solution in water) (2.8 mL, 20.95 mmol) was added. The reaction mixture was stirred at 93° C. overnight. After cooling, solid sodium bicarbonate was added until pH 7 was reached. The resulting oily residue was extracted with ethyl acetate, dried (magnesium sulphate) and evaporated to dryness to afford a brown residue. The obtained regioisomers were separated by flash column chromatography (hexane/ethyl acetate, 4:6) to give 1.05 g (45% yield) of 6-chloro-7-methylimidazo[1,2-b]pyridazine and 0.74 g (32% yield) of 6-chloro-8-methylimidazo[1,2-b]pyridazine.

6-chloro-7-methylimidazo[1,2-b]pyridazine: ¹H NMR (300 MHz, CDCl₃): δ 7.83 (1H, s), 7.74 (1H, s), 7.67 (1H, d, J=1.0 Hz), 2.41 (3H, d, J=1.0 Hz)

6-chloro-8-methylimidazo[1,2-b]pyridazine: ¹H NMR (300 MHz, CDCl₃) δ ppm: 7.88 (1H, d, J=1.0 Hz), 7.70 (1H, d, J=1.0 Hz), 6.88 (1H, d, J=1.0 Hz), 2.65 (3H, d, J=1.0 Hz).

LCMS: 168 [M+1]. (MW: 167.6)

### Intermediate 15

### N-(3,4-dichlorobenzyl)-7-methylimidazo[1,2-b]pyridazin-6-amine

To a mixture of 6-chloro-7-methylimidazo[1,2-b]pyridazine (0.20 g, 1.19 mmol) and 3,4-dichlorobenzylamine (0.24 mL, 1.78 mmol) in 1,4-dioxane (7 mL), were added sodium tert-butoxide (0.18 g, 1.91 mmol), (R)-(+)-2,2′-bis(diphenylphosphino)-1,1′-binaphthyl (0.06 g, 0.10 mmol) and tris(dibenzylideneacetone)dipalladium(0) (0.04 g, 0.06 mmol) at room temperature. The reaction mixture was heated at 100° C. for 1 hour under microwave irradiation. The crude mixture was diluted with ethyl acetate/water and acidified with hydrochloric acid (2N) to pH 3, then extracted with ethyl acetate. The combined organic layers were dried (magnesium sulphate) and concentrated in vacuo. The crude mixture was purified by flash column chromatography (hexane/ethyl acetate 1:4) followed by precipitation with diethyl ether to give N-(3,4-dichlorobenzyl)-7-methylimidazo[1,2-b]pyridazin-6-amine (0.25 g, 48% yield)

¹H NMR (300 MHz, CDCl₃): δ 7.51 (1H, s), 7.41 (2H, bs), 7.36 (1H, s), 7.32 (1H, d, J=8.2 Hz), 7.15 (1H, dd, J=2.3, 8.2 Hz), 4.47 (2H, d, J=5.2 Hz), 4.43 (bs, 1H), 2.14 (s, 3H).

LCMS: 307 [M+1]. (MW: 307.2)

### Intermediate 16

### N-(3,4-dichlorobenzyl)-8-methylimidazo[1,2-b]pyridazin-6-amine

To a mixture of 6-chloro-8-methylimidazo[1,2-b]pyridazine (0.25 g, 1.49 mmol) and 3,4-dichlorobenzylamine (0.29 mL, 2.23 mmol) in 1,4-dioxane (7 mL), were added sodium tert-butoxide (0.23 g, 2.38 mmol), (R)-(+)-2,2′-bis(diphenylphosphino)-1,1′-binaphthyl (0.08 g, 0.13 mmol) and tris(dibenzylideneacetone)dipalladium(0) (0.07 g, 0.07 mmol) at room temperature. The reaction mixture was heated at 100° C. for 1 hour under microwave irradiation. The crude mixture was diluted with ethyl acetate/water and acidified with hydrochloric acid (2N) to pH 3, then extracted with ethyl acetate. The combined organic layers were dried (magnesium sulphate) and concentrated in vacuo. The resulting residue was purified by flash column chromatography (hexane/ethyl acetate 1:4) followed by precipitation with diethyl ether to give N-(3,4-dichlorobenzyl)-8-methylimidazo[1,2-b]pyridazin-6-amine (0.26 g, 56% yield)

¹H NMR (300 MHz, CDCl₃): δ 7.61 (1H, d, J=0.9 Hz), 7.47 (2H, d, J=2.2 Hz), 7.40 (1H, d, J=8.2 Hz), 7.21 (1H, dd, J=2.0, 8.2 Hz), 6.22 (1H, d, J=1.1 Hz), 4.59 (1H, s), 4.46 (2H, d, J=5.8 Hz), 2.49 (3H, d, J=1.0 Hz)

LCMS: 307 [M+1]. (MW: 307.2)

### Intermediate 17

N-(3,4-dichlorobenzyl)-7-methyl-3-iodoimidazo[1,2-b]pyridazin-6-amine

A mixture of N-(3,4-dichlorobenzyl)-7-methylimidazo[1,2-b]pyridazin-6-amine (0.05 g, 0.16 mmol) and N-Iodosuccinimide (0.04 g, 0.18 mmol) in dimethylformamide (0.5 mL) were stirred at room temperature overnight. The reaction mixture was diluted with dichloromethane, washed with 10% sodium thiosulfate solution, dried (magnesium sulphate), filtered and the solvent removed in vacuo. The crude mixture was triturated from diethyl ether to give 0.05 g of N-(3,4-dichlorobenzyl)-7-methyl-3-iodoimidazo[1,2-b]pyridazin-6-amine (74%).

¹H NMR (300 MHz, CDCl₃): δ 7.66 (1H, d, J=1.8 Hz), 7.51 (1H, s), 7.43 (1H, s), 7.42 (1H, d, J=8.2 Hz), 7.36 (1H, dd, J=1.9, 8.2 Hz), 4.80 (1H, s), 4.61 (2H, d, J=5.7 Hz), 2.25 (3H, s).

LCMS: 433 [M+1]. (MW: 433.1)

### Intermediate 18

### N-(3,4-dichlorobenzyl)-8-methyl-3-iodoimidazo[1,2-b]pyridazin-6-amine

A mixture of N-(3,4-dichlorobenzyl)-8-methylimidazo[1,2-b]pyridazin-6-amine (0.10 g, 0.32 mmol) and N-Iodosuccinimide (0.08 g, 0.36 mmol) in dimethylformamide (1.0 mL). The mixture was diluted with dichloromethane, washed with 10% sodium thiosulfate solution, dried (magnesium sulphate), filtered and concentrated to reduce the amount of dimethylformamide. The crude mixture was triturated from diethyl ether to give 100 mg of N-(3,4-dichlorobenzyl)-8-methyl-3-iodoimidazo[1,2-b]pyridazin-6-amine (76% yield).

¹H NMR (300 MHz, CDCl₃): δ 7.55 (1H, d, J=1.8 Hz), 7.46 (1H, s), 7.33 (1H d, J=8.2 Hz), 7.25 (1H, dd, J=1.9, 8.2 Hz), 6.20 (1H, d, J=1.1 Hz), 4.77 (1H, bs), 4.46 (2H, d, J=5.8 Hz), 2.44 (3H, s)

LCMS: 433 [M+1]. (MW: 433.1)

### Example 70

### (3,4-Dichloro-benzyl)-(7-methyl-3-phenylethynyl-imidazo[1,2-b]pyridazin-6-yl)-amine

A mixture of N-(3,4-dichlorobenzyl)-7-methyl-3-iodoimidazo[1,2-b]pyridazin-6-amine (0.04 g, 0.09 mmol), phenylacetylene (0.034 mL, 0.30 mmol), dichlorobis(triphenylphosphine)palladium(II) (6.5 mg, 0.009 mmol), cupper iodide (1.76 mg, 0.009 mmol) and triethylamine (0.5 mL, 3.6 mmol) in dimethylformamide (0.4 mL) was heated at 60° C. for 3 hours. The reaction mixture was poured into water, extracted with dichloromethane, dried (sodium sulphate) and the solvent removed in vacuo. The crude product was purified by flash column chromatography (dichloromethane and mixtures of dichloromethane/methanol 9.9:0.1 to 9.6:0.4) to give 15.0 mg of (3,4-dichloro-benzyl)-(7-methyl-3-phenylethynyl-imidazo[1,2-b]pyridazin-6-yl)-amine (40% yield).

¹H NMR (300 MHz, CDCl₃): δ 7.57 (1H, d, J=1.5 Hz), 7.52 (3H, m,), 7.34 (5H, m), 4.67 (1H, bs), 4.60 (2H, d, J=5.4 Hz), 2.21 (3H, s).

LCMS: 407 [M+1]. (MW: 407.3)

### Example 71

### (3,4-Dichloro-benzyl)-(8-methyl-3-phenylethynyl-imidazo[1,2-b]pyridazin-6-yl)-amine

A mixture of N-(3,4-dichlorobenzyl)-7-methyl-3-iodoimidazo[1,2-b]pyridazin-6-amine (0.04 g, 0.09 mmol), phenylacetylene (0.034 mL, 0.30 mmol), dichlorobis(triphenylphosphine)palladium(II) (6.5 mg, 0.009 mmol), cupper iodide (1.76 mg, 0.009 mmol) and triethylamine (0.5 mL, 3.6 mmol) in dimethylformamide (0.4 mL) was heated at 60° C. for 3 hours. The reaction mixture was poured into water, extracted with dichloromethane, dried (sodium sulphate), filtered and the solvent removed in vacuo. The crude product was purified by flash column chromatography (dichloromethane/methanol 9.9:0.1 to 9.6:0.4) to give 20 mg of (3,4-dichloro-benzyl)-(8-methyl-3-phenylethynyl-imidazo[1,2-b]pyridazin-6-yl)-amine (54%).

¹H NMR (300 MHz, CDCl₃): δ 7.48 (3H, m), 7.25 (5H, m), 6.25 (1H, s), 4.72 (1H, s), 4.49 (2H, d, J=5.1 Hz), 2.49 (3H, s).

LCMS: 407 [M+1]. (MW: 407.3)

### Intermediate 19

### 6-Chloro-2-methyl-imidazo[1,2-b]pyridazine

(Stanovnik, B.; Tisler, M. Tetrahedron, 1967, 23, 2739-2746)

A mixture of 3-amine-6-chloropyridazine (2 g, 15.44 mmol) and chloroacetone (1.162 mL, 15.44 mmol) in ethanol (15.50 mL) was heated at reflux temperature for 16 hours. The solvent was removed in vacuo, and the residue was diluted with water, and then neutralized with solid sodium bicarbonate until pH 7. The yellow precipitate was filtered off, and washed with water. The obtained yellow solid was purified by column chromatography (Biotage™/Flash, silica, methanol:dichloromethane 9.9:0.1 to 9:1) to give 6-chloro-2-methyl-imidazo[1,2-b]pyridazine as a pale yellow solid (593 mg, 23% yield).

¹H NMR (300 MHz, CDCl₃): δ 7.75 (1H, d, J=9.0 Hz), 7.68 (1H, s), 6.96 (1H, d, J=9.0 Hz), 2.47 (3H, s).

LC-MS: 168.10 [M+1], tR=0.984 min, (MW: 167.60).

### Intermediate 20

### (3,4-Dichloro-benzyl)-(2-methyl-imidazo[1,2-b]pyridazin-6-yl)-amine

A mixture of 6-chloro-2-methyl-imidazo[1,2-b]pyridazine (50 mg, 0.29 mmol), 3,4-dichlorobenzylamine (0.06 mL, 0.45 mmol), tris(dibenzylideneacetone)dipalladium(0) (14 mg, 0.02 mmol), (r)-(+)-2,2′-bis(diphenylphosphino)-1,1′-binaphthyl (17 mg, 0.09 mmol), and sodium ethoxide (46 mg, 1.60 mmol) in 1,4-dioxane (2 mL) was heated at reflux for 16 hours. The reaction was diluted with dichloromethane, and washed with water. The organic layer was dried (sodium sulphate), filtered and concentrated. The crude mixture was purified by column chromatography (Biotage™/Flash, silica, methanol:dichloromethane 9.9:0.1 to 9:1) to give a yellow solid, which was washed with dichloromethane/hexanes to afford 23 mg of (3,4-dichloro-benzyl)-(2-methyl-imidazo[1,2-b]pyridazin-6-yl)-amine as a pale yellow solid (25% yield).

¹H NMR (300 MHz, CDCl₃): δ 7.54-7.39 (4H, m), 7.22 (1H, d, J=9.0 Hz), 6.39 (1H, d, J=9.0 Hz), 4.87 (1H, bs), 4.50 (2H, d, J=6.0 Hz), 2.41 (3H, s).

LC-MS: 307 [M+1], tR=3.543 min, (MW: 307.18).

### Intermediate 21

### (3,4-Dichloro-benzyl)-(3-iodo-2-methyl-imidazo[1,2-b]pyridazin-6-yl)-amine

A mixture of (3,4-dichloro-benzyl)-(2-methyl-imidazo[1,2-b]pyridazin-6-yl)-amine (20 mg, 0.065 mmol), and N-iodosuccinimide (16 mg, 0.072 mmol) in N,N-dimethylformamide (0.15 mL) was stirred at room temperature for 16 hours. The reaction was diluted with dichloromethane, and washed with 10% solution of sodium thiosulphate and saturated solution of sodium chloride. The organic layer was dried (sodium sulphate), filtered and concentrated. The crude mixture was purified by column chromatography (Biotage™/Flash, silica, methanol:dichloromethane 9.9:0.1 to 9:1) to give (3,4-dichloro-benzyl)-(3-iodo-2-methyl-imidazo[1,2-b]pyridazin-6-yl)-amine as a yellow solid (24 mg, 85% yield).

¹H NMR (300 MHz, CDCl₃): δ 7.55 (1H, s), 7.40 (1H, d, J=9.0 Hz), 7.32 (1H, d, J=6.0 Hz), 7.25 (1H, d, J=9.0 Hz), 6.40 (1H, d, J=12.0 Hz), 5.16 (1H, s), 4.46 (2H, d, J=6.0 Hz), 2.36 (3H, s).

LC-MS: 432, [M+1], tR=4.119 min, (MW: 433.08).

### Example 72

### (3,4-Dichloro-benzyl)-(2-methyl-3-phenylethynyl-imidazo[1,2-b]pyridazin-6-yl)-amine

A mixture of (3,4-dichloro-benzyl)-(3-iodo-2-methyl-imidazo[1,2-b]pyridazin-6-yl)-amine (20 mg, 0.046 mmol), phenylacetylene (0.017 mL, 0.15 mmol), dichlorobis(triphenylphosphine)palladium(II) (3 mg, 0.005 mmol), copper(I) iodide (1 mg, 0.005 mmol), and triethylamine (0.23 mL, 0.008 mmol) in N,N-dimethylformamide (0.22 mL) was heated in a sealed tube at 60° C. for 3 hours. The reaction mixture was diluted with dichloromethane, and washed with water. The organic layer was dried (sodium sulphate), filtered and concentrated. The crude mixture was purified by column chromatography (Biotage™/Flash, silica, methanol:dichloromethane 9.9:0.1 to 9:1) to give a yellow solid, which was washed with dichloromethane/hexanes to afford (3,4-dichloro-benzyl)-(2-methyl-3-phenylethynyl-imidazo[1,2-b]pyridazin-6-yl)-amine as a pale yellow solid (10 mg, 53% yield).

¹H NMR (300 MHz, CDCl₃): δ 7.68 (1H, brs), 7.60-7.57 (3H, m), 7.40-7.28 (5H, m), 6.70 (1H, bs), 5.41 (1H, bs), 4.58 (2H, d, J=3.0 Hz), 2.59 (3H, s).

LC-MS: 407 [M+1], tR=4.552 min, (MW: 407.30).

### Intermediate 22

### N-(3,4-dichlorobenzyl)imidazo[1,2-b]pyridazin-6-amine

A mixture of 6-chloroimidazo[1,2-b]pyridazine (100 mg, 0.65 mmol) and 3,4-dichlorobenzylamine (0.40 mL, 2.93 mmol) was stirred at 180° C. for 5 hours under microwave irradiation (200 W). The mixture was purified by flash column chromatography (dichloromethane/methanol, 9.9:0.1 to 9:1) to yield N-(3,4-dichlorobenzyl)imidazo[1,2-b]pyridazin-6-amine (171 mg, 90% yield) as a yellow solid.

¹H NMR (300 MHz, CDCl₃): δ 7.61 (1H, t, J=4.8 Hz), 7.47 (1H, d, J=4.5 Hz), 7.39 (1H, d, J=8.3 Hz), 7.20 (1H, d, J=8.2 Hz), 6.42 (1H, d, J=9.7 Hz), 4.80 (s, 1H), 4.50 (1H, d, J=5.7 Hz).

LCMS: 293 [M+1], tR=3.32 min (MW: 293.15).

### Intermediate 23

### N-(3,4-dichlorobenzyl)-3-iodoimidazo[1,2-b]pyridazin-6-amine

A mixture of N-(3,4-dichlorobenzyl)imidazo[1,2-b]pyridazin-6-amine (100 mg, 0.34 mmol) and N-iodosuccinimide (83 mg, 0.37 mmol) in dimethylformamide (0.77 mL) was stirred at room temperature overnight. The mixture was diluted with dichloromethane, washed with sodium thiosulfate (10% solution) and saturated sodium chloride solution. The organic layer was dried (sodium sulphate anh.), filtered, and concentrated. The residue was purified by flash column chromatography (dichloromethane/methanol, 9.9:0.1 to 9:1) to yield N-(3,4-dichlorobenzyl)-3-iodoimidazo[1,2-b]pyridazin-6-amine (135 mg, 95% yield) as a yellow solid.

¹H NMR (300 MHz, DMSO-d₆): δ 7.76 (2H, m), 7.70 (1H, d, J=9.6 Hz), 7.59 (1H, d, J=8.3 Hz), 7.51 (1H, s), 7.46 (1H, dd, J=1.8, 8.3 Hz), 6.72 (1H, d, J=9.6 Hz), 4.45 (2H, d, J=5.8 Hz).

LCMS: 4.19 (M+1), tR=4.5 min. (MW: 419.05).

### Example 73

### N-(3,4-dichlorobenzyl)-3-(3-phenylprop-1-ynyl)imidazo[1,2-b]pyridazin-6-amine

A mixture of N-(3,4-dichlorobenzyl)-3-iodoimidazo[1,2-b]pyridazin-6-amine (100 mg, 0.24 mmol), 3-phenyl-1-propyne (0.1 mL, 0.79 mmol), dichlorobis(triphenylphosphine)palladium(II) (17 mg, 0.024 mmol), and cupper(I) iodide (5 mg, 0.024 mmol) in triethylamine (0.75 mL) was heated at 60° C. for 7 hours. The reaction was poured into water and extracted with dichloromethane. The combined organic layers were dried (sodium sulphate), filtered and concentrated. The residue was purified by flash column chromatography (dichloromethane-methanol 9.9:0.1 to 9:1) to yield N-(3,4-dichlorobenzyl)-3-(3-phenylprop-1-ynyl)imidazo[1,2-b]pyridazin-6-amine (80 mg, 82% yield) as a yellow solid.

¹H NMR (300 MHz, CDCl₃) [symbol] 7.54-7.48 (4H, m), 7.36-7.25 (6H, m), 6.51 (1H, brs), 4.85 (1H, brs), 4.55 (2H, d, J=5.1 Hz), 4.03 (2H, s).

LCMS: 407.21 (M+1), tR=4.98 min. (MW: 407.3).

### Intermediate 24

### 6-Chloro-2-methyl-3-nitro-imidazo[1,2-b]pyridazine

6-Chloro-2-methyl-imidazo[1,2-b]pyridazine (500 mg, 2.90 mmol) was dissolved in concentrated sulfuric acid (23 mL) at room temperature. The mixture was cooled to 0° C., and nitric acid (16 mL) was added very slowly. The reaction was stirred at this temperature for 30 minutes, and then at room temperature for 3 hours. The reaction was neutralized with saturated sodium bicarbonate solution, and extracted twice with dichloromethane. The organic layers were combined and washed with water, dried (sodium sulphate), filtered and concentrated to give 563 mg 6-chloro-2-methyl-3-nitro-imidazo[1,2-b]pyridazine as a white solid (89% yield).

¹H NMR (300 MHz, CDCl₃): δ 7.91 (1H, d, J=9.0 Hz), 7.33 (1H, d, J=9.0 Hz), 2.79 (3H, s).

LC-MS: 213 [M+1], tR=3.700 min, (MW: 212.60).

### Intermediate 25

### (3,4-Dichloro-benzyl)-(2-methyl-3-nitro-imidazo[1,2-b]pyridazin-6-yl)-amine

A mixture of 6-chloro-2-methyl-3-nitro-imidazo[1,2-b]pyridazine (150 mg, 0.71 mmol), and 3,4-dichlorobenzylamine (0.3 mL, 2.12 mmol) in 1,4-dioxane (3 mL) was heated in a sealed tube at 200° C. for 40 hours. The solvent was eliminated in vacuo. The crude mixture was purified by trituration from dichloromethane followed by column chromatography (Biotage™/Flash, silica, methanol:dichloromethane 9.9:0.1 to 9:1) to give 188 mg of (3,4-dichloro-benzyl)-(2-methyl-3-nitro-imidazo[1,2-b]pyridazin-6-yl)-amine (76% yield).

¹H NMR (300 MHz, CDCl₃): δ 7.70 (1H, d, J=9.0 Hz), 7.59 (1H, s), 7.40-7.36 (2H, m), 6.79 (1H, d, J=12.0 Hz), 5.31 (1H, brs), 4.59 (2H, s), 2.78 (3H, s).

LC-MS: 352 [M+1]), t_{R}=4.826 min, (MW: 352.18).

### Intermediate 26

### (4-Fluoro-benzyl)-(2-methyl-3-nitro-imidazo[1,2-b]pyridazin-6-yl)-amine

A mixture of 6-chloro-2-methyl-3-nitro-imidazo[1,2-b]pyridazine (200 mg, 0.94 mmol) and 4-fluorobenzylamine (0.5 mL, 4.23 mmol) in 1,4-dioxane (3 mL) was heated under microwave irradiation at 150° C. for 3.5 hours. The reaction mixture was diluted with dichloromethane, and the resulting precipitate was filtered off and washed with dichloromethane. The filtrate was concentrated in vacuo. The residue was purified by column chromatography (Biotage™/Flash, silica, methanol:dichloromethane 9.9:0.1 to 9:1) to give 242 mg (4-fluoro-benzyl)-(2-methyl-3-nitro-imidazo[1,2-b]pyridazin-6-yl)-amine as a yellow solid (85% yield).

¹H NMR (300 MHz, CDCl₃): δ 7.65 (1H, d, J=9 Hz), 7.46 (2H, dd, J=6.0, 3.0 Hz), 7.04 (2H, dd, J=9.0, 4.0 Hz), 6.73 (1H, d, J=9.0 Hz), 5.13 (1H, bs), 4.60 (2H, d, J=6.0 Hz), 2.77 (3H, s).

LC-MS: 302.10 [M+1], tR=4.473 min, (MW: 301.28).

### Example 74

### N*6*-(3,4-Dichloro-benzyl)-2-methyl-imidazo[1,2-b]pyridazine-3,6-diamine

A mixture of (3,4-dichloro-benzyl)-(2-methyl-3-nitro-imidazo[1,2-b]pyridazin-6-yl)-amine (188 mg, 0.53 mmol), and tin(II) chloride dihydrate (602 mg, 2.67 mmol) in ethanol (18 mL) was heated at 70° C. under argon for 3 hours. The reaction was poured into ice-water, and neutralized with saturated sodium bicarbonate solution. The resulting precipitated was filtered off and washed with ethyl acetate. The solvent was evaporated in vacuo. The crude mixture was purified by column chromatography (Biotage™/Flash, silica, methanol:dichloromethane 9.9:0.1 to 9:1) to give 79 mg N*6*-(3,4-dichloro-benzyl)-2-methyl-imidazo[1,2-b]pyridazine-3,6-diamine as an orange solid (46% yield).

¹H NMR (300 MHz, CDCl₃): δ 7.50-7.39 (3H, m), 7.28-7.15 (1H, m), 6.21 (1H, d, J=9.0 Hz), 4.74 (1H, bs), 4.55 (2H, d, J=6.0 Hz), 2.36 (3H, s).

LC-MS: 322.32 [M+1], tR=3.477 min, (MW: 322.20).

### Example 75

### N-[6-(3,4-Dichloro-benzylamino)-2-methyl-imidazo[1,2-b]pyridazin-3-yl]-propionamide

N*6*-(3,4-Dichloro-benzyl)-2-methyl-imidazo[1,2-b]pyridazine-3,6-diamine (15 mg, 0.05 mmol) was dissolved in dichloromethane (0.5 mL) at room temperature. The reaction was cooled to 0° C., and pyridine (0.030 mL, 0.23 mmol) was added, followed by the addition of propionic anhydride (0.019 mL, 0.23 mmol). The mixture was stirred at this temperature for 15 minutes, and then at room temperature for 16 hours. The solvent was evaporated in vacuo. The crude mixture was triturated from diethyl ether to give N-[6-(3,4-dichloro-benzylamino)-2-methyl-imidazo[1,2-b]pyridazin-3-yl]-propionamide as a yellow solid (11 mg, 62% yield).

¹H NMR (300 MHz, DMSO-d₆): δ 7.64 (3H, m), 7.36 (1H, d, J=6.0 Hz), 6.68 (1H, d, J=9.0 Hz), 4.40 (2H, d, J=3.0 Hz), 2.35 (2H, q, J=6.0 Hz), 2.15 (3H, s), 1.09 (3H, t, J=6.0 Hz).

LC-MS: 378.38 [M+1], tR=3.470 min, (MW: 378.26).

### Example 76

### N-[6-(3,4-Dichloro-benzylamino)-2-methyl-imidazo[1,2-b]pyridazin-3-yl]-benzamide

N*6*-(3,4-Dichloro-benzyl)-2-methyl-imidazo[1,2-b]pyridazine-3,6-diamine (0.043 g, 0.13 mmol) was dissolved in dry pyridine (2 mL) at 0° C. Benzoyl chloride (0.02 mL, 0.146 mmol) was added to the solution, and the brown reaction mixture was stirred at 0° C., and then at room temperature for 16 hours. The solvent was evaporated in vacuo to provide a brown oily residue which was partitioned between dichloromethane and saturated sodium bicarbonate solution. The organic layer was dried (sodium sulphate), filtered and concentrated. The obtained crude residue was triturated from diethyl ether to afford 16 mg of N-[6-(3,4-Dichloro-benzylamino)-2-methyl-imidazo[1,2-b]pyridazin-3-yl]-benzamide as a dark orange-ochre solid (28% yield).

¹H NMR (300 MHz, DMSO-d₆): δ 10.04 (1H, s), 8.01 (2H, d, J=7.0 Hz), 7.64-7.45 (6H, m), 7.35 (1H, d, J=8.0 Hz), 7.24 (1H, d, J=8.0 Hz), 6.64 (1H, d, J=9.0 Hz), 4.30 (2H, d, J=5.0 Hz), 2.19 (3H, s).

LC-MS: 426 [M+1], tR=8.15 min, (MW: 426.31).

### Example 77

### N*6*-(4-Fluoro-benzyl)-2-methyl-imidazo[1,2-b]pyridazine-3,6-diamine

A mixture of (3,4-dichloro-benzyl)-(2-methyl-3-nitro-imidazo[1,2-b]pyridazin-6-yl)-amine (200 mg, 0.66 mmol) and tin(II) chloride dihydrate (749 mg, 3.32 mmol) in ethanol (5 mL) was heated at 70° C. under argon for 4 hours. The reaction mixture was poured into ice-water, and neutralized with saturated sodium bicarbonate solution. A precipitate was formed which was filtered off and washed with ethyl acetate. The two layers present in the filtrate were separated, and the aqueous layer was extracted with ethyl acetate (3×). The combined organic layers were dried (sodium sulphate), filtered and concentrated. The crude mixture was purified by column chromatography (Biotage™/Flash, silica, methanol:dichloromethane 9.9:0.1 to 9:1) to give N*6*-(4-fluoro-benzyl)-2-methyl-imidazo[1,2-b]pyridazine-3,6-diamine as a red solid (130 mg, 72% yield).

¹H NMR (300 MHz, CDCl₃): δ 7.31-7.23 (3H, m), 6.92 (2H, t, J=9.0 Hz), 6.12 (1H, d, J=9.0 Hz), 4.95 (1H, bs), 4.43 (2H, d, J=6.0 Hz), 3.58 (2H, bs), 2.25 (3H, s).

LC-MS: 272.10 [M+1], tR=2.891 min, (MW: 271.30).

### Example 78

### N-[6-(4-Fluoro-benzylamino)-2-methyl-imidazo[1,2-b]pyridazin-3-yl]-propionamide

N*6*-(4-Fluoro-benzyl)-2-methyl-imidazo[1,2-b]pyridazine-3,6-diamine (17 mg, 0.06 mmol) was dissolved in dichloromethane (0.7 mL) at 0° C. under argon. Pyridine (0.025 mL, 0.313 mmol) was added to the solution, followed by the addition of propionic anhydride (0.04 mL, 0.31 mmol). The reaction mixture was stirred at 0° C. for 15 minutes, and then at room temperature for 16 hours. The solvent was evaporated in vacuo. The crude mixture was purified by trituration from diethyl ether to give 14 mg of N-[6-(4-fluoro-benzylamino)-2-methyl-imidazo[1,2-b]pyridazin-3-yl]-propionamide as a yellow solid (68% yield).

¹H NMR (300 MHz, CDCl₃): δ 9.44 (1H, s), 7.55 (1H, d, J=12.0 Hz), 7.42 (1H, dd, J=6.0, 9.0 Hz), 7.32 (1H, t, J=6.0 Hz), 7.11 (2H, t, J=9.0 Hz), 6.60 (1H, d, J=9.0 Hz), 4.37 (2H, d, J=6.0 Hz), 2.36 (2H, q, J=9.0 Hz), 2.12 (3H, s), 2.60 (3H, t, J=9.0 Hz).

LC-MS: 328.14 [M+1], tR=6.19 min, (MW: 326.27).

### Example 79

### N-[6-(4-Fluoro-benzylamino)-2-methyl-imidazo[1,2-b]pyridazin-3-yl]-benzamide

N*6*-(4-Fluoro-benzyl)-2-methyl-imidazo[1,2-b]pyridazine-3,6-diamine (61 mg, 0.22 mmol) was dissolved in dry pyridine (2 mL) at 0° C. Benzoyl chloride (0.03 mL, 0.25 mmol) was added to the solution, and the brown reaction mixture was stirred at 0° C., and then at room temperature for 16 hours. The solvent was evaporated in vacuo to provide a brown oily residue which was partitioned between dichloromethane and saturated sodium bicarbonate solution. The organic layer was dried (sodium sulphate), filtered and concentrated. The obtained crude residue was triturated from diethyl ether to yield 22 mg of N-[6-(4-fluoro-benzylamino)-2-methyl-imidazo[1,2-b]pyridazin-3-yl]-benzamide as a dark orange-ochre solid (26% yield).

¹H NMR (300 MHz, DMSO-d₆): δ 10.07 (1H, s), 8.05 (2H, d, J=7.0 Hz), 7.71-7.49 (4H, m), 7.44-7.25 (3H, m), 6.96 (2H, t, J=9.0 Hz), 6.63 (1H, d, J=9.0 Hz), 4.28 (2H, d, J=6.0 Hz), 2.20 (3H, s).

LC-MS: 376.44 [M+1], tR=7.29 min, (MW: 375.41).

### Intermediate 27

### 6-Chloro-2-methyl-imidazo[1,2-b]pyridazine-3-carboxylic acid ethyl ester

(Abignente, E.; Arena, F.; Luraschi, E.; Saturnino, C.; Rossi, F. Farmaco 1992, 47 (6), 931-944).

A mixture of 3-amino-6-chloropyridazine (4 g, 30.87 mmol) and ethyl 2-chloroacetoacetate (5.6 mL, 40.14 mmol) in ethanol (31 mL) was heated at reflux for 7 hours. Two additional mL of ethyl 2-chloroacetoacetate were added, and the reaction mixture was stirred at reflux for 16 hours. The solvent was evaporated in vacuo. The crude mixture was purified by column chromatography (Biotage™/Flash, silica, methanol:dichloromethane 9.9:0.1 to 9:1) to give 3.65 g of 6-chloro-2-methyl-imidazo[1,2-b]pyridazine-3-carboxylic acid ethyl ester as a yellow solid (40% yield).

¹H NMR (300 MHz, CDCl₃): δ 7.80 (1H, d, J=9.0 Hz), 7.15 (1H, d, J=9.0 Hz), 4.39 (2H, q, J=6.0 Hz), 2.65 (3H, s), 1.37 (3H, t, J=6.0 Hz).

LC-MS: 240 [M+1], tR=3.942 min, (MW: 239.66).

### Intermediate 28

### 6-Chloro-2-methyl-imidazo[1,2-b]pyridazine-3-carboxylic acid phenylamide

A 2.0 M solution of trimethylaluminum in hexanes (1.25 mL, 2.50 mmol) was added slowly at room temperature to a mixture of phenylamine (0.23 mL, 2.50 mmol) in dichloromethane (6 mL), and the mixture was stirred at room temperature for 15 minutes. Then, 6-chloro-2-methyl-imidazo[1,2-b]pyridazine-3-carboxylic acid ethyl ester (400 mg, 1.67 mmol) in dichloromethane (2 mL) was added to the reaction, and stirred at 40° C. for 16 hours. Hydrochloride acid (2N) was added to quench the reaction, and the mixture was extracted twice with dichloromethane. The combined organic layers were dried (sodium sulphate), filtered and concentrated. The crude mixture was purified by column chromatography (Biotage™/Flash, silica, methanol:dichloromethane 9.9:0.1 to 9:1) to give 6-chloro-2-methyl-imidazo[1,2-b]pyridazine-3-carboxylic acid phenylamide as a yellow solid (350 mg, 73% yield).

¹H NMR (300 MHz, CDCl₃): δ 10.23 (1H, s), 7.89 (1H, d, J=12.0 Hz), 7.64 (2H, d, J=6.0 Hz), 7.30 (2H, dd, J=9.0, 6.0 Hz), 7.16 (1H, d, J=6.0 Hz), 7.06 (1H, dd, J=9.0, 6.0 Hz), 2.82 (3H, s).

LC-MS: 287.10 [M+1], tR=4.48 min, (MW: 286.72).

### Example 80

### 6-(4-Fluoro-benzylamino)-2-methyl-imidazo[1,2-b]pyridazine-3-carboxylic acid phenylamide

A mixture of 6-chloro-2-methyl-imidazo[1,2-b]pyridazine-3-carboxylic acid phenylamide (80 mg, 0.28 mmol) and 4-fluorobenzylamine (0.096 mL 0.84 mmol) in N,N-dimethylformamide (2.5 mL) was heated at 100° C. for 4 hours. The solvent was evaporated in vacuo. The crude mixture was purified by column chromatography (Biotage™/Flash, silica, methanol:dichloromethane 9.9:0.1 to 9:1) to give 6-(4-fluoro-benzylamino)-2-methyl-imidazo[1,2-b]pyridazine-3-carboxylic acid phenylamide as a white solid (20 mg, 19% yield) together with 6-(dimethylamino)-2-methyl-N-phenylimidazo[1,2-b]pyridazine-3-carboxamide (46 mg, 56% yield).

¹H NMR (300 MHz, CDCl₃): δ 10.63 (1H, s), 7.66 (1H, d, J=9.0 Hz), 7.41 (2H, d, J=9.0 Hz), 7.32-7.19 (4H, m), 7.04-6.96 (3H, m), 6.57 (1H, d, J=9.0 Hz), 4.98 (1H, bs), 4.58 (2H, d, J=6.0 Hz), 2.74 (3H, s).

LC-MS: 376.00 [M+1], tR=4.350 min, (MW: 375.41).

### Example 81

### 6-(4-Methoxy-benzylamino)-2-methyl-imidazo[1,2-b]pyridazine-3-carboxylic acid phenylamide

A mixture of 6-chloro-2-methyl-imidazo[1,2-b]pyridazine-3-carboxylic acid phenylamide (80 mg, 0.28 mmol) and 4-methoxybenzylamine (0.109 mL 0.84 mmol) in N,N-dimethylformamide (2.5 mL) was heated at 100° C. for 4 hours. The solvent was evaporated in vacuo. The residue was purified by column chromatography (Biotage™/Flash, silica, methanol:dichloromethane 9.9:0.1 to 9:1) to give 6-(4-methoxy-benzylamino)-2-methyl-imidazo[1,2-b]pyridazine-3-carboxylic acid phenylamide as a white solid (35 mg, 32%) together with 6-(dimethylamino)-2-methyl-N-phenylimidazo[1,2-b]pyridazine-3-carboxamide (53 mg, 64% yield).

¹H NMR (300 MHz, CDCl₃): δ 10.79 (1H, s), 7.76 (1H, d, J=9 Hz), 7.53 (2H, d, J=9.0 Hz), 7.34-7.28 (4H, m), 7.11 (1H, dd, J=6.0, 9.0 Hz), 6.90 (2H, d, J=9.0 Hz), 6.76 (1H, d, J=9.0 Hz), 5.39 (1H, s), 4.62 (2H, d, J=6.0 Hz), 3.81 (3H, s), 2.83 (3H, s).

LC-MS: 388.20 [M+1], tR=4.402 min, (MW: 387.44).

### Intermediate 29

### 6-Chloro-3-nitro-imidazo[1,2-b]pyridazine

To 6-chloro-imidazo[1,2-b]pyridazine [WO 2007/013673 A1 Page 42, Preparation 38], (5.0 g, 32.55 mmol) was added concentrated sulfuric acid (7.0 mL, 131.40 mmol). The resulting solution was cooled to 5° C. in an ice bath, and yellow fuming nitric acid (5.0 mL, 119.00 mmol) was added drop-wise at a rate such as to keep the internal temperature below 10° C. The ice bath was removed and the reaction continued for 3 hours at room temperature. After this time, the reaction was heated to 75° C. for 1 hour, before pouring the mixture onto crushed ice (60 g). The resulting aqueous slurry was allowed to stand until all the ice had melted, and the precipitate was collected on a Büchner funnel. The crude material was re-crystallized from hot ethanol/water (9:1) to yield 6-chloro-3-nitro-imidazo[1,2-b]pyridazine as a off-white crystalline (5.02 g; 78%).

¹H NMR (300 MHz, CDCl₃): δ 8.57 (1H, s), 8.08 (1H, d, J=9.5 Hz), 7.42 (1H, d, J=9.5 Hz).

LCMS: 199.04 [M+1], tR=3.33 min, (MW 198.57).

### Intermediate 30

### 3-Nitro-6-phenyl-imidazo[1,2-b]pyridazine

To a mixture of 6-chloro-3-nitroimidazo[1,2-b]pyridazine (3.0 g, 15.10 mmol), phenylboronic acid (2.03 g; 16.60 mmol), tetrakis(triphenylphosphine)-palladium(0) (0.87 g, 0.80 mmol) and sodium hydroxide (1.21 g, 30.2 mmol) was added water (20 mL) and de-oxygenated 1,2-dichloroethane (40 mL). The mixture was heated at 75° C. for 3 hours. The reaction was interrupted by pouring the reaction mixture onto crushed ice to give a dense, beige precipitate. The solid was filtered by vacuum filtration, washed with ethyl acetate/diethyl ether (1/2) and further dried by azeotrope evaporation with toluene to give 2.0 g of 3-nitro-6-phenyl-imidazo[1,2-b]pyridazine as a beige powdery solid (55% yield).

¹H NMR (300 MHz, DMSO-d₆): δ 8.79 (1H, s), 8.48 (1H, d, J=9.6 Hz), 8.23 (1H, d, J=9.7 Hz), 8.14 (2H, m), 6.68 (3H, m).

LCMS: 211.1 and 241.1 [M−29, M+1], tR=4.39 min, (MW 240.22).

### Example 82

### 6-Phenyl-imidazo[1,2-b]pyridazin-3-ylamine

3-Nitro-6-phenyl-imidazo[1,2-b]pyridazine (0.25 g, 1.04 mmol) was dissolved in ethyl acetate (100 mL), filtered and reacted on the H-cube™ hydrogenation apparatus (10% palladium/charcoal-cartridge, temperature=50° C., pressure=60 bar, flow rate: 1 mL/min) (two cycles were necessary). The system was flushed with ethanol (50 mL), which was combined with the ethyl acetate-solution. Evaporation of the combined organics gave 6-phenyl-imidazo[1,2-b]pyridazin-3-ylamine (0.21 g; 96% yield) as a red oil.

¹H NMR (300 MHz, CDCl₃): δ 7.92 (2H, dd, J=7.8, 1.9 Hz), 7.81 (1H, d, J=9.4 Hz), 7.59 (1H, m), 7.42 (2H, m), 7.20 (2H, m), 4.27 (2H, s)

### Example 83

### N-(6-Phenyl-imidazo[1,2-b]pyridazin-3-yl)-propionamide

To a dichloromethane (3 mL, dry) solution of 6-phenyl-imidazo[1,2-b]pyridazin-3-ylamine (70 mg, 0.29 mmol), anhydrous pyridine (0.13 mL, 1.7 mmol) was added, followed by a dichloromethane (2 mL) solution of propionic anhydride (0.22 mL, 1.7 mmol). The reaction mixture was stirred at room temperature overnight. Dichloromethane (10 mL) was added, together with saturated sodium bicarbonate (5 mL) and the organic layer was separated. The aqueous layer was extracted with dichloromethane. The combined organic layers were dried over sodium sulphate and evaporated to give a brown, gummy solid. The crude product was purified (Biotage™/Flash, silica, ethyl acetate:hexane 7:3 to 10:0) to yield 70 mg of N-(6-phenyl-imidazo[1,2-b]pyridazin-3-yl)-propionamide as a yellow solid (79% yield).

¹H NMR (300 MHz, CDCl₃): δ 8.27 (1H, s), 8.16 (1H, s), 7.92 (3H, m), 7.50 (3H, m), 7.35 (1H, d, J=9.5 Hz), 2.58 (2H, q, J=7.5 Hz), 1.31 (3H, t, J=7.5 Hz).

LCMS: 267.1 [M+1], tR=7.87 min, (MW 266.3).

### Example 84

### N-(6-Phenyl-imidazo[1,2-b]pyridazin-3-yl)-benzamide

3-Amino-6-phenylimidazo[1,2-b]pyridazine (0.14 g; 0.67 mmol) was dissolved in anhydrous pyridine (2.5 mL) and benzoyl chloride (85 μL, 0.733 mmol) was added. The reaction was stirred at room temperature for 60 hours. The solvent was evaporated and the crude material was purified by flash column chromatography (ethyl acetate 100%) to give a brownish solid, which was further purified by washing with a cold solution of diethyl ether/ethyl acetate (5/1). The desired compound N-(6-phenyl-imidazo[1,2-b]pyridazin-3-yl)benzamide was isolated as a brown-yellow solid (0.028 g; 13% yield).

¹H-NMR (300 MHz, DMSO-d₆): [symbol] 10.70 (1H, s), 8.24 (1H, d, J=9.5 Hz), 8.11 (4H, m), 7.93 (1H, s), 7.83 (1H, d, J=9.6 Hz), 7.59 (6H, m).

LCMS: 315.1 [M+1], tR=9.75 min, (MW 314.3).

### General Procedure M for the Preparation of Examples 85-86

3-Amino-6 phenyl-imidazo[1,2-b]pyridazine (1 eq) was dissolved in anhydrous pyridine (0.23 mmol/mL) and the appropriate sulphonyl chloride (1.1 eq) (e.g. ethylsulphonyl chloride) was added. The reaction was stirred at room temperature overnight. The solvent was evaporated in vacuo and the residue was treated with a mixture of dichloromethane and saturated sodium bicarbonate solution. The combined organic layers were dried over sodium sulphate and evaporated in vacuo. The crude material was dissolved in a few drops of dichloromethane and precipitated by addition of diethyl ether to yield the desired product (e.g. ethanesulfonic acid (6-phenyl-imidazo[1,2-b]pyridazin-3-yl)-amide). If necessary, the material was further purified on silica gel (ethyl acetate 100%).

### Example 85

### Ethanesulfonic acid (6-phenyl-imidazo[1,2-b]pyridazin-3-yl)-amide

The title compound was isolated by purification on flash silica gel (7% yield).

¹H NMR (300 MHz, CDCl₃): δ 7.99 (2H, m), 7.93 (1H, s), 7.71 (1H, d, J=9.5 Hz), 7.61 (1H, s), 7.44 (1H, d, J=2.0 Hz), 7.43 (2H, d, J=1.8 Hz), 3.13 (2H, q, J=7.3 Hz), 1.38 (3H, t, J=7.34 Hz).

LCMS: 303.1 [M+1], tR=8.69 min, (MW 302.4).

### Example 86

### N-(6-Phenyl-imidazo[1,2-b]pyridazin-3-yl)-benzenesulfonamide

The title compound was isolated by precipitation from dichloromethane and diethyl ether (41% yield).

¹H NMR (300 MHz, DMSO-d₆): δ 7.86 (1H, d, J=9.6 Hz), 7.72 (2H, dd, J=6.9, 2.9 Hz), 7.66 (2H, d, J=7.34 Hz), 7.59 (1H, d, J=9.6 Hz), 7.53 (1H, s), 7.39 (3H, m), 7.27 (3H, m).

LCMS: 351.1 [M+1], tR=10.43 min, (MW 350.4).

### General Procedure N for the Preparation of Intermediates 31-32

In a microwave reactor vessel, 6-chloro-3-nitroimidazo[1,2-b]pyridazine (1 eq) was dissolved in anhydrous 1,4-dioxane (1.33 mmol/mL) and N,N-diisopropylethylamine (1 eq) was added, followed by the appropriate benzylic amine (e.g. 4-fluorobenzylamine, 2 eq). The flask was flushed with argon and heated under microwave irradiation (6 hours, 150° C., 200 W). The solvent was evaporated and the residue was taken up in ethyl acetate and sodium bicarbonate and extracted with ethyl acetate. Drying (sodium sulphate) and evaporation of the solvent gave a brown oil residue that crystallized upon standing. Washing with a cold solution of ethyl acetate/diethyl ether (1/2) gave the desired product (e.g. (4-fluoro-benzyl)-(3-nitro-imidazo[1,2-b]pyridazin-6-yl)-amine).

### Intermediate 31

### (4-Fluoro-benzyl)-(3-nitro-imidazo[1,2-b]pyridazin-6-yl)-amine

The title compound was isolated in 72% yield.

¹H NMR (300 MHz, DMSO-d₆): δ 8.43 (1H, s), 8.04 (1H, t, J=5.6 Hz), 7.96 (1H, d, J=9.8 Hz), 7.52 (2H, dd, J=8.5, 5.7 Hz), 7.16 (2H, t, J=8.9 Hz), 7.05 (1H, d, J=9.8 Hz), 4.48 (2H, d, J=5.7 Hz).

### Intermediate 32

### (3,4-Dichloro-benzyl) (3-nitro-imidazo-[1,2-b]pyridazin-6-yl)amine

The title compound was isolated in 73% yield.

¹H NMR (300 MHz, CDCl₃): δ 8.35 (1H, s), 7.75 (1H, d, J=9.7 Hz), 7.58 (1H, s), 7.34-7.40 (2H, m), 6.72 (1H, d, J=9.7 Hz), 5.12 (1H, s), 4.59 (2H, d, J=4.0 Hz).

### General Procedure O for the Preparation of the Examples 87-88

The appropriate nitro compound (e.g (4-fluoro-benzyl)-(3-nitro-imidazo[1,2-b]pyridazin-6-yl)-amine, 1 eq) and tin(II)chloride dihydrate (5 eq) were suspended in absolute ethanol (0.9 mmol/mL). The reaction was heated under nitrogen at 75° C. for 90 minutes. The reaction mixture was allowed to cool, the solvent evaporated and the residue taken up in water. Saturated sodium bicarbonate solution was added to pH 8. The resulting precipitate was filtered off and washed with ethyl acetate (150 mL). The filtrate was extracted with ethyl acetate (3×150 mL), the combined organic layers dried and the solvent evaporated under vacuum to give the desired compound (e.g. N*6*-(4-fluoro-benzyl)-imidazo[1,2-b]pyridazine-3,6-diamine).

### Example 87

### N*6*-(4-Fluoro-benzyl)-imidazo[1,2-b]pyridazine-3,6-diamine

The title compound was isolated in 80% yield.

¹H NMR (300 MHz, DMSO-d₆): δ 8.01 (1H, t, J=5.6 Hz), 7.80 (1H, d, J=9.8 Hz), 7.50 (2H, dd, J=8.4, 5.7 Hz), 7.17 (2H, t, J=8.8 Hz), 7.00 (1H, s), 6.94 (1H, d, J=9.8 Hz), 5.77 (2H, brs), 4.54 (2H, d, J=5.6 Hz).

### Example 88

### N*6*-(3,4-Dichloro-benzyl)imidazo[1,2-b]pyridazine-3,6-diamine

The title compound was isolated in 56% yield.

¹H NMR (300 MHz, CDCl₃): δ 7.45 (2H, m), 7.21 (1H, m), 6.96 (1H, s), 6.23 (2H, d, J=9.5 Hz), 4.66 (1H, s), 4.52 (2H, d, J=5.7 Hz), 3.82 (2H, s).

### General Procedure P for the Preparation of Examples 89-92

N*6*-(4-Fluoro-benzyl)-imidazo[1,2-b]pyridazine-3,6-diamine (1 eq) was dissolved in anhydrous pyridine (0.17 mmol/mL) and the appropriate acid chloride (1.1 eq) or appropriate acid anhydride (1.1 eq) (e.g. propionic anhydride) was added. The reaction mixture was stirred at room temperature overnight. The solvent was evaporated and the residue was treated with a mixture of dichloromethane and saturated bicarbonate solution. The combined organic layers were dried over sodium sulphate and the solvent evaporated in vacuo. The crude material was purified by washing either with diethyl ether or a cold (4:1)-mixture of diethyl ether and ethanol to give the desired product (e.g. N-[6-(4-fluoro-benzylamino)-imidazo[1,2-b]pyridazin-3-yl]-propionamide).

### Example 89

### N-[6-(4-Fluoro-benzylamino)-imidazo[1,2-b]pyridazin-3-yl]-propionamide

The title compound was obtained after purification by trituration of the crude material from diethyl ether (13% yield).

¹H NMR (300 MHz, methanol-d₄): δ 8.57 (1H, s), 7.68 (2H, d, J=8.6 Hz), 7.46 (2H, dd, J=8.5, 5.5 Hz), 7.07 (2H, t, J=8.8 Hz), 6.87 (1H, d, J=9.8 Hz), 4.60 (2H, s), 2.57 (2H, q, J=7.6 Hz), 1.25 (3H, t, J=7.6 Hz).

LCMS: 314.2 [M+1], tR=6.57 min, (MW 313.3).

### Example 90

### N-[6-(4-Fluoro-benzylamino)-imidazo[1,2-b]pyridazin-3-yl]-benzamide

The title compound was obtained after washing the crude mixture with a cold mixture of diethyl ether and ethanol (4:1) (9% yield).

¹H NMR (300 MHz, DMSO-d₆): δ 10.14 (1H, s), 8.57 (1H, d, J=4.0 Hz), 8.01 (1H, d, J=6.8 Hz), 7.66 (5H, m), 7.40 (4H, m), 7.02 (1H, t, J=8.7 Hz), 6.71 (1H, d, J=9.7 Hz), 4.38 (2H, d, J=4.4 Hz).

LCMS: 362.1 [M+1], tR=7.63 min, (MW 361.4).

### Example 91

### N-[6-(4-Fluoro-benzylamino)-imidazo[1,2-b]pyridazin-3-yl]-4-methoxy-benzamide

The title compound was obtained after washing the crude mixture with diethyl ether (42% yield).

¹H NMR (300 MHz, methanol-d₄): δ 7.95 (2H, d, J=8.5 Hz), 7.56 (2H, m), 7.38 (2H, m), 7.08 (2H, d, J=8.6 Hz), 6.95 (2H, t, J=8.7 Hz), 6.69 (1H, d, J=9.7 Hz), 4.47 (2H, s), 3.90 (3H, s).

LCMS: 391.4 [M+1], tR=7.80 min, (MW 392.1).

### Example 92

### N-[6-(4-Fluoro-benzylamino)-imidazo[1,2-b]pyridazin-3-yl]-3-methoxy-benzamide

The title compound was obtained after washing the crude mixture with diethyl ether (49% yield).

LCMS: 392.1 [M+1], tR=3.64 min, (MW 391.4).

### Example 93

### N-[6-(3,4-Dichloro-benzylamino)-imidazo[1,2-b]pyridazin-3-yl]-propionamide

N*6*-(3,4-Dichloro-benzyl)imidazo[1,2-b]pyridazine-3,6-diamine (0.075 g, 0.24 mmol) was dissolved in anhydrous dichloromethane (2 mL). Pyridine (0.077 mL, 0.96 mmol) was added to the above solution, followed by dropwise addition of a solution of propionic anhydride (0.12 mL, 0.96 mmol) in anhydrous dichloromethane (1 mL). The resulting mixture was stirred for 6 hours at room temperature, and a dense precipitate was formed. The solid was isolated by vacuum filtration, washed with cold diethyl ether and dried in vacuo. The title product N-[6-(3,4-dichloro-benzylamino)-imidazo[1,2-b]pyridazin-3-yl]-propion-amide was isolated as a grey solid (65.2 mg; 70% yield).

¹H NMR (300 MHz, DMSO-d₆): δ 9.67 (1H, s), 7.67 (2H, m), 7.58 (2H, m), 7.42 (2H, s), 6.62 (1H, d, J=9.7 Hz), 4.51 (2H, d, J=5.8 Hz), 2.45 (2H, q, J=7.5 Hz), 1.10 (3H, t, J=7.5 Hz).

LCMS: 364.1 [M+1], t_{R}=7.59 min, (MW=363.3).

### Example 94

### N-[6-(3,4-Dichloro-benzylamino)-imidazo[1,2-b]pyridazin-3-yl]-4-methoxy-benzamide

To a solution of N*6*-(3,4-Dichloro-benzyl)-imidazo[1,2-b]pyridazine-3,6-diamine (0.15 g; 0.49 mmol) in dry pyridine (2.5 mL), 4-methoxybenzoyl chloride (0.072 mL, 0.54 mmol) was added. The reaction mixture was stirred at room temperature overnight. The solvent was evaporated in vacuo and the crude product was washed with dichloromethane, filtered and dried to yield 42 mg of N-[6-(3,4-dichloro-benzylamino)-imidazo[1,2-b]pyridazin-3-yl]-4-methoxy-benzamide (20% yield).

¹H-NMR (300 MHz, DMSO-d₆): δ 10.60 (1H, s), 8.61 (1H, s), 8.03 (4H, m), 7.60 (1H, s), 7.47 (1H, d, J=8.2 Hz), 7.30 (1H, m), 7.12 (2H, d, J=8.7 Hz), 4.48 (2H, s), 3.86 (3H, s).

LCMS: 442.1 [M], tR=8.61 min, (MW 442.3).

### General Procedure for Q the Preparation of Intermediates 33-34

The appropriate phenol or imidazole (2.2 eq) (e.g. phenol) was dissolved in 1,4-dioxane (0.21 mmol/mL) and added to a screwcap vial containing a suspension of sodium hydride (2.6 eq) in dry anhydrous 1,4-dioxane. The reaction mixture was stirred at room temperature for 30 minutes before addition of 6-chloro-3-nitro-imidazo[1,2-b]pyridazine (1 eq). Stirring was continued at room temperature overnight. The solution was evaporated and the residue was taken up in dichloromethane and brine and extracted with dichloromethane. The combined organic layers were dried (sodium sulphate) and evaporated in vacuo to give the final product (e.g. 3-nitro-6-phenoxy-imidazo[1,2-b]pyridazine), which was used without further purification.

### Intermediate 33

### 3-Nitro-6-phenoxy-imidazo[1,2-b]pyridazine

The title compound was obtained in 77% yield.

¹H NMR (300 MHz, CDCl₃): δ 8.50 (1H, s), 8.09 (1H, d, J=9.8 Hz), 7.50 (2H, m), 7.35 (3H, m), 7.25 (1H, d, J=9.7 Hz).

### Intermediate 34

### 6-Imidazol-1-yl-3-nitro-imidazo[1,2-b]pyridazine

The title compound was obtained in 62% yield.

¹H NMR (300 MHz, CDCl₃): δ 8.58 (1H, s), 8.32 (1H, s), 8.23 (1H, d, J=9.7 Hz), 7.74 (1H, s), 7.56 (1H, d, J=9.7 Hz), 7.25 (1H, s).

### General Procedure R for the Preparation of Examples 95-96

The appropriate nitro compound (1 eq) (e.g. 3-nitro-6-phenoxy-imidazo[1,2-b]pyridazine) was dissolved in ethyl acetate (0.012 mmol/mL), filtered and reacted on the H-cube™ hydrogenation apparatus (10% palladium/charcoal-cartridge, temperature=50° C., pressure=60 bar, flow rate: 1 mL/min) (two cycles were necessary). The system was flushed with ethanol (50 mL), which was combined with the ethyl acetate solution. Evaporation of the solution gave the reduced compound (e.g. 6-phenoxy-imidazo[1,2-b]pyridazin-3-ylamine).

### Example 95

### 6-Phenoxy-imidazo[1,2-b]pyridazin-3-ylamine

The title compound was isolated in 88% yield.

¹H NMR (300 MHz, DMSO-d₆): δ 8.80 (1H, s), 8.49 (1H, d, J=9.6 Hz), 8.25 (1H, d, J=9.6 Hz), 8.15 (2H, m), 7.57 (3H, m).

### Example 96

### 6-Imidazol-1-yl-imidazo[1,2-b]pyridazin-3-ylamine

The title compound 6-imidazol-1-yl-imidazo[1,2-b]pyridazin-3-ylamine was isolated in 72% yield.

¹H-NMR (300 MHz, CDCl₃): δ 8.64 (1H, s), 8.14 (2H, m), 7.38 (1H, d, J=9.5 Hz), 7.18 (1H, s), 7.11 (1H, s), 5.73 (2H, s).

### General Procedure S for the Preparation of Examples 97-100

The appropriate amino compound (1 eq) (e.g. 6-phenoxy-imidazo[1,2-b]pyridazin-3-ylamine) was dissolved in anhydrous pyridine (0.23 mmol/mL) and the appropriate acid chloride (1.1 eq) or acid anhydride (1.1 eq) (e.g. propionic anhydride) was added. The reaction was stirred at room temperature overnight. The solvent was eliminated in vacuo and the residue was treated with a mixture of dichloromethane and saturated sodium bicarbonate solution. The combined organic layers were dried (sodium sulphate) and the solvent evaporated in vacuo. The crude material was purified by precipitation from dichloromethane/diethyl ether to give the wanted product (e.g. N-(6-phenoxy-imidazo[1,2-b]pyridazin-3-yl)-propionamide). If necessary, the material was further purified by flash column chromatography (ethyl acetate:ethanol 9:1).

### Example 97

### N-(6-Phenoxy-imidazo[1,2-b]pyridazin-3-yl)-propionamide

The title compound was obtained after precipitation from dichloromethane/diethyl ether (44% yield).

¹H NMR (300 MHz, methanol-d₄): δ 8.05 (1H, s), 7.89 (1H, d, J=9.6 Hz), 7.82 (1H, s), 7.45 (2H, t, J=7.7 Hz), 7.30 (1H, d, J=7.3 Hz), 7.20 (2H, d, J=7.9 Hz), 6.77 (1H, d, J=9.6 Hz), 2.44 (2H, q, J=7.5 Hz), 1.38 (3H, t, J=7.5 Hz).

LCMS: 283.1 [M+1], tR=7.72 min, (MW 282.3).

### Example 98

### N-(6-Phenoxy-imidazo[1,2-b]pyridazin-3-yl)-benzamide

The title compound was obtained after precipitation from dichloromethane/diethyl ether (48% yield).

¹H NMR (300 MHz, CDCl₃): δ 8.34 (1H, s), 8.01 (1H, s), 7.77 (1H, d, J=9.6 Hz), 7.63 (2H, d, J=7.6 Hz), 7.41 (1H, t, J=7.3 Hz), 7.32 (4H, t, J=6.9 Hz), 7.17 (1H, t, J=7.3 Hz), 7.09 (2H, d, J=7.6 Hz), 6.67 (1H, d, J=9.6 Hz).

LCMS: 331.1 [M+1], tR=10.00 min, (MW 330.3).

### Example 99

### N-(6-Imidazol-1-yl-imidazo[1,2-b]pyridazin-3-yl)-propionamide

The title compound was obtained after purification by flash column chromatography on silica gel (25% yield).

¹H NMR (300 MHz, methanol-d₄): δ 8.67 (1H, s), 8.16 (1H, d, J=9.7 Hz), 8.04 (1H, s), 7.99 (1H, s), 7.67 (1H, d, J=9.7 Hz), 7.24 (1H, s), 2.63 (2H, q, J=7.6 Hz), 1.28 (3H, t, J=7.6 Hz).

LCMS: 257.2 [M+1], tR=4.48 min, (MW 256.3).

### Example 100

### N-(6-Imidazol-1-yl-imidazo[1,2-b]pyridazin-3-yl)-4-methoxy-benzamide

The title compound was obtained after precipitation from dichloromethane/diethyl ether (37% yield).

¹H NMR (300 MHz, methanol-d₄): δ 8.61 (1H, s), 8.18 (1H, d, J=9.7 Hz), 8.02 (4H, t, J=10.0 Hz), 7.68 (1H, d, J=9.7 Hz), 7.21 (1H, s), 7.07 (2H, d, J=8.6 Hz), 3.90 (3H, s).

LCMS: 335.1 [M+1], tR=6.34 min, (MW 334.3).

### General Procedure T for the Preparation of Examples 101-103

The appropriate methoxy phenyl amide derivative (1 eq) (e.g. N-(6-imidazol-1-yl-imidazo[1,2-b]pyridazin-3-yl)-4-methoxy-benzamide) was suspended in dry dichloromethane (0.063 mmol/mL). The suspension was cooled in an ice bath and stirred for 10 minutes before addition of boron tribromide (1M in dichloromethane, 7 eq). The reaction mixture was allowed to reach room temperature and stirred between 15 hours and 4 days. When the reaction was not complete after 15 hours, more boron tribromide solution was added. Evaporation of the solvent, followed by addition of methanol (2 mL), stirring and evaporation, gave a brown solid that was suspended in cold water and isolated by vacuum filtration. The crude solid was washed as specified and dried to give the desired product (e.g. 4-hydroxy-N-(6-imidazol-1-yl-imidazo[1,2-b]pyridazin-3-yl)-benzamide).

### Example 101

### N-[6-(4-Fluoro-benzylamino)-imidazo[1,2-b]pyridazin-3-yl]-4-hydroxy-benzamide

The title compound was isolated after 63 hours stirring at room temperature, and purified by washing with diethyl ether (63% yield).

¹H NMR (300 MHz, DMSO-d₆): δ 10.41 (1H, s), 10.31 (1H, s), 8.29 (1H, s), 8.09 (1H, s), 8.01 (1H, d, J=9.8 Hz), 7.90 (2H, d, J=8.3 Hz), 7.39 (2H, m), 7.21 (1H, d, J=9.9 Hz), 7.06 (2H, t, J=8.7 Hz), 6.94 (2H, m), 4.46 (2H, s).

LCMS: 378.1 [M+1], tR=6.73 min, (MW 377.4).

### Example 102

### N-[6-(4-Fluoro-benzylamino)-imidazo[1,2-b]pyridazin-3-yl]-3-hydroxy-benzamide

The title compound was isolated after 15 hours stirring at room temperature, and purified by washing with diethyl ether/ethanol (4:1) (78% yield).

¹H NMR (300 MHz, DMSO-d₆): δ 10.65 (1H, s), 9.91 (1H, s), 8.38 (1H, m), 8.15 (1H, s), 8.03 (1H, d, J=9.8 Hz), 7.42 (5H, m), 7.26 (1H, d, J=9.8 Hz), 7.08 (3H, s), 4.46 (2H, d, J=5.1 Hz).

LCMS: 378.2 [M+1], tR=6.87 min, (MW 377.4).

### Example 103

### 4-Hydroxy-N-(6-imidazol-1-yl-imidazo[1,2-b]pyridazin-3-yl)-benzamide

The title compound was isolated after stirring for 4 days at room temperature, and purified by washing with diethyl ether/ethanol (5:1) (75% yield).

¹H NMR (300 MHz, DMSO-d₆): δ 10.61 (1H, d, J=12.6 Hz), 9.91 (1H, m), 8.56 (2H, m), 8.16 (1H, d, J=10.5 Hz), 7.94 (4H, m), 6.93 (2H, d, J=7.5 Hz).

LCMS: 321.1 [M+1], tR=5.16 min, (MW 320.3).

### General Procedure U for the Preparation of Examples 104-107

The appropriate isocyanate (e.g. ethyl isocyanate) was added to a solution of N*6*-(3,4-dichloro-benzyl)-imidazo[1,2-b]pyridazine-3,6-diamine (1 eq) in anhydrous tetrahydrofuran (0.041 mmol/mL). The reaction mixture was heated at 65° C. for 24 hours, one more equivalent of isocyanate was added and the mixture was heated further (16 hours). On cooling, the reaction mixture was filtered. The obtained grey solid was washed with a cold mixture of diethyl ether and ethanol (1:1) and dried at 40° C. in the vacuum oven to give the desired product (e.g. 1-[6-(3,4-dichloro-benzylamino)-imidazo[1,2-b]pyridazin-3-yl]-3-ethyl-urea)

### Example 104

### 1-[6-(3,4-Dichloro-benzylamino)-imidazo[1,2-b]pyridazin-3-yl]-3-ethyl-urea

The title compound was prepared in 33% yield.

¹H NMR (300 MHz, DMSO-d₆): δ 8.19 (1H, s), 7.72 (1H, s), 7.60 (2H, dd, J=8.9, 4.6 Hz), 7.45 (2H, m), 7.32 (1H, s), 6.78 (1H, t, J=5.1 Hz), 6.53 (1H, d, J=9.6 Hz), 4.52 (2H, d, J=5.6 Hz), 3.13 (2H, m), 1.06 (1H, t, J=7.2 Hz).

LCMS: 379.0 [M], tR=7.31 min, (MW 379.2).

### Example 105

### 1-Cyclopentyl-3-[6-(3,4-dichloro-benzylamino)-imidazo[1,2-b]pyridazin-3-yl]-urea

The title compound was prepared in 35% yield.

¹H NMR (300 MHz, DMSO-d₆): δ 8.12 (1H, s), 7.74 (1H, s), 7.60 (2H, dd, J=8.9, 3.1 Hz), 7.44 (2H, dt, J=10.1, 3.9 Hz), 7.33 (1H, s), 6.85 (1H, d, J=7.1 Hz), 6.53 (1H, d, J=9.7 Hz), 4.53 (2H, d, J=5.8 Hz), 3.95 (1H, m), 1.95 (2H, dt, J=18.3, 5.9 Hz), 1.59 (4H, m), 1.39 (2H, m).

LCMS: 419.0 [M], tR=8.21 min, (MW 419.3).

### Example 106

### 1-(3-Acetyl-phenyl)-3-[6-(3,4-dichloro-benzylamino)-imidazo[1,2-b]pyridazin-3-yl]urea

The title compound was prepared in 69% yield.

¹H NMR (300 MHz, DMSO-d₆): [symbol] 9.56 (1H, s), 8.61 (1H, s), 8.08 (1H, s), 7.74 (2H, m), 7.67 (1H, d, J=9.6 Hz), 7.61 (1H, d, J=8.3 Hz), 7.55 (2H, m), 7.44 (3H, m), 6.60 (1H, d, J=9.7 Hz), 4.55 (2H, d, J=5.7 Hz), 2.57 (3H, s).

LCMS: 469.2 [M+1], tR=8.01 min, (MW 468.1).

### Example 107

### 4-{3-[6-(3,4-Dichloro-benzylamino)-imidazo[1,2-b]pyridazin-3-yl]-ureido}-benzoic acid methyl ester

The title compound was prepared in 70% yield.

¹H NMR (300 MHz, DMSO-d₆): [symbol] 9.77 (1H, s), 8.76 (1H, s), 7.90 (2H, d, J=8.7 Hz), 7.71 (1H, m), 7.64 (3H, m), 7.55 (2H, m), 7.42 (2H, m), 6.61 (1H, d, J=9.6 Hz), 4.55 (2H, d, J=5.7 Hz), 3.82 (3H, s).

LCMS: 485.2 [M+1], tR=8.44 min, (MW 484.1).

### Intermediate 35

### 6-Chloroimidazo[1,2-b]pyridazinyl-3-sulfonic acid

6-Chloroimidazo[1,2-b]pyridazine (1.0 g; 6.54 mmol) was dissolved in oleum (2.5 mL). The mixture was stirred at room temperature for 3 hours and at 80° C. for 3 more hours. On cooling, the reaction mixture was poured onto crushed ice (15 g) and sodium chloride was added. A dense precipitate appeared and it was isolated by filtration in vacuo. The obtained pale-yellow solid was washed with cold water and dried first on the Büchner funnel followed by azeotrope distillation with toluene. The expected 6-chloroimidazo[1,2-b]pyridazinyl-3-sulfonic acid was isolated as a beige solid (0.90 g; 59% yield).

¹H NMR (300 MHz, DMSO-d₆): δ 8.37 (1H, d, J=9.6 Hz), 8.09 (1H, s), 7.70 (2H, d, J=9.6 Hz).

LCMS: 234.0 [M+1], tR=0.95 min, (MW 233.6).

### Intermediate 36

### 6-Chloro-imidazo[1,2-b]pyridazine-3-sulfonic acid ethylamide

6-Chloroimidazo[1,2-b]pyridazinyl-3-sulfonic acid (0.30 g; 1.20 mmol) was suspended in dry chloroform (7 mL) and triethylamine (0.67 mL; 4.80 mmol; 4 eq) was added. Heat was evolved and a brown, transparent solution formed. Phosphorus oxychloride (0.50 mL; 5.40 mmol; 4.5 eq) was added and the resulting suspension was refluxed under nitrogen for 5 hours. After cooling, the reaction mixture was poured into water and extracted with chloroform (3×15 mL). Drying and evaporation of the organic layer gave an oily, yellow solid (0.11 g) that was taken up in dry acetonotrile (2 mL) and reacted with ethylamine (2.5 mL; 5 mmol; 10 eq) at room temperature overnight. 6-Chloro-imidazo[1,2-b]pyridazine-3-sulfonic acid ethylamide was isolated as a white solid (91 mg; 29% yield), which was further reacted in its crude state.

¹H NMR (300 MHz, CDCl₃): δ 8.18 (1H, s), 8.00 (1H, d, J=9.5 Hz), 7.23 (1H, d, J=9.5 Hz), 5.22 (1H, s), 3.04 (2H, quintet, J=7.2 Hz), 1.05 (3H, t, J=7.2 Hz).

LCMS: 261.0 [M+1], tR=3.18 min, (MW 260.7).

### Intermediate 37

### 6-Chloro-3-(morpholine-4-sulfonyl)-imidazo[1,2-b]pyridazine

6-Chloro-imidazo[1,2-b]pyridazine-3-sulfonic acid (0.25 g; 1.10 mmol) was dissolved in phosphorus oxychloride (1 mL) and heated at 100° C. for 5 hours under nitrogen. The excess of phosphorus oxychloride was distilled off under vacuum, and the residue taken up in acetonitrile (5 mL). Triethylamine (0.15 mL; 1.10 mmol) was added, and the mixture was cooled in an ice bath for 10 min before adding morpholine (0.93 mL; 1.10 mmol). The reaction was continued at 0° C. for 1 hour, then at room temperature over the weekend. The reaction mixture was evaporated in vacuo, the residue taken up in sodium bicarbonate and extracted with dichloromethane. The combined organic layers were dried and the solvent evaporated in vacuo. The residue was purified by flash column chromatography (ethyl acetate:hexane 3:1 to 4:0) to give 0.18 mg of a mixture of two products, 3-(morpholine-4-sulfonyl)-6-morpholin-4-yl-imidazo[1,2-b]pyridazine and 6-chloro-3-(morpholine-4-sulfonyl)-imidazo[1,2-b]pyridazine, that was further reacted in its crude state.

### General Procedure V for the Preparation of Examples 108-109

The appropriate sulphonamide (1 eq) (e.g. 6-Chloro-imidazo[1,2-b]pyridazine-3-sulfonic acid ethylamide) was dissolved in anhydrous 1,4-dioxane (2 mL). 4-Fluorobenzyl amine (2.5 eq) was added, followed by N,N-diisopropylethyl amine (2.5 eq), and the reaction was heated in the microwave reactor (10 hours; 200 W; 155° C.). The solvent was evaporated and the residue was taken up in dichloromethane and sodium bicarbonate solution. Extraction of the aqueous layer with dichloromethane, drying of the combined organic layers (sodium sulphate) and evaporation gave the crude product, which was purified as specified to yield the desired product (e.g. 6-(4-fluoro-benzylamino)-imidazo[1,2-b]pyridazine-3-sulfonic acid ethyl amide).

### Example 108

### 6-(4-Fluoro-benzylamino)-imidazo[1,2-b]pyridazine-3-sulfonic acid ethylamide

The title compound was isolated by trituration of the crude product with diethyl ether (37% yield).

¹H NMR (300 MHz, acetone-d₆): δ 7.72 (2H, d, J=9.5 Hz), 7.42 (2H, dd, J=8.1, 5.7 Hz), 7.12 (1H, s), 6.97 (3H, m), 5.92 (1H, s), 4.45 (2H, s), 2.80 (2H, bs, overlap with water signal), 0.77 (3H, t, J=7.2 Hz).

LCMS: 350.1 [M+1], tR=10.21 min, (MW 349.4).

### Example 109

### (4-Fluoro-benzyl)-[3-(morpholine-4-sulfonyl)-imidazo[1,2-b]pyridazin-6-yl]-amine

The title compound was isolated by flash column chromatography (ethyl acetate 100%) (5% yield).

¹H NMR (300 MHz, acetone-d₆): δ 7.64 (2H, m), 7.38 (2H, dd, J=8.4, 5.5 Hz), 6.94 (3H, m), 4.48 (2H, d, J=5.9 Hz), 3.41 (4H, m), 3.02 (4H, m).

LCMS: 392.3 [M+1], tR=10.15 min, (MW 391.4).

### Intermediate 38

### 6-Chloro-imidazo[1,2-b]pyridazine-3-sulfonyl chloride

To 6-chloroimidazo[1,2-b]pyridazinyl-3-sulfonic acid (1.00 g, 3.98 mmol) was added phosphorus oxychloride (4.85 mL, 52.12 mmol) and phosphorus pentachloride (0.58 g, 2.79 mmol). The mixture was heated under nitrogen at 100° C. overnight and the excess phosphorus oxychloride was removed by distillation in vacuo. The crude product was washed with diethyl ether and 0.85 g of 6-chloro-imidazo[1,2-b]pyridazine-3-sulfonyl chloride were isolated as a brown, wet solid that was used in its crude state. (Yield: 79%)

### General Procedure W for the Preparation of Intermediates 39-40

Imidazo[1,2-b]pyridazine-3-sulfonyl chloride (1 eq) was dissolved in dry acetonitrile (about 0.17 mmol/mL). Triethylamine (1 eq) was added and the solution was cooled to 0° C. The appropriate amine (e.g. aniline) (2.5 eq) was added as a solution in dry acetonitrile (about 2.5 mmol/mL). The reaction was stirred overnight and the solvent was removed in vacuo. The residue was partitioned between ethyl acetate and sodium bicarbonate solution. Drying of the organic layer over sodium sulphate and removal of the solvent in vacuo gave the crude product that was further purified to give the desired product (e.g. 6-chloro-imidazo[1,2-b]pyridazine-3-sulfonic acid phenylamide).

### Intermediate 39

### 6-Chloro-imidazo[1,2-b]pyridazine-3-sulfonic acid phenylamide

The title compound was obtained in 63% yield after purification by column chromatography on flash silica gel.

LCMS: 309 [M+1], (MW: 308.70).

### Intermediate 40

### 6-Chloro-imidazo[1,2-b]pyridazine-3-sulfonic acid (4-methoxy-phenyl)-amide

The title compound was obtained in 18% yield after purification by column chromatography on flash silica gel.

LCMS: 339 [M+1], (MW: 338.77).

### General Procedure X for the Preparation of Examples 110-112

6-Chloro-imidazo[1,2-b]pyridazine-3-sulfonic acid phenylamide (1 eq) was dissolved in dry dioxane (about 0.1 mmol/mL). The appropriate amine (e.g. 3,4-dichlorobenzylamine) (2.5 eq) was added, followed by N,N-diisopropylethylamine (1 eq), and the mixture was heated in the microwave reactor at 160° C. for 11 hours.

The solvent was removed in vacuo and the residue was partitioned between ethyl acetate and sodium bicarbonate solution. Drying of the organic layer over sodium sulphate and removal of the solvent in vacuo gave the crude product that was further purified by dissolving in a minimum volume of ethyl acetate followed by precipitation by addition of a (1:3)-mixture of diethyl ether and hexane to give the desired product (e.g. 6-(3,4-dichloro-benzylamino)-imidazo[1,2-b]pyridazine-3-sulfonic acid phenylamide).

### Example 110

### 6-(3,4-Dichloro-benzylamino)-imidazo[1,2-b]pyridazine-3-sulfonic acid phenylamide

The title compound was obtained in 55% yield after precipitation from ethyl acetate.

¹H-NMR (300 MHz, acetone-d₆): δ 8.68 (1H, s), 7.68 (1H, s), 7.60 (2H, m), 7.41 (2H, s), 7.12 (1H, t, J=5.6 Hz), 7.03 (2H, m), 6.89 (4H, m), 4.62 (2H, d, J=6.1 Hz).

LCMS: 448 [M], tR=5.89 min, (MW 448.33).

### Example 111

### 6-(4-Fluoro-benzylamino)-imidazo[1,2-b]pyridazine-3-sulfonic acid phenyl-amide

The title compound was obtained in 25% yield after precipitation from ethyl acetate.

¹H-NMR (300 MHz, MeOD): δ 7.67 (1H, s), 7.52 (1H, d, J=9.8 Hz), 7.41 (2H, dd, J=8.6, 5.5 Hz), 6.93 (8H, m), 6.76 (1H, d, J=9.8 Hz), 4.62 (2H, d, J=6.1 Hz).

LCMS: 398 [M+1], tR=4.71 min, (MW 397.43).

### Example 112

### 6-(4-Fluoro-benzylamino)-imidazo[1,2-b]pyridazine-3-sulfonic acid (4-methoxy-phenyl)-amide

The title compound was obtained in 48% yield after precipitation from ethyl acetate.

¹H-NMR (300 MHz, acetone-d₆): δ 8.21 (1H, s), 7.58 (2H, m), 7.46 (2H, dd, J=8.2, 5.6 Hz), 6.97 (4H, t, J=9.72 Hz), 6.78 (2H, d, J=8.84 Hz), 6.57 (2H, d, J=8.8 Hz), 4.56 (2H, d, J=5.8 Hz), 3.54 (3H, s).

LCMS: 428 [M+1], tR=4.55 min, (MW 427.46).

### Example 113

### 6-Phenoxy-imidazo[1,2-b]pyridazine-3-sulfonic acid phenylamide

To sodium hydride (0.034 g, 0.84 mmol) was added dry dioxane (2 mL). Phenol (0.067 g, 0.71 mmol) was added and the reaction was stirred for 30 minutes at room temperature before adding 6-chloro-imidazo[1,2-b]pyridazine-3-sulfonic acid phenylamide (0.10 g, 0.30 mmol). After continuing the reaction at 85° C. overnight, the solvent was removed in vacuo. The residue was partitioned between ethyl acetate and sodium bicarbonate solution. The organic layer was dried over sodium sulphate and the solvent removed in vacuo to give a crude product that was further purified on silica by flash column chromatography (ethyl acetate:hexane 1:1) to give 39.5 mg of 6-phenoxy-imidazo[1,2-b]pyridazine-3-sulfonic acid phenylamide as a white solid. (Yield: 33%).

¹H-NMR (300 MHz, acetone-d₆): δ 8.04 (1H, d, J=9.8 Hz), 7.95 (1H, s), 7.41 (2H, t, J=7.9 Hz), 7.22 (3H, m), 7.13 (1H, d, J=9.7 Hz), 7.03 (5H, m), 6.88 (1H, dd, J=10.5, 5.0 Hz).

LCMS: 367 [M+1], tR=5.14 min, (MW 366.40).

### Intermediate 41

### 6-(4-Methoxy-phenyl)-pyridazin-3-yl amine

3-Amino-6-chloropyridazine (2.0 g, 7.7 mmol) and 4-methoxyphenylboronic acid (1.76 g, 11.6 mmol) were placed in a dry, three-necked roundbottomed flask under nitrogen. Tetrakis(triphenylphosphine)palladium(0) (0.31 g, 0.27 mmol) was added, followed by toluene (20 mL) that had been de-oxygenated for 20 minutes with argon prior to use. A solution of sodium carbonate (1.72 g in 8 mL water, 2M) was added and Argon was bubbled through the mixture for 5 minutes before heating at 120° C. for 5 hours. The solvent was evaporated and the residue was taken up in ethyl acetate filtered and washed with ethyl acetate (100 mL). The product was further purified on silica by flash column chromatography to afford 1.30 g of 6-(4-methoxy-phenyl)-pyridazin-3-ylamine as a white solid. (Yield: 84%).

¹H-NMR (300 MHz, DMSO-d₆): δ 7.89 (2H, d, J=8.7 Hz), 7.74 (1H, d, J=9.2 Hz), 7.01 (2H, d, J=8.7 Hz), 6.83 (1H, d, J=9.2 Hz), 6.35 (2H, s), 3.80 (3H, s).

### Intermediate 42

### N′-[6-(4-Methoxy-benzyl)-pyridazin-3-yl]-N,N-dimethyl-formamidine

Dry toluene (25 mL) was added to a mixture of 6-(4-methoxy-phenyl)-pyridazin-3-ylamine (1.0 g, 4.94 mmol) and N,N-dimethylformamide dimethyl acetal (0.89 g, 7.41 mmol). The resulting suspension was refluxed under nitrogen for 3 hours, and the solvent was evaporated to give a pale-brown solid that was washed with a (1:3)-mixture of ethanol and diethyl ether to afford 1.05 g of N′-[6-(4-methoxy-benzyl)-pyridazin-3-yl]-N,N-dimethyl-formamidine as a grey solid. (Yield: 83%).

¹H-NMR (300 MHz, CDCl₃): δ 8.68 (1H, s), 7.96 (2H, d, J=8.8 Hz), 7.64 (1H, d, J=9.1 Hz), 7.12 (1H, d, J=9.8 Hz), 7.02 (2H, m), 3.84 (3H, s), 3.12 (6H, s).

### General Procedure Y for the Preparation of Examples 114-119

N′-[6-(4-methoxy-benzyl)-pyridazin-3-yl]-N,N-dimethyl-formamidine (1 eq) was placed in a dry screwcap vial. The appropriate alpha-bromoacetophenone (e.g. 2-bromoacetophenone) (1.10 eq) was added, followed by anhydrous N,N-dimethylformamide (about 0.17 mmol/mL). After flushing the vial with argon and closing it, the mixture was heated at 140° C. for 2 hours. The solvent was removed in vacuo and the residue was partitioned between sodium bicarbonate solution and ethyl acetate. Drying over sodium sulphate and evaporation of the organic solution gave the crude product that was purified on silica by flash column chromatography (ethyl acetate:cyclohexane 1:1 to ethyl acetate) to give the desired product (e.g. [6-(4-methoxy-phenyl)-imidazo[1,2-b]pyridazin-3-yl]-phenyl-methanone).

### Example 114

### [6-(4-methoxy-phenyl)-imidazo[1,2-b]pyridazin-3-yl]-phenyl-methanone)

The title compound was obtained in as a white solid after purification. (Yield: 66%).

¹H-NMR (300 MHz, acetone-d₆): δ 8.22 (1H, d, J=9.6 Hz), 8.19 (1H, s), 8.05 (3H, m), 7.95 (2H, m), 7.71 (1H, m), 7.60 (2H, m), 7.09 (2H, d, J=8.9 Hz), 3.89 (3H, s).

LCMS: 330 [M+1], tR=5.53 min, (MW 329.36).

### Example 115

### (4-Methoxy-phenyl)-[6-(4-methoxy-phenyl)-imidazo[1,2-b]pyridazin-3-yl]-methanone

The title compound was obtained as an off-white solid after purification. (Yield: 64%).

¹H NMR (300 MHz, acetone-d₆): δ 8.20 (1H, d, J=9.6 Hz), 8.15 (1H, s), 8.07 (2H, d, J=9.0 Hz), 8.00 (3H, m), 7.14 (1H, s), 7.10 (2H, d, J=2.7 Hz), 7.07 (1H, s), 3.94 (3H, s), 3.89 (3H, s).

LCMS: 360 [M+1], tR=5.65 min, (MW 359.38).

### Example 116

### (3-Methoxy-phenyl)-[6-(4-methoxy-phenyl)-imidazo[1,2-b]pyridazin-3-yl]-methanone

The title compound was obtained as a white solid after purification. (Yield: 46%).

¹H-NMR (300 MHz, acetone-d₆): δ 8.22 (2H, d, J=9.7 Hz), 8.06 (3H, m), 7.50 (3H, m), 7.26 (1H, dd, J=6.0, 2.8 Hz), 7.10 (2H, d, J=8.8 Hz), 3.89 (6H, s).

LCMS: 360 [M+1], tR=5.75 min, (MW 359.38).

### Example 117

### (4-Fluoro-phenyl)-[6-(4-methoxy-phenyl)-imidazo[1,2-b]pyridazin-3-yl]-methanone

The title compound was obtained as a white solid after purification. (Yield: 60%).

¹H-NMR (300 MHz, acetone-d₆): δ 8.08 (2H, t, J=4.8 Hz), 7.91 (5H, m), 7.23 (2H, t, J=8.8 Hz), 6.95 (2H, d, J=9.0 Hz), 3.76 (3H, s).

LCMS: 348 [M+1], tR=5.89 min, (MW 347.35).

### Example 118

### (3-Fluoro-phenyl)-[6-(4-methoxy-phenyl)-imidazo[1,2-b]pyridazin-3-yl]-methanone

The title compound was obtained as a white solid after purification. (Yield: 71%).

¹H-NMR (300 MHz, acetone-d₆): δ 8.24 (2H, t, J=9.4 Hz), 8.06 (3H, dd, J=9.3, 7.7 Hz), 7.79 (1H, d, J=8.5 Hz), 7.66 (2H, m), 7.48 (1H, m), 7.10 (2H, d, J=9.0 Hz), 3.90 (3H, s).

LCMS: 348 [M+1], tR=6.00 min, (MW 347.35).

### Example 119

### 4-[6-(4-Methoxy-phenyl)-imidazo[1,2-b]pyridazine-3-carbonyl]-benzonitrile

The title compound was obtained a white solid after purification. (Yield: 71%).

¹H-NMR (300 MHz, DMSO-d₆): δ 8.40 (1H, d, J=9.6 Hz), 8.31 (1H, s), 8.14 (1H, d, J=9.7 Hz), 8.03 (6H, m), 7.10 (2H, d, J=8.9 Hz), 3.85 (3H, s).

LCMS: 355 [M+1], tR=5.22 min, (MW 354.37).

### Intermediate 43

### (3,4-Dichloro-benzyl)-methyl-(3-nitro-imidazo[1,2-b]pyridazin-6-yl)-amine

6-Chloro-3-nitroimidazo[1,2-b]pyridazine (0.32 g, 1.61 mmol) and (3,4-dichlorobenzyl)methylamine (0.61 g, 3.22 mmol) were dissolved in anhydrous dioxane (12 mL). N,N-diisopropylethylamine (0.28 mL, 1.61 mmol) was added and the reaction vial was flushed with argon before heating at 160° C. for 8 hours in the microwave reactor. The solvent was evaporated and the residue was partitioned between ethyl acetate and sodium bicarbonate solution. Extraction, drying over sodium sulphate and evaporation gave a crude product that was purified by washing with diethyl ether and cyclohexane to give 0.47 g of (3,4-dichloro-benzyl)-methyl-(3-nitro-imidazo[1,2-b]pyridazin-6-yl)-amine. (Yield: 83%).

¹H-NMR (300 MHz, DMSO-d₆): δ 8.51 (1H, s), 8.11 (1H, d, J=10.0 Hz), 7.69 (1H, d, J=2.0 Hz), 7.59 (1H, d, J=8.3 Hz), 7.43 (1H, d, J=10.1 Hz), 7.37 (1H, dd, J=8.3, 2.0 Hz), 4.83 (2H, s), 3.22 (3H, s).

LCMS: 352 [M], (MW 352.18).

### Example 120

### N*6*-(3,4-Dichloro-benzyl)-N*6*-methyl-imidazo[1,2-b]pyridazine-3,6-diamine

(3,4-dichloro-benzyl)-methyl-(3-nitro-imidazo[1,2-b]pyridazin-6-yl)-amine (0.50 g; 1.42 mmol) was dissolved in a hot solution (400 mL) consisting of a (1:1)-mixture of ethyl acetate and ethanol. The solution was filtered and reacted on the H-cube (Ni-Raney cartridge, P_{H2}: 40 bar; T: 30° C.; flow rate: 1 mL/min). Three cycles were performed. Removal of the solvent in vacuo gave a crude material that was purified on silica by flash column chromatography to give 0.30 g of N*6*-(3,4-dichloro-benzyl)-N*6*-methyl-imidazo[1,2-b]pyridazine-3,6-diamine as a slowly-crystallizing yellow oil. (Yield: 62%).

¹H-NMR (300 MHz, DMSO-d₆): δ 7.57 (1H, d, J=9.8 Hz), 7.36 (2H, dd, J=11.7, 5.1 Hz), 7.09 (1H, dd, J=8.2, 2.0 Hz), 7.01 (1H, s), 6.50 (1H, d, J=9.8 Hz), 4.67 (2H, s), 3.86 (2H, s), 3.11 (3H, s).

LCMS: 322 [M], (MW 322.20).

### General Procedure Z for the Preparation of Examples 121-122

N*6*-(3,4-Dichloro-benzyl)-N*6*-methyl-imidazo[1,2-b]pyridazine-3,6-diamine (1 eq) was placed in a dry screwcap vial and dissolved in dry methanol (about 0.062 mmol/mL). The appropriate aldehyde (e.g. benzaldehyde) (2 eq) was added, followed by acetic acid (2 eq). The reaction was stirred at room temperature for 1 hour before adding sodium cyanoborohydride (2 eq). The reaction was continued overnight before evaporating the solvent and purifying the residue on silica by flash column chromatography to give the desired product (e.g. N*3*-benzyl-N*6*-(3,4-dichloro-benzyl)-N*6*-methyl-imidazo[1,2-b]pyridazine-3,6-diamine).

### Example 121

### N*3*-Benzyl-N*6*-(3,4-dichloro-benzyl)-N*6*-methyl-imidazo[1,2b]pyridazine-3,6-diamine

The title compound was obtained as a yellow solid after purification (cyclohexane:ethyl acetate 85:15 to ethyl acetate). (Yield: 23%)

¹H-NMR (300 MHz, CDCl₃): δ 7.67 (1H, d, J=9.8 Hz), 7.18 (1H, dd, J=8.2, 1.8 Hz), 6.97 (1H, s), 6.59 (1H, d, J=9.8 Hz), 7.44 (7H, m), 4.75 (2H, s), 4.53 (2H, d, J=4.6 Hz), 4.47 (1H, s), 3.21 (3H, s).

LCMS: 412 [M], tR=5.57 min, (MW 412.31).

### Example 122

### N*6*-(3,4-Dichloro-benzyl)-N*3*-(4-methoxy-benzyl)-N*6*-methyl-imidazo[1,2-b]pyridazine-3,6-diamine

The title compound was obtained as a yellow solid after purification (cyclohexane:ethyl acetate 85:15 to ethyl acetate). (Yield: 25%)

¹H-NMR (300 MHz, CDCl₃): δ 7.60 (1H, d, J=9.8 Hz), 7.36 (4H, m), 7.09 (1H, dd, J=8.2, 1.8 Hz), 6.90 (3H, m), 6.52 (1H, d, J=9.8 Hz), 4.67 (2H, s), 4.38 (2H, s), 3.82 (3H, s), 3.13 (3H, s), 2.06 (1H, s).

LCMS: 442 [M], tR=5.76 min, (MW 442.35).

### Intermediate 44

### 6-Phenethyl-pyridazin-3-ylamine

Dry dioxane (12 mL) was added to 1,3-bis(diphenylphosphino)propane nickel(ii) chloride (0.29 g, 0.54 mmol) and the system was flushed with nitrogen for 5 min before adding diethyl zinc (14.7 mL, 1.1 M in toluene, 16.17 mmol). The mixture was stirred at room temperature for 10 minutes, then (2-bromoethyl)benzene (3.0 g, 16.17 mmol) was added. The resulting mixture was refluxed for 4 hours before adding 3-amino-6-chloropyridazine (0.35 g, 2.70 mmol) as a suspension in warm dioxane (8 mL). The reaction was continued for 3 hours, then methanol (6 mL) was added and the mixture was stirred for further 10 minutes before evaporating the solution. The reaction mixture was taken up in ethyl acetate and sodium bicarbonate solution. Extraction, drying over sodium sulphate and removal of the solvent in vacuo gave the crude product that was further purified on silica by flash column chromatography to give 0.28 g of the title product 6-phenethyl-pyridazin-3-ylamine as a pale-grey solid. (Yield: 52%).

¹H-NMR (300 MHz, CDCl₃): δ 7.20 (2H, m), 7.12 (3H, m), 6.88 (1H, d, J=9.0 Hz), 6.58 (1H, d, J=9.0 Hz), 4.57 (2H, s), 3.02 (4H, m).

LCMS: 200 [M+1], (MW 199.26).

### Intermediate 45

### N,N-Dimethyl-N′-(6-phenethyl-pyridazin-3-yl)-formamidine

6-Phenethyl-pyridazin-3-ylamine (0.22 g, 1.10 mmol) was suspended in dry toluene (5 mL) and N,N-dimethylformamide-dimethylacetal (0.22 mL, 1.66 mmol) was added. The mixture was refluxed for 2 hours. The solvent was evaporated and the residue was partitioned between ethyl acetate and sodium bicarbonate solution. Drying over sodium sulphate and removal in vacuo of the solvent gave 0.13 g of the title product N,N-dimethyl-N′-(6-phenethyl-pyridazin-3-yl)-formamidine as a pale-brown oil. (Yield: 46.3%).

LCMS: 255 [M+1], (MW 254.34).

### Example 123

### 6-Phenethyl-imidazo[1,2-b]pyridazine-3-carboxylic acid ethyl ester

N,N-Dimethyl-N′-(6-phenethyl-pyridazin-3-yl)-formamidine (0.125 g, 0.49 mmol) was dissolved in dry N,N-dimethylformamide (3 mL). Ethyl bromoacetate (0.123 g, 0.74 mmol) was added and the reaction was heated at 120° C. After 4 hours, heating was interrupted and N,N-diisopropylethylamine (0.21 mL; 1.23 mmol) was added and the reaction was stirred at room temperature overnight. The following morning the solvent was evaporated and the residue was partitioned between ethyl acetate and sodium bicarbonate. Extractive workup followed by drying over sodium sulphate and removal of the solvent in vacuo gave a brown solid corresponding to 0.080 g of the title product 6-phenethyl-imidazo[1,2-b]pyridazine-3-carboxylic acid ethyl ester. (Yield: 55%)

LCMS: 296 [M+1], (MW 295.34).

### Example 124

### 6-Phenethyl-imidazo[1,2-b]pyridazine-3-carboxylic acid

6-Phenethyl-imidazo[1,2-b]pyridazine-3-carboxylic acid ethyl ester (0.080 g, 0.27 mmol) was dissolved in ethanol (4 mL). Solid potassium hydroxide (0.070 g, 1.08 mmol) was added and the mixture was refluxed for 3 hours. The solvent was evaporated and hydrochloric acid (2M) was added until pH 4. A yellow precipitate was collected by vacuum filtration. The aqueous layer was extracted with ethyl acetate, dried and evaporated to give 10 mg of a yellow powder that was added to the previous batch. 0.065 g of the title product 6-phenethyl-imidazo[1,2-b]pyridazine-3-carboxylic acid were isolated as a yellow powder. (Yield: 90%).

¹H-NMR (300 MHz, CDCl₃): δ 8.50 (1H, s), 8.15 (1H, d, J=9.4 Hz), 7.27 (6H, m), 7.15 (1H, d, J=9.4 Hz), 3.27 (4H, m).

### Example 125

### 6-Phenethyl-imidazo[1,2-b]pyridazine-3-carboxylic acid (4-methoxy-phenyl)-amide

6-Phenethyl-imidazo[1,2-b]pyridazine-3-carboxylic acid (0.055 g, 0.21 mmol) was dissolved in dry tetrahydrofuran (3 mL) and N,N′-carbonyldiimidazole (0.037 g, 0.226 mmol) was added. The solution was heated at 50° C. for 2 hours, then cooled to room temperature before adding para-anisidine (0.027 g, 0.23 mmol). The reaction was continued overnight. The solvent was evaporated and the crude product was purified on silica by flash column chromatography (cyclohexane:ethyl acetate 4:1) to give 0.025 g of 6-phenethyl-imidazo[1,2-b]pyridazine-3-carboxylic acid (4-methoxy-phenyl)-amide as a yellow solid. (Yield: 33%).

¹H-NMR (300 MHz, CDCl₃): δ 10.40 (1H, s), 8.53 (1H, s), 8.03 (1H, d, J=9.3 Hz), 7.61 (2H, d, J=8.9 Hz), 7.26 (5H, m), 7.09 (1H, d, J=9.3 Hz), 6.93 (2H, d, J=8.9 Hz), 3.83 (3H, s), 3.28 (4H, m).

LCMS: 373 [M+1], tR=7.31 min, (MW 372.43).

### Example 126

### 6-Phenethyl-imidazo[1,2-b]pyridazine-3-carboxylic acid phenylamide

Dry dioxane (2.5 mL) was added to 1,3-bis(diphenylphosphino)propane nickel(ii) chloride (0.048 g, 0.088 mmol) and the system was flushed with nitrogen for 5 minutes before adding diethyl zinc (2.4 mL; 1.1 M in toluene, 2.64 mmol). The mixture was stirred at room temperature for 10 minutes, then (2-bromoethyl)benzene (0.49 g; 2.66 mmol) was added. The resulting mixture was refluxed for 4 hours before adding 6-chloro-imidazo[1,2-b]pyridazine-3-carboxylic acid phenylamide (0.10 g, 0.44 mmol) as a solution in dioxane (2 mL). The reaction was continued for 2 hours, then methanol (3 mL) was added and the mixture was stirred for further 10 minutes before evaporating the solution. The reaction mixture was taken up in ethyl acetate and sodium bicarbonate solution. Extraction, drying over sodium sulphate and removal of the solvent in vacuo gave the crude product that was first washed with a (1:3)-mixture of diethyl ether and cyclohexane, then further purified on silica by flash column chromatography (cyclohexane:ethyl acetate 4:1) to give 0.010 g of the title product 6-phenethyl-imidazo[1,2-b]pyridazine-3-carboxylic acid phenylamide as a white solid. (Yield: 7%).

¹H-NMR (300 MHz, CDCl₃): δ 10.5 (1H, s), 8.54 (1H, s), 8.03 (1H, d, J=9.3 Hz), 7.70 (2H, d, J=7.8 Hz), 7.25 (9H, m), 3.29 (4H, m).

LCMS: 343 [M+1], tR=7.68 min, (MW 342.40).

### Intermediate 46

### (E)-4,4-Dimethoxy-but-2-enoic acid ethyl ester

To a suspension of potassium carbonate (5.0 g, 35.79 mmol) in cyclohexane (120 mL) was added triethyl phosphonoacetate (7.20 mL, 35.79 mmol) and the mixture was stirred vigorously for 5 minutes. Glyoxal dimethyl acetal (3.6 mL; 23.86 mmol; 60% wt in water) was added and the mixture was heated at 85° C. overnight. Removal in vacuo of the solvent left a yellow oily slurry that was purified on silica by flash column chromatography by column chromatography to give 3.3 g of the title product (E)-4,4-dimethoxy-but-2-enoic acid ethyl ester as a transparent oil. (Yield: 79%).

¹H-NMR (300 MHz, CDCl₃): δ 6.74 (1H, dd, J=15.9, 4.0 Hz), 6.11 (1H, dd, J=15.9, 1.4 Hz), 4.93 (1H, s), 4.19 (2H, q, J=7.1 Hz), 3.31 (6H, s), 1.27 (3H, t, J=7.1 Hz).

### Example 127

### (6-Phenethyl-imidazo[1,2-b]pyridazin-3-yl)-acetic acid

To 6-phenethyl-pyridazin-3-ylamine (0.050 g, 0.25 mmol) was added (E)-4,4-dimethoxy-but-2-enoic acid ethyl ester (0.066 g, 0.37 mmol), followed by water (2 mL). The mixture was heated until a transparent solution was obtained. Hydrochloric acid (2N) was added to pH 3, and heating was continued at 85° C. overnight. pH was adjusted to 5 by addition of solid sodium bicarbonate, and the resulting turbid solution was extracted with ethyl acetate. The organic layer was dried over sodium sulphate, evaporated and 0.048 g of the title product (6-phenethyl-imidazo[1,2-b]pyridazin-3-yl)-acetic acid was isolated as a brown solid. (Yield: 68%).

¹H-NMR (300 MHz, CDCl₃): δ 8.00 (1H, d, J=9.27 Hz), 7.62 (2H, m), 7.21 (6H, m), 3.97 (2H, s), 3.08 (4H, m).

LCMS: 282 [M+1], tR=7.68 min, (MW 281.32).

### Example 128

### 2-(6-Phenethyl-imidazo[1,2-b]pyridazin-3-yl)-N-phenyl-acetamide

(6-Phenethyl-imidazo[1,2-b]pyridazin-3-yl)-acetic acid (0.045 g, 0.16 mmol) was dissolved in dry tetrahydrofuran (3 mL) and N,N′-carbonyldiimidazole (0.029 g, 0.18 mmol) was added. The solution was heated at 50° C. for 2 hours, then cooled to room temperature before adding aniline (0.017 g, 0.18 mmol). The reaction was continued overnight. The solvent was evaporated and the crude product was purified on silica by flash column chromatography (cyclohexane:ethyl acetate 3:1) to give 0.012 g of the title product 2-(6-phenethyl-imidazo[1,2-b]pyridazin-3-yl)-N-phenyl-acetamide as a beige solid. (Yield: 21%).

¹H-NMR (300 MHz, CDCl₃): δ 8.24 (1H, s), 7.89 (1H, d, J=9.3 Hz), 7.78 (1H, s), 7.44 (2H, d, J=8.0 Hz), 7.26 (7H, m), 7.09 (1H, t, J=9.3 Hz), 6.94 (1H, d, J=9.3 Hz), 4.11 (2H, s), 3.20 (4H, m).

LCMS: 357 [M+1], tR=5.15 min, (MW 356.43).

### Example 129

The following compounds were prepared in accordance with the procedures described herein:
6-(4-Fluoro-benzylamino)-imidazo[1,2-b]pyridazine-3-carboxylic acid (2-fluoro-4-hydroxy-phenyl)-amide (396.1 [M+1]; MW 395.4);
6-(4-Fluoro-benzylamino)-imidazo[1,2-b]pyridazine-3-carboxylic acid (1H-indol-5-yl)-amide (401.4 [M+1]; MW 400.4);
6-(4-Fluoro-benzylamino)-imidazo[1,2-b]pyridazine-3-carboxylic acid (1H-indol-6-yl)-amide (401.1 [M+1]; MW 400.4);
6-(4-Fluoro-benzylamino)-imidazo[1,2-b]pyridazine-3-carboxylic acid (4-dimethylamino-phenyl)-amide (405.2 [M+1]; MW 404.4);
3-{[6-(4-Fluoro-benzylamino)-imidazo[1,2-b]pyridazine-3-carbonyl]-amino}-benzoic acid ethyl ester (434.1 [M+1]; MW 433.4);
6-(4-Fluoro-benzylamino)-imidazo[1,2-b]pyridazine-3-carboxylic acid (3-hydroxymethyl-phenyl)-amide (392.1 [M+1]; MW 391.4);
6-(4-Fluoro-benzylamino)-imidazo[1,2-b]pyridazine-3-carboxylic acid (2,5-dimethyl-2H-pyrazol-3-yl)-amide (380.1 [M+1]; MW 379.4);
6-(4-Fluoro-benzylamino)-imidazo[1,2-b]pyridazine-3-carboxylic acid (3-methanesulfonyl-phenyl)-amide (440.1 [M+1]; MW 439.5);
6-[(4-Fluoro-benzyl)-methyl-amino]-imidazo[1,2-b]pyridazine-3-carboxylic acid (4-acetylamino-phenyl)-amide (433.1 [M+1]; MW 432.5);
6-(3-Chloro-4-fluoro-benzylamino)-imidazo[1,2-b]pyridazine-3-carboxylic acid (4-acetylamino-phenyl)-amide (453.1 [M+1]; MW 452.9);
6-(4-Fluoro-benzylamino)-imidazo[1,2-b]pyridazine-3-carboxylic acid (4-acetylamino-phenyl)-amide (419.1 [M+1]; MW 418.4);
6-(4-Fluoro-benzylamino)-imidazo[1,2-b]pyridazine-3-carboxylic acid (4-hydroxymethyl-phenyl)-amide (392.0 [M+1]; MW 391.4);
6-(4-Fluoro-benzylamino)-imidazo[1,2-b]pyridazine-3-carboxylic acid (5-acetylamino-2-fluoro-phenyl)-amide (437.1 [M+1]; MW 436.4);
6-(4-Fluoro-benzylamino)-imidazo[1,2-b]pyridazine-3-carboxylic acid [3-(3-methyl-5-oxo-4,5-dihydro-pyrazol-1-yl)-phenyl]-amide (458.1 [M+1]; MW 457.5);
6-[(4-Fluoro-benzyl)-methyl-amino]-imidazo[1,2-b]pyridazine-3-carboxylic acid (4-hydroxy-phenyl)-amide (392.1 [M+1]; MW 391.4);
6-(4-Fluoro-benzylamino)-7-methyl-imidazo[1,2-b]pyridazine-3-carboxylic acid (2-fluoro-4-hydroxy-phenyl)-amide (410.1 [M+1]; MW 409.4);
6-(4-Fluoro-benzylamino)-7-methyl-imidazo[1,2-b]pyridazine-3-carboxylic acid (1H-indol-5-yl)-amide (415.1 [M+1]; MW 414.4);
6-(4-Fluoro-benzylamino)-7-methyl-imidazo[1,2-b]pyridazine-3-carboxylic acid (4-hydroxymethyl-phenyl)-amide (406.1 [M+1]; MW 405.4);
(4-{[6-(4-Fluoro-benzylamino)-imidazo[1,2-b]pyridazine-3-carbonyl]-amino}-phenyl)-acetic acid (420.1 [M+1]; MW 419.4);
6-(4-Fluoro-benzylamino)-imidazo[1,2-b]pyridazine-3-carboxylic acid (4-methyl-thiazol-2-yl)-amide (382.4 [M+1]; MW 383.1);
6-(4-Fluoro-benzylamino)-imidazo[1,2-b]pyridazine-3-carboxylic acid [4-(4-methyl-piperazin-1-yl)-phenyl]-amide (460.1 [M+1]; MW 459.5);
6-(4-Fluoro-benzylamino)-7-methyl-imidazo[1,2-b]pyridazine-3-carboxylic acid (4-acetylamino-phenyl)-amide (433.1 [M+1]; MW 432.5);
6-(4-Fluoro-benzylamino)-imidazo[1,2-b]pyridazine-3-carboxylic acid (4-hydroxy-phenyl)-methyl-amide (392.1 [M+1]; MW 391.4);
3-{[6-(4-Fluoro-benzylamino)-imidazo[1,2-b]pyridazine-3-carbonyl]-methyl-amino}-benzoic acid ethyl ester (448.1 [M+1]; MW 447.5);
6-(4-Fluoro-3-methyl-benzylamino)-imidazo[1,2-b]pyridazine-3-carboxylic acid (4-hydroxy-phenyl)-amide (392.1 [M+1]; MW 391.4);
6-(3-Chloro-4-fluoro-benzylamino)-imidazo[1,2-b]pyridazine-3-carboxylic acid (4-hydroxy-phenyl)-amide (412.1 [M+1]; MW 411.8);
6-(3,4-Dimethoxy-benzylamino)-imidazo[1,2-b]pyridazine-3-carboxylic acid (4-hydroxy-phenyl)-amide (420.1 [M+1]; MW 419.4);
6-(4-Fluoro-benzylamino)-imidazo[1,2-b]pyridazine-3-carboxylic acid (4-acetylamino-2-fluoro-phenyl)-amide (437.1 [M+1]; MW 436.4);
6-(4-Fluoro-benzylamino)-imidazo[1,2-b]pyridazine-3-carboxylic acid (4-methylamino-phenyl)-amide (391.1 [M+1]; MW 390.4);
(3-{[6-(4-Fluoro-benzylamino)-imidazo[1,2-b]pyridazine-3-carbonyl]-amino}-phenyl)-acetic acid (420.1 [M+1]; MW 419.4);
6-(4-Fluoro-benzylamino)-7-methyl-imidazo[1,2-b]pyridazine-3-carboxylic acid (4-dimethylamino-phenyl)-amide (419.1 [M+1]; MW 418.5);
6-(4-Fluoro-benzylamino)-7-methyl-imidazo[1,2-b]pyridazine-3-carboxylic acid (4-methylamino-phenyl)-amide (405.1 [M+1]; MW 404.4);
6-(4-Methoxy-benzylamino)-imidazo[1,2-b]pyridazine-3-carboxylic acid (4-hydroxy-phenyl)-amide; trifluoroacetate salt (390.1 [M+1]; MW 389.4);
6-(4-Methoxy-benzylamino)-imidazo[1,2-b]pyridazine-3-carboxylic acid (1H-indol-5-yl)-amide; trifluoroacetate salt (413.1 [M+1]; MW 412.5);
6-(4-Methoxy-benzylamino)-imidazo[1,2-b]pyridazine-3-carboxylic acid (1H-indol-6-yl)-amide; trifluoroacetate salt (413.1 [M+1]; MW 412.5);
6-(4-Methoxy-benzylamino)-imidazo[1,2-b]pyridazine-3-carboxylic acid (4-acetylamino-phenyl)-amide; trifluoroacetate salt (431.2 [M+1]; MW 430.5);
6-(4-Methoxy-benzylamino)-imidazo[1,2-b]pyridazine-3-carboxylic acid (4-hydroxy-phenyl)-methyl-amide; trifluoroacetate salt (404.1 [M+1]; MW 403.4);
3-{[6-(4-Methoxy-benzylamino)-imidazo[1,2-b]pyridazine-3-carbonyl]-methyl-amino}-benzoic acid ethyl ester; trifluoroacetate salt (460.1 [M+1]; MW 459.5);
6-(4-Methoxy-benzylamino)-imidazo[1,2-b]pyridazine-3-carboxylic acid (3-methanesulfonyl-phenyl)-amide; trifluoroacetate salt (452.1 [M+1]; MW 451.5);
6-(4-Fluoro-benzylamino)-7-methyl-imidazo[1,2-b]pyridazine-3-carboxylic acid
(1H-indol-6-yl)-amide (415.1 [M+1]; MW 414.4);
(4-{[6-(4-Methoxy-benzylamino)-imidazo[1,2-b]pyridazine-3-carbonyl]-amino}-phenyl)-acetic acid (431.2 [M+1]; MW 431.5);
6-[(4-Fluoro-benzyl)-methyl-amino]-imidazo[1,2-b]pyridazine-3-carboxylic acid
(1H-indol-5-yl)-amide; trifluoroacetate salt (415.1 [M+1]; MW 414.4);
6-[(4-Fluoro-benzyl)-methyl-amino]-imidazo[1,2-b]pyridazine-3-carboxylic acid
(1H-indol-6-yl)-amide; trifluoroacetate salt (415.1 [M+1]; MW 414.4);
6-[(4-Fluoro-benzyl)-methyl-amino]-imidazo[1,2-b]pyridazine-3-carboxylic acid (4-hydroxymethyl-phenyl)-amide; trifluoroacetate salt (406.1 [M+1]; MW 405.4);
6-[(4-Methoxy-benzyl)-methyl-amino]-imidazo[1,2-b]pyridazine-3-carboxylic acid
(4-hydroxy-phenyl)-amide; trifluoroacetate salt (404.1 [M+1]; MW 403.4);
3-({6-[(4-Methoxy-benzyl)-methyl-amino]-imidazo[1,2-b]pyridazine-3-carbonyl}-amino)-benzoic acid ethyl ester; trifluoroacetate salt (460.2 [M+1]; MW 459.5);
3-({6-[(4-Methoxy-benzyl)-methyl-amino]-imidazo[1,2-b]pyridazine-3-carbonyl}-methyl-amino)-benzoic acid ethyl ester; trifluoroacetate salt (474.1 [M+1]; MW 473.5);
6-(4-Methoxy-phenylamino)-imidazo[1,2-b]pyridazine-3-carboxylic acid (4-hydroxy-phenyl)-amide; trifluoroacetate salt (376.1 [M+1]; MW 375.4);
6-(4-Methoxy-phenylamino)-imidazo[1,2-b]pyridazine-3-carboxylic acid (4-hydroxymethyl-phenyl)-amide; trifluoroacetate salt (390.1 [M+1]; MW 389.4);
3-{[6-(4-Methoxy-phenylamino)-imidazo[1,2-b]pyridazine-3-carbonyl]-amino}-benzoic acid ethyl ester; trifluoroacetate salt (432.1 [M+1]; MW 431.5);
6-(4-Methoxy-phenylamino)-imidazo[1,2-b]pyridazine-3-carboxylic acid (3-methanesulfonyl-phenyl)-amide; trifluoroacetate salt (438.1 [M+1]; MW 437.5);
6-(4-Hydroxy-benzylamino)-imidazo[1,2-b]pyridazine-3-carboxylic acid phenylamide; trifluoroacetate salt (360.1 [M+1]; MW 359.4);
6-(4-Hydroxy-benzylamino)-imidazo[1,2-b]pyridazine-3-carboxylic acid (4-hydroxy-phenyl)-amide; trifluoroacetate salt (376.1 [M+1]; MW 375.4);
6-(4-Methoxy-benzylamino)-7-methyl-imidazo[1,2-b]pyridazine-3-carboxylic acid (4-hydroxy-phenyl)-amide; trifluoroacetate salt (404.1 [M+1]; MW 403.4)
3-{[6-(4-Methoxy-benzylamino)-7-methyl-imidazo[1,2-b]pyridazine-3-carbonyl]-amino}-benzoic acid ethyl ester; trifluoroacetate salt (460.1 [M+1]; MW 459.5);
6-(4-Methoxy-benzylamino)-7-methyl-imidazo[1,2-b]pyridazine-3-carboxylic acid (3-methanesulfonyl-phenyl)-amide; trifluoroacetate salt (466.1 [M+1]; MW 465.5);
(3-{[6-(4-Methoxy-benzylamino)-imidazo[1,2-b]pyridazine-3-carbonyl]-amino}-phenyl)-acetic acid (432.1 [M+1]; MW 431.5);
[4-({6-[(4-Methoxy-benzyl)-methyl-amino]-imidazo[1,2-b]pyridazine-3-carbonyl}-amino)-phenyl]-acetic acid (446.1 [M+1]; MW 445.5);
[3-({6-[(4-Methoxy-benzyl)-methyl-amino]-imidazo[1,2-b]pyridazine-3-carbonyl}-amino)-phenyl]-acetic acid (446.1 [M+1]; MW 445.5);
(4-{[6-(4-Methoxy-phenylamino)-imidazo[1,2-b]pyridazine-3-carbonyl]-amino}-phenyl)-acetic acid (418.0 [M+1]; MW 417.4);
(3-{[6-(4-Methoxy-phenylamino)-imidazo[1,2-b]pyridazine-3-carbonyl]-amino}-phenyl)-acetic acid (418.0 [M+1]; MW 417.4);
3-{[6-(3,4-Dimethoxy-benzylamino)-imidazo[1,2-b]pyridazine-3-carbonyl]-amino}-benzoic acid ethyl ester; trifluoroacetate salt (476.1 [M+1]; MW 475.5);
6-(3-Hydroxy-benzylamino)-imidazo[1,2-b]pyridazine-3-carboxylic acid phenylamide; trifluoroacetate salt (360.1 [M+1]; MW 359.4);
6-(3-Hydroxy-benzylamino)-imidazo[1,2-b]pyridazine-3-carboxylic acid (4-hydroxy-phenyl)-amide; trifluoroacetate salt (376.1 [M+1]; MW 375.4);
3-{[6-(3-Hydroxy-benzylamino)-imidazo[1,2-b]pyridazine-3-carbonyl]-amino}-benzoic acid ethyl ester; trifluoroacetate salt (432.1 [M+1]; MW 431.5);
3-{[6-(4-Hydroxy-benzylamino)-imidazo[1,2-b]pyridazine-3-carbonyl]-amino}-benzoic acid ethyl ester; trifluoroacetate salt (432.1 [M+1]; MW 431.5);
6-(2,6-Difluoro-benzylamino)-imidazo[1,2-b]pyridazine-3-carboxylic acid phenylamide; trifluoroacetate salt (380.1 [M+1]; MW 379.4);
6-(4-Fluoro-3-methyl-benzylamino)-7-methyl-imidazo[1,2-b]pyridazine-3-carboxylic acid (4-hydroxy-phenyl)-amide; trifluoroacetate salt (406.1 [M+1]; MW 405.4);
6-[(4-Fluoro-benzyl)-methyl-amino]-7-methyl-imidazo[1,2-b]pyridazine-3-carboxylic acid (4-hydroxy-phenyl)-amide; trifluoroacetate salt (406.1 [M+1]; MW 405.4);
6-(3-Chloro-4-fluoro-benzylamino)-7-methyl-imidazo[1,2-b]pyridazine-3-carboxylic acid (4-hydroxy-phenyl)-amide; trifluoroacetate salt (426.0 [M+1]; MW 425.8);
3-{[6-(4-Hydroxy-benzylamino)-7-methyl-imidazo[1,2-b]pyridazine-3-carbonyl]-amino}-benzoic acid ethyl ester; trifluoroacetate salt (446.1 [M+1]; MW 445.5);
3-{[6-(4-Methoxy-benzylamino)-imidazo[1,2-b]pyridazine-3-carbonyl]-amino}-4-methyl-benzoic acid (432.1 [M+1]; MW 431.5);
3-{[6-(4-Methoxy-benzylamino)-imidazo[1,2-b]pyridazine-3-carbonyl]-amino}-5-trifluoromethyl-benzoic acid (486.0 [M+1]; MW 485.4);
6-(3-Chloro-4-fluoro-benzylamino)-imidazo[1,2-b]pyridazine-3-carboxylic acid
(1H-indol-5-yl)-amide; trifluoroacetate salt (435.0 [M+1]; MW 434.9);
6-(3-Chloro-4-fluoro-benzylamino)-imidazo[1,2-b]pyridazine-3-carboxylic acid
(1H-indol-6-yl)-amide; trifluoroacetate salt (435.0 [M+1]; MW 434.9);
6-(3-Chloro-4-fluoro-benzylamino)-imidazo[1,2-b]pyridazine-3-carboxylic acid (4-hydroxymethyl-phenyl)-amide; trifluoroacetate salt (426.0 [M+1]; MW 425.8);
6-(4-Fluoro-phenylamino)-imidazo[1,2-b]pyridazine-3-carboxylic acid (4-hydroxy-phenyl)-amide; trifluoroacetate salt (364.0 [M+1]; MW 363.4);
6-(4-Fluoro-phenylamino)-imidazo[1,2-b]pyridazine-3-carboxylic acid (1H-indol-5-yl)-amide; trifluoroacetate salt (387.1 [M+1]; MW 386.4);
6-(4-Fluoro-phenylamino)-imidazo[1,2-b]pyridazine-3-carboxylic acid (1H-indol-6-yl)-amide; trifluoroacetate salt (387.1 [M+1]; MW 386.4)
6-(4-Fluoro-phenylamino)-imidazo[1,2-b]pyridazine-3-carboxylic acid (4-acetylamino-phenyl)-amide; trifluoroacetate salt (405.1 [M+1]; MW 404.4);
6-(4-Fluoro-phenylamino)-imidazo[1,2-b]pyridazine-3-carboxylic acid (4-methylamino-phenyl)-amide; trifluoroacetate salt (377.1 [M+1]; MW 376.4);
6-(4-Fluoro-phenylamino)-imidazo[1,2-b]pyridazine-3-carboxylic acid (4-hydroxymethyl-phenyl)-amide; trifluoroacetate salt (378.1 [M+1]; MW 377.4);
(5-Fluoro-1,3-dihydro-isoindol-2-yl)-[6-(4-fluoro-phenylamino)-imidazo[1,2-b]pyridazin-3-yl]-methanone; trifluoroacetate salt (392.1 [M+1]; MW 391.4);
6-(4-Fluoro-phenylamino)-imidazo[1,2-b]pyridazine-3-carboxylic acid (4-hydroxy-phenyl)-methyl-amide; trifluoroacetate salt (378.1 [M+1]; MW 377.4);
6-(4-Methoxy-phenylamino)-imidazo[1,2-b]pyridazine-3-carboxylic acid (4-acetylamino-phenyl)-amide; trifluoroacetate salt (417.1 [M+1]; MW 416.4);
3-{[6-(4-Methoxy-phenylamino)-imidazo[1,2-b]pyridazine-3-carbonyl]-methyl-amino}-benzoic acid ethyl ester; trifluoroacetate salt (446.1 [M+1]; MW 445.5);
6-(5-Fluoro-1,3-dihydro-isoindol-2-yl)-imidazo[1,2-b]pyridazine-3-carboxylic acid (4-hydroxy-phenyl)-amide; trifluoroacetate salt (390.1 [M+1]; MW 389.4);
(4-{[6-(4-Methoxy-benzylamino)-7-methyl-imidazo[1,2-b]pyridazine-3-carbonyl]-amino}-phenyl)-acetic acid (436.1 [M+1]; MW 431.5);
[6-(4-Fluoro-benzylamino)-imidazo[1,2-b]pyridazin-3-yl]-(5-fluoro-1,3-dihydro-isoindol-2-yl)-methanone; trifluoroacetate salt (406.1 [M+1]; MW 405.4);
6-(4-Fluoro-benzylamino)-imidazo[1,2-b]pyridazine-3-carboxylic acid (3-hydroxy-4-methoxy-phenyl)-amide; trifluoroacetate salt (408.1 [M+1]; MW 407.4);
3-{[6-(4-Fluoro-benzylamino)-imidazo[1,2-b]pyridazine-3-carbonyl]-amino}-4-methyl-benzoic acid (420.1 [M+1]; MW 419.4);
6-(4-Fluoro-phenylamino)-imidazo[1,2-b]pyridazine-3-carboxylic acid (4-dimethylamino-phenyl)-amide; trifluoroacetate salt (391.1 [M+1]; MW 390.4);
(5-Fluoro-1,3-dihydro-isoindol-2-yl)-[6-(4-methoxy-phenylamino)-imidazo[1,2-b]pyridazin-3-yl]-methanone; trifluoroacetate salt (404.1 [M+1]; MW 403.4);
6-[(6-Trifluoromethyl-pyridin-3-ylmethyl)-amino]-imidazo[1,2-b]pyridazine-3-carboxylic acid (4-hydroxy-phenyl)-amide; trifluoroacetate salt (425.1 [M+1]; MW 428.4);
[6-(4-Fluoro-benzylamino)-7-methyl-imidazo[1,2-b]pyridazin-3-yl]-(5-fluoro-1,3-dihydro-isoindol-2-yl)-methanone; trifluoroacetate salt (420.1 [M+1]; MW 419.4); (5-Fluoro-1,3-dihydro-isoindol-2-yl)-[6-(4-methoxy-benzylamino)-imidazo[1,2-b]pyridazin-3-yl]-methanone; trifluoroacetate salt (418.1 [M+1]; MW 417.4);
6-(4-Fluoro-3-methyl-benzylamino)-imidazo[1,2-b]pyridazine-3-carboxylic acid (4-acetylamino-phenyl)-amide; trifluoroacetate salt (433.1 [M+1]; MW 432.5);
6-(4-Fluoro-3-methyl-benzylamino)-imidazo[1,2-b]pyridazine-3-carboxylic acid (4-hydroxymethyl-phenyl)-amide; trifluoroacetate salt (406.1 [M+1]; MW 405.4);
6-(5-Fluoro-1,3-dihydro-isoindol-2-yl)-imidazo[1,2-b]pyridazine-3-carboxylic acid (4-hydroxymethyl-phenyl)-amide; trifluoroacetate salt (404.1 [M+1]; MW 403.4);
6-(3-Methanesulfonyl-benzylamino)-imidazo[1,2-b]pyridazine-3-carboxylic acid phenylamide; trifluoroacetate salt (422.0 [M+1]; MW 421.5);
6-(3-Methanesulfonyl-benzylamino)-imidazo[1,2-b]pyridazine-3-carboxylic acid (4-hydroxy-phenyl)-amide; trifluoroacetate salt (438.1 [M+1]; MW 437.5);
6-(5-Methoxy-1,3-dihydro-isoindol-2-yl)-imidazo[1,2-b]pyridazine-3-carboxylic acid (4-hydroxy-phenyl)-amide; trifluoroacetate salt (402.1 [M+1]; MW 401.4);
3-{[6-(5-Methoxy-1,3-dihydro-isoindol-2-yl)-imidazo[1,2-b]pyridazine-3-carbonyl]-amino}-benzoic acid ethyl ester; trifluoroacetate salt (458.1 [M+1]; MW 457.5);
6-(5-Methoxy-1,3-dihydro-isoindol-2-yl)-imidazo[1,2-b]pyridazine-3-carboxylic acid (3-methanesulfonyl-phenyl)-amide; trifluoroacetate salt (464.1 [M+1]; MW 463.5);
6-[(6-Chloro-pyridin-3-ylmethyl)-amino]-imidazo[1,2-b]pyridazine-3-carboxylic acid (4-hydroxy-phenyl)-amide; trifluoroacetate salt (395.1 [M+1]; MW 394.8);
6-(4-Fluoro-benzylamino)-7-methyl-imidazo[1,2-b]pyridazine-3-carboxylic acid (4-hydroxy-phenyl)-methyl-amide; trifluoroacetate salt (406.1 [M+1]; MW 405.4);
(3-{[6-(4-Methoxy-benzylamino)-7-methyl-imidazo[1,2-b]pyridazine-3-carbonyl]-amino}-phenyl)-acetic acid (446.1 [M+1]; MW 445.5);
3-{[6-(5-Methoxy-1,3-dihydro-isoindol-2-yl)-imidazo[1,2-b]pyridazine-3-carbonyl]-methyl-amino}-benzoic acid ethyl ester (472.2 [M+1]; MW 471.5);
6-(5-Fluoro-1,3-dihydro-isoindol-2-yl)-imidazo[1,2-b]pyridazine-3-carboxylic acid (4-hydroxy-phenyl)-methyl-amide; trifluoroacetate salt (404.1 [M+1]; MW 403.4);
6-[4-(4-Methyl-piperazin-1-yl)-benzylamino]-imidazo[1,2-b]pyridazine-3-carboxylic acid (4-hydroxy-phenyl)-amide; trifluoroacetate salt (458.1 [M+1]; MW 457.5).

### Example 130

The following examples are also prepared in accordance with the methods described herein.
[6-(3,4-Dichloro-benzylamino)-8-methyl-imidazo[1,2-b]pyridazin-3-yl]-morpholin-4-yl-methanone;
6-(3,4-Dichlorobenzylamino)-7-methyl-imidazo[1,2-b]pyridazine-3-carboxylic acid phenylamide;
6-(3,4-Dichlorobenzylamino)-8-methyl-imidazo[1,2-b]pyridazine-3-carboxylic acid (1H-indol-5-yl)amide;
6-(3,4-Dichlorobenzylamino)-2-methyl-imidazo[1,2-b]pyridazine-3-carboxylic acid (2-methoxy-ethyl)-amide;
8-Fluoro-6-(4-methoxy-benzylamino)-imidazo[1,2-b]pyridazine-3-carboxylic acid (2-acetylamino-ethyl)-amide;
2-Ethyl-6-[(pyridin-3-ylmethyl)-amino]-imidazo[1,2-b]pyridazine-3-carboxylic acid ethylamide;
6-(3-Chloro-benzylamino)-7-fluoro-imidazo[1,2-b]pyridazine-3-carboxylic acid (3-trifluoromethoxy-phenyl)-amide;
4-({6-[(Furan-2-ylmethyl)-amino]-7-methyl-imidazo[1,2-b]pyridazine-3-carbonyl}-amino)-benzoic acid;
2-Chloro-6-(1H-indol-6-ylamino)-imidazo[1,2-b]pyridazine-3-carboxylic acid propylamide;
6-(3,4-Dimethoxy-benzylamino)-imidazo[1,2-b]pyridazine-3-carboxylic acid (tetrahydro-pyran-4-yl)-amide;
6-(Pyridin-4-ylamino)-imidazo[1,2-b]pyridazine-3-carboxylic acid (3,4-dimethoxy-phenyl)-amide;
6-(3-Hydroxy-phenylamino)-imidazo[1,2-b]pyridazine-3-carboxylic acid (3,4-dimethoxy-phenyl)-amide;
6-(3,4-Dimethoxy-phenyl)-imidazo[1,2-b]pyridazine-3-carboxylic acid (2-methoxy-ethyl)-amide;
7-Methyl-6-[(thiophen-2-ylmethyl)-amino]imidazo[1,2-b]pyridazine-3-carboxylic acid cyclopentylamide;
4-{[6-(3,4-Dichloro-benzyloxy)-imidazo[1,2-b]pyridazine-3-carbonyl]-amino}-benzoic acid;
3,4-Dimethoxy-N-{3-[(tetrahydro-pyran-4-ylamino)-methyl]-imidazo[1,2-b]pyridazin-6-yl}-benzamide;
Thiophene-2-carboxylic acid (3-cyclopentylaminomethyl-imidazo[1,2-b]pyridazin-6-yl)-amide;
6-(3-Chloro-benzylsulfanyl)-imidazo[1,2-b]pyridazine-3-carboxylic acid(2-methoxy-ethyl)-amide;
6-Benzyl-imidazo[1,2-b]pyridazine-3-carboxylic acid (2-acetylamino-ethyl)-amide;
(3,4-Dichloro-benzyl)-(7-methyl-3-morpholin-4-ylmethyl-imidazo[1,2-b]pyridazin-6-yl)-amine;
(3-Chloro-phenyl)-(3-phenylaminomethyl-imidazo[1,2-b]pyridazin-6-yl)-amine;
{8-Fluoro-3-[(1H-indol-5-ylamino)-methyl]-imidazo[1,2-b]pyridazin-6-yl}-thiophen-2-yl-amine;
3,4-Dichloro-N-{3-[(2-methoxy-ethylamino)-methyl]-imidazo[1,2-b]pyridazin-6-yl}-benzamide;
N-[6-(3,4-Dichloro-benzylamino)-7-methyl-imidazo[1,2-b]pyridazin-3-yl]-benzamide;
1H-Indole-5-carboxylic acid [6-(3,4-dichloro-benzylamino)-8-methyl-imidazo[1,2-b]pyridazin-3-yl]-amide;
2-Methoxy-ethanesulfonic acid [6-(3,4-dichloro-benzylamino)-imidazo[1,2-b]pyridazin-3-yl]-amide;
2-Methylamino-ethanesulfonic acid 8-fluoro-6-(4-methoxy-phenylamino)-imidazo[1,2-b]pyridazin-3-yl)-amide;
N-{3-[(Furan-2-ylmethyl)-amino]-imidazo[1,2-b]pyridazin-6-yl}-nicotinamide;
1-Isopropyl-3-{6-[(pyridin-3-ylmethyl)-amino]-imidazo[1,2-b]pyridazin-3-yl}-urea;
4-Chloro-3-fluoro-N-[6-(furan-2-ylmethyl-methyl-amino)-imidazo[1,2-b]pyridazin-3-yl]-benzamide;
(2-Fluoro-3-furan-2-ylmethyl-imidazo[1,2-b]pyridazin-6-yl)-(1H-indol-6-yl)-amine; Furan-2-ylmethyl-methyl-(2-methyl-3-piperidin-1-yl-imidazo[1,2-b]pyridazin-6-yl)-amine;
[2-Fluoro-3-(2-methyl-morpholin-4-yl)-imidazo[1,2-b]pyridazin-6-yl]-(1H-indol-6-yl)-amine;
N-(3-Cyclohexyl-2-methoxy-imidazo[1,2-b]pyridazin-6-yl)-nicotinamide;
6-(3,4-Dichloro-phenylethynyl)-7-methyl-imidazo[1,2-b]pyridazine-3-carboxylic acid phenylamide;
2-Fluoro-N*6*-(4-fluoro-benzyl)-N*3*-(4-methoxy-phenyl)-imidazo[1,2-b]pyridazine-3,6-diamine;
2-Fluoro-N*3*-(4-methanesulfonyl-phenyl)-N*6*-(3-trifluoromethyl-benzyl)-imidazo[1,2-b]pyridazine-3,6-diamine;
1-(3-Hydroxymethyl-phenyl)-3-[6-(2-imidazol-1-yl-ethylamino)-imidazo[1,2-b]pyridazin-3-yl]-urea;
[2-Fluoro-6-(pyrazin-2-ylmethoxy)-imidazo[1,2-b]pyridazin-3-yl]-(4-trifluoromethoxy-phenyl)-amine;
2-Fluoro-N*6*-(5-methyl-1H-pyrazol-4-ylmethyl)-N*3*-naphthalen-2-yl-imidazo[1,2-b]pyridazine-3,6-diamine;
Benzofuran-2-yl-[2-fluoro-6-(5-methyl-thiophen-2-ylmethoxy)-imidazo[1,2-b]pyridazin-3-yl]-amine;
Pyrazine-2-carboxylic acid [2-fluoro-3-(6-methyl-pyridin-2-ylmethyl)-imidazo[1,2-b]pyridazin-6-yl]-amide;
4-Cyano-N-{8-methyl-6-[2-(1-methyl-1H-pyrazol-4-yl)-ethylamino]-imidazo[1,2-b]pyridazin-3-yl}-benzamide;
(3-Cyclopentylmethyl-2-fluoro-imidazo[1,2-b]pyridazin-6-yl)-[2-(1H-pyrrol-2-yl)-ethyl]-amine;
Pyridine-2-carboxylic acid [3-(cyclopropylmethyl-amino)-imidazo[1,2-b]pyridazin-6-yl]-amide;
3-[(3-Hydroxy-cyclohexylmethyl)-amino]-imidazo[1,2-b]pyridazine-6-carboxylic acid (4-fluoro-phenyl)-amide;
(3,4-Dichloro-benzyl)-(3-morpholin-4-ylmethyl-imidazo[1,2-b]pyridazin-6-yl)-amine;
(3,4-Dichloro-benzyl)-(3-phenylaminomethyl-imidazo[1,2-b]pyridazin-6-yl)-amine;
(3,4-Dichloro-benzyl)-{3-[(1H-indol-5-ylamino)-methyl]-imidazo[1,2-b]pyridazin-6-yl}-amine;
(3,4-Dichloro-benzyl)-{3-[(2-methoxy-ethylamino)-methyl]-imidazo[1,2-b]pyridazin-6-yl}-amine;
N-(2-{[6-(3,4-Dichloro-benzylamino)-imidazo[1,2-b]pyridazin-3-ylmethyl]-amino}-ethyl)-acetamide;
(3,4-Dichloro-benzyl)-(3-ethylaminomethyl-imidazo[1,2-b]pyridazin-6-yl)-amine;
1-(3-{[6-(3,4-Dichloro-benzylamino)-imidazo[1,2-b]pyridazin-3-ylmethyl]-amino}-phenyl)-ethanone;
4-{[6-(3,4-Dichloro-benzylamino)-imidazo[1,2-b]pyridazin-3-ylmethyl]-amino}-benzoic acid ethyl ester;
4-{[6-(3,4-Dichloro-benzylamino)-imidazo[1,2-b]pyridazin-3-ylmethyl]-amino}-benzoic acid;
(3,4-Dichloro-benzyl)-{3-[(1H-indol-5-ylamino)-methyl]-imidazo[1,2-b]pyridazin-6-yl}-amine;
4-{[6-(3,4-Dichloro-benzylamino)-7-methyl-imidazo[1,2-b]pyridazine-3-carbonyl]-amino}-benzoic acid;
4-{[6-(3,4-Dichloro-benzylamino)-8-methyl-imidazo[1,2-b]pyridazine-3-carbonyl]-amino}-benzoic acid;
4-{[6-(3,4-Dichloro-phenylamino)-7-methyl-imidazo[1,2-b]pyridazine-3-carbonyl]-amino}-benzoic acid;
4-{[6-(3,4-Dichloro-phenylamino)-8-methyl-imidazo[1,2-b]pyridazine-3-carbonyl]-amino}-benzoic acid;
4-{[6-(Furan-2-ylamino)-7-methyl-imidazo[1,2-b]pyridazine-3-carbonyl]-amino}-benzoic acid;
4-{[6-(Furan-2-ylamino)-8-methyl-imidazo[1,2-b]pyridazine-3-carbonyl]-amino}-benzoic acid;
6-(4-Fluoro-phenoxy)-8-methyl-3-phenylethynyl-imidazo[1,2-b]pyridazine;
N-[6-(3,4-Dichloro-benzylamino)-imidazo[1,2-b]pyridazin-3-yl]-terephthalamic acid methyl ester;
N-[6-(3,4-Dichloro-benzylamino)-imidazo[1,2-b]pyridazin-3-yl]-terephthalamic acid;

N-[6-(3,4-Dichloro-benzylamino)-imidazo[1,2-b]pyridazin-3-yl]-4-methoxy-benzamide;
N-[6-(3,4-Dichloro-benzylamino)-imidazo[1,2-b]pyridazin-3-yl]-4-hydroxy-benzamide;
4-[6-(3,4-Dichloro-benzylamino)-imidazo[1,2-b]pyridazin-3-ylsulfamoyl]-benzoic acid methyl ester;
4-[6-(3,4-Dichloro-benzylamino)-imidazo[1,2-b]pyridazin-3-ylsulfamoyl]-benzoic acid;
Ethanesulfonic acid [6-(3,4-dichloro-benzylamino)-imidazo[1,2-b]pyridazin-3-yl]-amide;
1H-Indole-5-carboxylic acid [6-(3,4-dichloro-benzylamino)-imidazo[1,2-b]pyridazin-3-yl]-amide;
N*6*-(3,4-Dichloro-benzyl)-N*3*-(4-methoxy-benzyl)-imidazo[1,2-b]pyridazine-3,6-diamine;
4-{[6-(3,4-Dichloro-benzylamino)-imidazo[1,2-b]pyridazin-3-ylamino]-methyl}-phenol;
N*6*-(3,4-Dichloro-benzyl)-N*3*-propyl-imidazo[1,2-b]pyridazine-3,6-diamine;
3-Acetyl-N-[6-(3,4-dichloro-benzylamino)-imidazo[1,2-b]pyridazin-3-yl]-benzamide;
N*6*-(3,4-Dichloro-benzyl)-N*3*-pentyl-imidazo[1,2-b]pyridazine-3,6-diamine;
N-[6-(4-Fluoro-benzylamino)-imidazo[1,2-b]pyridazin-3-yl]-propionamide;
N-[6-(4-Methoxy-benzylamino)-imidazo[1,2-b]pyridazin-3-yl]-propionamide;
N*6*-(3,4-Dichloro-benzyl)-N*3*, N*3*-dimethyl-imidazo[1,2-b]pyridazine-3,6-diamine;
1H-Indole-5-carboxylic acid [6-(4-methoxy-benzylamino)-2H-imidazo[1,2-b]pyridazin-3-yl]-amide;
1H-Indole-5-carboxylic acid [6-(4-fluoro-benzylamino)-imidazo[1,2-b]pyridazin-3-yl]-amide;
(3-Cyclohexyl-imidazo[1,2-b]pyridazin-6-yl)-(3,4-dichloro-benzyl)-amine;
(3-Cyclohexylmethyl-imidazo[1,2-b]pyridazin-6-yl)-(3,4-dichloro-benzyl)-amine;
(3-Cyclopentyl-imidazo[1,2-b]pyridazin-6-yl)-(3,4-dichloro-benzyl)-amine;
(3-Cyclobutyl-imidazo[1,2-b]pyridazin-6-yl)-(3,4-dichloro-benzyl)-amine;
(3-Cyclopropyl-imidazo[1,2-b]pyridazin-6-yl)-(3,4-dichloro-benzyl)-amine;
(3-Cyclobutylmethyl-imidazo[1,2-b]pyridazin-6-yl)-(3,4-dichloro-benzyl)-amine;
(3-Cyclopropylmethyl-imidazo[1,2-b]pyridazin-6-yl)-(3,4-dichloro-benzyl)-amine;
(3,4-Dichloro-benzyl)-[3-(4-phenyl-but-1-ynyl)-imidazo[1,2-b]pyridazin-6-yl]-amine;
3-Cyclopentylmethyl-imidazo[1,2-b]pyridazin-6-yl)-(3,4-dichloro-benzyl)-amine;
(3,4-Dichloro-benzyl)-(3-piperidin-1-yl-imidazo[1,2-b]pyridazin-6-yl)-amine; and
(3,4-Dichloro-benzyl)-(3-morpholin-4-yl-imidazo[1,2-b]pyridazin-6-yl-amine.

### Example 131

Compounds of the examples were tested in either one of the biological tests described above and were found to exhibit 50% inhibition of PIM-1 or PIM-2 (as appropriate) at a concentration of 50 μM or below. For example, the following representative compounds of the examples exhibited the following IC₅₀ values:

### Example 11: 0.08 μM

### Example 15: 4.03 μM

### Example 17: 0.43 μM

### Example 18: 0.06 μM

### Example 19: 4.66 μM

### Example 22: 33 μM

### Example 23: 0.13 μM

### Example 25: 2.2 μM

### Example 27: 1.14 μM

### Example 29: 0.26 μM

### Example 30: 0.38 μM

### Example 31: 7.38 μM

### Example 32: 0.07 μM

### Example 34: 11 μM

### Example 35: 0.12 μM

### Example 36: 2.26 μM

### Example 37: 0.53 μM

### Example 38: 4.63 μM

### Example 39: 0.04 μM

### Example 41: 0.06 μM

### Example 42: 0.06 μM

### Example 44: 0.18 μM

### Example 45: 0.49 μM

### Example 46: 0.38 μM

### Example 47: 0.47 μM

### Example 53: 33 μM

### Example 54: 0.07 μM

### Example 55: 0.07 μM

### Example 58: 0.3 μM

### Example 60: 0.06 μM

### Example 61: 0.06 μM

### Example 64: 0.16 μM

### Example 65: 19.32 μM

### Example 66: 3.13 μM

### Example 68: 0.08 μM

### Example 69: 0.19 μM

### Example 70: 2.22 μM

### Example 71: 5.19 μM

### Example 73: 2.85 μM

### Example 80: 4.55 μM

### Example 81: 8.87 μM

### Example 84: 5.23 μM

### Example 85: 17.39 μM

### Example 86: 14.51 μM

### Example 89: 19.04 μM

### Example 90: 1.15 μM

### Example 91: 0.31 μM

### Example 98: 4.65 μM

### Example 100: 19.07 μM

### Example 101: 0.41 μM

### Example 102: 2.25 μM

Example 103: 14.4 μM

1. A compound of formula I, 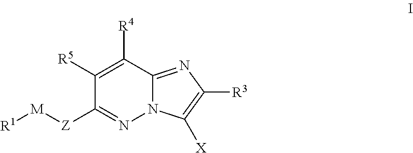
wherein:
Z represents a direct bond, —(CH₂)ₙ—O—, —(CH₂)ₙ—S—, —(CH₂)ₙ—N(R^{a})—, —(CH₂)ₙ—C(O)—, —(CH₂)ₙ—C(O)O—, —(CH₂)ₙ—S(O)—, —(CH₂)ₙ—SO₂—, —(CH₂)ₙ—N(R^{a})—SO₂—, —(CH₂)ₙ—SO₂—N(R^{a})—, —(CH₂)ₙ—N(R^{a})—CO—, —(CH₂)ₙ—NH—CO—NH— or —(CH₂)ₙ—CO—N(R^{a})—;
n represents, on each occasion when mentioned above, 0, 1 or 2
M represents a direct bond or C₁₋₈ alkylene optionally substituted by one or more substituents selected from halo, —OR^{b}, —SR^{b} and —N(R^{b})₂;
R¹ represents aryl or heteroaryl, both of which are optionally substituted by one or more substituents selected from B¹;
X represents C₃₋₆ cycloalkyl, heterocycloalkyl (which latter two groups are optionally substituted by one or more substituents selected from B⁴ and B⁵, respectively) or -G-R²;
G represents —(CH₂)ₘ—O—, —(CH₂)ₘ—S—, —(CH₂)ₘ—N(R^{d})—, —(CH₂)ₘ—C(O)—, —(CH₂)ₘ—C(O)O—, —(CH₂)ₘ—S(O)—, —(CH₂)ₘ—SO₂—, —(CH₂)ₘ—N(R^{d})—SO₂—, —(CH₂)ₘ—SO₂—N(R^{d})—, —(CH₂)ₘ—N(R^{d})—CO—, —(CH₂)ₘ—CO—N(R^{d})—, —(CH₂)ₘ—NH—CO—NH— or C₁₋₈ alkylene optionally substituted by one or more substituents selected from A¹;
m represents, on each occasion when used herein, 0, 1 or 2;
R² represents hydrogen, C₁₋₈ alkyl (optionally substituted by one or more substituents selected from A²) or -T-Q;
T represents a direct bond or a C₁₋₃ alkylene linker group optionally substituted by one or more substituents selected from A³;
Q represents C₃₋₆ cycloalkyl, heterocycloalkyl (which latter two groups are optionally substituted by one or more substituents selected from B⁶ and B⁷, respectively), aryl or heteroaryl (which latter two groups are optionally substituted by one or more substituents selected from B⁸ and B⁹, respectively);
A¹, A² and A³ independently represent halo, —OR^{e}, —S—C₁₋₄ alkyl, —N(R^{e})₂, —C(O)₂R^{e}, —C(O)N(R^{e})₂, —N(R^{e})—C(O)—R^{e}, —C(O)R^{e}, —CN, —SO₂N(R^{e})₂, phenyl (optionally substituted by one or more substituents selected from halo and —OR^{e}) and/or C₁₋₄ alkyl (optionally substituted by one or more substituents selected from halo);
B¹, B⁴, B⁵, B⁶, B⁷, B⁸ and B⁹ independently represent, on each occasion when used herein, halo, —OR^{e}, —C(O)₂R^{e}, —C(O)R^{e}, —C(O)N(R^{e})₂, —N(R^{e})—C(O)—R^{e}, —CN, —S(O)₂R^{e}, —S(O)₂N(R^{e})₂, —N(R^{e})₂ and/or C₁₋₄ alkyl (optionally substituted by one or more substituents selected from halo, —OR^{e} and —C(O)₂R^{e}); or,
B⁴, B⁵, B⁶ and B⁷ may alternatively and independently represent ═O;
R³, R⁴ and R⁵ independently represent hydrogen, halo, —R^{j}, —OR^{f}, —SR^{f}, cyano or —N(R^{f})₂;
R^{a}, R^{b}, R^{d}, R^{e} and R^{f} independently represent, on each occasion when used herein, hydrogen and/or C₁₋₄ alkyl optionally substituted with one or more substituents selected from halo and —OR^{h}; or
any two R^{e} groups, when attached to the same nitrogen atom may be linked together to form (together with the requisite nitrogen atom to which those R^{e} groups are necessarily attached) a 3- to 8-membered ring optionally containing a further one or two heteroatoms, which ring optionally contains one to three unsaturations and is optionally substituted by one or more substituents selected from ═O and C₁₋₃ alkyl (optionally substituted by one or more fluoro atoms);
R^{j} represents, on each occasion when used herein, hydrogen, aryl, heteroaryl, C₃₋₆ cycloalkyl, heterocycloalkyl and/or C₁₋₄ alkyl, which latter five groups are optionally substituted with one or more substituents selected from halo, C₁₋₄ alkyl and —OR^{h};
R^{h} represents, on each occasion when used herein, hydrogen or C₁₋₄ alkyl optionally substituted by one or more halo atoms,
or a pharmaceutically acceptable ester, amide, solvate or salt thereof,
provided that when:
(I) R⁴ and R⁵ represent hydrogen, Z represents —S—, R¹ represents unsubstituted phenyl, X represents -G-R²:
(i) M represents a direct bond, then when:
(A) R³ represents hydrogen and G represents —C(O)—;
(B) R³ represents —CH₃ and G represents —(CH₂)—NH—C(O)—,
then R² does not represent unsubstituted phenyl;
(ii) M represents —CH₂—, R³ represents tert-butyl and G represents —O—, then R² does not represent —CH₃;
(II) R⁴ and R⁵ represent hydrogen, Z and M represent direct bonds, R¹ represents (3,5-dimethyl)pyrazol-1-yl, R³ represents —OCH₃, X represents -G-R² and G represents —SO₂, then R² does not represent unsubstituted 1,3,4-triazol-2-yl or 1,2,4-triazol-3-yl substituted at the 1-position with B⁹, in which B⁹ represents —C(O)N(R^{e})₂ and each R^{e} represents ethyl;
(III) R⁴ and R⁵ represent hydrogen, Z represents —O—, R³ represents tert-butyl, X represents -G-R², G represents —O— and R² represents —CH₃, then:
(i) when M represents a direct bond, then R¹ does not represent 2-methoxyphenyl;
(ii) when M represents —CH₂—, then R¹ does not represent unsubstituted phenyl;
(IV) R⁴ and R⁵ represent hydrogen, R³ represents —CF₃, X represents -G-R², G represents —CH₂—:
(a) R² represents (4-n-propyl)pyrrolidin-2-one and M represents —CH₂—, then:
(i) when Z represents —N(H)—, R¹ does not represent (2,4-dimethoxy)phenyl;
(ii) when Z represents —O—, R¹ does not represent unsubstituted phenyl;
(b) R² represents (4-CH═CF₂)pyrrolidin-2-one (i.e. 4-(2,2-difluoroethenyl)pyrrolidin-2-one), and M and Z both represent direct bonds, then R¹ does not represent unsubstituted 3-pyridyl, 3-thienyl or phenyl;
(V) Z and M represent direct bonds, R³ and R⁵ represent hydrogen, X represents -G-R², G represents —CH₂— and R² represents 4-morpholinyl, then:
(i) when R⁴ represents methyl, then R¹ does not represent 3-methoxyphenyl or unsubstituted phenyl; or
(ii) when R⁴ represents hydrogen, then R¹ does not represent 4-chlorophenyl or unsubstituted phenyl;
(VI) Z and M represent direct bonds, R³ and R⁵ represent hydrogen, X represents -G-R², G represents —CH₂— substituted by A¹ in which A¹ represents —N(CH₃)₂, and R² represents hydrogen (so forming a —CH₂—N(CH₃)₂ group), then R¹ does not represent unsubstituted phenyl when R⁴ represents hydrogen or methyl;
(VII) Z and M represent direct bonds, R³ and R⁵ represent hydrogen, R⁴ represents —CF₃, R¹ represents 4-trifluoromethylphenyl, X represents -G-R², G represents —C≡C— (i.e. ethynylene), then R² does not represent 2-(NH₂)-pyrimidin-5-yl, 5-(S(O)₂NH₂)-thien-2-yl or 6-(NH₂)-pyrid-3-yl;
(VIII) Z and M represent direct bonds, R³, R⁴ and R⁵ represent hydrogen, X represents -G-R², G represents —CH₂— substituted by A¹ in which A¹ represents —C(O)₂R^{e}, R² represents unsubstituted phenyl, then:
(i) when R^{e} represents ethyl, then R¹ does not represent 4-chlorophenyl, 4-methoxyphenyl or unsubstituted phenyl;
(ii) when R^{e} represents hydrogen, then R¹ does not represent 4-chlorophenyl or 4-methoxyphenyl;
(IX) Z and M represent direct bonds, R³, R⁴ and R⁵ represent hydrogen, X represents -G-R², G represents —CH₂—, then when:
(i) R¹ represents 3-trifluoromethylphenyl, then R² does not represent H, or ethyl;
(ii) R¹ represents unsubstituted 3-pyridyl, then R² does not represent H or methyl;
(X) Z and M represent direct bonds, R³, R⁴ and R⁵ represent hydrogen, X represents -G-R², G represents —CH₂— and R² represents H, then R¹ does not represent unsubstituted phenyl.

2. A compound as claimed in claim 1, wherein Z represents a direct bond, —(CH₂)ₙ—O—, —(CH₂)ₙ—S—, —(CH₂)ₙ—N(R^{a})—, —(CH₂)ₙ—S(O)—, —(CH₂)ₙ—SO₂—, —(CH₂)ₙ—N(R^{a})—SO₂—, —(CH₂)ₙ—SO₂—N(R^{a})—, —(CH₂)ₙ—N(R^{a})—CO—, —(CH₂)ₙ—NH—CO—NH— or —(CH₂)ₙ—CO—N(R^{a})—.

3. A compound as claimed in claim 2, wherein Z represents a direct bond, —(CH₂)ₙ—O—, —(CH₂)ₙ—S—, —(CH₂)ₙ—N(R^{a})—, —(CH₂)ₙ—N(R^{a})—CO— or —(CH₂)ₙ—CO—N(R^{a})—.

4. A compound according to claim 1, wherein M represents a direct bond or C₁₋₃ alkylene.

5. A compound according to claim 1, wherein G represents —(CH₂)ₘ—O—, —(CH₂)ₘ—SO₂N(R^{d})—, —(CH₂)ₘ—N(R^{d})—SO₂—, —(CH₂)ₘ—SO₂—, —(CH₂)ₘ—N(R^{d})—, —(CH₂)ₘ—N(R^{d})—, —(CH₂)ₘ—N(R^{d})—, —(CH₂)ₘ, —C(O)—, —(CH₂)ₘ—C(O)O—, —(CH₂)ₘ—C(O)—N(R^{d})—, —(CH₂)ₘ—N(R^{d})—SO₂—, —(CH₂)ₘ—N(R^{d})—C(O)—, —(CH₂)ₘ—NH—C(O)—NH— or C₁₋₆ alkylene.

6. A compound according to claim 1, wherein R² represents hydrogen, optionally substituted (i.e. by A²) C₁₋₅ alkyl or -T-Q.

7. A compound according to claim 1, wherein T represents a direct bond or C₁₋₂ alkylene.

8. A compound according to claim 1, wherein A¹ to A³ independently represent —OR^{e}, —N(R^{e})—C(O)—R^{e} and/or —N(R^{e})₂.

9. A compound according to claim 1, wherein B¹ to B⁹ independently represent —N(R^{e})₂, —N(R^{e})C(O)R^{e}, —C(O)N(R^{e})₂, —S(O)₂N(R^{e})₂, halo, —OR^{e}, —C(O)₂R^{e}, —C(O)R^{e}, —CN, —S(O)₂R^{e} and/or C₁₋₃ alkyl optionally substituted by one or more substituents selected from —C(O)₂R^{e}, halo and —OR^{e}.

10. A compound according to claim 1, wherein R³, R⁴ and R⁵ independently represent hydrogen, halo, R^{j} or —OR^{f}.

11. A compound according to claim 1, wherein R^{a}, R^{b}, R^{d}, R^{e}, R^{f} and R^{j} independently represent hydrogen or C₁₋₃ alkyl optionally substituted by one or more halo atoms, R^{j} may alternatively and independently represent C₃₋₆ cycloalkyl, or, any two R^{e} groups may be linked together to form a 5- or 6-membered ring optionally containing a further nitrogen or oxygen heteroatom, which ring optionally contains a double bond, and is optionally substituted by one or more substituents selected from ═O and C₁₋₃ alkyl.

12. A compound according to claim 1, wherein R^{h} represents hydrogen or C₁₋₂ alkyl optionally substituted by one or more fluoro atoms.

13. (canceled)

14. A pharmaceutical formulation including a compound of formula I, or a pharmaceutically acceptable ester, amide, solvate or salt thereof, in admixture with a pharmaceutically acceptable adjuvant, diluent or carrier,
where the compound of formula I has the formula 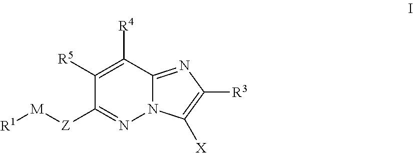
wherein:
Z represents a direct bond, —(CH₂)ₙ—O—, —(CH₂)ₙ—S—, —(CH₂)ₙ—N(R^{a})—, —(CH₂)ₙ—C(O)—, —(CH₂)ₙ—C(O)O—, —(CH₂)ₙ—S(O)—, —(CH₂)ₙ—SO₂—, —(CH₂)ₙ—N(R^{a})—SO₂—, —(CH₂)ₙ—SO₂—N(R^{a})—, —(CH₂)ₙ—N(R^{a})—CO—, —(CH₂)ₙ—NH—CO—NH— or —(CH₂)ₙ—CO—N(R^{a})—;
n represents, on each occasion when mentioned above, 0, 1 or 2
M represents a direct bond or C₁₋₈ alkylene optionally substituted by one or more substituents selected from halo, —OR^{b}—SR^{b} and —N(R^{b})₂;
R¹ represents aryl or heteroaryl, both of which are optionally substituted by one or more substituents selected from B¹;
X represents C₃₋₆ cycloalkyl, heterocycloalkyl groups (which latter two groups are optionally substituted by one or more substituents selected from B⁴ and B⁵, respectively) or -G-R²;
G represents —(CH₂)ₘ—O—, —(CH₂)ₘ—S—, —(CH₂)ₘ—N(R^{d})—, —(CH₂)ₘ—C(O)—, —(CH₂)ₘ—C(O)O—, —(CH₂)ₘ—S(O)—, —(CH₂)ₘ—SO₂—, —(CH₂)ₘ—N(R^{d})—SO₂—, —(CH₂)ₘ—SO₂—N(R^{d})—, —(CH₂)ₘ—N(R^{d})—CO—, —(CH₂)ₘ—CO—N(R^{d})—, —(CH₂)ₘ—NH—CO—NH— or C₁₋₈ alkylene optionally substituted by one or more substituents selected from A¹;
m represents, on each occasion when used herein, 0, 1 or 2;
R² represents hydrogen, C₁₋₈ alkyl optionally substituted by one or more substituents selected from A²) or -T-Q;
T represents a direct bond or a C₁₋₃ alkylene linker group optionally substituted by one or more substituents selected from A³;
Q represents C₃₋₆ cycloalkyl, heterocycloalkyl (which latter two groups are optionally substituted by one or more substituents selected from B⁶ and B⁷, respectively), aryl or heteroaryl (which latter two groups are optionally substituted by one or more substituents selected from B⁸ and B⁹, respectively);
A¹, A² and A³ independently represent halo, —OR^{e}, —S—C₁₋₄ alkyl, —N(R^{e})₂, —C(O)₂R^{e}, —C(O)N(R^{e})₂, —N(R^{e})—C(O)—R^{e}, —C(O)R^{e}, —CN, —SO₂N(R^{e})₂, phenyl (optionally substituted by one or more substituents selected from halo and —OR^{e}) and/or C₁₋₄ alkyl (optionally substituted by one or more substituents selected from halo);
B¹, B⁴, B⁵, B⁶, B⁷, B⁸ and B⁹ independently represent, on each occasion when used herein, halo, —OR^{e}, —C(O)₂R^{e}, —C(O)R^{e}, —C(O)N(R^{e})₂, —N(R^{e})—C(O)—R^{e}, —CN, —S(O)₂R^{e}, —S(O)₂N(R^{e})₂, —N(R^{e})₂ and/or C₁₋₄ alkyl (optionally substituted by one or more substituents selected from halo, —OR^{e} and —C(O)₂R^{e}); or,
B⁴, B⁵, B⁶ and B⁷ may alternatively and independently represent ═O;
R³, R⁴ and R⁵ independently represent hydrogen, halo, —R^{j}, —OR^{f}, —SR^{f}, cyano or —N(R^{f})₂;
R^{a}, R^{b}, R^{d}, R^{e} and R^{f} independently represent, on each occasion when used herein, hydrogen and/or C₁₋₄ alkyl optionally substituted with one or more substituents selected from halo and —OR^{h}; or
any two R^{e} groups, when attached to the same nitrogen atom may be linked together to form (together with the requisite nitrogen atom to which those R^{e} groups are necessarily attached) a 3- to 8-membered ring optionally containing a further one or two heteroatoms, which ring optionally contains one to three unsaturations and is optionally substituted by one or more substituents selected from ═O and C₁₋₃ alkyl (optionally substituted by one or more fluoro atoms);
R^{j} represents, on each occasion when used herein, hydrogen, aryl, heteroaryl, C₃₋₆ cycloalkyl, heterocycloalkyl and/or C₁₋₄ alkyl, which latter five groups are optionally substituted with one or more substituents selected from halo, C₁₋₄ and —OR^{h};
R^{h} represents, on each occasion when used herein, hydrogen or C₁₋₄ alkyl optionally substituted by one or more halo atoms,
provided that when:
(I) R⁴ and R⁵ represent hydrogen, Z represents —S—, R¹ represents unsubstituted phenyl, X represents -G-R²:
(i) M represents a direct bond, then when:
(A) R³ represents hydrogen and G represents —C(O)—;
(B) R³ represents —CH₃ and G represents —(CH₂)—NH—C(O)—,
then R² does not represent unsubstituted phenyl;
(ii) M represents —CH₂—, R³ represents tert-butyl and G represents —O—, then R² does not represent —CH₃;
(III) R⁴ and R⁵ represent hydrogen, Z represents —O—, R³ represents tert-butyl, X represents -G-R², G represents —O— and R² represents —CH₃, then:
(i) when M represents a direct bond, then R¹ does not represent 2-methoxyphenyl;
(ii) when M represents —CH₂—, then R¹ does not represent unsubstituted phenyl;
(IV) R⁴ and R⁵ represent hydrogen, R³ represents —CF₃, X represents -G-R², G represents —CH₂—:
(a) R² represents (4-n-propyl)pyrrolidin-2-one and M represents —CH₂—, then:
(i) when Z represents —N(H)—, R¹ does not represent (2,4-dimethoxy)phenyl;
(ii) when Z represents —O—, R¹ does not represent unsubstituted phenyl;
(b) R² represents (4-CH═CF₂)pyrrolidin-2-one (i.e. 4-(2,2-difluoroethenyl)pyrrolidin-2-one), and M and Z both represent direct bonds, then R¹ does not represent unsubstituted 3-pyridyl, 3-thienyl or phenyl;
(V) Z and M represent direct bonds, R³ and R⁵ represent hydrogen, X represents -G-R², G represents —CH₂— and R² represents 4-morpholinyl, then:
(i) when R⁴ represents methyl, then R¹ does not represent 3-methoxyphenyl or unsubstituted phenyl; or
(ii) when R⁴ represents hydrogen, then R¹ does not represent 4-chlorophenyl or unsubstituted phenyl;
(VI) Z and M represent direct bonds, R³ and R⁵ represent hydrogen, X represents -G-R², G represents —CH₂— substituted by A¹ in which A¹ represents —N(CH₃)₂, and R² represents hydrogen (so forming a —CH₂—N(CH₃)₂ group), then R¹ does not represent unsubstituted phenyl when R⁴ represents hydrogen or methyl;
(VII) Z and M represent direct bonds, R³ and R⁵ represent hydrogen, R⁴ represents —CF₃, R¹ represents 4-trifluoromethylphenyl, X represents -G-R², G represents —C≡O— (i.e. ethynylene), then R² does not represent 2-(NH₂)-pyrimidin-5-(S(O)₂NH₂)-thien-2-yl or 6-(NH₂)-pyrid-3-yl; and
(IX) Z and M represent direct bonds, R³, R⁴ and R⁵ represent hydrogen, X represents -G-R², G represents —CH₂—, then when:
(i) R¹ represents 3-trifluoromethylphenyl, then R² does not represent H, or ethyl;
(ii) R¹ represents unsubstituted 3-pyridyl, then R² does not represent H or methyl.

15-18. (canceled)

19. A method of treatment of a disease in which inhibition of a PIM family kinase and/or PI3-K is desired and/or required, which method comprises administration of a therapeutically effective amount of a compound of formula I, or a pharmaceutically-acceptable ester, amide, solvate or salt thereof, to a patient suffering from, or susceptible to, such a condition, where the compound of formula I has the formula 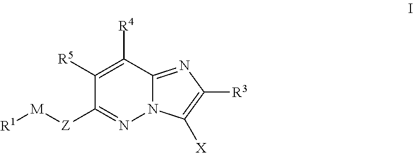
wherein:
Z represents a direct bond, —(CH₂)ₙ—O—, —(CH₂)ₙ—S—, —(CH₂)ₙ—N(R^{a})—, —(CH₂)ₙ—C(O)—, —(CH₂)ₙ—C(O)O—, —(CH₂)ₙ—S(O)—, —(CH₂)ₙ—SO₂—, —(CH₂)ₙ—N(R^{a})—SO₂—, —(CH₂)ₙ—SO₂—N(R^{a})—, —(CH₂)ₙ—N(R^{a})—CO—, —(CH₂)ₙ—NH—CO—NH— or —(CH₂)ₙ—CO—N(R^{a})—;
n represents, on each occasion when mentioned above, 0, 1 or 2
M represents a direct bond or C₁₋₈ alkylene optionally substituted by one or more substituents selected from halo, —OR^{b}, —SR^{b} and —N(R^{b})₂;
R¹ represents aryl or heteroaryl, both of which are optionally substituted by one or more substituents selected from B¹;
X represents C₃₋₆ cycloalkyl, heterocycloalkyl (which latter two groups are optionally substituted by one or more substituents selected from B⁴ and B⁵, respectively) or -G-R²;
G represents —(CH₂)ₘ—O—, —(CH₂)ₘ—S—, —(CH₂)ₘ—N(R^{d})—, —(CH₂)ₘ—C(O)—, —(CH₂)ₘ—C(O)O—, —(CH₂)ₘ—S(O)—, —(CH₂)ₘ—SO₂—, —(CH₂)ₘ—N(R^{d})—SO₂—, —(CH₂)ₘ—SO₂—N(R^{d})—, —(CH₂)ₘ—N(R^{d})—CO—, —(CH₂)ₘ—CO—N(R^{d})—, —(CH₂)ₘ—NH—CO—NH— or C₁₋₈ alkylene optionally substituted by one or more substituents selected from A¹;
m represents, on each occasion when used herein, 0, 1 or 2;
R² represents hydrogen, C₁₋₈ alkyl (optionally substituted by one or more substituents selected from A²) or -T-Q;
T represents a direct bond or a C₁₋₃ alkylene linker group optionally substituted by one or more substituents selected from A³;
Q represents C₃₋₆ cycloalkyl, heterocycloalkyl (which latter two groups are optionally substituted by one or more substituents selected from B⁶ and B⁷, respectively), aryl or heteroaryl (which latter two groups are optionally substituted by one or more substituents selected from B⁸ and B⁹, respectively);
A¹, A² and A³ independently halo, —OR^{e}, —S—C₁₋₄ alkyl, —N(R^{e})₂, —C(O)₂R^{e}, —C(O)N(R^{e})₂, —N(R^{e})—C(O)—R^{e}, —C(O)R^{e}, —CN, —SO₂N(R^{e})₂, phenyl (optionally substituted by one or more substituents selected from halo and —OR^{e}) and/or C₁₋₄ alkyl (optionally substituted by one or more substituents selected from halo);
B¹, B⁴, B⁵, B⁶, B⁷, B⁸ and B⁹ independently represent, on each occasion when used herein, halo, —OR^{e}, —C(O)₂R^{e}, —C(O)R^{e}, —C(O)N(R^{e})₂, —N(R^{e})—C(O)—R^{e}, —CN, —S(O)₂R^{e}, —S(O)₂N(R^{e})₂, —N(R^{e})₂ and/or C₁₋₄ alkyl (optionally substituted by one or more substituents selected from halo, —OR^{e} and —C(O)₂R^{e}); or,
B⁴, B⁵, B⁶ and B⁷ may alternatively and independently represent ═O;
R³, R⁴ and R⁵ independently represent hydrogen, halo, —R^{j}, —OR^{f}, —SR^{f}, cyano or —N(R^{f})₂;
R^{a}, R^{b}, R^{d}, R^{e} and R^{f} independently represent, on each occasion when used herein, hydrogen and/or C₁₋₄ alkyl optionally substituted with one or more substituents selected from halo and —OR^{h}; or
any two R^{e} groups, when attached to the same nitrogen atom may be linked together to form (together with the requisite nitrogen atom to which those R^{e} groups are necessarily attached) a 3- to 8-membered ring optionally containing a further one or two heteroatoms, which ring optionally contains one to three unsaturations and is optionally substituted by one or more substituents selected from ═O and C₁₋₃ alkyl (optionally substituted by one or more fluoro atoms);
R^{j} represents, on each occasion when used herein, hydrogen, aryl, heteroaryl, C₃₋₆ cycloalkyl, heterocycloalkyl and/or C₁₋₄ alkyl, which latter five groups are optionally substituted with one or more substituents selected from halo, C₁₋₄ alkyl and —OR^{h};
R^{h} represents, on each occasion when used herein, hydrogen or C₁₋₄ alkyl optionally substituted by one or more halo atoms,
or a pharmaceutically acceptable ester, amide, solvate or salt thereof.

20. A combination product comprising:
(A) a compound of formula I, or a pharmaceutically-acceptable ester, amide, solvate or salt thereof; and
(B) another therapeutic agent that is useful in the treatment of in the treatment of cancer and/or a proliferative disease,
wherein each of components (A) and (B) is formulated in admixture with a pharmaceutically-acceptable adjuvant, diluent or carrier,
where the compound of formula I has the formula 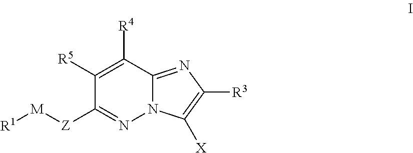
wherein:
Z represents a direct bond, —(CH₂)ₙ—O—, —(CH₂)ₙ—S—, —(CH₂)ₙ—N(R^{a})—, —(CH₂)ₙ—C(O)—, —(CH₂)ₙ—C(O)O—, —(CH₂)ₙ—S(O)—, —(CH₂)ₙ—SO₂—, —(CH₂)ₙ—N(R^{a})—SO₂—, —(CH₂)ₙ—SO₂—N(R^{a})—, —(CH₂)ₙ—N(R^{a})—CO—, —(CH₂)ₙ—NH—CO—NH— or —(CH₂)ₙ—CO—N(R^{a})—;
n represents, on each occasion when mentioned above, 0, 1 or 2
M represents a direct bond or C₁₋₈ alkylene optionally substituted by one or more substituents selected from halo, —OR^{b}, —SR^{b} and —N(R^{b})₂;
R¹ represents aryl or heteroaryl, both of which are optionally substituted by one or more substituents selected from B¹;
X represents C₃₋₆ cycloalkyl, heterocycloalkyl (which latter two groups are optionally substituted by one or more substituents selected from B⁴ and B⁵, respectively) or -G-R²;
G represents —(CH₂)ₘ—O—, —(CH₂)ₘ—S—, —(CH₂)ₘ—N(R^{d})—, —(CH₂)ₘ—C(O)—, —(CH₂)ₘ—C(O)O—, —(CH₂)ₘ—S(O)—, —(CH₂)ₘ—SO₂—, —(CH₂)ₘ—N(R^{d})—SO₂—, —(CH₂)ₘ—SO₂—N(R^{d})—, —(CH₂)ₘ—N(R^{d})—CO—, —(CH₂)ₘ—CO—N(R^{d})—, —(CH₂)ₘ—NH—CO—NH— or C₁₋₈ alkylene optionally substituted by one or more substituents selected from A¹;
m represents, on each occasion when used herein, 0, 1 or 2;
R² represents hydrogen, C₁₋₈ alkyl (optionally substituted by one or more substituents selected from A²) or -T-Q;
T represents a direct bond or a C₁₋₃ alkylene linker group optionally substituted by one or more substituents selected from A³;
Q represents C₃₋₆ cycloalkyl, heterocycloalkyl (which latter two groups are optionally substituted by one or more substituents selected from B⁶ and B⁷, respectively), aryl or heteroaryl (which latter two groups are optionally substituted by one or more substituents selected from B⁸ and B⁹, respectively);
A¹, A² and A³ independently represent halo, —OR^{e}, —S—C₁₋₄ alkyl, —N(R^{e})₂, —C(O)₂R^{e}, —C(O)N(R^{e})₂, —N(R^{e})—C(O)—R^{e}, —C(O)R^{e}, —CN, —SO₂N(R^{e})₂, phenyl (optionally substituted by one or more substituents selected from halo and —OR^{e}) and/or C₁₋₄ alkyl (optionally substituted by one or more substituents selected from halo);
B¹, B⁴, B⁵, B⁶, B⁷, B⁸ and B⁹ independently represent, on each occasion when used herein, halo, —OR^{e}, —C(O)₂R^{e}, —C(O)R^{e}, —C(O)N(R^{e})₂, —N(R^{e})—C(O)—R^{e}, —CN, —S(O)₂R^{e}, —S(O)₂N(R^{e})₂, —N(R^{e})₂ and/or C₁₋₄ alkyl (optionally substituted by one or more substituents selected from halo, —OR^{e} and —C(O)₂R^{e}); or,
B⁴, B⁵, B⁶ and B⁷ may alternatively and independently represent ═O;
R³, R⁴ and R⁵ independently represent hydrogen, halo, —R^{j}, —OR^{f}, —SR^{f}, cyano or —N(R^{f})₂;
R^{a}, R^{b}, R^{d}, R^{e} and R^{f} independently represent, on each occasion when used herein, hydrogen and/or C₁₋₄ alkyl optionally substituted with one or more substituents selected from halo and —OR^{h}; or
any two R^{e} groups, when attached to the same nitrogen atom may be linked together to form (together with the requisite nitrogen atom to which those R^{e} groups are necessarily attached) a 3- to 8-membered ring optionally containing a further one or two heteroatoms, which ring optionally contains one to three unsaturations and is optionally substituted by one or more substituents selected from ═O and C₁₋₃ alkyl (optionally substituted by one or more fluoro atoms);
R^{j} represents, on each occasion when used herein, hydrogen, aryl, heteroaryl, C₃₋₆ cycloalkyl, heterocycloalkyl and/or C₁₋₄ alkyl, which latter five groups are optionally substituted with one or more substituents selected from halo, C₁₋₄ alkyl and —OR^{h};
R^{h} represents, on each occasion when used herein, hydrogen or C₁₋₄ alkyl optionally substituted by one or more halo atoms.

21. A combination product as claimed in claim 20 which comprises a pharmaceutical formulation including a compound of formula I as defined in claim 20, or a pharmaceutically-acceptable ester, amide, solvate or salt thereof, another therapeutic agent that is useful in the treatment of cancer and/or a proliferative disease, and a pharmaceutically-acceptable adjuvant, diluent or carrier.

22. A combination product as claimed in claim 20 which comprises a kit of parts comprising components:
(a) a pharmaceutical formulation including a compound of formula I as defined in claim 20, or a pharmaceutically-acceptable ester, amide, solvate or salt thereof, in admixture with a pharmaceutically-acceptable adjuvant, diluent or carrier; and
(b) a pharmaceutical formulation including another therapeutic agent that is useful in the treatment of cancer and/or a proliferative disease in admixture with a pharmaceutically-acceptable adjuvant, diluent or carrier,
which components (a) and (b) are each provided in a form that is suitable for administration in conjunction with the other.

23. A process for the preparation of a compound of formula I as defined in claim 1, which process comprises:
(i) for compounds of formula I in which X represents C₃₋₆ cycloalkyl or heterocycloalkyl (both of which are optionally substituted as defined herein) or -G-R², reaction of a corresponding compound of formula II, 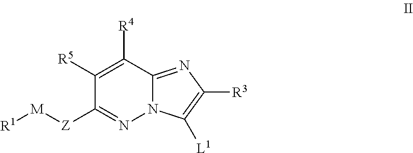
wherein L¹ represents a suitable leaving group, and Z, M, R¹, R³, R⁴ and R⁵ are as defined in claim 1, with a compound of formula III,
L²-X^{a} III
wherein L² represents a suitable group, and X^{a} represents C₃₋₆ cycloalkyl, heterocycloalkyl (which latter two groups are optionally substituted by one or more substituents selected from B⁴ and B⁵) or -G-R²;
(ii) for compounds of formula I in which X represents -G-R², G represents —(CH₂)ₘ—N(R^{d})— or —(CH₂)ₘ—O— and R² represents optionally substituted C₁₋₈ alkyl or -T-Q, reaction of a corresponding compound of formula I in which R² represents H, with a compound of formula IV,
R^{2x}-L¹ IV
wherein R^{2x} represents C₁₋₈ alkyl (optionally substituted by one or more substituents selected from A²) or -T-Q, and T and Q are as defined in claim 1 and L¹ is as defined above;
(iii) for compounds of formula I in which Z represents —(CH₂)ₙ—O—, —(CH₂)ₙ—S— or —(CH₂)ₙ—N(R^{a})— in which n represents 0, or, for compounds of formula I in which Z and M represent direct bonds and R¹ represents optionally substituted heteroaryl or heterocycloalkyl in which the point of attachment to the requisite 6,5-bicycle of formula I is via a heteroatom, reaction of a compound of formula V, 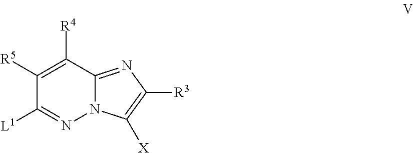
wherein X, R³, R⁴ and R⁵ are as defined in claim 1, and L¹ is as defined above, with a compound of formula VI,
H—Z^{a}-M-R¹ VI
wherein Z^{a} represents —O—, —S— or —N(R^{a})—, and R^{a}, R¹ and M are as defined in claim 1, or with a compound of formula VIA,
R^{1a}—H VIA
wherein R^{1a} represents a heteroaryl or heterocycloalkyl group, both of which are optionally substituted by one or more substituents selected from B¹, and in which the hydrogen atom depicted in the compound of formula VIA is attached to the heteroatom of the heteroaryl or heterocycloalkyl moiety, which heteroatom is to be attached to the requisite bicycle of the compound of formula I;
(iv) for compounds of formula I in which X represents -G-R², in which G represents —(CH₂)ₘ—N(R^{d})—SO₂—, —(CH₂)ₘ—N(R^{d})—CO— or —(CH₂)ₘ—NH—C(O)—NH—, reaction of a corresponding compound of formula I in which G represents —(CH₂)ₘ—N(R^{d})—, R² represents hydrogen and R^{d} is as hereinbefore defined (or, in the case of the formation of the urea compound, represents hydrogen), with either a compound of formula VII,
L¹-Q¹-R² VII
wherein Q¹ represents —S(O)₂—, —C(O)— or —C(O)NH—, L¹ is as defined above (or alternatively, in the case where Q¹ represents —C(O)—, L¹ may represent —O—C(O)—R²), and R² is as defined in claim 1; or, for the preparation of compounds of formula I in which X represents -G-R², and G represents —(CH₂)ₘ—NH—C(O)—NH—, with a compound of formula VIII,
O═C═N—R² VIII
wherein R² is as defined in claim 1;
(v) for compounds of formula I in which X represents -G-R², G represents —NH— and R² represents optionally substituted C₁₋₈ alkyl, reductive amination of a corresponding compound of formula I in which G represents —NH— and R² represents hydrogen, with a compound of formula IX,
R^{2b}—CHO IX
wherein R^{2b} represents C₁₋₇ alkyl optionally substituted by one or more substituents selected from A², and A² is as defined in claim 1;
(vi) compounds of formula I in which X represents -G-R² and G represents —CH₂—NH— may be prepared by a reductive amination of a compound of formula X, 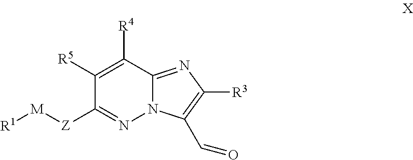
wherein Z, M, R¹, R³, R⁴ and R⁵ are as defined in claim 1, with a compound of formula XI,
R²—NH₂ XI
wherein R² is as defined in claim 1;
(vii) compounds of formula I in which X represents -G-R², G represents —CH₂—O— and R² represents hydrogen may be prepared by reduction of a corresponding compound of formula X as defined above;
(viii) compounds of formula I in which X represents -G-R², and G represents —(CH₂)ₘ—C(O)N(R^{d})— may be prepared by reaction of a compound corresponding to a compound of formula I but in which G represents —(CH₂)ₘ—C(O)O— (and R² represents optionally substituted C₁₋₈ alkyl or, preferably, hydrogen) with a compound of formula XII,
H(R^{d})N—R² XII
wherein R^{d} and R² are as defined in claim 1;
(ix) for compounds of formula I in which there is a —CH₂— group present, reduction of a corresponding compound of formula I in which there is a —CH(OH)— group present;
(x) for compounds of formula I in which X represents -G-R², G represents methylene substituted by —OH, and R² represents optionally substituted (i.e. by one or more A² substituents) C₁₋₈ alkyl or -T-Q, reaction of a compound of formula X as defined above with a compound of formula XII,
R^{2y}-M¹ XIII
wherein M¹ represents an appropriate alkali metal group, a —Mg-halide or a zinc-based group and R^{2y} represents C₁₋₈ alkyl (optionally substituted by one or more A² substituents) or -T-Q, and A², T and Q are as defined in claim 1;
(xi) compounds of formula I in which there is a —NH₂ group present may be prepared by the reduction of a corresponding compound of formula I in which there is a —NO₂ group present;
(xii) intramolecular cyclisation reaction of a compound of formula XIV, 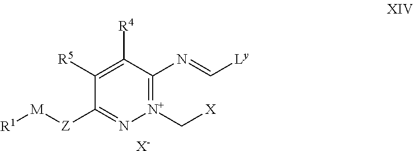
or a free base, or derivative thereof, wherein X⁻ represents an acid counterion, L^{y} represents an appropriate leaving group, and Z, M, R¹, R⁴, R⁵ and X are as defined in claim 1;
(xiii) for compounds of formula I in which there is a carboxylic acid group present, hydrolysis of a corresponding compound of formula I in which there is a corresponding ester group present;
(xiv) for compounds of formula I in which there is a hydroxy group present on an aromatic ring, methyl ether cleavage of a corresponding compound of formula I in which there is a methoxy group present on such an aromatic ring;
(xv) for compounds of formula I in which there is a —CH₂—NH₂ group present, reduction of a compound of formula I in which there is a corresponding cyano group;
(xvi) for compounds of formula I in which X represents -G-R², G represents —(CH₂)ₘ—N(R^{d})—C(O)—, and R² is other than hydrogen, reaction of a compound of formula I in which X represents —(CH₂)ₘ—N(R^{d})H, with a compound of formula XIVA,
R^{2a}—C(O)OH XIVA
wherein R^{2a} represents R², provided that it does not represent hydrogen;
(xvii) for compounds of formula I in which there is a —CH₂OH group present, reduction of a compound of formula I in which there is a corresponding —C(O)OR² group present;
(xviii) for compounds of formula I in which there is a —CH₂— moiety attached to a heteroaryl or heterocycloalkyl moiety via a heteroatom, such as a nitrogen heteroatom, reaction of a compound of formula I in which there is a corresponding —CH₂—OH moiety present with a compound of formula VIA as defined above;
(xix) for compounds of formula I in which X represents —C(O)OR², reaction of a compound of formula XIX, 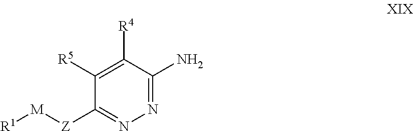
wherein R¹, R⁴, R⁵, Z and M are as defined in claim 1, with a compound of formula XIVB,
R³—C(═O)—C(L¹)(H)—C(O)OR² XIVB
wherein R² and R³ are as defined in claim 1 and L¹ is as defined above;
(xx) reaction of a corresponding compound of formula XVII, 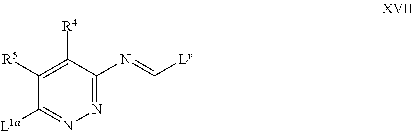
in which L^{1a} represents —Z-M-R¹ and wherein Z, M, R¹, R⁴ and R⁵ are as defined in claim 1, and L^{y} is as defined above, with a compound of formula XVIII,
L¹-CH₂—X XVIII
wherein X is as defined in claim 1 and L¹ is as defined above;
(xxi) for compounds of formula I in which X represents -G-R², and G represents —S(O)₂N(R^{d})—, reaction of a compound of formula XIVC, 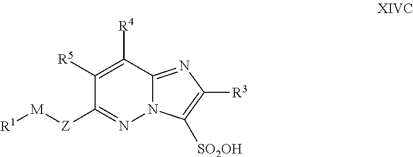
wherein R¹, R³, R⁴, R⁵, Z and M are as defined in claim 1, with a compound of formula XII as defined above.

24. A process for the preparation of a pharmaceutical formulation as defined in claim 14, which process comprises bringing into association a compound of formula I, or a pharmaceutically acceptable ester, amide, solvate or salt thereof with a pharmaceutically-acceptable adjuvant, diluent or carrier, where the compound of formula I has the formula 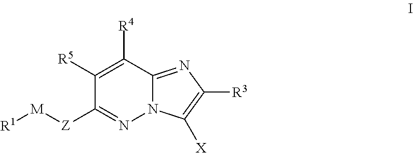
wherein:
Z represents a direct bond, —(CH₂)ₙ—O—, —(CH₂)ₙ—S—, —(CH₂)ₙ—N(R^{a})—, —(CH₂)ₙ—C(O)—, —(CH₂)ₙ—C(O)O—, —(CH₂)ₙ—S(O)—, —(CH₂)ₙ—SO₂—, —(CH₂)ₙ—N(R^{a})—SO₂—, —(CH₂)ₙ—SO₂—N(R^{a})—, —(CH₂)ₙ—N(R^{a})—CO—, —(CH₂)ₙ—NH—CO—NH— or —(CH₂)ₙ—CO—N(R^{a})—;
n represents, on each occasion when mentioned above, 0, 1 or 2
M represents a direct bond or C₁₋₈ alkylene optionally substituted by one or more substituents selected from halo, —OR^{b}, —SR^{b} and —N(R^{b})₂;
R¹ represents aryl or heteroaryl, both of which are optionally substituted by one or more substituents selected from B¹;
X represents C₃₋₆ cycloalkyl, heterocycloalkyl (which latter two groups are optionally substituted by one or more substituents selected from B⁴ and B⁵, respectively) or -G-R²;
G represents —(CH₂)ₘ—O—, —(CH₂)ₘ—S—, —(CH₂)ₘ—N(R^{d})—, —(CH₂)ₘ—C(O)—, —(CH₂)ₘ—C(O)O—, —(CH₂)ₘ—S(O)—, —(CH₂)ₘ—SO₂—, —(CH₂)ₘ—N(R^{d})—SO₂—, —(CH₂)ₘ—SO₂—N(R^{d})—, —(CH₂)ₘ—N(R^{d})—CO—, —(CH₂)ₘ—CO—N(R^{d})—, —(CH₂)ₘ—NH—CO—NH— or C₁₋₈ alkylene optionally substituted by one or more substituents selected from A¹;
m represents, on each occasion when used herein, 0, 1 or 2;
R² represents hydrogen, C₁₋₈ alkyl (optionally substituted by one or more substituents selected from A²) or -T-Q;
T represents a direct bond or a C₁₋₃ alkylene linker group optionally substituted by one or more substituents selected from A³;
Q represents C₃₋₆ cycloalkyl, heterocycloalkyl (which latter two groups are optionally substituted by one or more substituents selected from B⁶ and B⁷, respectively), aryl or heteroaryl (which latter two groups are optionally substituted by one or more substituents selected from B⁸ and B⁹, respectively);
A¹, A² and A³ independently represent halo, —OR^{e}, —S—C₁₋₄ alkyl, —N(R^{e})₂, —C(O)₂R^{e}, —C(O)N(R^{e})₂, —N(R^{e})—C(O)—R^{e}, —C(O)R^{e}, —CN, —SO₂N(R^{e})₂, phenyl (optionally substituted by one or more substituents selected from halo and —OR^{e}) and/or C₁₋₄ alkyl (optionally substituted by one or more substituents selected from halo);
B¹, B⁴, B⁵, B⁶, B⁷, B⁸ and B⁹ independently represent, on each occasion when used herein, halo, —OR^{e}, —C(O)₂R^{e}, —C(O)R^{e}, —C(O)N(R^{e})₂, —N(R^{e})—C(O)—R^{e}, —CN, —S(O)₂R^{e}, —S(O)₂N(R^{e})₂, —N(R^{e})₂ and/or C₁₋₄ alkyl (optionally substituted by one or more substituents selected from halo, —OR^{e} and —C(O)₂R^{e}); or,
B⁴, B⁵, B⁶ and B⁷ may alternatively and independently represent ═O;
R³, R⁴ and R⁵ independently represent hydrogen, halo, —R^{j}, —OR^{f}, —SR^{f}, cyano or —N(R^{f})₂;
R^{a}, R^{b}, R^{d}, R^{e} and R^{f} independently represent, on each occasion when used herein, hydrogen and/or C₁₋₄ alkyl optionally substituted with one or more substituents selected from halo and —OR^{h}; or
any two R^{e} groups, when attached to the same nitrogen atom may be linked together to form (together with the requisite nitrogen atom to which those R^{e} groups are necessarily attached) a 3- to 8-membered ring optionally containing a further one or two heteroatoms, which ring optionally contains one to three unsaturations and is optionally substituted by one or more substituents selected from ═O and C₁₋₃ alkyl (optionally substituted by one or more fluoro atoms);
R^{j} represents, on each occasion when used herein, hydrogen, aryl, heteroaryl, C₃₋₆ cycloalkyl, heterocycloalkyl and/or C₁₋₄ alkyl, which latter five groups are optionally substituted with one or more substituents selected from halo, C₁₋₄ alkyl and —OR^{h};
R^{h} represents, on each occasion when used herein, hydrogen or C₁₋₄ alkyl optionally substituted by one or more halo atoms,
provided that when:
(I) R⁴ and R⁵ represent hydrogen, Z represents —S—, R¹ represents unsubstituted phenyl, X represents -G-R²:
(i) M represents a direct bond, then when:
(A) R³ represents hydrogen and G represents —C(O)—;
(B) R³ represents —CH₃ and G represents —(CH₂)—NH—C(O)—,
then R² does not represent unsubstituted phenyl;
(ii) M represents —CH₂—, R³ represents tert-butyl and G represents —O—, then R² does not represent —CH₃;
(III) R⁴ and R⁵ represent hydrogen, Z represents —O—, R³ represents tert-butyl, X represents -G-R², G represents —O—, and R² represents —CH₃, then:
(i) when M represents a direct bond, then R¹ does not represent 2-methoxyphenyl;
(ii) when M represents —CH₂—, then R¹ does not represent unsubstituted phenyl;
(IV) R⁴ and R⁵ represent hydrogen, R³ represents —CF₃, X represents -G-R², G represents —CH₂—:
(a) R² represents (4-n-propyl)pyrrolidin-2-one and M represents —CH₂—, then:
(i) when Z represents —N(H)—, R¹ does not represent (2,4-dimethoxy)phenyl;
(ii) when Z represents —O—, R¹ does not represent unsubstituted phenyl;
(b) R² represents (4-CH═CF₂)pyrrolidin-2-one (i.e. 4-(2,2-difluoroethenyl)pyrrolidin-2-one), and M and Z both represent direct bonds, then R¹ does not represent unsubstituted 3-pyridyl, 3-thienyl or phenyl;
(V) Z and M represent direct bonds, R³ and R⁵ represent hydrogen, X represents -G-R², G represents —CH₂— and R² represents 4-morpholinyl, then:
(i) when R⁴ represents methyl, then R¹ does not represent 3-methoxyphenyl or unsubstituted phenyl; or
(ii) when R⁴ represents hydrogen, then R¹ does not represent 4-chlorophenyl or unsubstituted phenyl;
(VI) Z and M represent direct bonds, R³ and R⁵ represent hydrogen, X represents -G-R², G represents —CH₂— substituted by A¹ in which A¹ represents —N(CH₃)₂, and R² represents hydrogen (so forming a —CH₂—N(CH₃)₂ group), then R¹ does not represent unsubstituted phenyl when R⁴ represents hydrogen or methyl;
(VII) Z and M represent direct bonds, R³ and R⁵ represent hydrogen, R⁴ represents —CF₃, R¹ represents 4-trifluoromethylphenyl, X represents -G-R², G represents —C≡C— (i.e. ethynylene), then R² does not represent 2-(NH₂)-pyrimidin-5-yl, 5-S(O)₂NH₂)-thien-2-yl or 6-(NH₂)-pyrid-3-yl; and
(IX) Z and M represent direct bonds, R³, R⁴ and R⁵ represent hydrogen, X represents -G-R², G represents —CH₂—, then when:
(i) R¹ represents 3-trifluoromethylphenyl, then R² does not represent H, or ethyl;
(ii) R¹ represents unsubstituted 3-pyridyl, then R² does not represent H or methyl.

25. A process for the preparation of a combination product according to claim 20, which process comprises bringing into association a compound of formula I, or a pharmaceutically acceptable ester, amide, solvate or salt thereof with the other therapeutic agent that is useful in the treatment of cancer and/or a proliferative disease, and at least one pharmaceutically-acceptable adjuvant, diluent or carrier,
where the compound of formula I has the formula 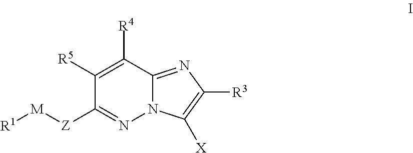
wherein:
Z represents a direct bond, —(CH₂)ₙ—O—, —(CH₂)ₙ—S—, —(CH₂)ₙ—N(R^{a})—, —(CH₂)ₙ—C(O)—, —(CH₂)ₙ—C(O)O—, —(CH₂)ₙ—S(O)—, —(CH₂)ₙ—SO₂—, —(CH₂)ₙ—N(R^{a})—SO₂—, —(CH₂)ₙ—SO₂—N(R^{a})—, —(CH₂)ₙ—N(R^{a})—CO—, —(CH₂)ₙ—NH—CO—NH— or —(CH₂)ₙ—CO—N(R^{a})—;
n represents, on each occasion when mentioned above, 0, 1 or 2
M represents a direct bond or C₁₋₈ alkylene optionally substituted by one or more substituents selected from halo, —OR^{b}—SR^{b} and —N(R^{b})₂;
R¹ represents aryl or heteroaryl, both of which are optionally substituted by one or more substituents selected from B¹;
X represents C₃₋₆ cycloalkyl, heterocycloalkyl (which latter two groups are optionally substituted by one or more substituents selected from B⁴ and B⁵, respectively) or -G-R²;
G represents —(CH₂)ₘ—O—, —(CH₂)ₘ—S—, —(CH₂)ₘ—N(R^{d})—, —(CH₂)ₘ—C(O)—, —(CH₂)ₘ—C(O)O—, —(CH₂)ₘ—S(O)—, —(CH₂)ₘ—SO₂—, —(CH₂)ₘ—N(R^{d})—SO₂—, —(CH₂)ₘ—SO₂—N(R^{d})—, —(CH₂)ₘ—N(R^{d})—CO—, —(CH₂)ₘ—CO—N(R^{d})—, —(CH₂)ₘ—NH—CO—NH— or C₁₋₈ alkylene optionally substituted by one or more substituents selected from A¹;
m represents, on each occasion when used herein, 0, 1 or 2;
R² represents hydrogen, C₁₋₈ alkyl (optionally substituted by one or more substituents selected from A²) or -T-Q;
T represents a direct bond or a C₁₋₃ alkylene linker group optionally substituted by one or more substituents selected from A³;
Q represents C₃₋₆ cycloalkyl, heterocycloalkyl (which latter two groups are optionally substituted by one or more substituents selected from B⁶ and B⁷, respectively), aryl or heteroaryl (which latter two groups are optionally substituted by one or more substituents selected from B⁸ and B⁹, respectively);
A¹, A² and A³ independently represent halo, —OR^{e}, —S—C₁₋₄ alkyl, —N(R^{e})₂, —C(O)₂R^{e}, —C(O)N(R^{e})₂, —N(R^{e})—C(O)—R^{e}, —C(O)R^{e}, —CN, —SO₂N(R^{e})₂, phenyl (optionally substituted by one or more substituents selected from halo and —OR^{e}) and/or C₁₋₄ alkyl (optionally substituted by one or more substituents selected from halo);
B¹, B⁴, B⁵, B⁶, B⁷, B⁸ and B⁹ independently represent, on each occasion when used herein, halo, —OR^{e}, —C(O)₂R^{e}, —C(O)R^{e}, —C(O)N(R^{e})₂, —N(R^{e})—C(O)—R^{e}, —CN, —S(O)₂R^{e}, —S(O)₂N(R^{e})₂, —N(R^{e})₂ and/or C₁₋₄ alkyl (optionally substituted by one or more substituents selected from halo, —OR^{e} and —C(O)₂R^{e}); or,
B⁴, B⁵, B⁶ and B⁷ may alternatively and independently represent ═O;
R³, R⁴ and R⁵ independently represent hydrogen, halo, —R^{j}, —OR^{f}, —SR^{f}, cyano or —N(R^{f})₂;
R^{a}, R^{b}, R^{d}, R^{e} and R^{f} independently represent, on each occasion when used herein, hydrogen and/or C₁₋₄ alkyl optionally substituted with one or more substituents selected from halo and —OR^{h}; or
any two R^{e} groups, when attached to the same nitrogen atom may be linked together to form (together with the requisite nitrogen atom to which those R^{e} groups are necessarily attached) a 3- to 8-membered ring optionally containing a further one or two heteroatoms, which ring optionally contains one to three unsaturations and is optionally substituted by one or more substituents selected from ═O and C₁₋₃ alkyl (optionally substituted by one or more fluoro atoms);
R^{j} represents, on each occasion when used herein, hydrogen, aryl, heteroaryl, C₃₋₆ cycloalkyl, heterocycloalkyl and/or C₁₋₄ alkyl, which latter five groups are optionally substituted with one or more substituents selected from halo, C₁₋₄ alkyl and —OR^{h};
R^{h} represents, on each occasion when used herein, hydrogen or C₁₋₄ alkyl optionally substituted by one or more halo atoms.
